(19) United States
(12) Patent Application Publication
(10) Pub. No.: US 2010/0311729 A1
(43) Pub. Date: Dec. 9, 2010

(54) Substituted imidazopyridazines and pyrrolopyrimidines as lipid kinase inhibitors

(76) Inventors: Hans-Georg Capraro, Basel (CH); Patricia Imbach, Basel (CH); Giorgio Caravatti, Basel (CH); Pascal Furet, Basel (CH); Jiong Lan, Emeryville, CA (US); Sabrina Pecchi, Emeryville, CA (US); Joseph Schoepfer, Basel (CH)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/599,143

(22) PCT Filed: May 9, 2008
(86) PCT No.: PCT/EP08/55751

§ 371 Date: Nov. 6, 2009

Related U.S. Application Data
(60) Provisional application No. 60/917,348, filed on May 11, 2007.

(51) Int. Cl. A61K 31/55; A61K 31/541; A61K 31/5377; A61K 31/5025; C07D 487/04

(52) U.S. Cl. 514/217.05; 514/228.5; 514/233.2; 514/248; 540/599; 544/61; 544/117; 544/238

(57) ABSTRACT

The present invention relates to compounds are of the formula I,

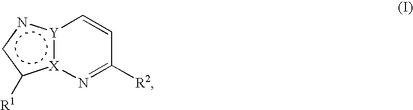

processes for the preparation thereof, more generally these compounds for use in the treatment of the human or animal body, in the treatment of an inflammatory or obstructive airway disease, disorders commonly occurring in connection with transplantation, or a proliferative disease, which disease responds to an inhibition of kinases of the PI3-kinase-related protein kinase family.

The invention relates to novel 3,6-disubstituted-imidazo[1,2-b]pyridazines and 3,5-disubstituted pyrazolo[1,5-a]pyrimidines, processes for the preparation thereof, more generally such compounds for use in the treatment of the human or animal body, yet more generally the use of such compounds or such compounds for use—alone or in combination with one or more other pharmaceutically active compounds—in the treatment (this term including prophylactic and/or therapeutic treatment) of an inflammatory or obstructive airway disease, such as asthma, disorders commonly occurring in connection with transplantation, or especially a proliferative disease, more especially a tumor disease, which may be solid and/or liquid, especially one or more of the mentioned diseases which respond to an inhibition of kinases of the PI3-kinase-related protein kinase family, especially lipid kinases and/or PI3 kinase (PI3K) and/or mTOR and/or DNA protein kinase and/or ATM and/or ATR and/or hSMG-1 activity; a method for the treatment of such a disease in animals, especially a human, comprising administering such a compound, alone or in combination, to a warm-blooded animal in need thereof and the use of such a compound—alone or in combination with one or more other pharmaceutically active compounds—for the manufacture of a pharmaceutical preparation for the treatment of said diseases in animals, especially a human. The invention also relates to pharmaceutical compositions comprising such compounds, especially for use in the treatment of a disorder or disease as described above or below.

In a first preferred aspect, the invention relates to a method of use or the USE of one or more compounds of the formula I,

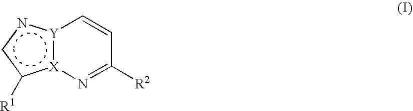

wherein

either X is N and Y is C, or X is C and Y is N,

the broken circle represents two conjugated double bonds within the five-membered ring with the proviso that the first of said bonds starts from either X═C or Y═C;

and each of R¹ and R² is, independently of the other, unsubstituted or substituted aryl or unsubstituted or substituted heterocyclyl;

and/or an N-oxide thereof, a solvate and/or a (preferably pharmaceutically acceptable) salt thereof,

in the treatment of one or more diseases or disorders where the disease(s) or disorder(s) respond or responds (especially in a beneficial way, e.g. by partial or complete removal of one or more of its symptoms up to complete cure or remission) to an inhibition of one or more kinases of the PI3-kinase-related protein kinase family, most especially PI3 kinase (PI3K), especially where the kinase shows (in the context of other regulatory mechanisms) inadequately high or more preferably higher than normal (e.g. constitutive) activity; or to a pharmaceutical composition for use in the treatment of said disorder or disease, comprising said compound(s);

where in said method the treatment comprises administering a compound of the formula I, and/or an N-oxide thereof, a solvate and or a pharmaceutically acceptable salt thereof, to a warm-blooded animal, especially a human, in need of such treatment, preferably in an effective amount for the treatment of said disease(s) or disorder(s).

The general terms used hereinbefore and hereinafter preferably have within the context of this disclosure the following meanings, unless otherwise indicated, where more general terms whereever used may, independently of each other, be replaced by more specific definitions or remain, thus defining more preferred embodiments of the invention:

The prefix “lower” or “C₁-C₇—” denotes a radical having up to and including a maximum of 7, especially up to and including a maximum of 4 carbon atoms, the radicals in question being either linear or branched with single or multiple branching.

Lower alkyl (or C₁-C₇-alkyl) is preferably alkyl with from and including 1 up to and including 7, preferably from and including 1 to and including 4, and is linear or branched; preferably, lower alkyl is butyl, such as n-butyl, sec-butyl, isobutyl, tert-butyl, propyl, such as n-propyl or isopropyl, ethyl or preferably methyl.

For compounds of the formula I wherein X is N and Y is C (imidazo[1,2-b]pyridazines), the position of the conjugated double bonds and the numbering used in the examples are as in the following formula IA:

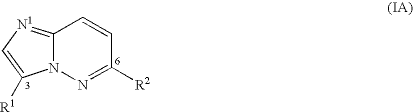

For compounds of the formula I wherein X is C and Y is N (pyrazolo[1,5-a]pyrimidines), the position of the conjugated double bonds and the numbering used in the examples are as in the following formula IB:

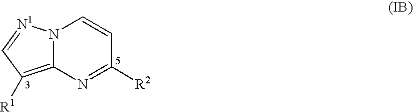

Halogen, halogeno (or halo) is especially fluoro, chloro, bromo, or iodo, especially fluoro, chloro or bromo.

In unsubstituted or substituted heterocyclyl (also in unsubstituted or substituted heterocyclyl-carbonyl (heterocyclyl-C(═O)—)), heterocyclyl is preferably a heterocyclic radical that is unsaturated (=carrying the largest possible number of conjugated double bonds in the ring(s), then heterocyclyl being heteroaryl; heteroaryl is preferably a moiety marked below in this paragraph by an asterisk *), saturated or partially saturated and is preferably a monocyclic or in a broader aspect of the invention bicyclic or tricyclic ring; and has 3 to 24, more preferably 4 to 16, most preferably 4 to 10 and most preferably 5 or 6 ring atoms; wherein one or more, preferably one to four, especially one or two carbon ring atoms are replaced by a heteroatom selected from the group consisting of nitrogen, oxygen and sulfur, the bonding ring preferably having 4 to 12, especially 5 to 7 ring atoms; which heterocyclic radical (heterocyclyl) is unsubstituted or substituted (at one or more N and/or C ring atoms) by one or more, especially 1 to 3, substituents independently selected from the group consisting of the substituents defined below for substituted aryl; and where heterocyclyl is especially a heterocyclyl radical selected from the group consisting of oxiranyl, azirinyl*, aziridinyl, 1,2-oxathiolanyl, *thienyl (=thiophenyl), *furanyl, tetrahydrofuryl, *pyranyl, *thiopyranyl, *thianthrenyl, *isobenzofuranyl, *benzofuranyl, *chromenyl, *2H-pyrrolyl, pyrrolyl, pyrrolinyl, pyrrolidinyl, imidazolyl, imidazolidinyl, *benzimidazolyl, *pyrazolyl, *pyrazinyl, pyrazolidinyl, thiazolyl, *isothiazolyl, *dithiazolyl, *oxazolyl, *isoxazolyl, *pyridinyl, *pyrazinyl, *pyrimidinyl, piperidinyl, piperazinyl, *pyridazinyl, morpholinyl, thiomorpholinyl, (S-oxo or S,S-dioxo)-thiomorpholinyl, *furazanyl, *indolizinyl, azepanyl, diazepanyl, especially 1,4-diazepanyl, *isoindolyl, *3H-indolyl, *indolyl, *benzimidazolyl, *indazolyl, *triazolyl, letrazolyl, *purinyl, *4H-quinolizinyl, *isoquinolyl, *quinolyl, tetrahydroquinolyl, tetrahydroisoquinolyl, decahydroquinolyl, octahydroisoquinolyl, *benzofuranyl, *dibenzofuranyl, *benzothiophenyl, *dibenzothiophenyl, *phthalazinyl, *naphthyridinyl, *pyrrolo-pyrimidinyl, especially pyrrolo[2,3-d]pyrimidin-(e.g.1-) yl, 1H,4H,5H-trihydropyrazolo[2,3-c]piperidin-1-yl, *pyrrolo-pyridinyl, e.g. *pyrrolo[2,3-c]pyridine-1-yl (meaning 5-aza-indol-1-yl) or preferably *pyrrolo[2,3-b]pyridinyl, especially 1H-pyrrolo[2,3-b]pyridine-5-yl, *quinoxalyl, *quinazolinyl, *cinnolinyl, *pteridinyl, *carbazolyl, *beta-carbolinyl, *phenanthridinyl, *acridinyl, *perimidinyl, *phenanthrolinyl, *phenazinyl, *phenothiazinyl, *phenoxazinyl, isochromanyl, chromanyl, benzo[1,3]dioxol-5-yl and 2,3-dihydro-benzo[1,4]dioxin-6-yl, each of these radicals being unsubstituted or substituted by one or more, preferably up to three, substituents independently selected from those mentioned below for substituted aryl and from oxo, especially from the group consisting of C₁-C₇-alkyl that is unsubstituted or substituted by hydroxyl, by C₁-C₇-alkoxy, by halo, e.g. in trifluoromethyl, or by cyano-C₁-C₇-alkyl, e.g. C₁-C₇-alkyl, such as methyl, hydroxy-C₁-C₇-alkyl, such as hydroxymethyl, or C₁-C₇-alkoxy-C₁-C₇-alkyl, such as methoxymethyl, or halo-C₁-C₇-alkyl, such as trifluoromethyl, from amino- or C₁-C₇-alkylamino-C₁-C₇-alkyl, halo, hydroxyl, (especially C₁-C₇-)alkoxy, hydroxyl-C₂-C₇-alkoxy, such as 2-hydroxyethoxy, amino-C₂-C₇-alkoxy, such as 2-aminoethoxy or 3-aminopropoxy, C₁-C₇-alkoxycarbonylamino-C₁-C₇-alkoxy, such as 2-(tert-butoxycarbonylamino)-ethoxy or 3-(tert-butoxycarbonylamino)-propoxy, carboxy-C₁-C₇-alkoxy, C₁-C₇-alkoxycarbonyl-C₁-C₇-alkoxy, such as methoxycarbonyl-methoxy; heterocyclyloxy (especially pyrrolyloxy, furanyloxy, thiophenyloxy, imidazolyloxy, pyrazolyloxy, thiazolyloxy, pyrazolidinyloxy, pyrrolidinyloxy, pyridinyloxy, piperidinyloxy, oxopiperidinyloxy, piperazinyloxy, triazolyloxy, morpholinyloxy, thiomorpholinyloxy, S-oxothiomorpholinyloxy, benzimidazolyloxy, pyrrolo-pyrimidinyloxy, or 1H,4H,5H-trihydropyrazolo[2,3-c]piperidin-1-yloxy (meaning 5-aza-3,4,5,6-tetrahydroindazol-1-yloxy)) bound to the “oxy” via a ring carbon and that is unsubstituted or substituted by one or more, especially up to three, substituents independently selected from C₁-C₇-alkyl, such as isopropyl, halo-C₁-C₇-alkyl, phenyl, halophenyl, hydroxy, C₁-C₇-alkoxy, halo, C₁-C₇-alkoxycarbonyl, carbamoyl, phenylsulfonyl wherein phenyl is unsubstituted or substituted by one or more, preferably up to three, substituents independently selected from C₁-C₇-alkyl, hydroxy, C₁-C₇-alkoxy, halo, nitro and cyano, heterocyclylcarbonyl (=heterocyclyl-C(═O)—) where heterocyclyl is bound via a ring nitrogen to the carbonyl, especially piperidinocarbonyl, morpholino-carbonyl, thiomorpholino-carbonyl or S-oxo- or S,S-dioxothiomorpholinocarbonyl, C₁-C₇-alkanoyl, unsubstituted or substituted benzoyl wherein the substituents are preferably one or more, e.g. up to three, substituents independently selected from the group consisting of hydroxy, C₁-C₇-alkoxy and cyano, C₁-C₇-alkanesulfonyl, unsubstituted or substituted benzenesulfonyl wherein the substituents are preferably one or more, e.g. up to three, substituents independently selected from the group consisting of hydroxy, C₁-C₇-alkoxy and cyano, sulfamoyl, N-mono- or N,N-disubstituted sulfamoyl, preferably N-mono- or N,N-di-(C₁-C₇-alkyl)-sulfamoyl, cyano and nitro, especially N-isopropyl-piperidinyloxy; oxo, amino, mono- or di-(C₁-C₇-alkyl, hydroxyl-C₁-C₇-alkyl, phenyl-C₁-C₇-alkyl and/or C₃-C₈-cyloalkyl)-amino, C₁-C₇-alkanoylamino, C₁-C₇-alkoxycarbonyl-amino, benzoylamino, am inobenzoylamino, C₁-C₇-alkoxycarbonylamino, (phenyl or naphthyl)-C₁-C₇-alkoxycarbonylamino; heterocyclylamino (especially pyrrolylamino, furanylamino, thiophenylamino, imidazolylamino, pyrazolylamino, thiazolylamino, pyrazolidinylamino, pyrrolidinylamino, pyridinylamino, piperidinylamino, oxopiperidinylamino, piperazinylamino, triazolylamino, morpholinylamino, thiomorpholinylamino, S-oxothiomorpholinylamino, benzimidazolylamino, pyrrolo-pyrimidinylamino, or 1H,4H,5H-trihydropyrazolo[2,3-c]piperidin-1-ylamino (meaning 5-aza-3,4,5,6-tetrahydroindazol-1-ylamino)) bound via a ring carbon to the “amino”and that is unsubstituted or substituted by one or more, especially up to three, substituents independently selected from C₁-C₇-alkyl, such as isopropyl, halo-C₁-C₇-alkyl, phenyl, halophenyl, hydroxy, C₁-C₇-alkoxy, halo, C₁-C₇-alkoxycarbonyl, carbamoyl, phenylsulfonyl wherein phenyl is unsubstituted or substituted by one or more, preferably up to three, substituents independently selected from C₁-C₇-alkyl, hydroxy, C₁-₇-alkoxy, halo, nitro and cyano, heterocyclylcarbonyl (=heterocyclyl-C(═O)—) where heterocyclyl is bound via a ring nitrogen to the carbonyl, especially piperidinocarbonyl, morpholino-carbonyl, thiomorpholino-carbonyl or S-oxo- or S,S-dioxothiomorpholinocarbonyl, C₁-C₇-alkanoyl, unsubstituted or substituted benzoyl wherein the substituents are preferably one or more, e.g. up to three, substituents independently selected from the group consisting of hydroxy, C₁-C₇-alkoxy and cyano, C₁-C₇-alkanesulfonyl, unsubstituted or substituted benzenesulfonyl wherein the substituents are preferably one or more, e.g. up to three, substituents independently selected from the group consisting of hydroxy, C₁-C₇-alkoxy and cyano, sulfamoyl, N-mono- or N,N-disubstituted sulfamoyl, preferably N-mono- or N,N-di-(C₁-C₇-alkyl)-sulfamoyl, cyano and nitro, such as 4-(phenyl)thiazol-2-yl-amino; C₁-C₇-alkanoyl, such as acetyl, carboxy, C₁-C₇-alkoxycarbonyl, such as ethoxycarbonyl, carbamoyl, N-mono or N,N-disubstituted carbamoyl, especially N-mono- or N,N-di-(C₁-C₇-alkyl, phenyl-C₁-C₇-alkyl and/or C₃-C₈-cycloalkyl)-aminocarbonyl, [heterocyclyl (especially pyrazolyl, such as pyrazolo, pyrrolidinyl, such as pyrrolidin-1-yl, pyridinyl, such as pyridin-(2-, 3- or 4-)yl, piperidinyl, such as piperidin-1-yl, oxopiperidinyl, such as 2-oxopiperidin-1-yl, piperazinyl, such as piperazin-1-yl, triazolyl, such as 1,2,4-triazol-1-yl, thiazolyl, morpholinyl, such as morpholino, thiomorpholinyl, such as thiomorpholino, S-oxothiomorpholinyl, such as S-oxothiomorpholino, benzimidazol(especially -1-)yl, pyrrolo-pyrimidinyl, especially pyrrolo[2,3-d]pyrimidin-(e.g. 1-)yl, or 1H,4H,5H-trihydropyrazolo[2,3-c]piperidin-1-yl) {wherein heterocyclyl is unsubstituted or substituted by one or more, especially up to three, substituents independently selected from C₁-C₇-alkyl, halo-C₁-C₇-alkyl, halophenyl, hydroxy, C₁-C₇-alkoxy, halo, C₁-C₇-alkoxycarbonyl, carbamoyl, phenylsulfonyl wherein phenyl is unsubstituted or substituted by one or more, preferably up to three, substituents independently selected from C₁-C_{T}-alkyl, hydroxy, C₁-C₇-alkoxy, halo, nitro and cyano, heterocyclylcarbonyl (=heterocyclyl-C(═O)—) where heterocyclyl is bound via a ring nitrogen to the carbonyl, especially piperidinocarbonyl, morpholino-carbonyl, thiomorpholino-carbonyl or S-oxo- or S,S-dioxothiomorpholinocarbonyl, C₁-C₇-alkanesulfonyl, such as methanesulfonyl, sulfamoyl, N-mono- or N,N-disubstituted sulfamoyl, preferably N-mono- or N,N-di-(C₁-C₇-alkyl)-sulfamoyl, cyano and nitro}]-aminocarbonyl, phenyl-aminocarbonyl, N-[N′-mono- or N′,N′-di-(C₁-C₇alkyl)-amino-C₁-C₇-alkyl]-aminocarbonyl, mono- or di-[C₁-C₇-alkoxy, pyrrolidino, piperidino, piperazino, thiazolyl (e.g. thiazol-5-yl), hydroxyl-C₁-C₇-alkylamino and/or N′-mono- or N′,N′-di-(C₁-C₇-alkyl)-amino]substituted phenyl-aminocarbonyl, cyano, nitro and heterocyclyl (especially pyrrolyl, furanyl, thiophenyl, imidazolyl, pyrazolyl, thiazolyl, pyrazolidinyl, pyrrolidinyl, pyridinyl, piperidinyl, oxopiperidinyl, piperazinyl, triazolyl, morpholinyl, thiomorpholinyl, S-oxothiomorpholinyl, benzimidazolyl, pyrrolo-pyrimidinyl, or 1H,4H,5H-trihydropyrazolo[2,3-c]piperidin-1-yl (meaning 5-aza-3,4,5,6-tetrahydroindazol-1-yl)) bound via a ring nitrogen atom (preferably in the case of saturated heterocyclyl) or preferably a ring carbon and that is unsubstituted or substituted by one or more, especially up to three, substituents independently selected from C₁-C₇-alkyl, such as isopropyl, halo-C₁-C₇-alkyl, phenyl, halophenyl, hydroxy, C₁-C₇-alkoxy, halo, C₁-C₇-alkoxy-carbonyl, carbamoyl, phenylsulfonyl wherein phenyl is unsubstituted or substituted by one or more, preferably up to three, substituents independently selected from C₁-C₇-alkyl, hydroxy, C₁-C₇-alkoxy, halo, nitro and cyano, heterocyclylcarbonyl (=heterocyclyl-C(═O)—) where heterocyclyl is bound via a ring nitrogen to the carbonyl, especially piperidinocarbonyl, morpholino-carbonyl, thiomorpholino-carbonyl or S-oxo- or S,S-dioxothiomorpholinocarbonyl, C₁-C₇-alkanoyl, unsubstituted or substituted benzoyl wherein the substituents are preferably one or more, e.g. up to three, substituents independently selected from the group consisting of hydroxy, C₁-C₇-alkoxy and cyano, C₁-C₇-alkanesulfonyl, unsubstituted or substituted benzenesulfonyl wherein the substituents are preferably one or more, e.g. up to three, substituents independently selected from the group consisting of hydroxy, C₁-C₇-alkoxy and cyano, sulfamoyl, N-mono- or N,N-disubstituted sulfamoyl, preferably N-mono- or N,N-di-(C₁-C₇-alkyl)-sulfamoyl, cyano and nitro. Preferably, unsubstituted or substituted heterocyclyl is bound via a ring carbon to the rest of the molecule of the formula I, especially IA or IB, in any of the embodiments of the present invention, and if both R¹ and R² are heterocyclyl, at least one of them is substituted by one or more substituents as described above or below.

In unsubstituted or substituted aryl, aryl preferably has 6 to 18 carbon atoms and is a mono-, di- or polycyclic (preferably up to tricyclic, more preferably up to bicyclic) unsaturated carbocyclic moiety with conjugated double bonds in the ring, especially phenyl, naphthyl, biphenylenyl, indacenyl, acenaphthylenyl, fluorenyl, phenalenyl, phenanthrenyl or anthracenyl. Naphthyl and preferably phenyl are especially preferred. Aryl is unsubstituted or (in the case of substituted aryl) substituted by one or more, e.g. one to three, substituents preferably independently selected from the group consisting of C₁-C₇-alkyl, such as methyl, ethyl, n-propyl, isopropyl, n-butyl, isobutyl, sec-butyl or tert-butyl; C₂-C₇-alkenyl; C₂-C₇-alkinyl; [pyrrolidinyl (especially pyrrolidino), piperidinyl (especially piperidino), piperazinyl (especially piperazino), morpholino, thiomorpholino, pyridinyl, pyrimidinyl, pyrazinyl, pyridazinyl, oxazolyl or thiazolyl]-C₁-C₇-alkyl wherein pyrrolidinyl, piperidinyl, piperazinyl, pyridinyl, pyrimidinyl, pyrazinyl, pyridazinyl, oxazolyl or thiazolyl are unsubstituted or substituted by C₁-C₇-alkyl, such as methyl or ethyl, by pyrrolidinyl, especially pyrrolidino, by piperazinyl, especially piperazino, by amino, by N-mono- and/or N,N-di-C₁-C₇-alkylamino, by halo, by hydroxyl, by C₁-C₇-alkoxy, such as methoxy, by oxo and/or by halo-C₁-C₇-alkyl, such as trifluoromethyl, for example pyrrolidino-C₁-C₇-alkyl, 2-oxopyrrolidino-C₁-C₇-alkyl piperidino-C₁-C₇-alkyl, morpholino-C₁-C₇-alkyl, thiomorpholino-C₁-C₇-alkyl, N—C₁-C₇-alkyl-piperazino-C₁-C₇-alkyl, or N-mono- or N,N-di-(C₁-C₇-alkyl)-amino-substituted or unsubstituted pyrrolidino-C₁-C₇-alkyl; [pyrrolidinyl (especially pyrrolidino), piperidinyl (especially piperidino), piperazinyl (especially piperazino), pyridinyl, pyrimidinyl, pyrazinyl, pyridazinyl, oxazolyl or thiazolyl]-oxy-C₁-C₇-alkyl wherein pyrrolidinyl, piperidinyl, piperazinyl, pyridinyl, pyrimidinyl, pyrazinyl, pyridazinyl, oxazolyl and thiazolyl are unsubstituted or substituted by C₁-C₇-alkyl, such as methyl or ethyl, by pyrrolidinyl, especially pyrrolidino, by piperazinyl, especially piperazino, by amino, by N-mono- and/or N,N-di-C₁-C₇-alkylamino, by halo, by hydroxyl, by C₁-C₇-alkoxy, such as methoxy, by oxo and/or by halo-C₁-C₇-alkyl, such as trifluoromethyl; [pyrrolidin (especially pyrrolidino), piperidin (especially piperidino), piperazin (especially piperazino), pyridin, pyrimidin, pyrazin, pyridazin, oxazoly or thiazol]-carbonyl-C₁-C₇-alkyl wherein pyrrolidin, piperidin, piperazin, pyridin, pyrimidin, pyridazin, oxazol or pyridazin are unsubstituted or substituted by C₁-C₇-alkyl, such as methyl or ethyl, by pyrrolidinyl, especially pyrrolidino, by piperazinyl, especially piperazino, by amino, by N-mono- and/or N,N-di-C₁-C₇-alkylamino, by halo, by hydroxyl, by C₁-C₇-alkoxy, such as methoxy, by oxo and/or by halo-C₁-C₇-alkyl, such as trifluoromethyl; halo-C₁-C₇-alkyl, such as trifluoromethyl; hydroxy-C₁-C₇-alkyl, such as hydroxymethyl; C₁-C₇-alkoxy-C₁-C₇-alkyl, such as 3-methoxypropyl or 2-methoxyethyl; C₁-C₇-alkoxy-C₁-C₇-alkoxy-C₁-C₇-alkyl; phenyloxy- or naphthyloxy-C₁-C₇-alkyl; phenyl-C₁-C₇-alkoxy- or naphthyl-C₁-C₇-alkoxy-C₁-C₇-alkyl; amino-C₁-C₇-alkyl, such as aminomethyl; N-mono- or N,N-di-(C₁-C₇-alkyl, C₁-C₇-alkoxy-C₁-C₇-alkyl and/or (mono- or di-(C₁-C₇alkyl)-amino)-C₁-C₇-alkyl)-amino-C₁-C₇-alkyl; C₁-C₇-alkoxy-C₁-C₇-alkylamino-C₁-C₇-alkyl; mono- or di-[C₆-C₁₈-aryl]-C₁-C₇-alkyl in which aryl is preferably phenyl, naphthyl, biphenylenyl, indacenyl, acenaphthylenyl, fluorenyl, phenalenyl, phenanthrenyl or anthracenyl and unsubstituted or substituted by C₁-C₇-alkyl, such as methyl or ethyl, by pyrrolidinyl, especially pyrrolidino, by piperazinyl, especially piperazino, by amino, by N-mono- and/or N,N-di-C₁-C₇-alkylamino, by halo, by hydroxyl, by C₁-C₇-alkoxy, such as methoxy, and/or by halo-C₁-C₇-alkyl, such as trifluoromethyl; (naphthyl- or phenyl-C₁-C₇-alkyl)-amino-C₁-C₇-alkyl; C₁-C₇-alkanoylamino-C₁-C₇-alkyl; carboxy-C₁-C₇-alkyl; benzoyl- or naphthoylamino-C₁-C₇-alkyl; C₁-C₇-alkylsulfonyl-amino-C₁-C₇-alkyl (═C₁-C₇-alkyl-S(═O)₂—C₁-C₇-alkyl); phenyl- or naphthylsulfonylamino C₁-C₇-alkyl wherein phenyl or naphthyl is unsubstituted or substituted by one or more, especially one to three, C₁-C₇-alkyl moieties; phenyl- or naphthyl-C₁-C₇-alkylsulfonylamino-C₁-C₇-alkyl; cyano-C₁-C₇-alkyl; halo, especially fluoro (preferred), chloro (preferred) or bromo; hydroxy; C₁-C₇-alkoxy such as methoxy, ethoxy or propoxy, which is unsubstituted or substituted by one or more substituents selected from pyrrolidinyl, especially pyrrolidino, by piperazinyl, especially piperazino, by amino, by N-mono- and/or N,N-di-C₁-C₇-alkylamino, by halo, by hydroxyl, by C₁-C₇-alkoxy, such as methoxy, by halo-C₁-C₇-alkyl, such as trifluoromethyl and/or by a cyclic ether radical such as oxiranyl, oxetanyl, tetrahydrofuranyl or tetrahydropyranyl, especially oxetan-2-yl or oxetan-3-yl, with each cyclic ether radical being unsubstituted or substituted at the same carbon which is attached to said C₁-C₇-alkoxy group (i.e. forming e.g. an oxetan-3-diyl radical in the case of oxetan-3-yl being substituted at the 3-position) with a substituent independently selected from, pyrrolidinyl, especially pyrrolidino, by piperazinyl, especially piperazino, by amino, by N-mono- and/or N,N-di-C₁-C₇-alkylamino, N-mono- and/or N,N-di-C₁-C₇-alkanecarbonylamino, (e.g methyl-, ethyl-, propyl-, isopropyl-carboxamido), N-mono- and/or N,N-di-C₃-C₇-cycloalkanecarbonylamino (e.g. cyclopropylcarboxamido), N-mono- and/or N,N-di-C₁-C₇-halo-alkanecarbonylamino (e.g. trifluoromethylcarboxamido), N-mono- and/or N,N-di-C₁-C₇-alkanoxycarbonylamino (e.g. methoxycarbonylamino, tert-butyloxycarbonylamino and the like), wherein the alkyl group of the N-mono- and/or N,N-di-C₁-C₇-alkanoxycarbonylamino radical is unsubstituted or substituted by aryl, especially phenyl, naphthyl, biphenylenyl, indacenyl, acenaphthylenyl, fluorenyl, phenalenyl, phenanthrenyl or anthracenyl (e.g. giving benzyloxycarbonylamino when the N-mono- and/or N,N-di-C₁-C₇-alkanoxycarbonylamino radical is methoxycarbonylamino and the methyl group thereof is substituted by aryl which is phenyl), pyrrolidinyl, especially pyrrolidino, by piperazinyl, especially piperazino, by amino, by N-mono- and/or N,N-di-C₁-C₇-alkylamino, by halo, by hydroxyl, by C₁-C₇-alkoxy, such as methoxy, and/or by halo-C₁-C₇-alkyl, such as trifluoromethyl, by halo, by hydroxyl, by C₁-C₇-alkoxy, such as methoxy, by halo-C₁-C₇-alkyl, such as trifluoromethyl; C₆-C₁₈-aryl-C₁-C₇-alkoxy in which aryl is preferably phenyl, naphthyl, biphenylenyl, indacenyl, acenaphthylenyl, fluorenyl, phenalenyl, phenanthrenyl or anthracenyl and unsubstituted or substituted by C₁-C₇-alkyl, such as methyl or ethyl, by C₁-C₇-alkoxy, by pyrrolidinyl, especially pyrrolidino, by piperazinyl, especially piperazino, by amino, by N-mono- and/or N,N-di-C₁-C₇-alkylamino, by halo, by hydroxyl, by C₁-C₇-alkoxy, such as methoxy, and/or by halo-C₁-C₇-alkyl, such as trifluoromethyl; hydroxy-C₂-C₇-alkoxy, such as 2-hydroxyethoxy; C₁-C₇-alkoxy-C₁-C₇-alkoxy; C₁-C₇-alkoxy-C₁-C₇-alkoxy-C₁-C₇-alkoxy; halo-C₁-C₇-alkoxy; amino-C₂-C₇-alkoxy, such as 2-aminoethoxy or 3-aminopropoxy; N-mono- or N,N-di-(C₁-C₇-alkyl)-amino-C₁-C₇-alkoxy; N—C₁-C₇-alkanoylamino-C₁-C₇-alkoxy; C₁-C₇-alkoxycarbonylamino-C₁-C₇-alkoxy, such as 2-(tert-butoxycarbonylamino)-ethoxy or 3-(tert-butoxycarbonylamino)-propoxy; C₆-C₁₄-aryl-carbonylamino-C₂-C₇-alkoxy (C₆-C₁₄-aryl-C(═O)—NH—C₂-C₇-alkoxy or C₆-C₁₄-aroyl-NH—C₂-C₇-alkoxy) wherein C₆-C₁₄-aryl is unsubstituted or substituted by one or more, especially up to three, substituents independently selected from the group consisting of C₁-C₇-alkyl, hydroxy, C₁-C₇-alkoxy, halo and cyano; N-unsubstituted-, N-mono- or N,N-di-(C₁-C₇-alkyl)carbamoyl-C₁-C₇-alkoxy; phenyl- or naphthyloxy; phenyl- or naphthyl-C₁-C₇-alkyloxy; [pyrrolyl, pyrrolidinyl (especially pyrrolidino), imidazolyl (especially imidazolo), imidazolidinyl (especially imidazolidino), piperidinyl (especially piperidino), piperazinyl (especially piperazino), pyridinyl, pyrimidinyl, pyrazinyl, pyridazinyl, oxazolyl, thiazolyl, morpholinyl (especially morpholino), thiomorpholinyl (especially thiomorpholino), S-oxothiomorpholinyl (especially S-oxothiomorpholino) or S,S-dioxothiomorpholinyl (especially S,S-dioxothiomorpholino)]-C₁-C₇-alkoxy wherein pyrrolidinyl, piperidinyl, piperazinyl, pyridinyl, pyrimidinyl, pyrazinyl, pyri-dazinyl, oxazolyl and thiazolyl are unsubstituted or substituted by C₁-C₇-alkyl, such as methyl or ethyl, by pyrrolidinyl, especially pyrrolidino, by piperazinyl, especially piperazino, by amino, by N-mono- and/or N,N-di-C₁-C₇-alkylamino, by halo, by hydroxyl, by C₁-C₇-alkoxy, such as methoxy, by oxo and/or by halo-C₁-C₇-alkyl, such as trifluoromethyl; [pyrrolyl, pyrrolidinyl (especially pyrrolidino), imidazolyl (especially imidazolo), imidazolidinyl (especially imidazolidino), piperidinyl (especially piperidino), piperazinyl (especially piperazino), pyridinyl, pyrimidinyl, pyrazinyl, pyridazinyl, oxazolyl, thiazolyl, morpholinyl (especially morpholino), thiomorpholinyl (especially thiomorpholino), S-oxothiomorpholinyl (especially S-oxothiomoprpholino) or S,S-dioxothiomorpholinyl (especially S,S-dioxothiomorpholino)]-oxy-C₁-C₇-alkoxy wherein pyrrolidinyl, piperidinyl, piperazinyl, pyridinyl, pyrimidinyl, pyrazinyl, pyridazinyl, oxazolyl and thiazolyl are unsubstituted or substituted by C₁-C₇-alkyl, such as methyl or ethyl, by pyrrolidinyl, especially pyrrolidino, by piperazinyl, especially piperazino, by amino, by N-mono- and/or N,N-di-C₁-C₇-alkylamino, by halo, by hydroxyl, by C₁-C₇-alkoxy, such as methoxy, by oxo and/or by halo-C₁-C₇-alkyl, such as trifluoromethyl; C₃-C₈-cyloalkoxy; pyridincarbonylamino-C₁-C₇-alkoxy, C₆-C₁₄-arylaminocarbonylamino-C₂-C₇-alkoxy (C₆-C₁₄-aryl-NH—C(═O)—NH—C₂-C₇-alkoxy) wherein C₆-C₁₄-aryl is unsubstituted or substituted by one or more, especially up to three, substituents independently selected from the group consisting of C₁-C₇-alkyl, halo-C₁-C₇-alkyl, hydroxy, C₁-C₇-alkoxy, halo and cyano; pyridinyl-aminocarbonylamino-C₁-C₇-alkoxy; C₁-C₇-alkanoyloxy; benzoyl- or naphthoyloxy; carboxy-C₁-C₇-alkoxy; C₁-C₇-alkoxycarbonyl-C₁-C₇-alkoxy, such as methoxycarbonylmethoxy; heterocyclyloxy (especially pyrrolyloxy, furanyloxy, thiophenyloxy, imidazolyloxy, pyrazolyloxy, thiazolyloxy, pyrazolidinyloxy, pyrrolidinyloxy, pyridinyloxy, piperidinyloxy, oxopiperidinyloxy, piperazinyloxy, triazolyloxy, morpholinyloxy, thiomorpholinyloxy, S-oxo-thiomorpholinyloxy, benzimidazolyloxy, pyrrolo-pyrimidinyloxy, or 1H,4H,5H-trihydro-pyrazolo[2,3-c]piperidin-1-yloxy (meaning 5-aza-3,4,5,6-tetrahydroindazol-1-yloxy)) bound to the “oxy” via a ring carbon and that is unsubstituted or substituted by one or more, especially up to three, substituents independently selected from C₁-C₇-alkyl, such as isopropyl, halo-C₁-C₇-alkyl, phenyl, halophenyl, hydroxy, C₁-C₇-alkoxy, halo, C₁-C₇-alkoxycarbonyl, carbamoyl, phenylsulfonyl wherein phenyl is unsubstituted or substituted by one or more, preferably up to three, substituents independently selected from C₁-C₇-alkyl, hydroxy, C₁-C₇-alkoxy, halo, nitro and cyano, heterocyclylcarbonyl (=heterocyclyl-C(═O)—) where heterocyclyl is bound via a ring nitrogen to the carbonyl, especially piperidinocarbonyl, morpholino-carbonyl, thiomorpholino-carbonyl or S-oxo- or S,S-dioxothiomorpholinocarbonyl, C₁-C₇-alkanoyl, unsubstituted or substituted benzoyl wherein the substituents are preferably one or more, e.g. up to three, substituents independently selected from the group consisting of hydroxy, C₁-C₇-alkoxy and cyano, C₁-C₇-alkanesulfonyl, unsubstituted or substituted benzenesulfonyl wherein the substituents are preferably one or more, e.g. up to three, substituents independently selected from the group consisting of hydroxy, C₁-C₇-alkoxy and cyano, sulfamoyl, N-mono- or N,N-disubstituted sulfamoyl, preferably N-mono- or N,N-di-(C₁-C₇-alkyl)-sulfamoyl, cyano and nitro, especially N-isopropyl-piperidinyloxy; amino; mono- or di-(C₁-C₇-alkyl, C₃-C₈-cyloalkyl and/or hydroxyl-C₁-C₇-alkyl)-amino; mono- or di-(naphthyl- or phenyl-C₁-C₇-alkyl)-amino; C₁-C₇-alkanoylamino; unsubstituted or amino-, N-mono- or N,N-di-(C₁-C₇-alkyl and/or phenyl- or naphthyl-C₁-C₇alkyl)amino-substituted benzoyl- or naphthoylamino; C₁-C₇-alkoxycarbonylamino; (phenyl or naphthyl)-C₁-C₇-alkoxycarbonylamino; C₁-C₇-alkylsulfonylamino (═C₁-C₇-alkyl-S(═O)₂—NH—); phenyl- or naphthylsulfonylamino wherein phenyl or naphthyl is unsubstituted or substituted by one or more, especially one to three, C₁-C₇-alkyl moieties; phenyl- or naphthyl-C₁-C₇-alkylsulfonylamino; heterocyclylamino (especially pyrrolylamino, furanylamino, thiophenylamino, imidazolylamino, pyrazolylamino, thiazolylamino, pyrazolidinylamino, pyrrolidinylamino, pyridinylamino, piperidinylamino, oxopiperidinylamino, piperazinylamino, triazolylamino, morpholinylamino, thiomorpholinylamino, S-oxothiomorpholinylamino, benzimidazolylamino, pyrrolo-pyrimidinylamino, or 1H,4H,5H-trihydropyrazolo[2,3-c]piperidin-1-ylamino (meaning 5-aza-3,4,5,6-tetrahydroindazol-1-ylamino)) bound via a ring carbon to the “amino”and that is unsubstituted or substituted by one or more, especially up to three, substituents independently selected from C₁-C₇-alkyl, such as isopropyl, halo-C₁-C₇-alkyl, phenyl, halophenyl, hydroxy, C₁-C₇-alkoxy, halo, C₁-C₇-alkoxycarbonyl, carbamoyl, phenylsulfonyl wherein phenyl is unsubstituted or substituted by one or more, preferably up to three, substituents independently selected from C₁-C₇-alkyl, hydroxy, C₁-C₇-alkoxy, halo, nitro and cyano, heterocyclylcarbonyl (=heterocyclyl-C(═O)—) where heterocyclyl is bound via a ring nitrogen to the carbonyl, especially piperidinocarbonyl, morpholino-carbonyl, thiomorpholino-carbonyl or S-oxo- or S,S-dioxothiomorpholinocarbonyl, C₁-C₇-alkanoyl, unsubstituted or substituted benzoyl wherein the substituents are preferably one or more, e.g. up to three, substituents independently selected from the group consisting of hydroxy, C₁-C₇-alkoxy and cyano, C₁-C₇-alkanesulfonyl, unsubstituted or substituted benzenesulfonyl wherein the substituents are preferably one or more, e.g. up to three, substituents independently selected from the group consisting of hydroxy, C₁-C₇-alkoxy and cyano, sulfamoyl, N-mono- or N,N-disubstituted sulfamoyl, preferably N-mono- or N,N-di-(C₁-C₇-alkyl)-sulfamoyl, cyano and nitro, such as 4-(phenyl)-thiazol-2-yl-amino; C₁-C₇-alkylthio; halo-C₁-C₇-alkylthio, such as trifluoromethylthio; C₁-C₇-alkane-sulfonyl; C₃-C₈-cyloalkyl-sulfonyl (═C₃-C₈-cycloalkyl-S(═O)₂—); C₁-C₇-alkoxy-C₁-C₇-alkylthio, phenyl- or naphthylthio; phenyl- or naphthyl-C₁-C₇-alkylthio; C₁-C₇-alkanoylthio; benzoyl- or naphthylthio; C₁-C₇-alkanoyl, especially acetyl (1-oxoethyl); C₁-C₇-alkoxy-C₁-C₇-alkanoyl; unsubstituted or substituted benzoyl wherein the substituents are preferably one or more, e.g. up to three, substituents independently selected from the group consisting of hydroxy, C₁-C₇-alkoxy and cyano; carboxyl (—COOH); carboxy, C₁-C₇-alkoxycarbonyl, such as ethoxycarbonyl; phenoxy- or naphthoxycarbonyl; phenyl- or naphthyl-C₁-C₇-alkoxy-carbonyl; C₁-C₁₀- especially C₁-C₄-alkylendioxy, such as methylendioxy or 1,2-ethylendioxy; carbamoyl; N-mono- or N,N-di-[C₁-C₇-alkyl, naphthyl-C₁-C₇-alkyl, phenyl-C₁-C₇-alkyl, N′-mono- or N′,N′-di-(C₁-C₇alkyl)amino-C₁-C₇-alkyl, pyrrolidinyl (especially pyrrolidino)-C₁-C₇-alkyl, piperidinyl (especially piperidino)-C₁-C₇-alkyl, piperazinyl- or N—(C₁-C₇-alkyl)piperazinyl (especially piperazino or 4-C₁-C₇-alkylpiperazino)-C₁-C₇-alkyl, mono-C₁-C₇-alkoxy-C₁-C₇-alkyl, (N′-mono- or N′,N′-di-(C₁-C₇-alkyl)-amino)-C₁-C₇-alkyl, phenyl, pyridinyl, oxazolyl or thiazolyl each of which is unsubstituted or substituted by C₁-C₇-alkoxy, by halo, especially fluoro, by pyrrolidino, by piperidino, by piperazino, by hydroxyl-C₁-C₇-alkylamino, by hydroxyl-C₁-C₇-alkyl, by amino or by N-mono- or N,N-di-(C₁-C₇-alkyl)amino, C₃-C₈-cyloalkyl, pyrrolidinyl, piperidinyl, morpholinyl, piperazinyl, pyrimidinyl, pyrazinyl and/or pyridazinyl]-amino-carbonyl, such as N-mono- or N,N-di-(C₁-C₇-alkyl)-aminocarbonyl; N—C₁-C₇-alkoxy-C₁-C₇-alkylcarbamoyl; pyrrolidin-1-carbonyl; amino-N-pyrrolidin-1-carbonyl; N-mono- or N,N-di(C₁-C₇-alkyl)amino-pyrrolidin-1-carbonyl; piperidin-1-carbonylmorpholin-4-carbonyl; morpholinocarbonyl, thiomorpholinocarbonyl, S-oxo- or S,S-dioxo-thiomorpholino-carbonyl, thiomorpholin-4-carbonyl; S-oxo-thiomorpholin-4-carbonyl; S,S-dioxothiomorpholin-4-carbonyl; piperazin-1-carbonyl; N—C₁-C₇-alkyl-piperazin-1-carbonyl; N—C₁-C₇-alkoxycarbonyl-piperazin-1-carbonyl; N-mono- or N,N-di-(C₁-C₇-alkyl)-amino-substituted or unsubstituted pyrrolidinyl-C₁-C₇-alkyl-carbonyl; cyano; C₁-C₇-alkenylene or -alkinylene; C₁-C₇-alkylsulfonyl (═C₁-C₇-alkane-sulfonyl); phenyl- or naphthylsulfonyl wherein phenyl or naphthyl is unsubstituted or substituted by one or more, especially one to three, moieties independently selected from the group consisting of C₁-C₇-alkyl, hydroxy, C₁-C₇-alkoxy and cyano; phenyl- or naphthyl-C₁-C₇-alkylsulfonyl; sulfamoyl; N-mono or N,N-di-[C₁-C₇-alkyl, phenyl-, naphthyl-, phenyl-C₁-C₇-alkyl-, pyrrolidinyl (especially pyrrolidino)-C₁-C₇-alkyl, piperidinyl (especially piperidino)-C₁-C₇-alkyl, piperazinyl (especially piperazino)-C₁-C₇-alkyl, N—C₁-C₇-alkyl-piperazinyl (especially 4-C₁-C₇-alkylpiperazino)-C₁-C₇-alkyl, naphthyl-C₁-C₇-alkyl, phenyl which is unsubstituted or substituted by C₁-C₇-alkoxy, by halo, especially fluoro, by pyrrolidino, by piperidino, by piperazino, by hydroxyl-C₁-C₇-alkyl or by N-mono- or N,N-di-(C₁-C₇-alkyl)-C₁-C₇-alkyl; pyrrolidinyl (especially pyrrolidino), piperidinyl (especially piperidino), piperazinyl (especially piperazino), pyridinyl, pyrimidinyl, pyrazinyl, pyridazinyl, oxazolyl and/or thiazolyl]-aminosulfonyl; heterocyclyl (preferably pyrrolyl, especially 2-pyrrolyl, furanyl, especially 3-furanyl, thiophenyl, especially thiophen-3-yl, pyrazolyl, pyrazolidinyl, pyridinyl that is unsubstituted or substituted by preferably C₁-C₇-alkoxy, such as methoxy, by halo-C₁-C₇-alkyl, such as trifluoromethyl, and/or by cyano, pyrrolidinyl, such as pyrrolidin-1-yl, oxopyrrolidinyl, such as 2-oxo-pyrrolidin-1-yl, piperidinyl, oxo-piperidinyl, such as 2-oxopiperidin-1-yl, N—C₁-C₇-alkylpiperidinyl, such as 1-isopropyl-piperidin-4-yl, morpholinyl, such as morpholino, thiomorpholinyl, such as thiomorpholino, S-oxo-thiomorpholinyl, such as S-oxo-thiomorpholino, S,S-dioxothiomorpholinyl, such as S,S-dioxo-thiomorpholino, piperazinyl, N—C₁-C₇-alkyl-piperazinyl, 4-(phenyl-C₁-C₇-alkyl)-piperazinyl; 4-(naphthyl-C₁-C₇-alkyl)-piperazinyl; 4-(C₁-C₇-alkoxycarbonyl)-piperazinyl, 4-(phenyl-C₁-C₇-alkoxycarbonyl)-piperazinyl, 4-(naphthyl-C₁-C₇-alkoxycarbonyl)-piperazinyl, oxazolyl, thiazolyl, phenyithiazolyl, such as 4-phenyl-thiazol-2-yl, triazolyl, e.g. 1,2,4-triazol-1-yl, carbamoyl-triazolyl, e.g. carbamoyl-1,2,4-triazol-1-yl, such as 3-carbamoyl-1,2,4-triazol-1-yl; pyrazolyl, such as pyrazol-1-yl; halo-C₁-C₇alkyl-pyrazolyl, such as 3-trifluoromethyl-pyrazol-1-yl, halophenyl-pyrazolyl, such as 3-(halophenyl)-pyrazol-1-yl, e.g. 3-(4-chlorophenyl)-pyrazol-1-yl, pyrimidin-(2-, 4- or 5-)yl, benzimidazol(especially -1-)yl, (e.g. 5-)C₁-C₇-alkoxy-substituted benzimidazol (especially -1-)yl, pyrrolo-pyrimidinyl, especially pyrrolo[2,3-d]pyrimidin-(e.g. 1-)yl, C₁-C₇-alkyl-substituted pyrrolo-pyrimidinyl, e.g. 2-C₁-C₇-alkyl-pyrrolo[2,3-d]pyrimidin-(e.g.1-)yl (meaning 2-C₁-C₇-alkyl-5,7-diazaindol-1-yl), 1H,4H,5H-trihydropyrazolo[2,3-c]piperidin-1-yl (meaning 5-aza-3,4,5,6-tetrahydroindazol-1-yl) which is unsubstituted or substituted by 1 or 2 substituents independently selected from C₁-C₇-alkyl (e.g. methyl, especially in 5-position) and halo-C₁-C₇-alkyl (e.g. trifluoromethyl, especially in 3-position)), which heterocyclyl is bound via a ring nitrogen atom or preferably via a ring carbon and is unsubstituted or substituted by one or more, especially up to three, substituents independently selected from C₁-C₇-alkyl, such as isopropyl, halo-C₁-C₇-alkyl, phenyl, halophenyl, hydroxy, C₁-C₇-alkoxy, halo, C₁-C₇-alkoxycarbonyl, carbamoyl, phenylsulfonyl wherein phenyl is unsubstituted or substituted by one or more, preferably up to three, substituents independently selected from hydroxy, C₁-C₇-alkoxy, halo, nitro and cyano, heterocyclylcarbonyl (=heterocyclyl-C(═O)-) where heterocyclyl is bound via a ring nitrogen to the carbonyl, especially piperidinocarbonyl, morpholino-carbonyl, thiomorpholino-carbonyl or S-oxo- or S,S-dioxothiomorpholinocarbonyl, C₁-C₇-aikanoyl, unsubstituted or substituted benzoyl wherein the substituents are preferably one or more, e.g. up to three, substituents independently selected from the group consisting of hydroxy, C₁-C₇-alkoxy and cyano, C₁-C₇-alkanesulfonyl, unsubstituted or substituted benzenesulfonyl wherein the substituents are preferably one or more, e.g. up to three, substituents independently selected from the group consisting of hydroxy, C₁-C₇-alkoxy and cyano, sulfamoyl, N-mono- or N,N-disubstituted sulfamoyl, preferably N-mono- or N,N-di-(C₁-C₇-alkyl)-sulfamoyl, cyano and nitro, preferably being substituted as given specifically.

Further aryl substituents may be selected from C₃-C₈-cycloalkyl, phenyl and naphthyl each of which is unsubstituted or substituted by one or more, e.g. up to 2, moieties independently selected from the group consisting of halo, C₁-C₇-alkoxy, C₁-C₇-alkanesulfonyl, nitro and cyano; tetrazolyl, e.g. tetrazol-5-yl; indol-(e.g.5-)yl; indazolyl, e.g. indazol-5-yl; (e.g. 3-) C₁-C₇-alkyl-indazoyl-(e.g. 5-)yl; and pyrrolo-pyridinyl, e.g. pyrrolo[2,3-c]pyridine-1-yl (meaning 5-aza-indol-1-yl). Especially preferably unsubstituted or substituted aryl is naphthyl or especially phenyl, each of which is unsubstituted or substituted as just described, more preferably by one or more, e.g. up to three, substituents independently selected from those mentioned above.

Where R¹ and/or R² comprise a six-membered ring (as total or part of aryl or heterocyclyl each of which is unsubstiotuted or subsstituted) bound to the rest of the molecule of the formula I, preferably a substituent is present in the meta-position and/or in the para-position.

An N-oxide derivative or pharmaceutically acceptable salt of each of the compounds of the formula I is also within the scope of this invention. For example, a nitrogen ring atom of a nitrogen-containing heterocyclic (e.g. heteroaryl or the central bicyclic core of the compound of the formula I) can form an N-oxide in the presence of a suitable oxidizing agent, e.g. a peroxide, such as m-chloro-perbenzoic acid or hydrogen peroxide.

Wherever a compound or compounds of the formula I are mentioned, this is further also intended to include (as alternative to the compound or in addition) one or more N-oxides of such compounds, also where not stated explicitly.

The term “an N-oxide thereof, a solvate thereof and/or a pharmaceutically acceptable salt thereof” especially means that a compound of the formula I may be present as such or in mixture with its N-oxide or as essentially pure N-oxide, as a solvate of the compound or the N-oxide, or as a salt of the compound of the formula I or an N-oxide thereof, or as a solvate of such salt and/or N-oxide, either each of these forms in essentially pure form or as a mixture with one or more of the other forms.

Compounds of the formula I can also be modified by appending appropriate functionalities to enhance selective biological properties. Modifications of this kind are known in the art and include those that increase penetration into a given biological system (e.g. blood, lymphatic system, central nervous system, testis), increase bioavailability, increase solubility to allow parenteral administration (e.g. injection, infusion), alter metabolism and/or alter the rate of secretion. Examples of this type of modifications include but are not limited to esterification, e.g. with polyethylene glycols, derivatisation with pivaloyloxy or fatty acid substituents, conversion to carbamates, hydroxylation of aromatic rings and heteroatom substitution in aromatic rings. Whereever compounds of the formula I, N-oxides, solvates and/or (especially pharmaceutically acceptable) salts thereof are mentioned, this comprises such modified formulae, while preferably the molecules of the formula I, N-oxides, solvates and/or (especially pharmaceutically acceptable) salts thereof as such are meant.

In view of the close relationship between the compounds of the formula I in free form and those in the form of their salts, including those salts that can be used as intermediates, for example in the purification or identification of the novel compounds, any reference to a compound or compounds of the formula I hereinbefore and hereinafter is to be understood as referring also to one or more salts, as appropriate and expedient, as well as to one or more solvates, e.g. hydrates.

Solvate means a (at least partially) crystalline compound of the formula I or a salt thereof in crystalline form with solvent molecules included in the crystal structure—the term solvate here includes hydrates (crystals including water molecules) and/or any other (preferably pharmaceutically acceptable) solvates with one or more other solvents.

Salts are formed, for example, as acid addition salts, preferably with organic or inorganic acids, from compounds of formula I with a basic nitrogen atom, and are especially pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, for example acetic acid, propionic acid, octanoic acid, decanoic acid, dodecanoic acid, glycolic acid, lactic acid, fumaric acid, succinic acid, malonic acid, adipic acid, pimelic acid, suberic acid, azelaic acid, malic acid, tartaric acid, citric acid, amino acids, such as glutamic acid or aspartic acid, maleic acid, hydroxymaleic acid, methylmaleic acid, cyclohexanecarboxylic acid, adamantanecarboxylic acid, benzoic acid, salicylic acid, 4-aminosalicylic acid, phthalic acid, phenylacetic acid, mandelic acid, cinnamic acid, methane- or ethane-sulfonic acid, 2-hydroxyethanesulfonic acid, ethane-1,2-disulfonic acid, benzenesulfonic acid, 4-toluenesulfonic acid, 2-naphthalenesulfonic acid, 1,5-naphthalene-disulfonic acid, 2- or 3-methylbenzenesulfonic acid, methylsulfuric acid, ethylsulfuric acid, dodecylsulfuric acid, N-cyclohexylsulfamic acid, N-methyl-, N-ethyl- or N-propyl-sulfamic acid, or other organic protonic acids, such as ascorbic acid.

For isolation or purification purposes it is also possible to use pharmaceutically unacceptable salts, for example picrates or perchlorates. For therapeutic use, only pharmaceutically acceptable salts or free compounds are employed (where applicable in the form of pharmaceutical preparations), and these are therefore preferred.

Preferred is the USE (especially in the diagnostic or preferably therapeutic, including prophylactic treament of one or more diseases or disorders where the disease(s) or disorder(s) respond or responds to an inhibition of one or more kinases of the PI3-kinase-related protein kinase family) of one or more compounds of the formula I,

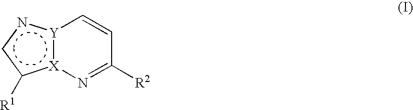

wherein

either X is N and Y is C, or X is C and Y is N,

the broken circle represents two conjugated double bonds within the five-membered ring with the proviso that the first of said bonds starts from either X═C or Y═C;

and each of R¹ and R² is, independently of the other, unsubstituted or substituted aryl or unsubstituted or substituted heterocyclyl;

and/or an N-oxide thereof, a solvate and/or a (preferably pharmaceutically acceptable) salt thereof, especially where as a warm-blooded animal to be treated a human is to be treated.

Preferred is the USE of a compound of the formula IB already shown, wherein R¹ and R² are as defined in the preceding paragraph, and/or an N-oxide thereof, a solvate and/or a pharmaceutically acceptable salt thereof.

More preferred is the use according to any of the two preceding embodiments where the disease to be treated is a benign or malignant tumor, carcinoma of the brain, kidney, liver, adrenal gland, bladder, breast, stomach, gastric tumors, ovaries, colon, rectum, prostate, pancreas, lung, vagina or thyroid, sarcoma, glioblastomas, multiple myeloma or gastrointestinal cancer, especially colon carcinoma or colorectal adenoma or a tumor of the neck and head, a neoplasia, especially of epithelial character, lymphomas, a mammary carcinoma or a leukemia, or Cowden syndrome, Lhermitte-Dudos disease or Bannayan-Zonana syndrome.

Yet more preferred is the USE according to any one of the three preceding embodiments where in the compound of the formula I or IA

unsubstituted or substituted heterocyclyl is a heterocyclic radical selected from the group consisting of oxiranyl, azirinyl, aziridinyl, 1,2-oxathiolanyl, thienyl, furanyl, tetrahydrofuryl, pyranyl, thiopyranyl, thianthrenyl, isobenzofuranyl, benzofuranyl, chromenyl, 2H-pyrrolyl, pyrrolyl, pyrrolinyl, pyrrolidinyl, imidazolyl, imidazolidinyl, benzimidazolyl, pyrazolyl, pyrazinyl, pyrazolidinyl, thiazolyl, isothiazolyl, dithiazolyl, oxazolyl, isoxazolyl, pyridinyl, pyrazinyl, pyrimidinyl, piperidinyl, piperazinyl, pyridazinyl, morpholinyl, thiomorpholinyl, (S-oxo or S,S-dioxo)-thiomorpholinyl, furazanyl, indolizinyl, azepanyl, diazepanyl, isoindolyl, 3H-indolyl, indolyl, benzimidazolyl, indazolyl, triazolyl, tetrazolyl, purinyl, 4H-quinolizinyl, isoquinolyl, quinolyl, tetrahydroquinolyl, tetrahydroisoquinolyl, decahydroquinolyl, octahydroisoquinolyl, benzofuranyl, dibenzofuranyl, benzothiophenyl, dibenzothiophenyl, phthalazinyl, naphthyridinyl, pyrrolo-pyrimidinyl, 1H,4H,5H-trihydropyrazolo[2,3-c]piperidin-1-yl, pyrrolo-pyridinyl, quinoxalyl, quinazolinyl, cinnolinyl, pteridinyl, carbazolyl, beta-carbolinyl, phenanthridinyl, acridinyl, perimidinyl, phenanthrolinyl, phenazinyl, phenothiazinyl, phenoxazinyl, isochromanyl, chromanyl, benzo[1,3]dioxol-5-yl and 2,3-dihydro-benzo[1,4]dioxin-6-yl, each of these radicals being unsubstituted or substituted by one or more substituents independently selected from those mentioned below for substituted aryl;

and unsubstituted or substituted aryl is phenyl, naphthyl, biphenylenyl, indacenyl, acenaphthylenyl, fluorenyl, phenalenyl, phenanthrenyl or anthracenyl which is unsubstituted or substituted by one or more substituents preferably independently selected from the group consisting of C₁-C₇-alkyl, C₂-C₇-alkenyl; C₂-C₇-alkinyl; [pyrrolidinyl, piperidinyl, piperazinyl, morpholino, thiomorpholino, pyridinyl, pyrimidinyl, pyrazinyl, pyridazinyl, oxazolyl or thiazolyl]-C₁-C₇-alkyl wherein pyrrolidinyl, piperidinyl, piperazinyl, pyridinyl, pyrimidinyl, pyrazinyl, pyridazinyl, oxazolyl or thiazolyl are unsubstituted or substituted by C₁-C₇-alkyl, by pyrrolidinyl, by piperazinyl, by amino, by N-mono- and/or N,N-di-C₁-C₇-alkylamino, by halo, by hydroxyl, by C₁-C₇-alkoxy, by oxo and/or by halo-C₁-C₇-alkyl; [pyrrolidinyl, piperidinyl, piperazinyl, pyridinyl, pyrimidinyl, pyrazinyl, pyridazinyl, oxazolyl or thiazolyl]-oxy-C₁-C₇-alkyl wherein pyrrolidinyl, piperidinyl, piperazinyl, pyridinyl, pyrimidinyl, pyrazinyl, pyridazinyl, oxazolyl and thiazolyl are unsubstituted or substituted by C₁-C₇-alkyl, by pyrrolidinyl, by piperazinyl, by amino, by N-mono- and/or N,N-di-C₁-C₇-alkylamino, by halo, by hydroxyl, by C₁-C₇-alkoxy, by oxo and/or by halo-C₁-C₇-alkyl; [pyrrolidin, piperidin, piperazin, pyridin, pyrimidin, pyrazin, pyridazin, oxazoly or thiazol]-carbonyl-C₁-C₇-alkyl wherein pyrrolidin, piperidin, piperazin, pyridin, pyrimidin, pyridazin, oxazol or pyridazin are unsubstituted or substituted by C₁-C₇-alkyl, by pyrrolidinyl, by piperazinyl, by amino, by N-mono- and/or N,N-di-C₁-C₇-alkylamino, by halo, by hydroxyl, by C₁-C₇-alkoxy, by oxo and/or by halo-C₁-C₇-alkyl; halo-C₁-C₇-alkyl; hydroxy-C₁-C₇-alkyl; C₁-C₇-alkoxy-C₁-C₇-alkyl; C₁-C₇-alkoxy-C₁-C₇-alkoxy-C₁-C₇-alkyl; phenyloxy- or naphthyloxy-C₁-C₇-alkyl; phenyl-C₁-C₇-alkoxy- or naphthyl-C₁-C₇-alkoxy-C₁-C₇-alkyl; amino-C₁-C₇-alkyl; N-mono- or N,N-di-(C₁-C₇-alkyl, C₁-C₇-alkoxy-C₁-C₇-alkyl and/or (mono- or di-(C₁-C₇-alkyl)-amino)-C₁-C₇-alkyl)-amino-C₁-C₇-alkyl; C₁-C₇-alkoxy-C₁-C₇-alkylamino-C₁-C₇-alkyl; mono- or di-[C₆-C₁₈-aryl]-C₁-C₇-alkyl in which aryl is phenyl, naphthyl, biphenylenyl, indacenyl, acenaphthylenyl, fluorenyl, phenalenyl, phenanthrenyl or anthracenyl and is unsubstituted or substituted by C₁-C₇-alkyl, by pyrrolidinyl, by piperazinyl, by amino, by N-mono- and/or N,N-di-C₁-C₇-alkylamino, by halo, by hydroxyl, by C₁-C₇-alkoxy and/or by halo-C₁-C₇-alkyl; (naphthyl- or phenyl-C₁-C₇-alkyl)-amino-C₁-C₇-alkyl; C₁-C₇-alkanoylamino-C₁-C₇-alkyl; carboxy-C₁-C₇-alkyl; benzoyl- or naphthoylamino-C₁-C₇-alkyl; C₁-C₇-alkylsulfonylamino-C₁-C₇-alkyl; phenyl- or naphthylsulfonylamino-C₁-C₇-alkyl wherein phenyl or naphthyl is unsubstituted or substituted by one or more C₁-C₇-alkyl moieties; phenyl- or naphthyl-C₁-C₇-alkylsulfonylamino-C₁-C₇-alkyl; cyano-C₁-C₇-alkyl; halo; hydroxy; C₁-C₇-alkoxy; C₆-C₁₅-aryl-C₁-C₇-alkoxy in which aryl is phenyl, naphthyl, biphenylenyl, indacenyl, acenaphthylenyl, fluorenyl, phenalenyl, phenanthrenyl or anthracenyl and is unsubstituted or substituted by C₁-C₇-alkyl, by C₁-C₇-alkoxy, by pyrrolidinyl, by piperazinyl, by amino, by N-mono- and/or N,N-di-C₁-C₇-alkylamino, by halo, by hydroxyl, by C₁-C₇-alkoxy and/or by halo-C₁-C₇-alkyl; hydroxy-C₂-C₇-alkoxy, C₁-C₇-alkoxy-C₁-C₇-alkoxy; C₁-C₇-alkoxy-C₁-C₇-alkoxy-C₁-C₇-alkoxy; halo-C₁-C₇-alkoxy; amino-C₂-C₇-alkoxy; N-mono- or N,N-di-(C₁-C₇-alkyl)-amino-C₁-C₇-alkoxy; N—C₁-C₇-alkanoylamino-C₁-C₇-alkoxy; C₁-C₇-alkoxycarbonyl-amino-C₁-C₇-alkoxy; C₆-C₁₄-arylcarbonylamino-C₂-C₇-alkoxy wherein C₆-C₁₄-aryl is phenyl, naphthyl, biphenylenyl, indacenyl, acenaphthylenyl, fluorenyl, phenalenyl, phenanthrenyl or anthracenyl and is unsubstituted or substituted by one or more, especially up to three, substituents independently selected from the group consisting of C₁-C₇-alkyl, halo-C₁-C₇-alkyl, hydroxy, C₁-C₇-alkoxy, halo and cyano; N-unsubstituted-, N-mono- or N,N-di-(C₁-C₇-alkyl)carbamoyl-C₁-C₇-alkoxy; phenyl- or naphthyloxy; phenyl- or naphthyl-C₁-C₇-alkyloxy; [pyrrolyl, pyrrolidinyl, imidazolyl, imidazolidinyl, piperidinyl, piperazinyl, pyridinyl, pyrimidinyl, pyrazinyl, pyridazinyl, oxazolyl, thiazolyl, morpholinyl, thiomorpholinyl, S-oxothiomorpholinyl or S,S-dioxothiomorpholinyl]-C₁-C₇-alkoxy wherein pyrrolidinyl, piperidinyl, piperazinyl, pyridinyl, pyrimidinyl, pyrazinyl, pyridazinyl, oxazolyl and thiazolyl are unsubstituted or substituted by C₁-C₇-alkyl, by pyrrolidinyl, by piperazinyl, by amino, by N-mono- and/or N,N-di-C₁-C₇-alkylamino, by halo, by hydroxyl, by C₁-C₇-alkoxy, by oxo and/or by halo-C₁-C₇-alkyl; [pyrrolyl, pyrrolidinyl, imidazolyl, imidazolidinyl, piperidinyl, piperazinyl, pyridinyl, pyrimidinyl, pyrazinyl, pyridazinyl, oxazolyl, thiazolyl, morpholinyl, thiomorpholinyl, S-oxothiomorpholinyl or S,S-dioxothiomorpholinyl]-oxy-C₁-C₇-alkoxy wherein pyrrolidinyl, piperidinyl, piperazinyl, pyridinyl, pyrimidinyl, pyrazinyl, pyridazinyl, oxazolyl and thiazolyl are unsubstituted or substituted by C₁-C₇-alkyl, by pyrrolidinyl, by piperazinyl, by amino, by N-mono- and/or N,N-di-C₁-C₇-alkylamino, by halo, by hydroxyl, by C₁-C₇-alkoxy, by oxo and/or by halo-C₁-C₇-alkyl; C₃-C₈-cyloalkoxy; pyridincarbonylamino-C₁-C₇-alkoxy, C₆-C₁₄-arylaminocarbonylamino-C₂-C₇-alkoxy in which aryl is phenyl, naphthyl, biphenylenyl, indacenyl, acenaphthylenyl, fluorenyl, phenalenyl, phenanthrenyl or anthracenyl and is unsubstituted or substituted by one or more substituents independently selected from the group consisting of C₁-C₇-alkyl, halo-C₁-C₇-alkyl, hydroxy, C₁-C₇-alkoxy, halo and cyano; pyridinylaminocarbonylamino-C₁-C₇-alkoxy; C₁-C₇-alkanoyloxy; benzoyl- or naphthoyloxy; carboxy-C₁-C₇-alkoxy; C₁-C₇-alkoxycarbonyl-C₁-C₇-alkoxy; pyrrolyloxy, furanyloxy, thiophenyloxy, imidazolyloxy, pyrazolyloxy, thiazolyloxy, pyrazolidinyloxy, pyrrolidinyloxy, pyridinyloxy, piperidinyloxy, oxopiperidinyloxy, piperazinyloxy, triazolyloxy, morpholinyloxy, thiomorpholinyloxy, S-oxothiomorpholinyloxy, benzimidazolyloxy, pyrrolo-pyrimidinyloxy, or 1H,4H,5H-trihydropyrazolo[2,3-c]piperidin-1-yloxy bound to the “oxy” via a ring carbon and each of which is unsubstituted or substituted by one or more substituents independently selected from C₁-C₇-alkyl, halo-C₁-C₇-alkyl, phenyl, halo-phenyl, hydroxy, C₁-C₇-alkoxy, halo, C₁-C₇-alkoxycarbonyl, carbamoyl, phenylsulfonyl wherein phenyl is unsubstituted or substituted by one or more substituents independently selected from C₁-C₇-alkyl, hydroxy, C₁-C₇-alkoxy, halo, nitro and cyano, piperidinocarbonyl, morpholino-carbonyl, thiomorpholino-carbonyl or S-oxo- or S,S-dioxothiomorpholinocarbonyl, C₁-C₇-alkanoyl, unsubstituted or substituted benzoyl wherein the substituents are preferably one or more substituents independently selected from the group consisting of hydroxy, C₁-C₇-alkoxy and cyano, C₁-C₇-alkanesulfonyl, unsubstituted or substituted benzenesulfonyl wherein the substituents are preferably one or more substituents independently selected from the group consisting of hydroxy, C₁-C₇-alkoxy and cyano, sulfamoyl, N-mono- or N,N-di-(C₁-C₇-alkyl)-sulfamoyl, cyano and nitro; amino; mono- or di-(C₁-C₇-alkyl, C₃-C₈-cyloalkyl and/or hydroxyl-C₁-C₇-alkyl)-amino; mono- or di-(naphthyl- or phenyl-C₁-C₇-alkyl)-amino; C₁-C₇-alkanoylamino; unsubstituted or amino-, N-mono- or N,N-di-(C₁-C₇-alkyl and/or phenyl- or naphthyl-C₁-C₇-alkyl)amino-substituted benzoyl- or naphthoylamino; C₁-C₇-alkoxycarbonylamino; (phenyl or naphthyl)-C₁-C₇-alkoxycarbonylamino; C₁-C₇-alkylsulfonylamino; phenyl- or naphthylsulfonylamino wherein phenyl or naphthyl is unsubstituted or substituted by one or more, especially one to three, C₁-C₇-alkyl moieties; phenyl- or naphthyl-C₁-C₇-alkylsulfonylamino; pyrrolylamino, furanylamino, thiophenylamino, imidazolylamino, pyrazolylamino, thiazolylamino, pyrazolidinylamino, pyrrolidinylamino, pyridinylamino, piperidinylamino, oxopiperidinylamino, piperazinylamino, triazolylamino, morpholinylamino, thiomorpholinylamino, S-oxothiomorpholinylamino, benzimidazolylamino, pyrrolo-pyrimidinylamino or 1H,4H,5H-trihydropyrazolo[2,3-c]piperidin-1-ylamino bound via a ring carbon to the “amino”and each of which is unsubstituted or substituted by one or more substituents independently selected from C₁-C₇-alkyl, halo-C₁-C₇-alkyl, phenyl, halophenyl, hydroxy, C₁-C₇-alkoxy, halo, C₁-C₇-alkoxycarbonyl, carbamoyl, phenylsulfonyl wherein phenyl is unsubstituted or substituted by one or more substituents independently selected from C₁-C₇-alkyl, hydroxy, C₁-C₇-alkoxy, halo, nitro and cyano, piperidinocarbonyl, morpholino-carbonyl, thiomorpholino-carbonyl or S-oxo- or S,S-dioxothiomorpholinocarbonyl, C₁-C₇-alkanoyl, unsubstituted or substituted benzoyl wherein the substituents are one or more substituents independently selected from the group consisting of hydroxy, C₁-C₇-alkoxy and cyano, C₁-C₇-alkanesulfonyl, unsubstituted or substituted benzenesulfonyl wherein the substituents are preferably one or more substituents independently selected from the group consisting of hydroxy, C₁-C₇-alkoxy and cyano, sulfamoyl, N-mono- or N,N-disubstituted sulfamoyl, preferably N-mono- or N,N-di-(C₁-C₇-alkyl)-sulfamoyl, cyano and nitro; C₁-C₇-alkylthio; halo-C₁-C₇-alkylthio; C₁-C₇-alkanesulfonyl; C₃-C₈-cyloalkyl-sulfonyl; C₁-C₇-alkoxy-C₁-C₇-alkylthio; phenyl- or naphthylthio; phenyl- or naphthyl-C₁-C₇-alkylthio; C₁-C₇-alkanoylthio; benzoyl- or naphthylthio; C₁-C₇-alkanoyl; C₁-C₇-alkoxy-C₁-C₇-alkanoyl; unsubstituted or substituted benzoyl wherein the substituents are one or more substituents independently selected from the group consisting of hydroxy, C₁-C₇-alkoxy and cyano; carboxyl; C₁-C₇-alkoxycarbonyl; phenoxy- or naphthoxycarbonyl; phenyl- or naphthyl-C₁-C₇-alkoxycarbonyl; C₁-C₁₀-alkylendioxy; carbamoyl; N-mono- or N,N-di-[C₁-C₇-alkyl, naphthyl-C₁-C₇-alkyl, phenyl-C₁-C₇-alkyl, N′-mono- or N′,N′-di-(C₁-C₇alkyl)amino-C₁-C₇-alkyl, pyrrolidinyl-C₁-C₇-alkyl, piperidinyl-C₁-C₇-alkyl, piperazinyl- or N—(C₁-C₇-alkyl)piperazinyl-C₁-C₇-alkyl, mono-C₁-C₇-alkoxy-C₁-C₇-alkyl, (N′-mono- or N′,N′-di-(C₁-C₇-alkyl)-amino)-C₁-C₇-alkyl, phenyl, pyridinyl, oxazolyl or thiazolyl each of which is unsubstituted or substituted by C₁-C₇-alkoxy, by halo, especially fluoro, by pyrrolidino, by piperidino, by piperazino, by hydroxyl-C₁-C₇-alkylamino, by hydroxyl-C₁-C₇-alkyl, by amino or by N-mono- or N,N-di-(C₁-C₇-alkyl)amino, C₃-C₈-cyloalkyl, pyrrolidinyl, piperidinyl, morpholinyl, piperazinyl, pyrimidinyl, pyrazinyl and/or pyridazinyl]-amino-carbonyl; N—C₁-C₇-alkoxy-C₁-C₇-alkylcarbamoyl; pyrrolidin-1-carbonyl; amino-N-pyrrolidin-1-carbonyl; N-mono- or N,N-di(C₁-C₇-alkyl)amino-pyrrolidin-1-carbonyl; piperidin-1-carbonylmorpholin-4-carbonyl; morpholino-carbonyl, thiomorpholinocarbonyl, S-oxo- or S,S-dioxo-thiomorpholino-carbonyl, thiomorpholin-4-carbonyl; S-oxo-thiomorpholin-4-carbonyl; S,S-dioxothiomorpholin-4-carbonyl; piperazin-1-carbonyl; N—C₁-C₇-alkyl-piperazin-1-carbonyl; N—C₁-C₇-alkoxycarbonyl-piperazin-1-carbonyl; N-mono- or N,N-di-(C₁-C₇-alkyl)-amino-substituted or unsubstituted pyrrolidinyl-C₁-C₇-alkyl-carbonyl; cyano; C₁-C₇-alkenylene or -alkinylene; C₁-C₇-alkylsulfonyl; phenyl- or naphthylsulfonyl wherein phenyl or naphthyl is unsubstituted or substituted by one or more moieties independently selected from the group consisting of C₁-C₇-alkyl, hydroxy, C₁-C₇-alkoxy and cyano; phenyl- or naphthyl-C₁-C₇-alkylsulfonyl; sulfamoyl; N-mono or N,N-di-[C₁-C₇-alkyl, phenyl-, naphthyl-, phenyl-C₁-C₇-alkyl-, pyrrolidinyl-C₁-C₇-alkyl, piperidinyl-C₁-C₇-alkyl, piperazinyl-C₁-C₇-alkyl, N—C₁-C₇-alkylpiperazinyl-C₁-C₇-alkyl, naphthyl-C₁-C₇-alkyl, phenyl which is unsubstituted or substituted by C₁-C₇-alkoxy, by halo, especially fluoro, by pyrrolidino, by piperidino, by piperazino, by hydroxyl-C₁-C₇-alkyl or by N-mono- or N,N-di-(C₁-C₇-alkyl)-C₁-C₇-alkyl; pyrrolidinyl, piperidinyl, piperazinyl, pyridinyl, pyrimidinyl, pyrazinyl, pyridazinyl, oxazolyl and/or thiazolyl]-aminosulfonyl; unsubstituted or substituted heterocyclyl selected from pyrrolyl, furanyl, thiophenyl, pyrazolyl, pyrazolidinyl, pyridinyl that is unsubstituted or substituted by C₁-C₇-alkoxy, by halo-C₁-C₇-alkyl and/or by cyano, pyrrolidinyl, oxopyrrolidinyl, piperidinyl, oxo-piperidinyl, N—C₁-C₇-alkylpiperidinyl, morpholinyl, thiomorpholinyl, S-oxo-thiomorpholinyl, S,S-dioxothiomorpholinyl, piperazinyl, N—C₁-C₇-alkyl-piperazinyl, 4-(phenyl-C₁-C₇-alkyl)-piperazinyl; 4-(naphthyl-C₁-C₇-alkyl)-piperazinyl; 4-(C₁-C₇-alkoxycarbonyl)-piperazinyl, 4-(phenyl-C₁-C₇-alkoxycarbonyl)-piperazinyl, 4-(naphthyl-C₁-C₇-alkoxycarbonyl)-piperazinyl, oxazolyl, thiazolyl, phenylthiazolyl, triazolyl, carbamoyl-triazolyl; pyrazolyl; halo-C₁-C₇alkyl-pyrazolyl; halophenyl-pyrazolyl; pyrimidin-(2-, 4- or 5-)yl, benzimidazolyl, C₁-C₇-alkoxy-substituted benzimidazolyl, pyrrolo-pyrimidinyl, C₁-C₇-alkyl-substituted pyrrolo-pyrimidinyl, 1H,4H,5H-trihydropyrazolo[2,3-c]piperidin-1-ylwhich is unsubstituted or substituted by 1 or 2 substituents independently selected from C₁-C₇-alkyl and halo-C₁-C₇-alkyl, which heterocyclyl is bound via a ring nitrogen atom or via a ring carbon and is unsubstituted or substituted by one or more substituents independently selected from C₁-C₇-alkyl, halo-C₁-C₇-alkyl, phenyl, halophenyl, hydroxy, C₁-C₇-alkoxy, halo, C₁-C₇-alkoxycarbonyl, carbamoyl, phenylsulfonyl wherein phenyl is unsubstituted or substituted by one or more substituents independently selected from C₁-C₇-alkyl, hydroxy, C₁-C₇-alkoxy, halo, nitro and cyano, piperidinocarbonyl, morpholino-carbonyl, thiomorpholino-carbonyl or S-oxo- or S,S-dioxothiomorpholinocarbonyl, C₁-C₇-alkanoyl, unsubstituted or substituted benzoyl wherein the substituents are one or more substituents independently selected from the group consisting of hydroxy, C₁-C₇-alkoxy and cyano, C₁-C₇-alkanesulfonyl, unsubstituted or substituted benzenesulfonyl wherein the substituents are one or more substituents independently selected from the group consisting of hydroxy, C₁-C₇-alkoxy and cyano, sulfamoyl, N-mono- or N,N-di-(C₁-C₇-alkyl)-sulfamoyl, cyano and nitro.

Preferred is a novel compound of the formula I, wherein

X is N, Y is C, (that is a compound of the formula IA given above)

and each of R¹ and R², independently of the other, is unsubstituted or substituted aryl or unsubstituted or substituted heterocyclyl; with the proviso that the compound is different from a compound of the formula IA wherein each of R¹ and R² is unsubstituted 4-pyridyl or from a compound of the formula IA wherein R¹ is 4-pyridyl and R² is morpholino;

or an N-oxide thereof, a solvate and/or a (preferably pharmaceutically acceptable) salt thereof.

More preferred is a novel compound of the formula IA according to the preceding paragraph, wherein especially at least one of R¹ and R² is substituted aryl or substituted heterocyclyl, while the other is unsubstituted or substituted aryl or unsubstituted or substituted heterocyclyl, or an N-oxide thereof, a solvate and/or a (preferably pharmaceutically acceptable) salt thereof.

Also preferred is a novel compound of the formula IA according to the two preceding paragraphs, wherein

unsubstituted or substituted heterocyclyl is a heterocyclic radical selected from the group consisting of oxiranyl, azirinyl, aziridinyl, 1,2-oxathiolanyl, thienyl, furanyl, tetrahydrofuryl, pyranyl, thiopyranyl, thianthrenyl, isobenzofuranyl, benzofuranyl, chromenyl, 2H-pyrrolyl, pyrrolyl, pyrrolinyl, pyrrolidinyl, imidazolyl, imidazolidinyl, benzimidazolyl, pyrazolyl, pyrazinyl, pyrazolidinyl, thiazolyl, isothiazolyl, dithiazolyl, oxazolyl, isoxazolyl, pyridinyl, pyrazinyl, pyrimidinyl, piperidinyl, piperazinyl, pyridazinyl, morpholinyl, thiomorpholinyl, (S-oxo or S,S-dioxo)-thiomorpholinyl, furazanyl, indolizinyl, azepanyl, diazepanyl, isoindolyl, 3H-indolyl, indolyl, benzimidazolyl, indazolyl, triazolyl, tetrazolyl, purinyl, 4H-quinolizinyl, isoquinolyl, quinolyl, tetrahydroquinolyl, tetrahydroisoquinolyl, decahydroquinolyl, octahydroisoquinolyl, benzofuranyl, dibenzofuranyl, benzothiophenyl, dibenzothiophenyl, phthalazinyl, naphthyridinyl, pyrrolo-pyrimidinyl, 1H,4H,5H-trihydropyrazolo[2,3-c]piperidin-1-yl, pyrrolo-pyridinyl, quinoxalyl, quinazolinyl, cinnolinyl, pteridinyl, carbazolyl, beta-carbolinyl, phenanthridinyl, acridinyl, perimidinyl, phenanthrolinyl, phenazinyl, phenothiazinyl, phenoxazinyl, isochromanyl, chromanyl, benzo[1,3]dioxol-5-yl and 2,3-dihydro-benzo[1,4]dioxin-6-yl, 3a,7a-dihydro-3H-imidazo[4,5-b]pyridine-5-yl, 3a,7a-dihydro-1H-pyrrolo[2,3-b]pyridine-5-yl, and 3a,7a-dihydro-1H-pyrazolo[3,4-b]pyridine-5-yl,

each of these radicals being unsubstituted or substituted by one or more substituents independently selected from those mentioned below for substituted aryl;

and unsubstituted or substituted aryl is phenyl, naphthyl, biphenylenyl, indacenyl, acenaphthylenyl, fluorenyl, phenalenyl, phenanthrenyl or anthracenyl which is unsubstituted or substituted by one or more substituents preferably independently selected from the group consisting of C₁-C₇-alkyl, C₂-C₇-alkenyl; C₂-C₇-alkinyl; [pyrrolidinyl, piperidinyl, piperazinyl, morpholino, thiomorpholino, pyridinyl, pyrimidinyl, pyrazinyl, pyridazinyl, oxazolyl or thiazolyl]-C₁-C₇-alkyl wherein pyrrolidinyl, piperidinyl, piperazinyl, pyridinyl, pyrimidinyl, pyrazinyl, pyridazinyl, oxazolyl or thiazolyl are unsubstituted or substituted by C₁-C₇-alkyl, by pyrrolidinyl, by piperazinyl, by amino, by N-mono- and/or N,N-di-C₁-C₇-alkylamino, by halo, by hydroxyl, by C₁-C₇-alkoxy, by oxo and/or by halo-C₁-C₇-alkyl; [pyrrolidinyl, piperidinyl, piperazinyl, pyridinyl, pyrimidinyl, pyrazinyl, pyridazinyl, oxazolyl or thiazolyl]-oxy-C₁-C₇-alkyl wherein pyrrolidinyl, piperidinyl, piperazinyl, pyridinyl, pyrimidinyl, pyrazinyl, pyridazinyl, oxazolyl and thiazolyl are unsubstituted or substituted by C₁-C₇-alkyl, by pyrrolidinyl, by piperazinyl, by amino, by N-mono- and/or N,N-di-C₁-C₇-alkylamino, by halo, by hydroxyl, by C₁-C₇-alkoxy, by oxo and/or by halo-C₁-C₇-alkyl; [pyrrolidin, piperidin, piperazin, pyridin, pyrimidin, pyrazin, pyridazin, oxazoly or thiazol]-carbonyl-C₁-C₇-alkyl wherein pyrrolidin, piperidin, piperazin, pyridin, pyrimidin, pyridazin, oxazol or pyridazin are unsubstituted or substituted by C₁-C₇-alkyl, by pyrrolidinyl, by piperazinyl, by amino, by N-mono- and/or N,N-di-C₁-C₇-alkylamino, by halo, by hydroxyl, by C₁-C₇-alkoxy, by oxo and/or by halo-C₁-C₇-alkyl; halo-C₁-C₇-alkyl; hydroxy-C₁-C₇-alkyl; C₁-C₇-alkoxy-C₁-C₇-alkyl; C₁-C₇-alkoxy-C₁-C₇-alkoxy-C₁-C₇-alkyl; phenyloxy- or naphthyloxy-C₁-C₇-alkyl; phenyl-C₁-C₇-alkoxy- or naphthyl-C₁-C₇-alkoxy-C₁-C₇-alkyl; amino-C₁-C₇-alkyl; N-mono- or N,N-di-(C₁-C₇-alkyl, C₁-C₇-alkoxy-C₁-C₇-alkyl and/or (mono- or di-(C₁-C₇-alkyl)-amino)-C₁-C₇-alkyl)-amino-C₁-C₇-alkyl; C₁-C₇-alkoxy-C₁-C₇-alkylamino-C₁-C₇-alkyl; mono- or di-[C₆-C₁₈-aryl]-C₁-C₇-alkyl in which aryl is phenyl, naphthyl, biphenylenyl, indacenyl, acenaphthylenyl, fluorenyl, phenalenyl, phenanthrenyl or anthracenyl and is unsubstituted or substituted by C₁-C₇-alkyl, by pyrrolidinyl, by piperazinyl, by amino, by N-mono- and/or N,N-di-C₁-C₇-alkylamino, by halo, by hydroxyl, by C₁-C₇-alkoxy and/or by halo-C₁-C₇-alkyl; (naphthyl- or phenyl-C₁-C₇-alkyl)-amino-C₁-C₇-alkyl; C₁-C₇-alkanoylamino-C₁-C₇-alkyl; carboxy-C₁-C₇-alkyl; benzoyl- or naphthoylamino-C₁-C₇-alkyl; C₁-C₇-alkylsulfonylamino-C₁-C₇-alkyl; phenyl- or naphthylsulfonylamino-C₁-C₇-alkyl wherein phenyl or naphthyl is unsubstituted or substituted by one or more C₁-C₇-alkyl moieties; phenyl- or naphthyl-C₁-C₇-alkylsulfonylamino-C₁-C₇-alkyl; cyano-C₁-C₇-alkyl; halo; hydroxy; C₁-C₇-alkoxy; C₆-C₁₈-aryl-C₁-C₇-alkoxy in which aryl is phenyl, naphthyl, biphenylenyl, indacenyl, acenaphthylenyl, fluorenyl, phenalenyl, phenanthrenyl or anthracenyl and is unsubstituted or substituted by C₁-C₇-alkyl, by C₁-C₇-alkoxy, by pyrrolidinyl, by piperazinyl, by amino, by N-mono- and/or N,N-di-C₁-C₇-alkylamino, by halo, by hydroxyl, by C₁-C₇-alkoxy and/or by halo-C₁-C₇-alkyl; hydroxy-C₂-C₇-alkoxy; C₁-C₇-alkoxy-C₁-C₇-alkoxy; C₁-C₇-alkoxy-C₁-C₇-alkoxy-C₁-C₇-alkoxy; halo-C₁-C₇-alkoxy; amino-C₂-C₇-alkoxy; N-mono- or N,N-di-(C₁-C₇-alkyl)-amino-C₁-C₇-alkoxy; N—C₁-C₇-alkanoylamino-C₁-C₇-alkoxy; C₁-C₇-alkoxycarbonyl-amino-C₁-C₇-alkoxy; C₆-C₁₄-arylcarbonylamino-C₂-C₇-alkoxy wherein C₆-C₁₄-aryl is phenyl, naphthyl, biphenylenyl, indacenyl, acenaphthylenyl, fluorenyl, phenalenyl, phenanthrenyl or anthracenyl and is unsubstituted or substituted by one or more, especially up to three, substituents independently selected from the group consisting of C₁-C₇-alkyl, halo-C₁-C₇-alkyl, hydroxy, C₁-C₇-alkoxy, halo and cyano; N-unsubstituted-, N-mono- or N,N-di-(C₁-C₇-alkyl)carbamoyl-C₁-C₇-alkoxy; phenyl- or naphthyloxy; phenyl- or naphthyl-C₁-C₇-alkyloxy; [pyrrolyl, pyrrolidinyl, imidazolyl, imidazolidinyl, piperidinyl, piperazinyl, pyridinyl, pyrimidinyl, pyrazinyl, pyridazinyl, oxazolyl, thiazolyl, morpholinyl, thiomorpholinyl, S-oxothiomorpholinyl or S,S-dioxothiomorpholinyl]-C₁-C₇-alkoxy wherein pyrrolidinyl, piperidinyl, piperazinyl, pyridinyl, pyrimidinyl, pyrazinyl, pyridazinyl, oxazolyl and thiazolyl are unsubstituted or substituted by C₁-C₇-alkyl, by pyrrolidinyl, by piperazinyl, by amino, by N-mono- and/or N,N-di-C₁-C₇-alkylamino, by halo, by hydroxyl, by C₁-C₇-alkoxy, by oxo and/or by halo-C₁-C₇-alkyl; [pyrrolyl, pyrrolidinyl, imidazolyl, imidazolidinyl, piperidinyl, piperazinyl, pyridinyl, pyrimidinyl, pyrazinyl, pyridazinyl, oxazolyl, thiazolyl, morpholinyl, thiomorpholinyl, S-oxothiomorpholinyl or S,S-dioxothiomorpholinyl]-oxy-C₁-C₇-alkoxy wherein pyrrolidinyl, piperidinyl, piperazinyl, pyridinyl, pyrimidinyl, pyrazinyl, pyridazinyl, oxazolyl and thiazolyl are unsubstituted or substituted by C₁-C₇-alkyl, by pyrrolidinyl, by piperazinyl, by amino, by N-mono- and/or N,N-di-C₁-C₇-alkylamino, by halo, by hydroxyl, by C₁-C₇-alkoxy, by oxo and/or by halo-C₁-C₇-alkyl; C₃-C₈-cyloalkoxy; pyridincarbonylamino-C₁-C₇-alkoxy, C₆-C₁₄-arylaminocarbonylamino-C₂-C₇-alkoxy in which aryl is phenyl, naphthyl, biphenylenyl, indacenyl, acenaphthylenyl, fluorenyl, phenalenyl, phenanthrenyl or anthracenyl and is unsubstituted or substituted by one or more substituents independently selected from the group consisting of C₁-C₇-alkyl, halo-C₁-C₇-alkyl, hydroxy, C₁-C₇-alkoxy, halo and cyano; pyridinylaminocarbonylamino-C₁-C₇-alkoxy; C₁-C₇-alkanoyloxy; benzoyl- or naphthoyloxy; carboxy-C₁-C₇-alkoxy; C₁-C₇-alkoxycarbonyl-C₁-C₇-alkoxy; pyrrolyloxy, furanyloxy, thiophenyloxy, imidazolyloxy, pyrazolyloxy, thiazolyloxy, pyrazolidinyloxy, pyrrolidinyloxy, pyridinyloxy, piperidinyloxy, oxopiperidinyloxy, piperazinyloxy, triazolyloxy, morpholinyloxy, thbmorpholinyloxy, S-oxothiomorpholinyloxy, benzimidazolyloxy, pyrrolo-pyrimidinyloxy, or 1H,4H,5H-trihydropyrazolo[2,3-c]piperidin-1-yloxy bound to the “oxy” via a ring carbon and each of which is unsubstituted or substituted by one or more substituents independently selected from C₁-C₇-alkyl, halo-C₁-C₇-alkyl, phenyl, halophenyl, hydroxy, C₁-C₇-alkoxy, halo, C₁-C₇-alkoxycarbonyl, carbamoyl, phenylsulfonyl wherein phenyl is unsubstituted or substituted by one or more substituents independently selected from C₁-C₇-alkyl, hydroxy, C₁-C₇-alkoxy, halo, nitro and cyano, piperidino-carbonyl, morpholino-carbonyl, thiomorpholino-carbonyl or S-oxo- or S,S-dioxothiomorpholinocarbonyl, C₁-C₇-alkanoyl, unsubstituted or substituted benzoyl wherein the substituents are preferably one or more substituents independently selected from the group consisting of hydroxy, C₁-C₇-alkoxy and cyano, C₁-C₇-alkanesulfonyl, unsubstituted or substituted benzenesulfonyl wherein the substituents are preferably one or more substituents independently selected from the group consisting of hydroxy, C₁-C₇-alkoxy and cyano, sulfamoyl, N-mono- or N,N-di-(C₁-C₇-alkyl)-sulfamoyl, cyano and nitro; amino; mono- or di-(C₁-C₇-alkyl, C₃-C₈-cyloalkyl and/or hydroxyl-C₁-C₇-alkyl)-amino; mono- or di-(naphthyl- or phenyl-C₁-C₇-alkyl)-amino; C₁-C₇-alkanoylamino; unsubstituted or amino-, N-mono- or N,N-di-(C₁-C₇-alkyl and/or phenyl- or naphthyl-C₁-C₇alkyl)amino-substituted benzoyl- or naphthoylamino; C₁-C₇-alkoxycarbonylamino; (phenyl or naphthyl)-C₁-C₇-alkoxycarbonylamino; C₁-C₇-alkylsulfonylamino; phenyl- or naphthylsulfonylamino wherein phenyl or naphthyl is unsubstituted or substituted by one or more, especially one to three, C₁-C₇-alkyl moieties; phenyl- or naphthyl-C₁-C₇-alkylsulfonylamino; pyrrolylamino, furanylamino, thiophenylamino, imidazolylamino, pyrazolylamino, thiazolylamino, pyrazolidinylamino, pyrrolidinylamino, pyridinylamino, piperidinylamino, oxopiperidinylamino, piperazinylamino, triazolylamino, morpholinylamino, thiomorpholinylamino, S-oxothiomorpholinylamino, benzimidazolylamino, pyrrolo-pyrimidinylamino or 1H,4H,5H-trihydropyrazolo[2,3-c]piperidin-1-ylamino bound via a ring carbon to the “amino”and each of which is unsubstituted or substituted by one or more substituents independently selected from C₁-C₇-alkyl, halo-C₁-C₇-alkyl, phenyl, halophenyl, hydroxy, C₁-C₇-alkoxy, halo, C₁-C₇-alkoxycarbonyl, carbamoyl, phenylsulfonyl wherein phenyl is unsubstituted or substituted by one or more substituents independently selected from C₁-C₇-alkyl, hydroxy, C₁-C₇-alkoxy, halo, nitro and cyano, piperidinocarbonyl, morpholino-carbonyl, thiomorpholino-carbonyl or S-oxo- or S,S-dioxothiomorpholinocarbonyl, C₁-C₇-alkanoyl, unsubstituted or substituted benzoyl wherein the substituents are one or more substituents independently selected from the group consisting of hydroxy, C₁-C₇-alkoxy and cyano, C₁-C₇-alkanesulfonyl, unsubstituted or substituted benzenesulfonyl wherein the substituents are preferably one or more substituents independently selected from the group consisting of hydroxy, C₁-C₇-alkoxy and cyano, sulfamoyl, N-mono- or N,N-disubstituted sulfamoyl, preferably N-mono- or N,N-di-(C₁-C₇-alkyl)-sulfamoyl, cyano and nitro; C₁-C₇-alkylthio; halo-C₁-C₇-alkylthio; C₁-C₇-alkane-sulfonyl; C₃-C₈-cyloalkyl-sulfonyl; C₁-C₇-alkoxy-C₁-C₇-alkylthio; phenyl- or naphthylthio; phenyl- or naphthyl-C₁-C₇-alkylthio; C₁-C₇-alkanoylthio; benzoyl- or naphthylthio; C₁-C₇-alkanoyl; C₁-C₇-alkoxy-C₁-C₇-alkanoyl; unsubstituted or substituted benzoyl wherein the substituents are one or more substituents independently selected from the group consisting of hydroxy, C₁-C₇-alkoxy and cyano; carboxyl; C₁-C₇-alkoxycarbonyl; phenoxy- or naphthoxycarbonyl; phenyl- or naphthyl-C₁-C₇-alkoxycarbonyl; C₁-C₁₀-alkylendioxy; carbamoyl; N-mono- or N,N-di-[C₁-C₇-alkyl, naphthyl-C₁-C₇-alkyl, phenyl-C₁-C₇-alkyl, N′-mono- or N′,N′-di-(C₁-C₇alkyl)amino-C₁-C₇-alkyl, pyrrolidinyl-C₁-C₇-alkyl, piperidinyl-C₁-C₇-alkyl, piperazinyl- or N—(C₁-C₇-alkyl)piperazinyl-C₁-C₇-alkyl, mono-C₁-C₇-alkoxy-C₁-C₇-alkyl, (N′-mono- or N′,N′-di-(C₁-C₇-alkyl)-amino)-C₁-C₇-alkyl, phenyl, pyridinyl, oxazolyl or thiazolyl each of which is unsubstituted or substituted by C₁-C₇-alkoxy, by halo, especially fluoro, by pyrrolidino, by piperidino, by piperazino, by hydroxyl-C₁-C₇-alkylamino, by hydroxyl-C₁-C₇-alkyl, by amino or by N-mono- or N,N-di-(C₁-C₇-alkyl)amino, C₃-C₈-cyloalkyl, pyrrolidinyl, piperidinyl, morpholinyl, piperazinyl, pyrimidinyl, pyrazinyl and/or pyridazinyl]-amino-carbonyl; N—C₁-C₇-alkoxy-C₁-C₇-alkylcarbamoyl; pyrrolidin-1-carbonyl; amino-N-pyrrolidin-1-carbonyl; N-mono- or N,N-di(C₁-C₇-alkyl)amino-pyrrolidin-1-carbonyl; piperidin-1-carbonylmorpholin-4-carbonyl; morpholinocarbonyl, thiomorpholinocarbonyl, S-oxo- or S,S-dioxo-thiomorpholino-carbonyl, thiomorpholin-4-carbonyl; S-oxo-thiomorpholin-4-carbonyl; S,S-dioxothiomorpholin-4-carbonyl; piperazin-1-carbonyl; N—C₁-C₇-alkyl-piperazin-1-carbonyl; N—C₁-C₇-alkoxycarbonyl-piperazin-1-carbonyl; N-mono- or N,N-di-(C₁-C₇-alkyl)-amino-substituted or unsubstituted pyrrolidinyl-C₁-C₇-alkyl-carbonyl; cyano; C₁-C₇-alkenylene or -alkinylene; C₁-C₇-alkylsulfonyl; phenyl- or naphthylsulfonyl wherein phenyl or naphthyl is unsubstituted or substituted by one or more moieties independently selected from the group consisting of C₁-C₇-alkyl, hydroxy, C₁-C₇-alkoxy and cyano; phenyl- or naphthyl-C₁-C₇-alkylsulfonyl; sulfamoyl; N-mono or N,N-di-[C₁-C₇-alkyl, phenyl-, naphthyl-, phenyl-C₁-C₇-alkyl-, pyrrolidinyl-C₁-C₇-alkyl, piperidinyl-C₁-C₇-alkyl, piperazinyl-C₁-C₇-alkyl, N—C₁-C₇-alkylpiperazinyl-C₁-C₇-alkyl, naphthyl-C₁-C₇-alkyl, phenyl which is unsubstituted or substituted by C₁-C₇-alkoxy, by halo, especially fluoro, by pyrrolidino, by piperidino, by piperazino, by hydroxyl-C₁-C₇-alkyl or by N-mono- or N,N-di-(C₁-C₇-alkyl)-C₁-C₇-alkyl; pyrrolidinyl, piperidinyl, piperazinyl, pyridinyl, pyrimidinyl, pyrazinyl, pyridazinyl, oxazolyl and/or thiazolyl]-aminosulfonyl; unsubstituted or substituted heterocyclyl selected from pyrrolyl, furanyl, thiophenyl, pyrazolyl, pyrazolidinyl, pyridinyl that is unsubstituted or substituted by C₁-C₇-alkoxy, by halo-C₁-C₇-alkyl and/or by cyano, pyrrolidinyl, oxo-pyrrolidinyl, piperidinyl, oxo-piperidinyl, N—C₁-C₇-alkylpiperidinyl, morpholinyl, thiomorpholinyl, S-oxo-thiomorpholinyl, S,S-dioxothiomorpholinyl, piperazinyl, N—C₁-C₇-alkyl-piperazinyl, 4-(phenyl-C₁-C₇-alkyl)-piperazinyl; 4-(naphthyl-C₁-C₇-alkyl)-piperazinyl; 4-(C₁-C₇-alkoxycarbonyl)-piperazinyl, 4-(phenyl-C₁-C₇-alkoxycarbonyl)-piperazinyl, 4-(naphthyl-C₁-C₇-alkoxycarbonyl)-piperazinyl, oxazolyl, thiazolyl, phenylthiazolyl, triazolyl, carbamoyl-triazolyl; pyrazolyl; halo-C₁-C₇-alkyl-pyrazolyl; halophenyl-pyrazolyl; pyrimidin-(2-, 4- or 5-)yl, benzimidazolyl, C₁-C₇-alkoxy-substituted benzimidazolyl, pyrrolo-pyrimidinyl, C₁-C₇-alkyl-substituted pyrrolo-pyrimidinyl, 1H,4H,5H-trihydropyrazolo[2,3-c]piperidin-1-yl which is unsubstituted or substituted by 1 or 2 substituents independently selected from C₁-C₇-alkyl and halo-C₁-C₇-alkyl, which heterocyclyl is bound via a ring nitrogen atom or via a ring carbon and is unsubstituted or substituted by one or more substituents independently selected from C₁-C₇-alkyl, halo-C₁-C₇-alkyl, phenyl, halophenyl, hydroxy, C₁-C₇-alkoxy, halo, C₁-C₇-alkoxycarbonyl, carbamoyl, phenylsulfonyl wherein phenyl is unsubstituted or substituted by one or more substituents independently selected from C₁-C₇-alkyl, hydroxy, C₁-C₇-alkoxy, halo, nitro and cyano, piperidinocarbonyl, morpholino-carbonyl, thiomorpholino-carbonyl or S-oxo- or S,S-dioxothiomorpholinocarbonyl, C₁-C₇-alkanoyl, unsubstituted or substituted benzoyl wherein the substituents are one or more substituents independently selected from the group consisting of hydroxy, C₁-C₇-alkoxy and cyano, C₁-C₇-alkanesulfonyl, unsubstituted or substituted benzenesulfonyl wherein the substituents are one or more substituents independently selected from the group consisting of hydroxy, C₁-C₇-alkoxy and cyano, sulfamoyl, N-mono- or N,N-di-(C₁-C₇-alkyl)-sulfamoyl, cyano and nitro;

and/or an N-oxide thereof, a solvate and/or a (preferably pharmaceutically acceptable) salt thereof.

Highly preferred is a novel compound of the formula IA, wherein

each of R¹ and R², independently of the other, is phenyl, pyridinyl, especially 3-pyridinyl, or pyrrolo[2,3-b]pyridinyl, especially 1H-pyrrolo[2,3-b]pyridine-5-yl, each of which is unsubstituted or substituted by one or more, preferably up to three, substituents independently selected from the group consisting of C₁-C₇-alkyl, especially methyl, halo-C₁-C₇-alkyl, such as trifluoromethyl, furanyl, especially furan-3-yl, pyrrolyl, especially 1H-pyrrol-2-yl, thiophenyl, especially thiophen-3-yl, unsubstituted or cyano-substituted pyridinyl, such as 2-cyano-pyridin-5-yl, morpholinyl, especially morpholino, thiomorpholinyl, especially thiomorpholinyl, S-oxo-thiomorpholinyl, especially S-oxo-thiomorpholino, S,S-dioxo-thiomorpholinyl, especially S,S-dioxothiomorpholino, hydroxyl, C₁-C₇-alkoxy, especially methoxy, hydroxyl-C₂-C₇-alkoxy, such as 2-hydroxyethoxy or 3-hydroxypropoxy, amino-C₂-C₇-alkoxy, such as 2-aminoethoxy or 3-aminopropoxy, C₁-C₇-alkylcarbonylamino-C₁-C₇-alkoxy, such as 3-(cyclopropylcarbonylamino)-propoxy, C₁-C₇-alkoxycarbonylamino-C₁-C₇-alkoxy, such as 2-(tert-butoxycarbonylamino)-ethoxy or 3-(tert-butoxycarbonylamino)-propoxy, C₁-C₇-alkoxycarbonyl-C₁-C₇-alkoxy, such as methoxycarbonylmethoxy, unsubstituted or C₁-C₇-alkyl-substituted piperidinyloxy, such as 1-isopropyl-piperidin-4-yloxy, halo, especially fluoro or chloro, amino, phenyl-C₁-C₇-alkylamino, especially benzylamino, unsubstituted or phenyl-substituted thiazolylamino, especially 4-phenyl-thiazol-2-ylamino, C₁-C₇-alkanoyl, such as acetyl (1-oxoethyl), carboxy, C₁-C₇-alkoxycarbonyl, such as ethoxycarbonyl, carbamoyl, especially N-substituted carbamoyl such as [2-(N-morpholino)ethyl]carbamoyl, C₁-C₇-alkanesulfonyl (C₁-C₇-alkyl-S(═O)₂—) and sulfamoyl, with the proviso that if one of R¹ and R² is 4-pyridyl, the other is phenyl, 3-pyridinyl, 2-pyridinyl or pyrrolo[2,3-b]pyridinyl that is unsubstituted or prefereably substituted as just defined, or the other is 4-pyridyl that is substituted as just defined;

or an N-oxide thereof, a solvate and/or a (preferably pharmaceutically acceptable) salt thereof, or especially the USE thereof according to the invention.

Highly preferred is also the USE according to the invention of a compound of a novel compound of the formula IA as defined in the preceding paragraphs or in the Examples, and/or an N-oxide thereof, a solvate and/or a pharmaceutically acceptable salt thereof.

Highly preferred is also a USE according to the invention of a compound of the formula IB, wherein each of R¹ and R², independently of the other, is phenyl, pyridinyl, especially 3-pyridinyl, or pyrrolo[2,3-b]pyridinyl, especially 1H-pyrrolo[2,3-b]pyridine-5-yl, each of which is unsubstituted or substituted by one or more, preferably up to three, substituents independently selected from the group consisting of C₁-C₇-alkyl, especially methyl, halo-C₁-C₇-alkyl, such as trifluoromethyl, furanyl, especially furan-3-yl, pyrrolyl, especially 1H-pyrrol-2-yl, thiophenyl, especially thiophen-3-yl, unsubstituted or cyano-substituted pyridinyl, such as 2-cyano-pyridin-5-yl, morpholinyl, especially morpholino, thiomorpholinyl, especially thiomorpholinyl, S-oxo-thiomorpholinyl, especially S-oxo-thiomorpholino, S,S-dioxo-thiomorpholinyl, especially S,S-dioxothiomorpholino, hydroxyl, C₁-C₇-alkoxy, especially methoxy, hydroxyl-C₂-C₇-alkoxy, such as 2-hydroxyethoxy or 3-hydroxypropoxy, amino-C₂-C₇-alkoxy, such as 2-aminoethoxy or 3-aminopropoxy, C₁-C₇-alkoxycarbonylamino-C₁-C₇-alkoxy, such as 2-(tert-butoxycarbonylamino)-ethoxy or 3-(tert-butoxycarbonylamino)-propoxy, C₁-C₇-alkoxycarbonyl-C₁-C₇-alkoxy, such as methoxycarbonylmethoxy,unsubstituted or C₁-C₇-alkyl-substituted piperidinyloxy, such as 1-isopropyl-piperidin-4-yloxy, halo, especially fluoro or chloro, amino, phenyl-C₁-C₇-alkylamino, especially benzylamino, unsubstituted or phenyl-substituted thiazolylamino, especially 4-phenyl-thiazol-2-ylamino, C₁-C₇-alkanoyl, such as acetyl (1-oxoethyl), carboxy, C₁-C₇alkoxycarbonyl, such as ethoxycarbonyl, carbamoyl, C₁-C₇-alkanesulfonyl (C₁-C₇-alkyl-S(═O)₂—) and sulfamoyl, or of an N-oxide thereof, a solvate and/or a (preferably pharmaceutically acceptable) salt thereof.

Highly preferred is also a novel compound of the formula IB, wherein

R¹ is phenyl, pyridinyl, especially 3-pyridinyl, or pyrrolo[2,3-b]pyridinyl, especially 1H-pyrrolo[2,3-b]pyridine-5-yl, each of which is substituted by one or more, especially up to three, substituents independently selected from the group consisting of C₁-C₇-alkyl, especially methyl, halo-C₁-C₇-alkyl, such as trifluoromethyl, furanyl, especially furan-3-yl, pyrrolyl, especially 1H-pyrrol-2-yl, thiophenyl, especially thiophen-3-yl, unsubstituted or cyano-substituted pyridinyl, such as 2-cyano-pyridin-5-yl, morpholinyl, especially morpholino, thiomorpholinyl, especially thiomorpholinyl, S-oxo-thiomorpholinyl, especially S-oxo-thiomorpholino, S,S-dioxo-thiomorpholinyl, especially S,S-dioxothiomorpholino, hydroxyl, C₁-C₇-alkoxy, especially methoxy, hydroxyl-C₂-C₇-alkoxy, such as 2-hydroxyethoxy or 3-hydroxypropoxy, amino-C₂-C₇-alkoxy, such as 2-aminoethoxy or 3-aminopropoxy, C₁-C₇-alkoxycarbonylamino-C₁-C₇-alkoxy, such as 2-(tert-butoxycarbonylamino)-ethoxy or 3-(tert-butoxycarbonylamino)-propoxy, C₁-C₇-alkoxycarbonyl-C₁-C₇-alkoxy, such as methoxycarbonylmethoxy, unsubstituted or C₁-C₇-alkyl-substituted piperidinyloxy, such as 1-isopropyl-piperidin-4-yloxy, amino, phenyl-C₁-C₇-alkylamino, especially benzylamino, unsubstituted or phenyl-substituted thiazolylamino, especially 4-phenyl-thiazol-2-ylamino, C₁-C₇-alkanoyl, such as acetyl (1-oxoethyl), carboxy, C₁-C₇-alkoxycarbonyl, such as ethoxycarbonyl, carbamoyl, C₁-C₇-alkanesulfonyl (C₁-C₇-alkyl-S(═O)₂—), sulfamoyl and, in the case of substituted pyridinyl or pyrrolo[2,3-b]pyridinyl (that is, not in the case of substituted phenyl), halo, especially fluoro or chloro, and

R² is phenyl or pyridinyl (the latter especially 3-pyridinyl), each of which is substituted by one or more, especially up to three, substituents independently selected from the group consisting of C₁-C₇-alkyl, especially methyl, halo-C₁-C₇-alkyl, such as trifluoromethyl, furanyl, especially furan-3-yl, pyrrolyl, especially 1H-pyrrol-2-yl, thiophenyl, especially thiophen-3-yl, unsubstituted or cyano-substituted pyridinyl, such as 2-cyano-pyridin-5-yl, morpholinyl, especially morpholino, thiomorpholinyl, especially thiomorpholinyl, S-oxo-thiomorpholinyl, especially S-oxo-thiomorpholino, S,S-dioxo-thiomorpholinyl, especially S,S-dioxothiomorpholino, C₁-C₇-alkoxy, especially methoxy, hydroxyl-C₂-C₇-alkoxy, such as 2-hydroxyethoxy or 3-hydroxypropoxy, amino-C₂-C₇-alkoxy, such as 2-aminoethoxy or 3-aminopropoxy, C₁-C₇-alkoxycarbonylamino-C₁-C₇-alkoxy, such as 2-(tert-butoxycarbonylamino)-ethoxy or 3-(tert-butoxycarbonylamino)-propoxy, C₁-C₇-alkoxycarbonyl-C₁-C₇-alkoxy, such as methoxycarbonylmethoxy, unsubstituted or C₁-C₇-alkyl-substituted piperidinyloxy, such as 1-isopropyl-piperidin-4-yloxy, amino, phenyl-C₁-C₇-alkylamino, especially benzylamino, unsubstituted or phenyl-substituted thiazolylamino, especially 4-phenyl-thiazol-2-ylamino, C₁-C₇-alkanoyl, such as acetyl (1-oxoethyl), carboxy, C₁-C₇-alkoxycarbonyl, such as ethoxycarbonyl, carbamoyl, C₁-C₇-alkanesulfonyl (C₁-C₇-alkyl-S(═O)₂—), sulfamoyl and, in the case of substituted pyridyl (that is, not in the case of substituted phenyl), from hydroxyl and halo, especially fluoro or chloro,

or an N-oxide thereof, a solvate and/or a (preferably pharmaceutically acceptable) salt thereof.

Especially highly preferred is a novel compound of the formula IA or of the formula IB, wherein

R¹ is 1H-pyrrol-2-yl-phenyl, 4-furan-3-yl-phenyl, 4-thiophen-3-yl-phenyl, 4-methoxyphenyl, 3,4-dimethoxyphenyl, 4-(3-amino-propoxy)-3-methoxyphenyl, 4-(3-tert-butoxycarbonylamino-propoxy)-3-methoxyphenyl, 6-(4-phenyl-thiazol-2-ylamino)-pyridin-3-yl, 4-carbamoylphenyl, 4-methanesulfonyl-phenyl, 4-(2-cyanopyridin-5-yl)-phenyl, 6-fluoro-pyridin-3-yl, 6-amino-5-trifluormethyl-pyridin-3-yl, 6-hydroxy-pyridin-3-yl, 6-(1-isopropyl-piperidin-4-yloxy)-pyridin-3-yl, 6-benzylamino-pyridin-3-yl, 6-morpholin-4-yl-pyridin-3-yl or 1H-pyrrolo[2,3-b]pyridin-5-yl, 4-[N-(2-morpholin-4-yl-ethyl)]benzamide, 4-[3-fluoro-N-(2-morpholin-4-yl-ethyl)]benzamide, and

R² is 2-methoxyphenyl, 3,4-dimethoxyphenyl, 4-(3-amino-propoxy)-3-methoxyphenyl, 4-(3-tert-butoxycarbonylamino-propoxy)-3-methoxyphenyl, 3-carbamoyl-4-methoxycarbonylmethoxy-phenyl, 5-ethoxycarbonyl-4-methoxy-phenyl, 3-acetyl-4-(2-hydroxyethoxy)-phenyl, 4-carbamoylphenyl, 3-carbamoyl-4-methoxycarbonylmethoxy-phenyl, 4-sulfamoyl-phenyl or 6-amino-5-trifluormethyl-pyridin-3-yl, 4-[3-(cyclopropylcarbonylamino)-propoxy]phenyl, 2[3-(cyclopropylcarbonylamino)-propoxy]pyridin-5-yl, 3-[phenoxymethyl-4-yl]-oxetan-3-ylamine, cyclopropanecarboxylic acid [3-(phenoxymethyl-4-yl)-oxetan-3-yl]-amide, N-[3-(phenoxymethyl-4-yl)-oxetan-3-yl]-isobutyramide, cyclopropanecarboxylic acid [3-(phenoxymethyl-4-yl)-oxetan-3-ylmethyl]-amide, C-[3-(phenoxynnethyl-4-yl)-oxetan-3-yl]-methylamine, cyclopropanecarboxylic acid ((3-phenoxy-4-yl)-oxetan-3-ylmethyl)-amide, or an N-oxide thereof, a solvate and/or a (preferably pharmaceutically acceptable) salt thereof.

Highly preferred is also the USE according to the invention of a compound of a novel compound of the formula IB as defined in the preceding paragraphs or in the Examples, and/or an N-oxide thereof, a solvate and/or a pharmaceutically acceptable salt thereof.

Very preferred are also embodiments of the invention represented in the claims which are therefore incorporated by reference herein.

Any formula given herein is intended to represent compounds having structures depicted by the structural formula as well as certain variations or forms. In particular, compounds of any formula given herein may have asymmetric centers and therefore exist in different enantiomeric forms. If at least one asymmetrical carbon atom is present in a compound of the formula I, such a compound may exist in optically active form or in the form of a mixture of optical isomers, e. g. in the form of a racemic mixture. All optical isomers and their mixtures, including the racemic mixtures, are part of the present invention. Thus, any given formula given herein is intended to represent a racemate, one or more enantiomeric forms, one or more diastereomeric forms, one or more atropisomeric forms, and mixtures thereof. Furthermore, certain structures may exist as geometric isomers (i.e. cis and trans isomers), as tautomers, or as atropisomers.

Any formula given herein is intended to represent hydrates, solvates, and polymorphs of such compounds, and mixtures thereof.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F ³¹P, ³²P, ³⁵S, ³⁶Cl, ¹²⁵I respectively. Various isotopically labeled compounds of the present invention, for example those into which radioactive isotopes such as ³H, ¹³C, and ¹⁴C are incorporated. Such isotopically labelled compounds are useful in metabolic studies (preferably with ¹⁴C), reaction kinetic studies (with, for example ²H or ³H), detection or imaging techniques [such as positron emission tomography (PET) or single-photon emission computed tomography (SPECT) including drug or substrate tissue distribution assays, or in radioactive treatment of patients. In particular, an ¹⁸F or labeled compound may be particularly preferred for PET or SPECT studies. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this invention and prodrugs thereof can generally be prepared by carrying out the procedures disclosed in the schemes or in the examples and preparations described below by substituting a. readily available isotopically labeled reagent for a non-isotopically labeled reagent.

The invention in one embodiment relates especially to a compound of the formula I as mentioned below in the examples by their names, or a pharmaceutically acceptable salt thereof, and/or a solvate thereof, or its USE according to the invention (that is, not the N-oxides), or, in an alternative embodiment, to an N-oxide of a compound of the formula I, a pharmaceutically acceptable salt thereof and/or a solvate thereof or its USE according to the invention.

In all embodiments of the invention, compounds of the formula I or their USE according to the invention are preferred as such or in the form of pharmaceutically acceptable salts are especially preferred.

Especially preferred is a (novel) compound of the formula I as given in the examples, an N-oxide thereof, a solvate thereof and/or a pharmaceutically acceptable salt thereof.

Quite unexpectedly, it has now been found that the compounds of formula I have advantageous pharmacological properties and inhibit the activity of the lipid kinases, such as the PI3-kinase and/or members of the PI3-kinase-related protein kinase family (also called PIKK and include DNA-PK, ATM, ATR, hSMG-1 and mTOR), such as the DNA protein-kinase, and may be used to treat disease or disorders which depend on the activity of said kinases.

The phosphatidylinositol-3′-OH kinase (PI3K) pathway is one of the central signaling pathways that exerts its effect on numerous cellular functions including cell cycle progression, proliferation, motility, metabolism and survival. An activation of receptor tyrosine kinases causes PI3K to phosphorylate phosphatidylinositol-(4,5)-diphosphate, resulting in membrane-bound phosphatidylinositol-(3,4,5)-triphosphate. The latter promotes the transfer of a variety of protein kinases from the cytoplasm to the plasma membrane by binding of phosphatidylinositol-(3,4,5)-triphosphate to the pleckstrin-homology (PH) domain of the kinase. Kinases that are key downstream targets of PI3K include phosphoinositide-dependent kinase 1 (PDK1) and AKT (also known as Protein Kinase B). Phosphorylation of such kinases then allows for the activation or deactivation of numerous other pathways, involving mediators such as GSK3, mTOR, PRAS40, FKHD, NF-KB, BAD, Caspase-9, and the like. An important negative feedback mechanism for the PI3K pathway is PTEN, a phosphatase that catalyses the dephosphorylation of phosphatidylinositol-(3,4,5)-triphosphate to phosphorylate phosphatidylinositol-(4,5)-diphosphate. In more than 60% of all solid tumors, PTEN is mutated into an inactive form, permitting a constitutive activation of the PI3K pathway. As most cancers are solid tumors, such an observation provides evidence that a targeting of PI3k itself or individual downstream kinases in the PI3K pathway provide a promising approach to mitigate or even abolish the dysregulation in many cancers and thus restore normal cell function and behaviour. This, however, does not exclude that other mechanisms may be responsible for the beneficial effects of PI3K activity modifying agents such as those in the present invention.

Having regard to their inhibitory effect on phosphatidylinositol 3-kinase enzymes, compounds of formula (I) in free or pharmaceutically acceptable salt form, are useful in the treatment of conditions which are mediated by the activation (including normal activity or especially over-activity) of one or more of the members of the PI3 kinase family, especially PI3 kinase enzyme, such as proliferative (especially preferred), inflammatory or allergic conditions, obstructive airways diseases and/or disorders commonly occurring in connection with transplantation.

“Treatment” in accordance with the invention may be therapeutic, e.g. symptomatic, palliative or partially or fully curative, and/or prophylactic. Preferred is the treatment of warm-blooded animals, especially humans.

Preferred is a compound of formula I for use or the USE thereof in the treatment of a proliferative disease selected from a benign or malignant tumor, carcinoma of the brain, kidney, liver, adrenal gland, bladder, breast, stomach, gastric tumors, ovaries, colon, rectum, prostate, pancreas, lung, vagina or thyroid, sarcoma, glioblastomas, multiple myeloma or gastrointestinal cancer, especially colon carcinoma or colorectal adenoma or a tumor of the neck and head, a neoplasia, especially of epithelial character, lymphomas, a mammary carcinoma or a leukemia. Other diseases include Cowden syndrome, Lhermitte-Dudos disease and Bannayan-Zonana syndrome, or in a broader sense an epidermal hyperproliferation, psoriasis or prostate hyperplasia, or diseases in which the PI3K/PKB pathway is aberrantly activated.

Compounds according to the invention are also, in a broader sense, of USE in the treatment of inflammatory or obstructive airways (respiratory tract) diseases, resulting, for example, in reduction of tissue damage, airways inflammation, bronchial hyperreactivity, remodeling or disease progresssion. Inflammatory or obstructive airways diseases to which the present invention is applicable include asthma of whatever type or genesis including both intrinsic (non-allergic) asthma and extrinsic (allergic) asthma, e.g. mild asthma, moderate asthma, severe asthma, bronchitic asthma, exercise-induced asthma, occupational asthma and asthma induced following bacterial infection. Treatment of asthma is also to be understood as embracing treatment of subjects, e.g. of less than 4 or 5 years of age, exhibiting wheezing symptoms and diagnosed or diagnosable as “wheezy infants”, an established patient category of major medical concern and now often identified as incipient or early-phase asthmatics. (For convenience this particular asthmatic condition is referred to as “wheezy-infant syndrome”.)

Prophylactic efficacy in the treatment of asthma can be evidenced by reduced frequency or severity of symptomatic attack, e.g. of acute asthmatic or bronchoconstrictor attack, improvement in lung function or improved airways hyperreactivity. It may further be evidenced by reduced requirement for other, symptomatic therapy, i.e. therapy for or intended to restrict or abort symptomatic attack when it occurs, for example anti-inflammatory (e.g. corticosteroid) or bronchodilatory. Prophylactic benefit in asthma may in particular be apparent in subjects prone to “morning dipping”. “Morning dipping” is a recognised asthmatic syndrome, common to a substantial percentage of asthmatics and characterised by asthma attack, e.g. between the hours of about 4 to 6 am, i.e. at a time normally substantially distant form any previously administered symptomatic asthma therapy.

Compounds of the formula I can, in a broader sense, be of USE for other inflammatory or obstructive airways diseases and conditions to which the present invention is applicable and include acute lung injury (ALI), adult/acute respiratory distress syndrome (ARDS), chronic obstructive pulmonary, airways or Jung disease (COPD, COAD or COLD), including chronic bronchitis or dyspnea associated therewith, emphysema, as well as exacerbation of airways hyperreactivity consequent to other drug therapy, in particular other inhaled drug therapy. The invention, in a broader embodiment, also relates to the USE in treatment of bronchitis of whatever type or genesis including, e.g., acute, arachidic, catarrhal, croupus, chronic or phthinoid bronchitis. Further inflammatory or obstructive airways diseases to which the present invention is applicable include pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis.

Having regard to their anti-inflammatory activity, in particular in relation to inhibition of eosinophil activation, compounds of the invention are, in a broader aspect of the invention, also of USE in the treatment of eosinophil related disorders, e.g. eosinophilia, in particular eosinophil related disorders of the airways (e.g. involving morbid eosinophilic infiltration of pulmonary tissues) including hypereosinophilia as it effects the airways and/or lungs as well as, for example, eosinophil-related disorders of the airways consequential or concomitant to Löffler's syndrome, eosinophilic pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction.

Compounds of the invention are also, in a broader sense of the invention, of USE in the treatment of inflammatory or allergic conditions of the skin, for example psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, pemphigus, epidermolysis bullosa acquisita, and other inflammatory or allergic conditions of the skin.

Compounds of the invention may also, in a broader aspect of the invention, be of USE for the treatment of other diseases or conditions, such as diseases or conditions having an inflammatory component, for example, treatment of diseases and conditions of the eye such as conjunctivitis, keratoconjunctivitis sicca, and vernal conjunctivitis, diseases affecting the nose including allergic rhinitis, and inflammatory disease in which autoimmune reactions are implicated or having an autoimmune component or aetiology, including autoimmune haematological disorders (e.g. haemolytic anaemia, aplastic anaemia, pure red cell anaemia and idiopathic thrombocytopenia), systemic lupus erythematosus, polychondritis, sclerodoma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), endocrine opthalmopathy, Grave's disease, sarcoidosis, alveolitis, chronic hypersensitivity pneumonitis, multiple sclerosis, primary billiary cirrhosis, uveitis (anterior and posterior), keratoconjunctivitis sicca and vernal keratoconjunctivitis, interstitial lung fibrosis, psoriatic arthritis and glomerulonephritis (with and without nephrotic syndrome, e.g. including idiopathic nephrotic syndrome or minimal change nephropathy).

Furthermore, the invention provides the use of a compound according to the definitions herein, an N-oxide, a pharmaceutically acceptable salt, and/or a hydrate or solvate thereof for the preparation of a medicament for the treatment of a proliferative disease, an inflammatory disease, an obstructive respiratory disease, or a disorder commonly occurring in connection with transplantation.

The invention expecially relates to the USE of a compound of the formula I (or a pharmaceutical formulation comprising a compound of the formula I) in the treatment of one or more of the diseases or disorders (especially the preferred ones) mentioned above and below where the disease(s) respond or responds (in a beneficial way, e.g. by partial or complete removal of one or more of its symptoms up to complete cure or remission) to an inhibition of one or more kinases of the PI3-kinase-related protein kinase family, most especially PI3 kinase (PI3K), especially where the kinase shows (in the context of other regulatory mechanisms) inadequately high or more preferably higher than normal (e.g. constitutive) activity.

Whereever the term “use” or “used” or especially USE is mentioned, this is intended to include a compound of the formula I (also the one excluded from the compound per se protection above and in the claims) for use in the prophylactic and/or therapeutic treatment of a disease of a warm-blooded animal, especially a human, preferably of one or more diseases mentioned above or below, a method of use or a method of treatment comprising administering a compound of the formula I to a person in need of such treatment in an effective amount for the prophylactic and/or therapeutic treatment of a disease as mentioned above and below, the preparation or a method for the preparation of a pharmaceutical formulation/preparation for use in the prophylactic and therapeutic treatment of a disease or disorder mentioned above and below, especially involving combining a compound of the formula I (as therapeutically active ingredient) with at least one pharmaceutically acceptable carrier material, preferably including making it ready for use in such treatment (e.g. adding an instruction insert (e.g. package leaflet or the like), formulation, appropriate preparation, adaptation for specific uses, customizing and the like), a pharmaceutical preparation for use or useful in the treatment of a disease or disorder mentioned above or below comprising a compound of the formula I, especially in an amount effective in the treatment of a disease or disorder as mentioned hereinbefore and hereinafter, and/or the use of a compound of the formula I for such preparation, and/or all other prophylactic or therapeutic uses mentioned hereinbefore or below. All these aspects are embodiments of the present invention.

The efficacy of the compounds of formula I and salts thereof as PI3 kinase inhibitors can be demonstrated as follows:

The kinase reaction is performed in a final volume of 50 μL per well of a half area COSTAR, 96 well plate. The final concentrations of ATP and phosphatidyl inositol in the assay are 5 μM and 6 μg/mL respectively. The reaction is started by the addition of PI3 kinase, e.g. PI3 kinase.

p110β. The components of the assay are added per well as follows:
10 μL test compound in 5% DMSO per well in columns 2-1.
Total activity is determined by addition 10 μL of 5% vol/vol DMSO in the first 4 wells of column 1 and the last 4 wells of column 12.
The background is determined by addition of 10 μM control compound to the last 4 wells of column 1 and the first 4 wells of column 12.
2 mL ‘Assay mix’ are prepared per plate:
   1.912 mL of HEPES assay buffer
   8.33 μL of 3 mM stock of ATP giving a final concentration of 5 μM per well
   1 μL of [³³P]ATP on the activity date giving 0.05 μCi per well
   30 μL of 1 mg/mL PI stock giving a final concentration of 6 μg/mL per well
   5 μL of 1 M stock MgCl₂ giving a final concentration of 1 mM per well
20 μL of the assay mix are added per well.
2 mL ‘Enzyme mix’ are prepared per plate (x* μL PI3 kinase p110β in 2 mL of kinase buffer). The ‘Enzyme mix’ is kept on ice during addition to the assay plates.
20 μl ‘Enzyme mix’ are added/well to start the reaction.
The plate is then incubated at room temperature for 90 minutes.
The reaction is terminated by the addition of 50 μL WGA-SPA bead (wheat germ agglutinin-coated Scintillation Proximity Assay beads) suspension per well.
The assay plate is sealed using TopSeal-S)heat seal for polystyrene microplates, PerkinElmer LAS (Deutschland) GmbH, Rodgau, Germany) and incubated at room temperature for at least 60 minutes.
The assay plate is then centrifuged at 1500 rpm for 2 minutes using the Jouan bench top centrifuge (Jouan Inc., Nantes, France).
The assay plate is counted using a Packard TopCount, each well being counted for 20 seconds.
   The volume of enzyme is dependent on the enzymatic activity of the batch in use.

In a more preferred assay, the kinase reaction is performed in a final volume of 10 μL per well of a low volume non binding CORNING, 384 well black plate (Cat. No. #3676). The final concentrations of ATP and phosphatidyl inositol (PI) in the assay are 1 μM and 10 μg/mL respectively. The reaction is started by the addition of ATP.

The components of the assay are added per well as follows:

50 nL test compounds in 90% DMSO per well, in columns 1-20, 8 concentrations (1/3 and 1/3.33 serial dilution step) in single.
Low control: 50 nL of 90% DMSO in half the wells of columns 23-24 (0.45% in final).
High control: 50 nL of reference compound (e.g. compound of Example 7 in WO 2006/122806, incorporated by reference hereinin that regard) in the other half of columns 23-24 (2.5 μM in final).
Standard: 50 nL of reference compound as just mentioned diluted as the test compounds in columns 21-22
20 mL ‘buffer’ are prepared per assay:
   200 μL of 1M TRIS HCl pH7.5 (10 mM in final)
   60 μL of 1M MgCl₂ (3 mM in final)
   500 μL of 2M NaCl (50 mM in final)
   100 μL of 10% CHAPS (0.05% in final)
   200 μL of 100 mM DTT (1 mM in final)
   18.94 mL of nanopure water
10 mL ‘PI’ are prepared per assay:
   200 μL of 1 mg/ml L-alpha-Phosphatidylinositol (Liver Bovine, Avanti Polar Lipids Cat. No. 840042C MW=909.12) prepared in 3% OctylGlucoside (10 μg/ml in final)
   9.8 mL of ‘buffer’
10 mL ‘ATP’ are prepared per assay:
   6.7 μL of 3 mM stock of ATP giving a final concentration of 1 μM per well
   10 mL of ‘buffer’
2.5 mL of each PI3K construct are prepared per assay in ‘PI’ with the following final concentration :
   10 nM PI3K alfa B-1075
   25 nM beta BV-949
   10 nM delta BV-1060
   150 nM gamma BV-950
5 μL of ‘PI/PI3K’ are added per well.
5 μl ‘ATP’ are added per well to start the reaction.
The plates are then incubated at room temperature for 60 minutes (alfa, beta, delta) or 120 minutes (gamma).
The reaction is terminated by the addition of 10 μL Kinase-Glo (Promega Cat. No. #6714).
The assay plates are read after 10 minutes in Synergy 2 reader (BioTek, Vermont USA) with an integration time of 100 milliseconds and sensitivity set to 191.
Output : The High control is around 60'000 counts and the Low control is 30'000 or lower
This luminescence assay gives a useful Z′ ratio between 0.4 and 0.7

The Z′ value is a universal measurement of the robustness of an assay. A Z′ between 0.5 and 1.0 is considered an excellent assay.

For this assay, the PI3K constructs mentioned are prepared as follows:

Molecular Biology:

Two different constructs, BV-1052 and BV-1075, are used to generate the PI3 Kinase a proteins for compound screening.

PI3Kα BV-1052 p85(iSH2)-Gly Linker-p110a(D2Oaa)-C-Term His Tag

PCR products for the inter SH2 domain (iSH2) of the p85 subunit and for the p110-a subunit (with a deletion of the first 20 amino acids) are generated and fused by overlapping PCR. The iSH2 PCR product is generated from first strand cDNA using initially primers gwG130-p01 (5′-CGAGAATATGATAGATTATATGAAGAAT-3′) (SEQ ID NO: 1) and gwG130-p02 (5′-TGGTTT-AATGCTGTTCATACGTTTGTCAAT-3′) (SEQ ID NO: 2).

Subsequently in a secondary PCR reaction, Gateway (Invitrogen AG, Basel, Switzerland) recombination AttB1 sites and linker sequences are added at the 5′end and 3′end of the p85 iSH2 fragment respectively, using primers

gwG130-p03 (5′-GGGACAAGTTTGTACAAAAAAGCAGGCTACGAAGGAGATATACATAT-GCGAGAATATGATAGATTATATGAAGAAT-3′) (SEQ ID NO: 3) and

gwG152-p04 (5′-TACCATAATTCCACCACCACCACCGGAAATTCCCCCTGGTTT-AATGCTGTTCATACGTTTGTCAAT-3′) (SEQ ID NO: 4).

The p110-a fragment is also generated from first strand cDNA, initially using primers

gwG152-p01 (5′-CTAGTGGAATGTTTACTACCAAATGG-3′) (SEQ ID NO: 5) and

gwG152-p02 (5′-GTTCAATG-CATGCTGTTTAATTGTGT-3′) (SEQ ID NO: 6).

In a subsequent PCR reaction, linker sequence and a Histidine tag are added at the 5′end and 3′end of the p110-a fragment respectively, using primers

gw152-p03 (5′-GGGGGAATTTCCGGTGGTGGTGGTGGAATTATGGTAC-TAGTGGAATGTTTACTACC-AAATGGA-3′) (SEQ ID NO: 7) and

gwG152-p06 (5′-AGCTCCGTGATGGTGATGGTGATGTGCTCCGTTCAATG-CATGCTGTTTAATTGTGT-3′) (SEQ ID NO: 8).

The p85-iSH2/p110-a fusion protein is assembled in a third PCR reaction by the overlapping linkers at the 3′end of the iSH2 fragment and the 5′end of the p110-a fragment, using the above mentioned gwG130-p03 primer and a primer containing an overlapping Histidine tag and the AttB2 recombination sequences (5′-GGGACCACTTTGTACAAGAAAGCTGGGTTTAAGCTCCGTGATGGTGATGGTGAT-GTGCTCC-3′) (SEQ ID NO: 9).

This final product is recombined in a (Invitrogen) OR reaction into the donor vector pDONR201 to generate the ORF318 entry clone. This clone is verified by sequencing and used in a Gateway LR reaction to transfer the insert into the Gateway adapted pBlueBac4.5 (Invitrogen) vector for generation of the baculovirus expression vector LR410.

PI3Kα BV-1075 p85(iSH2)-12 XGIv Linker-p110a(D2Oaa)-C-Term His Tap

The construct for Baculovirus BV-1075 is generated by a three-part ligation comprised of a p85 fragment and a p110-a fragment cloned into vector pBlueBac4.5. The p85 fragment is derived from plasmid p1661-2 digested with Nhe/Spe. The p110-a fragment derived from LR410 (see above) as a SpeI/HindIII fragment. The cloning vector pBlueBac4.5 (Invitrogen) is digested with Nhe/HindIII. This results in the construct PED 153.8

The p85 component (iSH2) is generated by PCR using ORF 318 (described above) as a template and one forward primer

KAC1028 (5′-GCTAGCATGCGAGAATATGATAGATTATATGAAGAATATACC) (SEQ ID NO: 10) and two reverse primers,

KAC1029 (5′-GCCTCCACCACCTCCGCCTGGTTTAATGCTGTTCATACGTTTGTC) (SEQ ID NO: 11) and

KAC 1039 (5′-TACTAGTCCGCCTCCACCACCTCCGCCTCCACCACCTCCGCC) (SEQ ID NO: 12).

The two reverse primers overlap and incorporate the 12× Gly linker and the N-terminal sequence of the p110a gene to the Spel site. The 12× Gly linker replaces the linker in the BV1052 construct. The PCR fragment is cloned into pCR2.1 TOPO (Invitrogen). Of the resulting clones, p1661-2 is determined to be correct. This plasmid is digested with Nhe and Spel and the resulting fragment is gel-isolated and purified for sub-cloning.

The p110-a cloning fragment is generated by enzymatic digest of clone LR410 (see above) with Spe I and Hindlll. The Spel site is in the coding region of the p110a gene. The resulting fragment is gel-isolated and purified for sub-cloning.

The cloning vector, pBlueBac4.5 (Invitrogen) is prepared by enzymatic digestion with Nhe and HindIII. The cut vector is purified with Qiagen (Quiagen N.V, Venlo, Netherlands) column and then dephosphorylated with Calf Intestine alkaline phosphatase (CIP) (New England BioLabs, Ipswich, Mass.). After completion of the CIP reaction the cut vector is again column purified to generate the final vector. A 3 part ligation is performed using Roche Rapid ligase and the vendor specifications.

PI3Kβ BV-949 p85(iSH2)-Gly Linker-p110b(Full-Length)-C-Term His Tag

PCR products for the inter SH2 domain (iSH2) of the p85 subunit and for the full-length p110-b subunit are generated and fused by overlapping PCR.

The iSH2 PCR product is generated from first strand cDNA initially using primers

gwG130-p01 (5′-CGAGAATATGATAGATTATATGAAGAAT-3′) (SEQ ID NO: 1) and

gwG130-p02 (5′-TGGTTT-AATGCTGTTCATACGTTTGTCAAT-3′) (SEQ ID NO: 2).

Subsequently, in a secondary PCR reaction Gateway (Invitrogen) recombination AttB1 sites and linker sequences are added at the 5′end and 3′end of the p85 iSH2 fragment respectively, using primers

gwG130-p03 (5′-GGGACAAGTTTGTACAAAAAAGCAGGCTACGAAGGAGATA-TACATATGCGAGAATATGATAGATTATATGAAGAAT-3′) (SEQ ID NO: 3) and

gwG130-p05 (5′-ACTGAAGCATCCTCCTCCTCCTCCTCCTGGTTTAAT-GCTGTTCATACGTTTGTC-3′) (SEQ ID NO: 13).

The p110-b fragment is also generated from first strand cDNA initially using primers

gwG130-p04 (5′-ATTAAACCAGGAGGAGGAGGAGGAGGATGCTTCAGTTTCATAATGCC-TCCTGCT-3′) (SEQ ID NO: 4)

which contains linker sequences and the 5′end of p110-b and

gwG130-p06 (5′-AGCTCCGTGATGGTGATGGTGATGTGCTCCAGATCTGTAGTCTTT-CCGAACTGTGTG-3′) (SEQ ID NO: 14)

which contains sequences of the 3′end of p110-b fused to a Histidine tag.

The p85-iSH2/p110-b fusion protein is assembled by an overlapping PCR a reaction of the linkers at the 3′end of the iSH2 fragment and the 5′end of the p110-b fragment, using the above mentioned gwG130-p03 primer and a primer containing an overlapping Histidine tag and the AttB2 recombination sequences (5′-GGGACCACTTTGTACAAGAAAGCTGGGTTT-AAGCTCCGTGATGGTGATGGTGATGTGCTCC-3′) (SEQ ID NO: 15).

This final product is recombined in a Gateway (Invitrogen) OR reaction into the donor vector pDONR201 to generate the ORF253 entry clone. This clone is verified by sequencing and used in a Gateway LR reaction to transfer the insert into the Gateway adapted pBlueBac4.5 (Invitrogen) vector for generation of the baculovirus expression vector LR280.

PI3Kδ BV-1060 p85(iSH2)-Gly Linker-p110d(Full-Length)-C-Term His Tag

PCR products for the inter SH2 domain (iSH2) of the p85 subunit and for the full-length p110-d subunit are generated and fused by overlapping PCR.

The iSH2 PCR product is generated from first strand cDNA using initially primers

gwG130-p01 (5′-CGAGAATATGATAGATTATATGAAGAAT-3′) (SEQ ID NO: 1) and

gwG130-p02 (5′-TGGTTT-AATGCTGTTCATACGTTTGTCAAT-3′) (SEQ ID NO: 2).

Subsequently, in a secondary PCR reaction Gateway (Invitrogen) recombination AttB1 sites and linker sequences are added at the 5′end and 3′end of the p85 iSH2 fragment respectively, using primers

gwG130-p03 (5′-GGGACAAGTTTGTACAAAAAAGCAGGCTACGAAGGAGATATACAT-ATGCGAGAATATGATAGATTATATGAAGAAT-3′) (SEQ ID NO: 3) and

gwG154-p04 (5′-TCCTCCTCCTCCTCCTCCTGGTTTAATGCTGTTCATACGTTTGTC-3′) (SEQ ID NO: 16).

The p110-a fragment is also generated from first strand cDNA using initially primers

gwG154-p01 (5′-ATGCCCCCTGGGGTGGACTGCCCCAT-3′) (SEQ ID NO: 17) and

gwG154-p02 (5′-CTACTG-CCTGTTGTCTTTGGACACGT-3′) (SEQ ID NO: 18).

In a subsequent PCR reaction linker sequences and a Histidine tag is added at the 5′end and 3′end of the p110-d fragment respectively, using primers

gw154-p03 (5′-ATTAAACCAGGAGGAGGAGGAGGAGGACCCCCTGGGGTGGAC-TGCCCCATGGA-3′) (SEQ ID NO: 19) and gwG154-p06 (5′-AGCTCCGTGATGGTGAT-GGTGATGTGCT-CCCTGCCTGTTGTCTTTGGACACGTTGT-3′) (SEQ ID NO: 20).

The p85-iSH2/p110-d fusion protein is assembled in a third PCR reaction by the overlapping linkers at the 3′end of the iSH2 fragment and the 5′end of the p110-d fragment, using the above mentioned gwG130-p03 primer and a primer containing an overlapping Histidine tag and the Gateway (Invitrogen) AttB2 recombination sequences (5′-GGGACCACTTTGTA-CAAGAAAGCTGGGTTT-AAGCTCCGTGATGGTGATGGTGATGTGCTCC-3′) (SEQ ID NO: 21).

This final product is recombined in a Gateway (Invitrogen) OR reaction into the donor vector pDONR201 to generate the ORF319 entry clone. This clone is verified by sequencing and used in a Gateway LR reaction to transfer the insert into the Gateway adapted pBlueBac4.5 (Invitrogen) vector for generation of the baculovirus expression vector LR415.

PI3Kγ BV-950 p110g(D144aa)-C-Term His Tag

This construct is obtained from Roger Williams lab, MRC Laboratory of Molecular Biology, Cambridge, UK (November, 2003). Description of the construct in: Pacold M. E. et al. (2000) Cell 103, 931-943.

Expression

Methods to generate recombinant baculovirus and protein for PI3K isoforms:

The pBlue-Bac4.5 (for a, b, and d isoforms) or pVL1393 (for g) plasmids containing the different PI3 kinase genes are co-transfected with BaculoGold WT genomic DNA (BD Biosciences, Franklin Lakes, N.J., USA) using methods recommended by the vendor. Subsequently, the recombinant baculovirus obtained from the transfection is plaque-purified on Sf9 insect cells to yield several isolates expressing recombinant protein. Positive clones are selected by anti-HIS or anti-isoform antibody western. For PI3K alpha and delta isoforms, a secondary plaque-purification is performed on the first clonal virus stocks of PI3K. Amplification of all baculovirus isolates is performed at low multiplicity of infection (moi) to generate high-titer, low passage stock for protein production. The baculoviruses are designated BV1052 (a) and BV1075 (a), BV949 ((3), BV1060 (6) and BV950 (y). Protein production involves infection (passage 3 or lower) of suspended Tn5 (Trichoplusia ni) or TiniPro (Expression Systems, LLC, Woodland, Calif., USA) cells in protein-free media at moi of 2-10 for 39-48 hours in 2 L glass Erlenmyer flasks (110 rpm) or wave-bioreactors (22-25 rpm). Initially, 10 L working volume wave-bioreactors are seeded at a density of 3e5 cells/ml at half capacity (5 L). The reactor is rocked at 15 rpm during the cell growth phase for 72 hours, supplemented with 5% oxygen mixed with air (0.2 L per minute). Immediately prior to infection, the wave-reactor cultures are analyzed for density, viability and diluted to approximately 1.5e6 cell/ml. 100-500 ml of high titer, low passage virus is added following 2-4 hours of additional culture. Oxygen is increased to 35% for the 39-48 hour infection period and rocking platform rpm increased to 25. During infection, cells are monitored by Vicell viability analyzer (Beckman Coulter, Inc, Fullerton, Calif., USA) bioprocess for viability, diameter and density. Nova Bioanalyzer (NOVA Biomedical Corp., Waltham, Mass., USA) readings of various parameters and metabolites (pH, O₂ saturation, glucose, etc.) are taken every 12-18 hours until harvest. The wave-bioreactor cells are collected within 40 hours post infection. Cells are collected by centrifugation (4 degrees C. at 1500 rpm), and subsequently maintained on ice during pooling of pellets for lysis and purification. Pellet pools are made with small amounts of cold, un-supplemented Grace's media (w/o protease inhibitors).

PI3K Alpha Purification Protocol For HTS (BV1052)

PI3K alpha is purified in three chromatographic steps: immobilized metal affinity chromatography on a Ni Sepharose resin (GE Healthcare, belonging to General Electric Company, Fairfield, Conn., USA), gel filtration utilizing a Superdex 200 26/60 column (GE Healthcare), and finally a cation exchange step on a SP-XL column (GE Healthcare). All buffers are chilled to 4° C. and lysis is performed chilled on ice. Column fractionation is performed rapidly at room temperature.

Typically frozen insect cells are lysed in a hypertonic lysis buffer and applied to a prepared IMAC column. The resin is washed with 3-5 column volumes of lysis buffer, followed by 3-5 column volumes wash buffer containing 45 mM imidazole, and the target protein is then eluted with a buffer containing 250 mM imidazole. Fractions are analyzed by Coomassie stained SDS-PAGE gels, and fractions containing target protein are pooled and applied to a prepared GFC column. Fractions from the GFC column are analyzed by Coomassie stained SDS-PAGE gels, and fractions containing target protein are pooled. The pool from the GFC column is diluted into a low salt buffer and applied to a prepared SP-XL column. The column is washed with low salt buffer until a stable A280 baseline absorbance is achieved, and eluted using a 20 column volume gradient from 0 mM NaCl to 500 mM NaCl. Again, fractions from the SP-XL column are analyzed by Coomassie stained SDS-PAGE gels, and fractions containing the target protein are pooled. The final pool is dialyzed into a storage buffer containing 50% glycerol and stored at −20° C. The final pool is assayed for activity in a phosphoinosititol kinase assay.

PI3K Beta Purification Protocol For HTS (BV949)

PI3K beta is purified in two chromatographic steps: immobilized metal affinity chromatography (IMAC) on a Ni Sepharose resin (GE Healthcare) and gel filtration (GFC) utilizing a Superdex 200 26/60 column (GE Healthcare). All buffers are chilled to 4° C. and lysis is performed chilled on ice. Column fractionation is performed rapidly at room temperature.

Typically frozen insect cells are lysed in a hypertonic lysis buffer and applied to a prepared IMAC column. The resin is washed with 3-5 column volumes of lysis buffer, followed by 3-5 column volumes wash buffer containing 45 mM imidazole, and the target protein is then eluted with a buffer containing 250 mM imidazole. Fractions are analyzed by Coomassie stained SDS-PAGE gels, and fractions containing target protein are pooled and applied to a prepared GFC column. Fractions from the GFC column are analyzed by Coomassie stained SDS-PAGE gels, and fractions containing target protein are pooled. The final pool is dialyzed into a storage buffer containing 50% glycerol and stored at −20° C. The final pool is assayed for activity in the phosphoinostitol kinase assay.

PI3K Gamma Purification Protocol For HTS (BV950)

PI3K gamma is purified in two chromatographic steps: immobilized metal affinity chromatography (IMAC) on a Ni Sepharose resin (GE Healthcare) and gel filtration (GFC) utilizing a Superdex 200 26/60 column (GE Healthcare). All buffers are chilled to 4° C. and lysis is performed chilled on ice. Column fractionation is performed rapidly at room temperature. Typically frozen insect cells are lysed in a hypertonic lysis buffer and applied to a prepared IMAC column. The resin is washed with 3-5 column volumes of lysis buffer, followed by 3-5 column volumes wash buffer containing 45 mM imidazole, and the target protein is then eluted with a buffer containing 250 mM imidazole. Fractions are analyzed by Coomassie stained SDS-PAGE gels, and fractions containing target protein are pooled and applied to a prepared GFC column. Fractions from the GFC column are analyzed by Coomassie stained SDS-PAGE gels, and fractions containing target protein are pooled. The final pool is dialyzed into a storage buffer containing 50% glycerol and stored at −20° C. The final pool is assayed for activity in the phosphoinostitol kinase assay.

PI3K Delta Purification Protocol For HTS (BV1060)

PI3K delta is purified in three chromatographic steps: immobilized metal affinity chromatography on a Ni Sepharose resin (GE Healthcare), gel filtration utilizing a Superdex 200 26/60 column (GE Healthcare), and finally a anion exchange step on a Q-HP column (GE Healthcare). All buffers are chilled to 4° C. and lysis is performed chilled on ice. Column fractionation is performed rapidly at room temperature. Typically frozen insect cells are lysed in a hypertonic lysis buffer and applied to a prepared IMAC column. The resin is washed with 3-5 column volumes of lysis buffer, followed by 3-5 column volumes wash buffer containing 45 mM imidazole, and the target protein is then eluted with a buffer containing 250 mM imidazole. Fractions are analyzed by Coomassie stained SDS-PAGE gels, and fractions containing the target protein are pooled and applied to a prepared GFC column. Fractions from the GFC column are analyzed by Coomassie stained SDS-PAGE gels, and fractions containing the target protein are pooled. The pool from the GFC column is diluted into a low salt buffer and applied to a prepared Q-HP column. The column is washed with low salt buffer until a stable A280 baseline absorbance is achieved, and eluted using a 20 column volume gradient from 0 mM NaCl to 500 mM NaCl. Again, fractions from the Q-HP column are analyzed by Coomassie stained SDS-PAGE gels, and fractions containing the target protein are pooled. The final pool is dialyzed into a storage buffer containing 50% glycerol and stored at −20° C. The final pool is assayed for activity in the phosphoinostitol kinase assay.

1050 is determined by a four parameter curve fitting routine that comes aloneg with “excel fit”. A 4 Parameter logistic equation is used to calculate IC₅₀ values (IDBS XLfit) of the percentage inhibition of each compound at 8 concentrations (usually 10, 3.0, 1.0, 0.3, 0.1, 0.030, 0.010 and 0.003 pM). Alternatively, IC50 values are calculated using idbsXLfit model 204, which is a 4 parameter logistic model.

Yet alternatively, for an ATP depletion assay, compounds of the formula I to be tested are dissolved in DMSO and directly distributed into a white 384-well plate at 0.5 μL per well. To start the reaction, 10 μL of 10 nM PI3 kinase and 5 μg/mL 1-alpha-phosphatidylinositol (PI) are added into each well followed by 10 μL of 2 μM ATP. The reaction is performed until approx 50% of the ATP is depleted, and then stopped by the addition of 20 μL of Kinase-Glo solution (Promega Corp., Madison, Wis., USA). The stopped reaction is incubated for 5 minutes and the remaining ATP is then detected via luminescence. IC50 values are then determined.

Some of the compounds show a certain level of selectivity against the different paralogs PI3K alpha, beta, gamma and delta.

The range of activity, expressed as IC50, in these assays is preferably between 1 nM and 10 μM, more preferably between 1 nM and about 5 μM.

Description of Biochemical Assay for DNA-PK:

The assay is conducted using the kit V7870 from Promega (SignaTECT® DNA-Dependent Protein Kinase Syste, comprises DNA-PK, biotinylated peptide substrate and further ingredients, Promega, Madison, Wis., USA), that quantitates DNA-dependent protein kinase activity, both in purified enzyme preparations and in cell nuclear extracts. DNA-PK is a nuclear serine/threonine protein kinase that requires double-stranded DNA (dsDNA) for activity. The binding of dsDNA to the enzyme results in the formation of the active enzyme and also brings the substrate closer to the enzyme, allowing the phosphorylation reaction to proceed.

DNA-PK ×5 reaction buffer (250 mM HEPES, 500 mM KCl, 50 mM MgCl₂, 1 mM EGTA, 0.5 mM EDTA, 5 mM DTT, pH to 7.5 with KOH) is diluted 1/5 in deionised water and BSA (stock=10 mg/ml) is added to a final concentration of 0.1 mg/ml.

The activation buffer is made from 100 μg/ml of calf thymus DNA in control buffer (10 mM Tris-HCl (pH 7.4), 1 mM EDTA (pH 8.0)). Per tube, the reaction mix is composed of: 2.5 μl of activation or control buffers, 5 μl of ×5 reaction buffer, 2.5 pl of p53-derived biotinylated peptide substrate (stock=4 mM), 0.2 μl of BSA (stock at 10 mg/ml) and 5 μl of [γ-³²P] ATP (5 μl of 0.5 mM cold ATP+0.05 pl of Redivue [γ-³²P] ATP =Amersham AA0068-250 μCi, 3000Ci/mmol, 10 μCi/μl (now GE Gealthcare Biosciences AB, Uppsala, Sweden).

The DNA-PK enzyme (Promega V5811, concentration=100 UμL) is diluted 1/10 in ×1 reaction buffer and kept on ice until imminent use. 10.8 pl of the diluted enzyme is incubated with 1.2 μl of 100 μM compounds (diluted 1/100 in water from 10 mM stock in neat DMSO) for 10 minutes, at room temperature. During that time, 15.2 μl of the reaction mix is added to screw-capped tubes, behind Perspex glass. 9.8 μl of the enzyme is then transferred to the tubes containing the reaction mix and after 5 minutes incubation, at 30° C., the reaction is stopped by adding 12.5 μl of termination buffer (7.5 M guanidine hydrochloride).

After mixing well, a 10 μl aliquot of each tube is spotted onto a SAM2® biotin capture membrane (Promega, Madison, Wis., USA), which is left to dry for a few minutes. The membrane is then washed extensively to remove the excess free [γ-³²P] ATP and nonbiotinylated proteins: once for 30 seconds in 200 ml of 2M NaCl, 3 times for 2 minutes each in 200 ml of 2M NaCl, 4 times for 2 minutes each in 2M NaCl in 1% H₃PO₄ and twice for 30 seconds each in 100 ml of deionised water. The membrane is subsequently left to air-dry at room temperature for 30-60 minutes.

Each membrane square is separated using forceps and scissors and placed into a scintillation vial, then 8 ml of scintillation liquid (Flo-Scint 6013547 from Perkin-Elmer) is added. The amount of ³²P incorporated into the DNA-PK biotinylated peptide substrate is then determined by liquid scintillation counting. In this test system, compounds of the formula I can be shown to have IC₅₀values in the range from 10 nM to 50 μM, e.g. from 10 nM to 10 μM.

The efficacy of the compounds of the invention in blocking the activation of the PI3K/PKB pathway can be demonstrated in cellular settings as follows:

Protocol for the Detection of Phospho-PKB in U87MG Cells by Elisa:

U87MG cells (human glioblastoma, ATCC No. HTB-14) are trypsinized, counted in a CASY cell counter (Schärffe systems, Gottingen, Germany), diluted in fresh complete DMEM high glucose medium to load, per well, 150 μL cell suspension containing 4×10⁴ cells, and test plates incubated for 18 hours. In parallel, 50 μL of coating antibody, at the desired concentration in PBS/O is loaded in each well of the ELISA plates, and plates are kept for 2 h at room temperature. This ELISA assays is performed in black flat-bottom 96-well plates (Microtest™, Falcon Becton-Dickinson, Ref: 353941) sealed with Plate Sealers (Costar-Corning, Ref: 3095). Medium in plates is discarded and replaced by complete DMEM high glucose medium containing either 0.1% DMSO or 0.1% inhibitor at titers (7) between 10 mM and 0.156 mM in DMSO. After 30 minutes of contact, the medium is quickly removed by aspiration, plates are then placed on ice and immediately cells lyzed with 70 μL of Lysis buffer. In parallel, the 96 wells plates prepared with the coating antibody (1/250 diluted (in PBS/O) Anti-Akt1 C-20, goat, Santa-Cruz-1618, Santa Cruz Biotechnology, Inc., Santa Cruz, Calif., USA) are washed 3 times 1 min with PBS/O containing 0.05% Tween 20 and 0.1% Top-Block® (derivative of gelatine that blocks unspecific binding sites on surfaces; Sigma-Aldrich, Fluka, Buchs, Switzerland, Ref.: 37766), and remaining protein binding sites blocked to prevent non-specific interactions with 200 μL of PBS containing 3% Top Block®, for 2 h at room temperature. Well content is replaced with 50 μL of samples from treated cells, and plates are incubated for 3 h at 4° C. The ELISA assays are always done in parallel with the following controls, in 6 replicates: U87MG (untreated control) or Lysis buffer alone (LB). After 3×15 minutes washes, all wells received 50 μL of the secondary antibody (1/250 diluted (in 3% top block) Anti-S473P-PKB, rabbit, Cell Signaling-9271, Cell Signaling Technologies, Inc., Danvers, Mass., USA)), and are incubated for 16 h at 4° C. After three washes, plates are incubated with the third and conjugated antibody (1/1000 diluted (in 3% top block) anti rabbit (HRP) Jackson Immuno Research 111-035-144) for 2 hours at room temperature. Finally, the immune-complexes are washed 2 times 15 seconds with PBS/O/tween20/top block, 1 time with 200 μl of water and finally 200 μl of water are left in each test well before a for 45 min incubation in darkness. The plates are then assayed with (SuperSignal® ELISA pico Chemiluminescent substrate, Pierce, Ref: 27070, Pierce Biotechnology, Inc., Rockford, Ill., USA). 100 μL of substrate are added, and plates shacked for 1 min. The luminescence is read immediately on a Top-Count NXT (Packard Bioscience) luminometer. Using this test system, IC₅₀ values in the range from 10 μM to 5 nM, more preferably from 5 μM to 10 nM can be found for compounds of the formula I as test compounds.

There are also experiments that can demonstrate the antitumor activity of compounds of the formula I in vivo.

For example, female Harlan (Indianapolis, Ind., USA) athymic nu/nu mice with s.c. transplanted human glioblastoms U87MG tumors can be used to determine the anti-tumor activity of PI3 kinase inhibitors. On day 0, with the animals under peroral Forene® (1-chloro-2,2,2-trifluoroethyldifluormethylether, Abbot, Wiesbaden, Germany) narcosis, a tumor fragment of approximately 25 mg is placed under the skin on the animals' left flank and the small incised wound is closed by means of suture clips. When tumors reach a volume of 100 mm³, the mice are divided at random into groups of 6-8 animals and treatment commences. The treatment is carried out for a 2-3 weeks period with peroral, intravenous or intra-peritoneal administration once daily (or less frequently) of a compound of formula (I) in a suitable vehicle at defined doses. The tumors are measured twice a week with a slide gauge and the volume of the tumors is calculated.

As an alternative to cell line U87MG, other cell lines may also be used in the same manner, for example,
the MDA-MB 468 breast adenocarcinoma cell line (ATCC No. HTB 132; see also In Vitro 14, 911-15 [1978]);
the MDA-MB 231 breast carcinoma cell line (ATCC No. HTB-26; see also In Vitro 12, 331 [1976]);
the MDA-MB 453 breast carcinoma cell line (ATCC No. HTB-131);
the Colo 205 colon carcinoma cell line (ATCC No. CCL 222; see also Cancer Res. 38, 1345-55 [1978]);
the DU145 prostate carcinoma cell line DU 145 (ATCC No. HTB 81; see also Cancer Res. 37, 4049-58 [1978]),
the PC-3 prostate carcinoma cell line PC-3 (especially preferred; ATCC No. CRL 1435; see also Cancer Res. 40, 524-34 [1980]) and the PC-3M prostate carcinoma cell line;
the A549 human lung adenocarcinoma (ATCC No. CCL 185; see also Int. J. Cancer 17, 62-70 [1976]),
the NCI-H596 cell line (ATCC No. HTB 178; see also Science 246, 491-4 [1989]);
the pancreatic cancer cell line SUIT-2 (see Tomioka et al., Cancer Res. 61, 7518-24 [2001]).

Compounds of the invention exhibit T cell inhibiting activity. More particular the compounds of the invention prevent T cell activation and/or proliferation in e.g. aqueous solution, e.g. as demonstrated in accordance with the following test method. The two-way MLR is performed according to standard procedures (J. Immunol. Methods, 1973, 2, 279 and Meo T. et al., Immunological Methods, New York, Academic Press, 1979, 227-39). Briefly, spleen cells from CBA and BALB/c mice (1.6×105 cells from each strain per well in flat bottom tissue culture microtiter plates, 3.2×105 in total) are incubated in RPMI medium containing 10% FCS, 100 U/ml penicillin, 100 μg/ml streptomycin (Gibco BRL, Basel, Switzerland), 50 μM 2-mercaptoethanol (Fluka, Buchs, Switzerland) and serially diluted compounds. Seven three-fold dilution steps in duplicates per test compound are performed. After four days of incubation, 1 μCi 3H-thymidine is added. Cells are harvested after an additional five-hour incubation period, and incorporated 3H-thymidine is determined according to standard procedures. Background values (low control) of the MLR are the proliferation of BALB/c cells alone. Low controls are subtracted from all values. High controls without any sample are taken as 100% proliferation. Percent inhibition by the samples is calculated, and the concentrations required for 50% inhibition (IC50 values) are determined. In this assay, the compounds of the invention preferably have IC50 values in the range of 10 nM to 5 μM, preferably from 10 nM to 500 nM.

A compound of the formula I may also be used to advantage in combination with other anti-proliferative compounds. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibittors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics; temozolomide (TEMODAL®); kinesin spindle protein inhibitors, such as SB715992 or SB743921 from GlaxoSmithKline, or pentamidine/chlorpromazine from CombinatoRx; MEK inhibitors such as ARRY142886 from Array PioPharma, AZD6244 from AstraZeneca, PD181461 from Pfizer, leucovorin, EDG binders, antileukemia compounds, ribonucleotide reductase inhibitors, S-adenosylmethionine decarboxylase inhibitors, antiproliferative antibodies or other chemotherapeutic compounds. Further, alternatively or in addition they may be used in combination with other tumor treatment approaches, including surgery, ionizing radiation, photodynamic therapy, implants, e.g. with corticosteroids, hormones, or they may be used as radiosensitizers. Also, in anti-inflammatory and/or antiproliferative treatment, combination with anti-inflammatory drugs is included. Combination is also possible with antihistamine drug substances, bronchodilatatory drugs, NSAID or antagonists of chemokine receptors.

The term “aromatase inhibitor” as used herein relates to a compound which inhibits the estrogen production, i.e. the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane can be administered, e.g., in the form as it is marketed, e.g. under the trademark AROMASIN. Formestane can be administered, e.g., in the form as it is marketed, e.g. under the trademark LENTARON. Fadrozole can be administered, e.g., in the form as it is marketed, e.g. under the trademark AFEMA. Anastrozole can be administered, e.g., in the form as it is marketed, e.g. under the trademark ARIMIDEX. Letrozole can be administered, e.g., in the form as it is marketed, e.g. under the trademark FEMARA or FEMAR. Aminoglutethimide can be administered, e.g., in the form as it is marketed, e.g. under the trademark ORIMETEN. A combination of the invention comprising a chemotherapeutic agent which is an aromatase inhibitor is particularly useful for the treatment of hormone receptor positive tumors, e.g. breast tumors.

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen can be administered, e.g., in the form as it is marketed, e.g. under the trademark NOLVADEX. Raloxifene hydrochloride can be administered, e.g., in the form as it is marketed, e.g. under the trademark EVISTA. Fulvestrant can be formulated as disclosed in U.S. Pat. No. 4,659,516 or it can be administered, e.g., in the form as it is marketed, e.g. under the trademark FASLODEX. A combination of the invention comprising a chemotherapeutic agent which is an antiestrogen is particularly useful for the treatment of estrogen receptor positive tumors, e.g. breast tumors.

The term “anti-androgen” as used herein relates to any substance which is capable of inhibiting the biological effects of androgenic hormones and includes, but is not limited to, bicalutamide (CASODEX), which can be formulated, e.g. as disclosed in U.S. Pat. No. 4,636,505.

The term “gonadorelin agonist” as used herein includes, but is not limited to abarelix, goserelin and goserelin acetate. Goserelin is disclosed in U.S. Pat. No. 4,100,274 and can be administered, e.g., in the form as it is marketed, e.g. under the trademark ZOLADEX. Abarelix can be formulated, e.g. as disclosed in U.S. Pat. No. 5,843,901.

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148 (compound A1 in WO99/17804). Irinotecan can be administered, e.g. in the form as it is marketed, e.g. under the trademark CAMPTOSAR. Topotecan can be administered, e.g., in the form as it is marketed, e.g. under the trademark HYCAMTIN.

The term “topoisomerase H inhibitor” as used herein includes, but is not limited to the anthracyclines such as doxorubicin (including liposomal formulation, e.g. CAELYX), daunorubicin, epirubicin, idarubicin and nemorubicin, the anthraquinones mitoxantrone and losoxantrone, and the podophillotoxines etoposide and teniposide. Etoposide can be administered, e.g. in the form as it is marketed, e.g. under the trademark ETOPOPHOS. Teniposide can be administered, e.g. in the form as it is marketed, e.g. under the trademark VM 26-BRISTOL. Doxorubicin can be administered, e.g. in the form as it is marketed, e.g. under the trademark ADRIBLASTIN or ADRIAMYCIN. Epirubicin can be administered, e.g. in the form as it is marketed, e.g. under the trademark FARMORUBICIN. Idarubicin can be administered, e.g. in the form as it is marketed, e.g. under the trademark ZAVEDOS. Mitoxantrone can be administered, e.g. in the form as it is marketed, e.g. under the trademark NOVANTRON.

The term “microtubule active compound” relates to microtubule stabilizing, microtubule destabilizing compounds and microtublin polymerization inhibitors including, but not limited to taxanes, e.g. paclitaxel and docetaxel, vinca alkaloids, e.g., vinblastine, especially vinblastine sulfate, vincristine especially vincristine sulfate, and vinorelbine, discodermolides, colchicine and epothilones and derivatives thereof, e.g. epothilone B or D or derivatives thereof. Paclitaxel may be administered e.g. in the form as it is marketed, e.g. TAXOL. Docetaxel can be administered, e.g., in the form as it is marketed, e.g. under the trademark TAXOTERE. Vinblastine sulfate can be administered, e.g., in the form as it is marketed, e.g. under the trademark VINBLASTIN R.P. Vincristine sulfate can be administered, e.g., in the form as it is marketed, e.g. under the trademark FARMISTIN. Discodermolide can be obtained, e.g., as disclosed in U.S. Pat. No. 5,010,099. Also included are Epothilone derivatives which are disclosed in WO 98/10121, U.S. Pat. No. 6,194,181, WO 98/25929, WO 98/08849, WO 99/43653, WO 98/22461 and WO 00/31247. Especially preferred are Epothilone A and/or B.

The term “alkylating compound” as used herein includes, but is not limited to, cyclophosphamide, ifosfamide, melphalan or nitrosourea (BCNU or Gliadel). Cyclophosphamide can be administered, e.g., in the form as it is marketed, e.g. under the trademark CYCLOSTIN. Ifosfamide can be administered, e.g., in the form as it is marketed, e.g. under the trademark HOLOXAN.

The term “histone deacetylase inhibitors” or “HDAC inhibitors” relates to compounds which inhibit the histone deacetylase and which possess antiproliferative activity. This includes compounds disclosed in WO 02/22577, especially N-hydroxy-3-[4-[[(2-hydroxyethyl)[2-(1H-indol-3-yl)ethyl]-amino]methyl]phenyl]-2E-2-propenamide, N-hydroxy-3-[4-[[[2-(2-methyl-1H-indol-3-yl)-ethyl]-amino]methyl]phenyl]-2E-2-propenamide and pharmaceutically acceptable salts thereof. It further especially includes Suberoylanilide hydroxamic acid (SAHA).

The term “antineoplastic antimetabolite” includes, but is not limited to, 5-Fluorouracil or 5-FU, capecitabine, gemcitabine, DNA demethylating compounds, such as 5-azacytidine and decitabine, methotrexate and edatrexate, and folic acid antagonists such as pemetrexed. Capecitabine can be administered, e.g., in the form as it is marketed, e.g. under the trademark XELODA. Gemcitabine can be administered, e.g., in the form as it is marketed, e.g. under the trademark GEMZAR.

The term “platin compound” as used herein includes, but is not limited to, carboplatin, cisplatin, cisplatinum and oxaliplatin. Carboplatin can be administered, e.g., in the form as it is marketed, e.g. under the trademark CARBOPLAT. Oxaliplatin can be administered, e.g., in the form as it is marketed, e.g. under the trademark ELOXATIN.

The term “compounds targeting/decreasing a protein or lipid kinase activity”; or a “protein or lipid phosphatase activity”; or “further anti-angiogenic compounds” as used herein includes, but is not limited to, protein tyrosine kinase and/or serine and/or threonine kinase inhibitors or lipid kinase inhibitors, e.g.,
a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, e.g. a N-phenyl-2-pyrimidine-amine derivative, e.g. imatinib, SU101, SU6668 and GFB-111;
b) compounds targeting, decreasing or inhibiting the activity of the fibroblast growth factor-receptors (FGFR);
c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, such as those compounds disclosed in WO 02/092599, or antibodies that target the extracellular domain of IGF-I receptor or its growth factors;
d) compounds targeting, decreasing or inhibiting the activity of the Trk receptor tyrosine kinase family, or ephrin B4 inhibitors;
e) compounds targeting, decreasing or inhibiting the activity of the Axl receptor tyrosine kinase family;
f) compounds targeting, decreasing or inhibiting the activity of the Ret receptor tyrosine kinase;
g) compounds targeting, decreasing or inhibiting the activity of the Kit/SCFR receptor tyrosine kinase, e.g. imatinib;
h) compounds targeting, decreasing or inhibiting the activity of the C-kit receptor tyrosine kinases—(part of the PDGFR family), such as compounds which target, decrease or inhibit the activity of the c-Kit receptor tyrosine kinase family, especially compounds which inhibit the c-Kit receptor, e.g. imatinib;
i) compounds targeting, decreasing or inhibiting the activity of members of the c-Abl family, their gene-fusion products (e.g. BCR-Abl kinase) and mutants, such as compounds which target decrease or inhibit the activity of c-Abl family members and their gene fusion products, e.g. a N-phenyl-2-pyrimidine-amine derivative, e.g. imatinib or nilotinib (AMN107); PD180970; AG957; NSC 680410; PD173955 from ParkeDavis; or dasatinib (BMS-354825)
j) compounds targeting, decreasing or inhibiting the activity of members of the protein kinase C (PKC) and Raf family of serine/threonine kinases, members of the MEK, SRC, JAK, FAK, PDK1, PKB/Akt, and Ras/MAPK family members, and/or members of the cyclin-dependent kinase family (CDK) and are especially those staurosporine derivatives disclosed in U.S. Pat. No. 5,093,330, e.g. midostaurin; examples of further compounds include e.g. UCN-01, safingol, BAY 43-9006, Bryostatin 1, Perifosine; Ilmofosine; RO 318220 and RO 320432; GO 6976; Isis 3521; LY333531/LY379196; isochinoline compounds such as those disclosed in WO 00/09495; FTIs; PD184352 or QAN697 (a PI3K inhibitor) or AT7519 (CDK inhibitor);
k) compounds targeting, decreasing or inhibiting the activity of protein-tyrosine kinase inhibitors, such as compounds which target, decrease or inhibit the activity of protein-tyrosine kinase inhibitors include imatinib mesylate (GLEEVEC) or tyrphostin. A tyrphostin is preferably a low molecular weight (Mr<1500) compound, or a pharmaceutically acceptable salt thereof, especially a compound selected from the benzylidenemalonitrile class or the S-arylbenzenemalonirile or bisubstrate quinoline class of compounds, more especially any compound selected from the group consisting of Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin);
l) compounds targeting, decreasing or inhibiting the activity of the epidermal growth factor family of receptor tyrosine kinases (EGFR, ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, e.g. EGF receptor, ErbB2, ErbB3 and ErbB4 or bind to EGF or EGF related ligands, and are in particular those compounds, proteins or monoclonal antibodies generically and specifically disclosed in WO 97/02266, e.g. the compound of ex. 39, or in EP 0 564 409, WO 99/03854, EP 0520722, EP 0 566 226, EP 0 787 722, EP 0 837 063, U.S. Pat. No. 5,747,498, WO 98/10767, WO 97/30034, WO 97/49688, WO 97/38983 and, especially, WO 96/30347 (e.g. compound known as CP 358774), WO 96/33980 (e.g. compound ZD 1839) and WO 95/03283 (e.g. compound ZM105180); e.g. trastuzumab (Herceptin™), cetuximab (Erbitux™), Iressa, Tarceva, OSI-774, CI-1033, EKB-569, GW-2016, E1.1, E2.4, E2.5, E6.2, E6.4, E2.11, E6.3 or E7.6.3, and 7H-pyrrolo-[2,3-d]pyrimidine derivatives which are disclosed in WO 03/013541; and
m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF.

Further anti-angiogenic compounds include compounds having another mechanism for their activity, e.g. unrelated to protein or lipid kinase inhibition e.g. thalidomide (THALOMID) and TNP-470.

Compounds which target, decrease or inhibit the activity of a protein or lipid phosphatase are e.g. inhibitors of phosphatase 1, phosphatase 2A, or CDC25, e.g. okadaic acid or a derivative thereof. Compounds which induce cell differentiation processes are e.g. retinoic acid, α- γ- or δ-tocopherol or α- γ- or δ-tocotrienol.

The term cyclooxygenase inhibitor as used herein includes, but is not limited to, e.g. Cox-2 inhibitors, 5-alkyl substituted 2-arylaminophenylacetic acid and derivatives, such as celecoxib (CELEBREX), rofecoxib (VIOXX), etoricoxib, valdecoxib or a 5-alkyl-2-arylaminophenylacetic acid, e.g. 5-methyl-2-(2′-chloro-6′-fluoroanilino)phenyl acetic acid, lumiracoxib. The term “bisphosphonates” as used herein includes, but is not limited to, etridonic, clodronic, tiludronic, pamidronic, alendronic, ibandronic, risedronic and zoledronic acid. “Etridonic acid” can be administered, e.g., in the form as it is marketed, e.g. under the trademark DIDRONEL. “Clodronic acid” can be administered, e.g., in the form as it is marketed, e.g. under the trademark BONEFOS. “Tiludronic acid” can be administered, e.g., in the form as it is marketed, e.g. under the trademark SKELID. “Pamidronic acid” can be administered, e.g. in the form as it is marketed, e.g. under the trademark AREDIA™. “Alendronic acid” can be administered, e.g., in the form as it is marketed, e.g. under the trademark FOSAMAX. “Ibandronic acid” can be administered, e.g., in the form as it is marketed, e.g. under the trademark BONDRANAT. “Risedronic acid” can be administered, e.g., in the form as it is marketed, e.g. under the trademark ACTONEL. “Zoledronic acid” can be administered, e.g. in the form as it is marketed, e.g. under the trademark ZOMETA.

The term “mTOR inhibitors” relates to compounds which inhibit the mammalian target of rapamycin (mTOR) and which possess antiproliferative activity such as sirolimus (Rapamune®), everolimus (CerticanT™), CCI-779 and ABT578.

The term “heparanase inhibitor” as used herein refers to compounds which target, decrease or inhibit heparin sulfate degradation. The term includes, but is not limited to, PI-88.

The term “ biological response modifier” as used herein refers to a lymphokine or interferons, e.g. interferon γ.

The term “inhibitor of Ras oncogenic isoforms”, e.g. H-Ras, K-Ras, or N-Ras, as used herein refers to compounds which target, decrease or inhibit the oncogenic activity of Ras e.g. a “farnesyl transferase inhibitor” e.g. L-744832, DK8G557 or R115777 (Zarnestra).

The term “telomerase inhibitor” as used herein refers to compounds which target, decrease or inhibit the activity of telomerase. Compounds which target, decrease or inhibit the activity of telomerase are especially compounds which inhibit the telomerase receptor, e.g. telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase are e.g. bengamide or a derivative thereof.

The term “proteasome inhibitor” as used herein refers to compounds which target, decrease or inhibit the activity of the proteasome. Compounds which target, decrease or inhibit the activity of the proteasome include e.g. Bortezomid (Velcade™) and MLN 341.

The term “matrix metalloproteinase inhibitor” or (“MMP” inhibitor) as used herein includes, but is not limited to, collagen peptidomimetic and nonpeptidomimetic inhibitors, tetracycline derivatives, e.g. hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “compounds used in the treatment of hematologic malignancies” as used herein includes, but is not limited to, FMS-like tyrosine kinase inhibitors e.g. compounds targeting, decreasing or inhibiting the activity of FMS-like tyrosine kinase receptors (Flt-3R); interferon, 1-b-D-arabinofuransylcytosine (ara-c) and bisulfan; and ALK inhibitors e.g. compounds which target, decrease or inhibit anaplastic lymphoma kinase.

Compounds which target, decrease or inhibit the activity of FMS-like tyrosine kinase receptors (Flt-3R) are especially compounds, proteins or antibodies which inhibit members of the Flt-3R receptor kinase family, e.g. PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway.

Compounds targeting, decreasing or inhibiting the intrinsic ATPase activity of HSP90 are especially compounds, proteins or antibodies which inhibit the ATPase activity of HSP90 e.g., 17-allylamino,17-demethoxygeldanamycin (17AAG), a geldanamycin derivative; other geldanamycin related compounds; radicicol and HDAC inhibitors.

The term “antiproliferative antibodies” as used herein includes, but is not limited to, trastuzumab (Herceptin™), trastuzumab-DM1, erbitux, bevacizumab (Avastin™), rituximab (Rituxan®), PRO64553 (anti-CD40) and 2C4 Antibody. By antibodies is meant e.g. intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds of formula (I) can be used in combination with standard leukemia therapies, especially in combination with therapies used for the treatment of AML. In particular, compounds of formula (I) can be administered in combination with, e.g., farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412.

The term “antileukemic compounds” includes, for example, Ara-C, a pyrimidine analog, which is the 2′-alpha-hydroxy ribose (arabinoside) derivative of deoxycytidine. Also included is the purine analog of hypoxanthine, 6-mercaptopurine (6-MP) and fludarabine phosphate. Compounds which target, decrease or inhibit activity of histone deacetylase (HDAC) inhibitors such as sodium butyrate and suberoylanilide hydroxamic acid (SAHA) inhibit the activity of the enzymes known as histone deacetylases. Specific HDAC inhibitors include MS275, SAHA, FK228 (formerly FR901228), Trichostatin A and compounds disclosed in U.S. Pat. No. 6,552,065, in particular, N-hydroxy-3-[4-[[[2-(2-methyl-1H-indol-3-yl)-ethyl]-amino]methyl]phenyl]-2E-2-propenamide, or a pharmaceutically acceptable salt thereof and N-hydroxy-3-[4-[(2-hydroxyethyl){2-(1H-indol-3-yl)ethyl]-amino]methyl]phenyl]-2E-2-propenamide, or a pharmaceutically acceptable salt thereof, especially the lactate salt.

Somatostatin receptor antagonists as used herein refers to compounds which target, treat or inhibit the somatostatin receptor such as octreotide, and SOM230 (pasireotide).

Tumor cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4^{th} Edition, Vol. 1, pp. 248-275 (1993).

The term “EDG binders” as used herein refers a class of immunosuppressants that modulates lymphocyte recirculation, such as FTY720.

The term “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives, such as PL-1, PL-2, PL-3, PL-4, PL-5, PL-6, PL-7 or PL-8 mentioned in Nandy et al., Acta Oncologica, Vol. 33, No. 8, pp. 953-961 (1994).

The term “S-adenosylmethionine decarboxylase inhibitors” as used herein includes, but is not limited to the compounds disclosed in U.S. Pat. No. 5,461,076.

Also included are in particular those compounds, proteins or monoclonal antibodies of VEGF disclosed in WO 98/35958, e.g. 1-(4-chloroanilino)-4-(4-pyridylmethyl)phthalazine or a pharmaceutically acceptable salt thereof, e.g. the succinate, or in WO 00/09495, WO 00/27820, WO 00/59509, WO 98/11223, WO 00/27819 and EP 0 769 947; those as described by Prewett et al, Cancer Res, Vol. 59, pp. 5209-5218 (1999); Yuan et al., Proc Natl Acad Sci USA, Vol. 93, pp. 14765-14770 (1996); Zhu et al., Cancer Res, Vol. 58, pp. 3209-3214 (1998); and Mordenti et al., Toxicol Pathol, Vol. 27, No. 1, pp. 14-21 (1999); in WO 00/37502 and WO 94/10202; ANGIOSTATIN, described by O'Reilly et al., Cell, Vol. 79, pp. 315-328 (1994); ENDOSTATIN, described by O'Reilly et al., Cell, Vol. 88, pp. 277-285 (1997); anthranilic acid amides; ZD4190; ZD6474; SU5416; SU6668; bevacizumab; or anti-VEGF antibodies or anti-VEGF receptor antibodies, e.g. rhuMAb and RHUFab, VEGF aptamer e.g. Macugon; FLT-4 inhibitors, FLT-3 inhibitors, VEGFR-2 IgG1 antibody, Angiozyme (RPI 4610) and Bevacizumab (Avastin™)

Photodynamic therapy as used herein refers to therapy which uses certain chemicals known as photosensitizing compounds to treat or prevent cancers. Examples of photodynamic therapy includes treatment with compounds, such as e.g. VISUDYNE and porfimer sodium. Angiostatic steroids as used herein refers to compounds which block or inhibit angiogenesis, such as, e.g., anecortave, triamcinolone. hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to compounds, such as e.g. fluocinolone, dexamethasone.

“Other chemotherapeutic compounds” include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

The compounds of the invention are also useful as co-therapeutic compounds for use in combination with other drug substances such as anti-inflammatory, bronchodilatory or anti-histamine drug substances, particularly in the treatment of obstructive or inflammatory airways diseases such as those mentioned hereinbefore, for example as potentiators of therapeutic activity of such drugs or as a means of reducing required dosaging or potential side effects of such drugs. A compound of the invention may be mixed with the other drug substance in a fixed pharmaceutical composition or it may be administered separately, before, simultaneously with or after the other drug substance. Accordingly the invention includes a combination of a compound of the invention as hereinbefore described with an anti-inflammatory, bronchodilatory, antihistamine or anti-tussive drug substance, said compound of the invention and said drug substance being in the same or different pharmaceutical composition.

Suitable anti-inflammatory drugs include steroids, in particular glucocorticosteroids such as budesonide, beclamethasone dipropionate, fluticasone propionate, ciclesonide or mometasone furoate, or steroids described in WO 02/88167, WO 02/12266, WO 02/100879, WO 02/00679 (especially those of Examples 3, 11, 14, 17, 19, 26, 34, 37, 39, 51, 60, 67, 72, 73, 90, 99 and 101), WO 03/035668, WO 03/048181, WO 03/062259, WO 03/06444 5, WO 03/072592, non-steroidal glucocorticoid receptor agonists such as those described in WO 00/00531, WO 02/10143, WO 03/082280, WO 03/082787, WO 03/104195, WO 04/005229;

LTB4 antagonists such LY293111, CGS025019C, CP-195543, SC-53228, BIIL 284, ONO 4057, SB 209247 and those described in U.S. Pat. No. 5451700; LTD4 antagonists such as montelukast and zafirlukast; PDE4 inhibitors such cilomilast (Ariflo® GlaxoSmithKline), Roflumilast (Byk Gulden), V-11294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), SeICID™ CC-10004 (Celgene), VM554/UM565 (Vernalis), T-440 (Tanabe), KW-4490 (Kyowa Hakko Kogyo), and those disclosed in WO 92/19594, WO 93/19749, WO 93/19750, WO 93/19751, WO 98/18796, WO 99/16766, WO 01/13953, WO 03/104204, WO 03/104205, WO 03/39544, WO 04/000814, WO 04/000839, WO 04/005258, WO 04/018450, WO 04/018451, WO 04/018457, WO 04/018465, WO 04/018431, WO 04/018449, WO 04/018450, WO 04/018451, WO 04/018457, WO 04/018465, WO 04/019944, WO 04/019945, WO 04/045607 and WO 04/037805; A2a agonists such as those disclosed in EP 409595A2, EP 1052264, EP 1241176, WO 94/17090, WO 96/02543, WO 96/02553, WO 98/28319, WO 99/24449, WO 99/24450, WO 99/24451, WO 99/38877, WO 99/41267, WO 99/67263, WO 99/67264, WO 99/67265, WO 99/67266, WO 00/23457, WO 00/77018, WO 00/78774, WO 01/23399, WO 01/27130, WO 01/27131, WO 01/60835, WO 01/94368, WO 02/00676, WO 02/22630, WO 02/96462, WO 03/086408, WO 0 4/039762, WO 04/039766, WO 04/045618 and WO 04/046083; A2b antagonists such as those described in WO 02/42298; and beta-2 adrenoceptor agonists such as albuterol (salbutamol), metaproterenol, terbutaline, salmeterol fenoterol, procaterol, and especially, formoterol and pharmaceutically acceptable salts thereof, and compounds (in free or salt or solvate form) of formula I of WO 0075114, which document is incorporated herein by reference, preferably compounds of the Examples thereof, especially a compound of formula

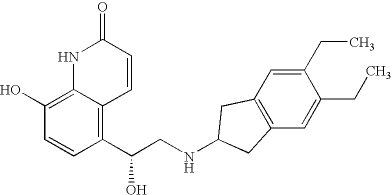

and pharmaceutically acceptable salts thereof, as well as compounds (in free or salt or solvate form) of formula I of WO 04/16601, and also compounds of WO 04/033412.

Suitable bronchodilatory drugs include anticholinergic or antimuscarinic compounds, in particular ipratropium bromide, oxitropium bromide, tiotropium salts and CHF 4226 (Chiesi), and glycopyrrolate, but also those described in WO 01/04118, WO 02/51841, WO 02/53564, WO 03/00840, WO 03/87094, WO 04/05285, WO 02/00652, WO 03/53966, EP 424021, U.S. Pat. No. 5,171,744, U.S. Pat. No. 3,714,357, WO 03/33495 and WO 04/018422.

Suitable antihistamine drug substances include cetirizine hydrochloride, acetaminophen, clemastine fumarate, promethazine, loratidine, desloratidine, diphenhydramine and fexofenadine hydrochloride, activastine, astemizole, azelastine, ebastine, epinastine, mizolastine and tefenadine as well as those disclosed in WO 03/099807, WO 04/026841 and JP 2004107299.

Other useful combinations of compounds of the invention with anti-inflammatory drugs are those with antagonists of chemokine receptors, e.g. CCR-1, CCR-2, CCR-3, CCR-4, CCR-5, CCR-6, CCR-7, CCR-8, CCR-9 and CCR10, CXCR1, CXCR2, CXCR3, CXCR4, CXCR5, particularly CCR-5 antagonists such as Schering-Plough antagonists SC-351125, SCH55700 and SCH-D, Takeda antagonists such as N-[[4-[[[6,7-dihydro-2-(4-methylphenyl)-5H-benzo-cyclohepten-8-yl]carbonyl]amino]phenyl]-methyl]tetrahydro-N,N-dimethyl-2H-pyran-4-amin-ium chloride (TAK-770), and CCR-5 antagonists described in U.S. Pat. No. 6166037 (particularly claims 18 and 19), WO 00/66558 (particularly claim 8), WO 00/66559 (particularly claim 9), WO 04/018425 and WO 04/026873.

The structure of the active compounds identified by code nos., generic or trade names may be taken from the actual edition of the standard compendium “The Merck Index” or from databases, e.g. Patents International (e.g. IMS World Publications).

The above-mentioned compounds, which can be used in combination with a compound of the formula (I), can be prepared and administered as described in the art, such as in the documents cited above.

By “combination”, there is meant either a fixed combination in one dosage unit form, or a kit of parts for the combined administration where a compound of the formula (I) and a combination partner may be administered independently at the same time or separately within time intervals that especially allow that the combination partners show a cooperative, e.g. synergistic effect.

The invention also provides a pharmaceutical preparation, comprising a compound of formula I as defined herein, or an N-oxide or a tautomer thereof, or a pharmaceutically acceptable salt of such a compound, or a hydrate or solvate thereof, and at least one pharmaceutically acceptable carrier.

A compound of formula I can be administered alone or in combination with one or more other therapeutic compounds, possible combination therapy taking the form of fixed combinations or the administration of a compound of the invention and one or more other therapeutic (including prophylactic) compounds being staggered or given independently of one another, or the combined administration of fixed combinations and one or more other therapeutic compounds. A compound of formula I can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

The dosage of the active ingredient depends upon a variety of factors including type, species, age, weight, sex and medical condition of the patient; the severity of the condition to be treated; the route of administration; the renal and hepatic function of the patient; and the particular compound employed. A physician, clinician or veterinarian of ordinary skill can readily determine and prescribe the effective amount of the drug required to prevent, counter or arrest the progress of the condition. Optimal precision in achieving concentration of drug within the range that yields efficacy requires a regimen based on the kinetics of the drug's availability to target sites. This involves a consideration of the distribution, equilibrium, and elimination of a drug.

The dose of a compound of the formula I or a pharmaceutically acceptable salt thereof to be administered to warm-blooded animals, for example humans of approximately 70 kg body weight, is preferably from approximately 3 mg to approximately 5 g, more preferably from approximately 10 mg to approximately 1.5 g per person per day, divided preferably into 1 to 3 single doses which may, for example, be of the same size. Usually, children receive half of the adult dose.

The compounds of the invention may be administered by any conventional route, in particular parenterally, for example in the form of injectable solutions or suspensions, enterally, e.g. orally, for example in the form of tablets or capsules, topically, e.g. in the form of lotions, gels, ointments or creams, or in a nasal or a suppository form. Topical administration is e.g. to the skin. A further form of topical administration is to the eye. Pharmaceutical compositions comprising a compound of the invention in association with at least one pharmaceutical acceptable carrier or diluent may be manufactured in conventional manner by mixing with a pharmaceutically acceptable carrier or diluent.

The invention relates also to pharmaceutical compositions comprising an effective amount, especially an amount effective in the treatment of one of the above-mentioned disorders, of a compound of formula I or an N-oxide or a tautomer thereof together with one or more pharmaceutically acceptable carriers that are suitable for topical, enteral, for example oral or rectal, or parenteral administration and that may be inorganic or organic, solid or liquid. There can be used for oral administration especially tablets or gelatin capsules that comprise the active ingredient together with diluents, for example lactose, dextrose, mannitol, and/or glycerol, and/or lubricants and/or polyethylene glycol. Tablets may also comprise binders, for example magnesium aluminum silicate, starches, such as corn, wheat or rice starch, gelatin, methylcellulose, sodium carboxymethylcellulose and/or polyvinylpyrrolidone, and, if desired, disintegrators, for example starches, agar, alginic acid or a salt thereof, such as sodium alginate, and/or effervescent mixtures, or adsorbents, dyes, flavorings and sweeteners. It is also possible to use the pharmacologically active compounds of the present invention in the form of parenterally administrable compositions or in the form of infusion solutions. The pharmaceutical compositions may be sterilized and/or may comprise excipients, for example preservatives, stabilizers, wetting compounds and/or emulsifiers, solubilizers, salts for regulating the osmotic pressure and/or buffers. The present pharmaceutical compositions, which may, if desired, comprise other pharmacologically active substances are prepared in a manner known per se, for example by means of conventional mixing, granulating, confectioning, dissolving or lyophilizing processes, and comprise approximately from 1% to 99% by weight, especially from approximately 1% to approximately 60%, active ingredient(s).

Additionally, the present invention provides a compound of formula I or an N-oxide or a tautomer thereof, or a pharmaceutically acceptable salt of such a compound, for use in a method for the treatment of the human or animal body, especially for the treatment of a disease mentioned herein, most especially in a patient in need of such treatment.

The present invention also relates to the use of a compound of formula I or a tautomer thereof, or a pharmaceutically acceptable salt of such a compound, for the preparation of a medicament for the treatment of a proliferative disease, an inflammatory disease, or an obstructtive airway disease, or disorders commonly occurring in connection with transplantation.

Furthermore, the invention relates to a method for the treatment of a proliferative disease which responds to an inhibition of lipid kinases and/or PI3-kinase-related protein kinases, in particular the PI3 kinase, and/or mTOR, and/or DNA protein kinase activity, which comprises administering a compound of formula I or a pharmaceutically acceptable salt thereof, wherein the radicals and symbols have the meanings as defined above, especially in a quantity effective against said disease, to a warm-blooded animal requiring such treatment.

Furthermore, the invention relates to a pharmaceutical composition for treatment of solid or liquid tumours in warm-blooded animals, including humans, comprising an antitumor effecttive dose of a compound of the formula I as described above or a pharmaceutically acceptable salt of such a compound together with a pharmaceutical carrier.

Manufacturing Process:

The invention relates also to a process for the manufacture of a compound of the formula I, an N-oxide thereof, a solvate thereof and/or a salt thereof.

Compounds of the formula I (especially the novel compounds) can be prepared according to or in analogy to methods that, in principle but with other educts, intermediates and/or final products, are known in the art, especially and according to the invention by a novel process comprising

a) reacting a compound of the formula II,

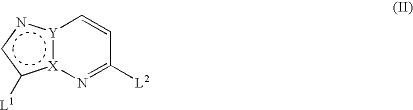

wherein

X is N and Y is C, or X is C and Y is N,

the broken circle represents two conjugated double bonds within the five-membered ring with the proviso that the first of said bonds starts from either X═C or Y═C; or the moieties X, N and the broken circle are as defined otherwise (especially as preferred) within this specification; and

each of L¹ and L², independently of the other, is halo, especially chloro, bromo or iodo, or is trifluoromethansulfonyloxy, under cross coupling conditions with a boronic acid or boronic acid ester or organotin compound of the formula III,

R^{1,2}-D (III)

wherein R^{1,2} is unsubstituted or substituted aryl or unsubstituted or substituted heterocyclyl; as defined for R¹ and R² for a compound of the formula I and D is —B(OH₂) in free form or in esterified form, e.g. as a group of the formula A

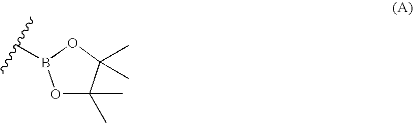

or as a di-C₁-C₇-alkyl ester, or is —Sn(alk)₃ wherein alk is alkyl, preferably C₁-C₇-alkyl, more preferably methyl, or

b) reacting a compound of the formula IV,

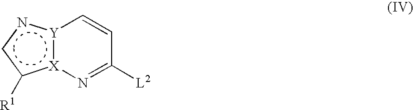

wherein

X is N and Y is C, or X is C and Y is N,

the broken circle represents two conjugated double bonds within the five-membered ring with the proviso that the first of said bonds starts from either X═C or Y═C;

and R¹ is unsubstituted or substituted aryl or unsubstituted or substituted heterocyclyl; or the moieties R¹, X, N and the broken circle are as defined otherwise (especially as preferred) within this specification; and

L² is halo, especially chloro, iodo or preferably bromo, or is trifluoromethansulfonyloxy, under cross coupling conditions with a boronic acid or boronic acid ester or organotin compound of the formula V,

R²-D (V)

wherein R² is unsubstituted or substituted aryl or unsubstituted or substituted heterocyclyl; as defined for R² for a compound of the formula I and D is —B(OH₂) in free form or in esterified form, e.g. as a group of the formula A

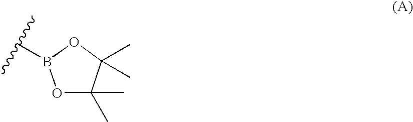

or as a di-C₁-C₇-alkyl ester, or is —Sn(alk)₃ wherein alk is alkyl, preferably C₁-C₇-alkyl, more preferably methyl, or

c) reacting a compound of the formula VI,

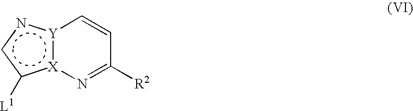

wherein

X is N and Y is C, or X is C and Y is N,

the broken circle represents two conjugated double bonds within the five-membered ring with the proviso that the first of said bonds starts from either X═C or Y═C;

and R² is unsubstituted or substituted aryl or unsubstituted or substituted heterocyclyl; or the moieties R², X, N and the broken circle are as defined otherwise (especially as preferred) within this specification; and

L¹ is halo, especially chloro, iodo or preferably bromo, or is trifluoromethansulfonyloxy, under cross coupling conditions with a boronic acid or boronic acid ester or organotin compound of the formula VII,

R¹-D (VII)

wherein R¹ is unsubstituted or substituted aryl or unsubstituted or substituted heterocyclyl, or as otherwise as defined for R¹ for a compound of the formula I, and D is —B(OH₂) in free form or in esterified form, e.g. as a group of the formula A

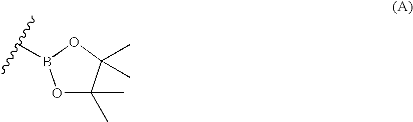

or as a di-C₁-C₇-alkyl ester, or is —Sn(alk)₃ wherein alk is alkyl, preferably C₁-C₇-alkyl, more preferably methyl, or

d) reacting a compound of the formula VIII,

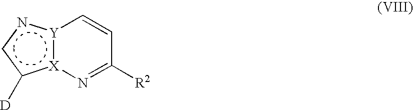

wherein

X is N and Y is C, or X is C and Y is N,

the broken circle represents two conjugated double bonds within the five-membered ring with the proviso that the first of said bonds starts from either X═C or Y═C;

R² is unsubstituted or substituted aryl or unsubstituted or substituted heterocyclyl; or the moieties R², X, N and the broken circle are as defined otherwise (especially as preferred) within this specification; and

D is —B(OH₂) in free form or in esterified form, e.g. as a group of the formula A

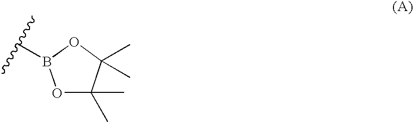

or as a di-C₁-C₇-alkyl ester, or is —Sn(alk)₃ wherein alk is alkyl, preferably C₁-C₇-alkyl, more preferably methyl; under cross-coupling conditions with a compound of the formula IX,

R¹-L¹ (IX)

wherein

L¹ is halo, especially chloro, iodo or preferably bromo, or is trifluoromethansulfonyloxy, and R¹ is unsubstituted or substituted aryl or unsubstituted or substituted heterocyclyl, or as otherwise defined for R¹ for a compound of the formula I;

and, if desired, a compound of the formula I obtainable according to any one of the reactions a) to d) given above is converted into a different compound of the formula I, an obtainable salt of a compound of the formula I is converted into a different salt thereof, an obtainable free compound of the formula I is converted into a salt thereof, and/or an obtainable isomer of a compound of the formula I is separated from one or more different obtainable isomers of the formula I.

In the following more detailed description of preferred variants of the processes, optional reactions and conversions, synthesis of starting materials and intermediates and the like, R¹, R², X, Y and the broken circle have the meanings given for a compound of the formula I or the compound mentionned specifically, while D is as defined for a compound of the formula Ill, R^{1,2} is as defined for a compound of the formula III, L¹ and L² are as defined for a compound of the formula II, X as for a compound of the formula II, Het as defined for a compound of the formula X, Hyl as described for a compound of the formula XI and Hea as defined for a compound of the formula XII, or preferably as mentioned otherwise.

The symbol alk is as defined for a compound of the formula III, if not indicated otherwise.

Where useful or required, the reactions can take place under an inert gas, such as nitrogen or argon. Heating can, for example, be effected by means or microwaves or (e.g. oil) baths or the like, where required in sealed reaction vessels to avoid evaporation at the temperatures used.

The reaction given under process variants a), b), c) and d), respectively, is, if D is —B(OH)₂ in free form or in esterified form, preferably carried out under the conditions of a Suzuki-reaction or in analogy thereto, preferably in one or more aprotic solvents, such as dimethylformamide (DMF), in an alcohol such as ethanol, in a cyclic ether such as tetrahydrofurane or dioxane, in an acyclic ether, such as dimethylether, in a cyclic hydrocarbon such as toluene, in a haloalkane, e.g. dichloromethane, or in a mixture of two or more such solvents and optionally water in the presence of a catalyst for the cross-coupling, especially a noble metal catalyst, preferably a palladium catalyst, such as palladium(II) complex, for example bis(triphenylphosphine)palladium (II) dichloride or [1,1′-bis(diphenylphosphino)ferrocene]dichloropalladium(II) (e.g. as dichloromethane complex), in the presence of a base, such as potassium carbonate, an alkalimetal C₁-C₇-alkanoate, such as sodium or potassium acetate, sodium hydroxide or sodium carbonate, at a preferred temperature in the range from 70° C. to 150° C.; or according to another preferred method in a cyclic ether solvent, e.g. tetrahydrofurane, with or without the presence of water, in the presence of a catalyst for the cross coupling, especially a noble metal catalyst, preferably a palladium (0) complex, for example tris(dibenzylideneacetone)-dipalladium(0), or of palladium dibenzylideneacetone as precursor, where useful in the presence an appropriate ligand, such as 2-dicyclo-hexylphosphino-2′,6′-dimethoxybiphenyl (SPhos) or 2-dicyclohexylphosphino-2′-(N,N-dimethylamino)-biphenyl (P1), and in the presence of a base, e.g. as mentioned above or potassium phosphate, and at a preferred temperatures in the range from 80 to 160° C.; if required conducting the reaction in a sealed vessel (e.g. a seal reactor or a microwave vessel) if the boiling point of the reaction mixture is exceeded and/or especially if (as is a preferred embodiment) the heating is effected by microwave excitation. Where required, other or additional catalyst(s) can be added, e.g. (PdCl₂(PPh₂).Fe.CH₂Cl₂), or mixtures of catalysts can be used.

The reaction given under process variants a), b), c) and d) respectively, is, if D is —Sn(alk)₃ wherein alk is alkyl, preferably C₁-C₇-alkyl, more preferably methyl, is preferably conducted under Stille coupling conditions, or in analogy thereto, preferably in an appropriate polar solvent, such as N,N-dimethylacetamide or N,N-dimethylformamide, an ether, such as tetrahydrofurane, and/or a mixture of two or more such solvents, in the presence of a a palladium catalyst, especially a palladium (0) complex, for example tetrakistriphenylpalladium, e.g. at temperatures in the range from 80 to 160° C., if required conducting the reaction in a sealed vessel (e.g. a seal reactor or a microwave vessel) if the boiling point of the reaction mixture is exceeded and/or especially if (as is a preferred embodiment) the heating is effected by microwave excitation.

Where temperatures are given hereinbefore or hereinafter, “about” has to be added, as minor deviations from the numeric values given, e.g. variations of ±10%, are tolerable.

Protecting Groups

If one or more other functional groups, for example carboxy, hydroxy, amino, or mercapto, are or need to be protected in a starting material, e.g. in any one or more starting materials of the formula II to IX or other starting materials, intermediates and educts mentioned below, because they should not take part in the reaction or disturb the reaction, these are such groups as are usually used in the synthesis of peptide compounds, and also of cephalosporins and penicillins, as well as nucleic acid derivatives and sugars. Protecting groups are such groups that are no longer present in the final compounds once they are removed, while groups that remain as substituents are not protecting groups in the sense used here which is groups that are added at a certain intermediate stage and removed to obtain a final compound. For example, tert-butoxy if remaining in a compound of the formula I is a substituent, while if it is removed to obtain the final compound of the formula I it is a protecting group.

The protecting groups may already be present in precursors and should protect the functional groups concerned against unwanted secondary reactions, such as acylations, etherifications, esterifications, oxidations, solvolysis, and similar reactions. It is a characteristic of protecting groups that they lend themselves readily, i.e. without undesired secondary reactions, to removal, typically by acetolysis, protonolysis, solvolysis, reduction, photolysis or also by enzyme activity, for example under conditions analogous to physiological conditions, and that they are not present in the end-products. The specialist knows, or can easily establish, which protecting groups are suitable with the reactions mentioned above and below.

The protection of such functional groups by such protecting groups, the protecting groups themselves, and their removal reactions are described for example in standard reference works, such as J. F. W. McOmie, “Protective Groups in Organic Chemistry”, Plenum Press, London and New York 1973, in T. W. Greene, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of organic chemistry), Houben Weyl, 4th edition, Volume 15/I, Georg Thieme Verlag, Stuttgart 1974, in H.-D. Jakubke and H. Jescheit, “Aminosauren, Peptide, Proteine” (Amino acids, peptides, proteins), Verlag Chemie, Weinheim, Deerfield Beach, and Basel 1982, and in Jochen Lehmann, “Chemie der Kohlenhydrate: Monosaccharide and Derivate” (Chemistry of carbohydrates: monosaccharides and derivatives), Georg Thieme Verlag, Stuttgart 1974.

An example for an amino (or imino) protecting group is tert-butoxycarbonyl which can be introduced used to protect amino or imino groups and can be removed e.g. by hydrolysis, e.g. with an acid, such as trifluoroacetic acid or hydrochloric acid, in an appropriate solvent, e.g. methylene chloride or dioxane, e.g. at temperatures in the range from 0 to 50° C.

Optional Reactions and Conversions

A compound of the formula I may be converted into a different compounds of the formula I according to standard reaction procedures, e.g. as described in the following:

For example, in a compound of the formula I wherein R¹ and/or R² is heteroaryl (meaning unsaturated heterocylyl), such as pyridyl (=pyridinyl), that is substituted by halo, especially by chloro or bromo or fluoro, e.g. in the p-position, the halo can be replaced by a unsubstituted or substituted ring nitrogen comprising unsaturated heterocyclyl bound via a ring nitrogen atom by reaction with a compound of the formula X,

H-Het (X)

wherein Het is an unsubstituted or substituted unsaturated heterocyclyl moiety bound to the hydrogen via a ring nitrogen atom, such as 1,2,4-triazol, pyrazole, benzimidazole, 3-trifluoromethyl-pyrazol, under Ullman-type reaction conditions, e.g. as in see e.g. Chem. Eur. J. (2004), 10, 5607 on the general Ullmann-type arylation of nucleophiles, preferably by reacting the corresponding compound of the formula I and the compound of the formula XI in the presence of Cu₂O, a ligand such as salicylaldehyde hydrazone, a base such as caesium carbonate and a solvent such as acetonitrile at preferred temperatures in the range from 100 to 180° C., e.g. at 160 to 150° C., for example in a microwave oven. This leads to a compound of the formula I wherein R¹ and/or R² is heteroaryl, e.g. pyridinyl, substituted by unsubstituted or substituted ring nitrogen comprising unsaturated heterocyclyl bound via a ring nitrogen atom.

Alternatively, for example, in a compound of the formula I wherein R¹ and/or R² is heteroaryl, such as pyridyl, that is substituted by halo, especially by chloro or bromo or most preferably fluoro, e.g. in the p-position, the halo can be replaced by an unsubstituted or substituted saturated heterocyclyl comprising a nitrogen atom or by amino substituted e.g. with phenyl-lower alkyl by reaction with a compound of the formula XI,

H-Hyl (XI)

wherein Hyl is an unsubstituted or substituted saturated heterocyclyl moiety bound to the hydrogen via a ring nitrogen atom, such as valerolactame, morpholine, 2-pyrrolidinone or N-methylpiperazine, or a substituted amino, such as phenyl-C₁-C₇-alkylamino, under reaction conditions such as those described in Example 28, that is in the presence of a base, especially cesium carbonate, in an appropriate solvent, such as 1-methylpyrrolidin-2-one, or as described in Example 31 in the presence or absence of a base and a further solvent, in both cases e.g.at temperatures in the range from 100 to 170° C., or e.g. reacting the heterocyclic compound of the formula XI and the corresponding compound of the formula I in the presence of Cul, a base, such as potassium carbonate, and of proline in an appropriate solvent, such as dimethylsulfoxide, preferably at temperatures in the range from 80 to 130° C. Also Buchwald-Hartwig reaction conditions may be useful.

Yet alternatively, in a compound of the formula I wherein R¹ and/or R² is heteroaryl, such as pyridyl, or phenyl that is substituted by halo, especially by chloro or bromo, e.g. in the p-position, the halo can be replaced by an unsubstituted or substituted saturated heterocyclyl bound via a ring carbon atom by reaction with a compound of the formula XII,

D*-Hea (XII)

wherein Hea is unsaturated heterocyclyl (heteroaryl) and D* has the meaning of D given above for compounds of the formula III, by reaction under conditions analogous to those mentioned above for reaction variants a), b), c) and d).

In the preceding and subsequent paragraphs on conversions, heterocyclyl or heteroaryl Het, Hyl and Hea can be unsubstituted or substituted as described above for unsubstituted or substituted heterocyclyl, preferably by substituents other than halo.

In a compound of the formula I wherein R¹ and/or R² is 3-pyridinyl substituted by fluoro, the fluoro may be converted to unsubstituted or substituted heterocylyloxy by reaction with the corresponding unsubstituted or substituted heterocyclyl-hydroxide (hydroxy hetero-cycle), such as 4-hydroxy-1-isopropylpiperidine, to the corresponding unsubstituted or substituted heterocyclyloxy-substituted compound of the formula I, e.g. in the presence of a strong base, such as sodium hydride, and an appropriate solvent, e.g. 1-methylpyrrolidine-2-one, e.g. at temperatures in the range from 0 to 50° C.

In a compound of the formula I wherein R¹ and/or R² is halo-substituted heterocyclyl, e.g. 6-fluoro-pyridin-3-yl, this can be converted to the corresponding hydroxy-substituted heterocyclyl, e.g. 6-hydroxypyridin-3-yl, e.g. by reaction with a base, such as potassium acetate, in the presence of water, e.g. at temperatures in the range from 50 to 170° C.

In a compound of the formula I wherein an amino or imino group carries a C₁-C₇-alkoxycarbonyl, such as tert-butoxycarbonyl group, this group may be removed under conditions analogous to those decribed above unter “Protecting groups”.

In a compound of the formula I wherein R¹ is heterocyclyl (especially unsaturated heterocyclyl=heteroaryl, e.g. pyrazolyl, pyrazinyl or pyridyl) carrying a hydroxy group, the hydroxy group can be converted into halo, e.g. chloro, by reaction, e.g. with an inorganic acid halide, such as phosphorus oxychloride, under customary conditions, e.g. in the absence or presence of a solvent at elevated temperatures, such as reflux temperature.

In a compound of the formula I wherein R¹ is heterocyclyl comprising an imino group (that is, —NH—), e.g. in pyrazol-3-yl or pyrazin-2-yl, the hydrogen in the imino group may be acylated to C₁-C₇-alkanoylimino, unsubstituted or substited benzoylimino, C₁-C₇-alkanesulfonylimino or unsubstituted or substituted benzenesulfonylino, by reaction with a corresponding acid halogenide, e.g. acid chloride, or with the help of an in situ activating agent (coupling agent), such as HATU or HBTU or the like, see e.g. below for further coupling agents and conditions, under customary reaction conditions, e.g. in the presence of a solvent, such as tetrahydrofurane, or in its absence, in the presence of a tertiary nitrogen base, such as pyridine or triethylamine, at temperatures e.g. in the range from 0 to 50° C.

In a compound of the formula I wherein R² carries an C₁-C₇-alkoxycarbonylamino-C₁-C₇-alkoxy substituent, this may be converted to the free amino-C₁-C₇-alkoxy substituent e.g. as described above for the deprotection of C₁-C₇-alkoxycarbonylamino to amino.

In a compound of the formula I wherein R² carries an amino-C₁-C₇-alkoxy substituent, this substituent can be converted into C₆-C₁₄-arylcarbonylamino-C₂-C₇-alkoxy wherein C₆-C₁₄-aryl is unsubstituted or substituted by one or more substituents independently selected from the group consisting of C₁-C₇-alkyl, halo-C₁-C₇-alkyl, hydroxy, C₁-C₇-alkoxy and halo, or into heterocyclylcarbonylamino-C₁-C₇-alkoxy wherein heterocyclyl has 3 to 10 ring atoms and has one or more hetero ring atoms selected from O, S and N, especially N, by reaction with a corresponding acid or a reactive acid derivative (such as acid halogenide, e.g. acid chloride) which can also be formed in situ, e.g. by means of a coupling agent that forms a reactive derivative of the carboxyl group in situ, for example dicyclohexylcarbodiimide/1-hydroxybenzotriazole (DCC/HOBT); bis(2-oxo-3-oxazolidinyl)phosphinic chloride (BOPCI); O-(1,2-dihydro-2-oxo-1-pyridyl)-N,N,N′,N′-tetramethyluronium tetrafluoroborate (TPTU); O-benzotriazol-1-yl)-N,N,N′,N′-tetramethyluronium tetrafluoroborate (TBTU); (benzotriazol-1-yloxy)-tripyrrolidinophosphonium-hexafluorophosphate (PyBOP), O-(1H-6-chlorobenzotriazole-1-yl)-1,1,3,3-tetramethyluronium hexafluorophosphate, 1-(3-dimethylaminopropyl)-3-ethylcarbodiimide hydrochloride/hydroxybenzotriazole, O-(7-azabenzotriazol-1-yl)-N,N,N′, N′-tetramethyluronium-hexafluorophosphat (HATU) or/1-hydroxy-7-azabenzotriazole (EDC/HOBT or EDC/HOAt) or HOAt alone, or with (1-chloro-2-methyl-propenyl)-dimethylamine. For review of some other possible coupling agents, see e.g. Klauser; Bodansky, Synthesis (1972), 453-463. The reaction mixture, which advantageously can comprise an appropriate solvent, e.g. dimethyl formamide or dioxane, and/or N-methylmorpholine, is preferably kept, e.g. stirred, at a temperature of between approximately -20 and 80° C., especially between 0° C. and 60° C., e.g. at room temperature or at about 50° C.

In a compound of the formula I wherein R² carries an amino-C₁-C₇-alkoxy substituent, this substituent can be converted into C₆-C₁₄-arylaminocarbonylamino-C₂-C₇-alkoxy (C₆-C₁₄-aryl-NH—C(═O)—NH—C₂-C₇-alkoxy) wherein C₆-C₁₄-aryl is defined as above, preferably is phenyl or naphthyl, and is in each case unsubstituted or substituted by one or more, especially up to three, substituents independently selected from the group consisting of C₁-C₇-alkyl, especially methyl or ethyl, halo-C₁-C₇-alkyl, especially trifluoromethyl, hydroxy, C₁-C₇-alkoxy, especially methoxy, and halo, especially fluoro, or into heterocyclylaminocarbonylamino-C₁-C₇-alkoxy wherein heterocyclyl has 3 to 10 ring atoms and has one or more hetero ring atoms selected from O, S and N, especially N, by reaction with a corresponding isocyanate under customary conditions.

A compound of the formula I wherein R¹ is heterocyclyl, such as pyridyl, that is substituted by cyano can be converted to a corresponding compound of the formula I wherein instead of the cyano an 1H-tetrazol-5-yl moiety is present by reaction with an azide salt, such as sodium azide, preferably in the presence of an ammonium salt, such as ammonium chloride, at a temperature e.g. from 120 to 160° C.

A compound of the formula I wherein R¹ is heterocyclyl, such as pyrazolyl, pyrazinyl or pyridyl, substituted by nitro can be reduced to a corresponding compound of the formula I wherein instead of the nitro an amino group is present, e.g. by reduction by hydrogenation in the presence of a hydrogennation catalyst, e.g. a noble metal catalyst, such as palladium, which can preferably be bound to a carrier, such as charcoal, in an appropriate solvent, such as an alcohol, e.g. methanol, preferably at temperatures in the range from 0 to 50° C., e.g. at room temperature. As by-product, the alkylation product resulting from the alcohol can be obtained, e.g. in the case of methanol the corresponding methylamino compound of the formula I, which can be isolated according to standard procedures, such as chromatography.

In a compound of the formula I wherein R¹ or R² is aryl, such as phenyl, or heteroaryl, such as pyrazolyl, pyrazinyl or pyridyl, substituted by chloro, bromo or iodo, the chloro, bromo or iodo can be converted into a group D as described above for a compound of the formula Ill, for example by reaction first with n-butylllithium (replacing the chloro, bromo or iodo by Li) and subsequent reaction with a corresponding trialkoxyborane, such as triisopropylborane; or by reaction of the chloro, bromo or iodo compound in the presence of a transition metal catalyst (e.g. PdCl(dppf) with alkoxydiborone), or the like. Alternatively, also triflate (trifluoromethanesulfonyloxy) substituents instead of halo can be substituted accordingly in corresponding starting materials.The free boronic acids (unesterified) can be obtained e.g. by working up in the presence of an inorganic acid, such as hydrochloric acid.

The compound of the formula I carrying a group D as just described can then be reacted with an unsubstituted or substituted aryl or unsaturated heterocyclyl compound under conditions as described above for reaction a) (e.g. cross coupling, such as Suzuki coupling) to a corresponding compound of the formula I wherein instead of the original chloro, bromo or iodo an aryl or unsaturated heterocyclyl substituent is present (each of which may be substituted as well as described above).

Alternatively, in a compound of the formula I wherein R¹ or R² is aryl, such as phenyl, or heteroaryl, such as pyrazolyl, pyrazinyl or pyridyl, substituted by chloro, bromo or iodo, the chloro, bromo or iodo can be converted into a group unsubstituted or substituted aryl or unsubstituted or substituted unsaturated heterocyclyl by reaction with a corresponding unsubsituted or substituted (aryl or unsaturated heterocyclyl)-boronic acid or boronic acid ester under reaction conditions analogous to those mentioned above for reaction a), e.g. in an appropriate solvent, such as a cyclic ether, e.g. tetrandrofurane, in the presence of a base, such as potassium phosphate, and a catalyst, e.g. palladium dibenzylidenacetone and 2-dicyclohexylphosphino-2′,6′-dimethoxybiphenyl, preferably at elevated temperatures, e.g. in the range from 100 to 160° C.

D as described above for a compound of the formula III, for example by reaction first with n-butylllithium (replacing the chloro, bromo or iodo by Li) and subsequent reaction with a corresponding trialkoxyborane, such as triisopropylborane; or by reaction of the chloro, bromo or iodo compound in the presence of a transition metal catalyst (e.g. PdCl(dppf) with alkoxydiborone), or the like. Alternatively, also triflate (trifluoromethanesulfonyloxy) substituents instead of halo can be substituted accordingly in corresponding starting materials. The free boronic acids (unesterified) can be obtained e.g. by working up in the presence of an inorganic acid, such as hydrochloric acid.

A nitrogen ring atom of the imidazo[1,2-b]pyridazine core or a nitrogen-containing heterocyclyl substituent can form an N-oxide in the presence of a suitable oxidizing agent, e.g. a peroxide, such as m-chloro-perbenzoic acid or hydrogen peroxide.

Also in the optional process steps, carried out “if desired”, functional groups of the starting compounds which should not take part in the reaction may be present in unprotected form or may be protected for example by one or more of the protecting groups mentioned herein-above under “protecting groups”. The protecting groups are then wholly or partly removed according to one of the methods described there.

Salts of a compound of formula I with a salt-forming group may be prepared in a manner known per se. Acid addition salts of compounds of formula I may thus be obtained by treatment with an acid or with a suitable anion exchange reagent, salt with bases by treatment with a corresponding base or a suitable cation exchange reagent.

Salts can usually be converted to free compounds, e.g. acid addition salts by treating with suitable basic compounds, for example with alkali metal carbonates, alkali metal hydrogen-carbonates, or alkali metal hydroxides, typically potassium carbonate or sodium hydroxide, salt with bases by treating with suitable acid compounds, such as hydrochloric acid, sulfuric acid or the like.

Mixtures of constitutional isomers or of products and by-products can be separated according to standard procedures, e.g. by distribution, chromatography or the like.

Stereoisomeric mixtures, e.g. mixtures of diastereomers, can be separated into their corresponding isomers in a manner known per se by means of suitable separation methods. Diastereomeric mixtures for example may be separated into their individual diastereomers by means of fractionated crystallization, chromatography, solvent distribution, and similar procedures. This separation may take place either at the level of a starting compound or in a compound of formula I itself. Enantiomers may be separated through the formation of diastereomeric salts, for example by salt formation with an enantiomer-pure chiral acid, or by means of chromatography, for example by HPLC, using chromatographic substrates with chiral ligands. Separation may take place in solutions and/or in emulsions, e.g. macro- or microemulsions.

It should be emphasized that reactions analogous to the conversions mentioned in this chapter may also take place at the level of appropriate intermediates (and are thus useful in the preparation of corresponding starting materials).

Starting Materials:

The starting materials of the formulae II, III, IV, V, VI, VII, VIII, IX, X, XI an XII, as well as other starting materials, intermediates or educts mentioned herein, e.g. below, can be prepared according to or in analogy to methods that are known in the art, the materials are known in the art and/or are commercially available, or by or in analogy to methods mentioned in the Examples. Novel starting materials, as well as processes for the preparation thereof, are likewise an embodiment of the present invention. In the preferred embodiments, such starting materials are used and the reaction chosen are selected so as to enable the preferred compounds to be obtained.

Starting materials of the formula II are known in the art, commercially available or can be prepared according to or in analogy to methods known in the art.

For example, a compound of the formula II can be obtained by reacting a compound of the formula XIII,

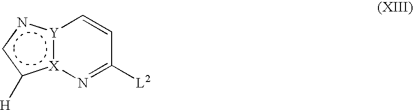

wherein L² is as defined for a compound of the formula II, with an agent capable of introducing L¹ as defined in a compound of the formula II, e.g. an N-halo succinimide, in an appropriate solvent, such as an organic amide, e.g. dimethyl formamide, preferably at temperatures in the range from −20 to 50° C.

A compound of the formula XIII can, for example, be prepared by reacting a pyridazine compound of the formula XIV,

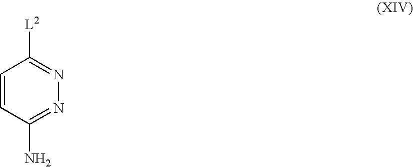

with an L²-substituted acetone of the formula XV,

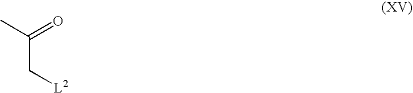

wherein L² is as defined for a compound of the formula II, preferably is halo, especially chloro, e.g. in the presence of a polar solvent, such as an alcohol, e.g. ethanol, and of a base, such as an alkalimetal carbonate, e.g. sodium carbonate, at preferably elevated temperatures, e.g. from 50° C. to the reflux temperature of the solvent mixture.

A compound of the formula IV and a compound of the formula VI can, for example, be obtained as by-product of the reaction described above under a) between a compound of the formula II and a compound of the formula III, followed by isolation, e.g. using silica gel chromatography followed by preparative high performance liquid chromatography with a silica gel or a reversed phase silica based chromatography gel.

Alternatively, a compound of the formula IV wherein L² is halo, X is carbon and Y is nitrogen can be obtained by halogenation of a compound of the formula XVI,

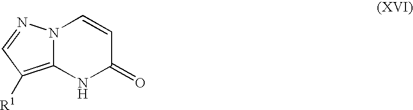

with a halogenating agent, especially an inorganic acid halogenide, such as phosphorus oxychloride (POCl₃), in the absence or presence of an appropriate solvent and preferably at elevated temperatures, e.g. in the range from 80 to 130° C.

A compound of the formula XVI may, for example, be obtained by reacting a pyrazoleamine compound of the formula XVII,

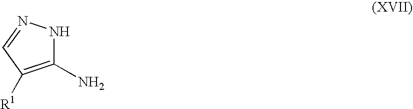

with propiolic acid methyl ester in an appropriate solvent, such as dioxane, preferably at elevated temperatures, e.g. in the range from 20 to 120° C.

A pyrazoleamine of the formula XVII may, for example, be obtained by reacting a cyanoaldehyde compound of the formula XVIII,

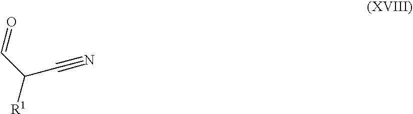

with a hydrazine salt, e.g. hydrazine hydroxide (H₂N—NH₃⁺OH⁻), in the presence of an acid, especially acetic acid, and an appropriate solvent, e.g. toluene, preferably at a temperature in the range from −20° C. to the reflux temperature of the reaction mixture.

A compound of the formula XVIII may, for example, be obtained by reacting a cyano compound of the formula XIX (see WO 2005/070431 Example 93)

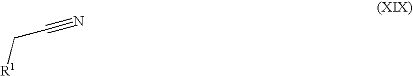

with an alkali metal methylate, e.g. sodium methylate, in an appropriate solvent, e.g. toluene, e.g. as described in WO 2005/070431.

Yet alternatively, a compound of the formula VI wherein L¹ is e.g. bromo can be obtained by reacting a compound of the formula II wherein L¹ is e.g. bromo and L² is chloro, with a compound of the formula VII given above in an appropriate solvent, e.g. an ether, such as dioxane, in the presence of a base, such as an alkali metal carbonate, e.g. sodium carbonate, preferably at temperatures in the range from 50° C. to the reflux temperature of the reaction mixture.

A compound of the formula VI wherein X is carbon and Y is nitrogen can alternatively, for example, be obtained by reacting an oxopropionaldehyde compound of the formula XX,

R²—C(═O)—CH₂—CHO (XX)

(obtainable e.g. in accordance with the method described in Wright, S. W., et al., J. Med. Chem. 35, 4061-4068, 1992), with a halopyrazolamine of the formula XXI,

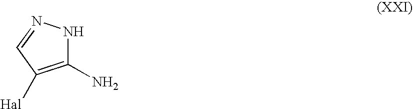

wherein Hal is halo, preferably bromo, in an appropriate solvent, such as an alcohol, e.g. ethanol, in the presence of an acid, such as hydrogen chloride, preferably at temperatures in the range from 0 to 50° C.

A compound of the formula VIII can, for example, be obtained starting from a compound of the formula VI by replacing the group L² with a group —B(OH)₂ in free (obtainable in the presence of an acid, such as hydrochloric acid, from an esterified form) or esterified form e.g. under reaction conditions analogous to those mentioned under the conversions for a compound of the formula I wherein R¹ is unsaturated heterocyclyl (=heteroaryl), such as pyrazolyl, pyrazinyl or pyridyl, substituted by chloro, bromo or iodo, the chloro, bromo or iodo, into the correspondding compound wherein the chloro, bromo or iodo is replaced with a group —B(OH)₂ in free or preferably esterified form; or with a group —Sn(alk)₃ wherein alk is as defined above for a compound of the formula II by reaction with a bis(trialkylstannane), such as bis(tributylstannane) or bis(trimethylstannane), in an appropriate solvent, such as toluene, preferably at elevated temperatures, e.g. from 100° C. to 150° C. For example, a compound of the formula VIII wherein D is a group —B(O—C₁-C₇-alkyl)₂ can be prepared by reacting a compound of the formula VI by reacting it with a tri-(C₁-C₇-alkyl)-borate and alkyllithium, especially butyllithium, in an appropriate solvent, e.g. tetrahydrofurane, hexane or a mixture thereof, at low temperatures, e.g. in the range from −100 to −50° C.

All remaining starting materials, including other starting materials of the formulae for which ways of synthesis are described above, such as compounds of the formula III, V, VII, IX, X, XI, XII, XIV, XV, XIX, XX and XXI, are known, capable of being prepared according to known processes, and/or they are commercially obtainable; in particular, they can be prepared using processes as described or in analogy to those described in the Examples.

### EXAMPLES

The following examples illustrate the invention without limiting the scope thereof.

Temperatures are measured in degrees Celsius. Unless otherwise indicated, the reactions take place at RT.

The R_{f} values in TLC indicate the ratio of the distance moved by each substance to the distance moved by the eluent front. R_{f} values for TLC are measured on 5×20 cm TLC plates, silica gel F₂₅₄, Merck, Darmstadt, Germany.

Starting materials, unless noted otherwise, are from commercial sources including but not limited to

ABCR: ABCR GmbH & Co. KG, Karlsruhe, Germany

Acros: Acros Organics, Geel, Belgium;

Aldrich: Sigma-Aldrich Corp., St. Louis, Mo., USA;

Alfa Aesar: ALFA AESAR, Ward Hill, Mass., USA;

Avocado (belongs to ALFA AESAR);

Boron Molecular: Boron Molecular, Inc., Research Triangle Park, N.C., USA;

ChemBridge: ChemBridge Corporation, San Diego, Calif., USA;

Combi Blocks: Combi-Blocks, Inc., San Diego, Calif., USA;

Fluka: Fluka, Buchs, Switzerland (belongs to Sigma-Aldrich);

Fluorochem: Fluorochem Ltd., Old Glossop, Derbyshire, United Kingdom;

Frontier Scientific: Frontier Scientific, Inc., Logan, Utah, USA;

Lancaster (belongs to ALFA AESAR);

Maybridge: Maybridge, Trevillett and Tintagel, United Kingdom (belongs to Thermo Fischer Scientific, Inc., Waltham, Mass., USA);

Merck: Merck KGaA, Darmstadt, Germany

Ryscor: Ryscor Science, Inc., Wake Forest, N.C., USA

Sigma-Aldrich: Sigma-Aldrich Corp., St. Louis, Mo., USA;

“Emrys Optimizer” is a microwave oven from Personal Chemistry, Biotage AB, Uppsala, Sweden.

CombiFlash® Companion® system is a flash chromatography system from Teledyne Isco, Inc., Lincoln, Nebr., USA; also the RediSep® silica gel column is from Teledyne Isco.

Analytical HPLC Conditions:

System 1

Linear gradient 2-100% CH₃CN (0.1% TFA) and H₂O (0.1% TFA) in 7 min+2 min 100% CH₃CN (0.1% TFA); detection at 215 nm, flow rate 1 mL/min at 30° C. Column: Nucleosil 100-3 C18HD (125×4 mm)

System 2

Linear gradient 2-100% CH₃CN (0.1% TFA) and H₂O (0.1% TFA) in 4 min+2 min 100% CH₃CN (0.1% TFA); back to −100% CH₃CN (0.1% TFA) in 3 min.; detection at 215 nm, flow rate 2 mL/min at RT. Column: Nucleosil OD-5-100 C18 (150×4.6 mm)

All columns are reversed phase columns.

Chromolith Column is from Merck KGaA, Darmstadt, Germany.

Nucleosil Columns are from Macherey § Nagel, Düren, Germany.

Abbreviations:
Boc tert-butoxycarbonyl
brine saturated sodium chloride solution (saturated at RT)
DCM dichloromethane (with palladium catalysts as complex)
DME dimethyl ether
DMF N,N′-dimethyl formamide
EtOH ethanol
EtOAc ethyl acetate
h hour(s)
HPLC high performance liquid chromatography
LC/MS liquid chromatography/mass spectrometry coupling
mL milliliter(s)
min minute(s)
MS(ESI⁺) or MS-ES electrospray ionization mass spectrometry
NEt₃ triethylamine
NMP 1-methyl-2-pyrrolidinone
Pd(dba)2 palladium dibenzylidenacetone
PdCl₂(PPh₃) bis(triphenylphosphine) palladium(II)dichloride
R_{f} ratio of fronts in TLC
RT room temperature
SPhos 2-dicyclohexylphosphino-2′,6′-dimethoxybephenyl
TBME tert. butyl methyl ether
TFA trifluoro acetic acid
THF tetrahydrofurane
TLC thin layer chromatography
TPTU O-(2-Oxo-1(2H)Pyridyl)-N,N,N′,N′-tetraMethyluronium tetrafluoroborate
tRet or t_{R} retention time
UV ultraviolet

General Synthesis

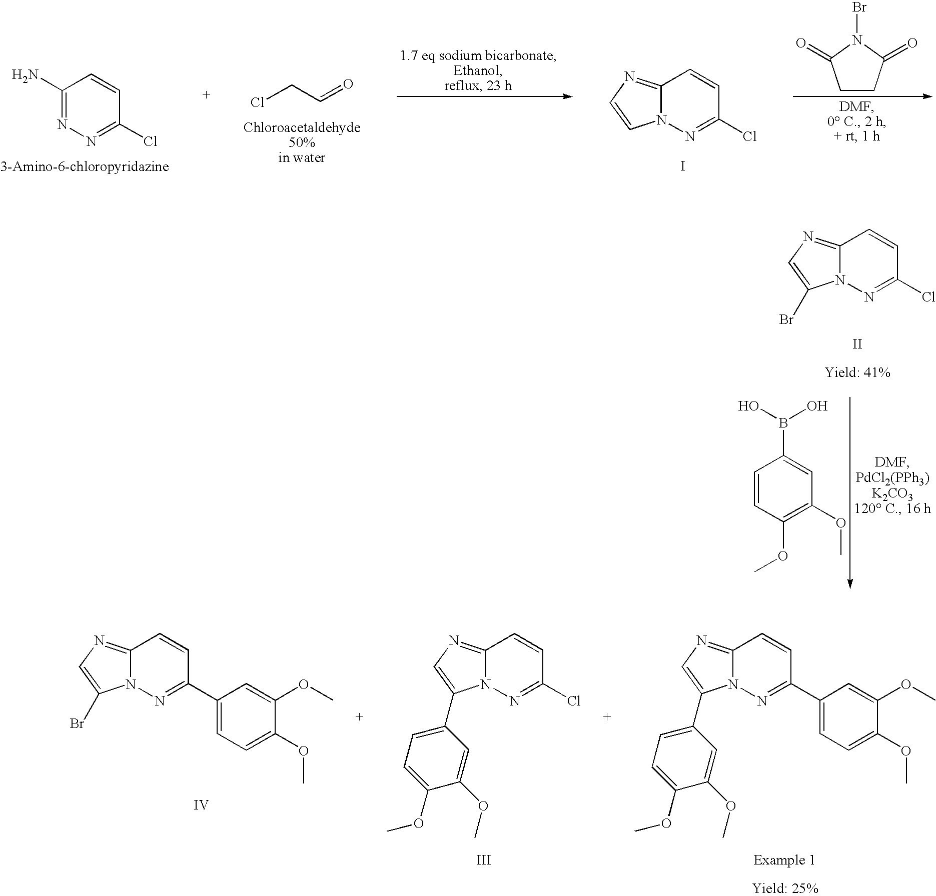

### Example 1

### 3,6-Bis-(3,4-dimethoxy-phenyl)-imidazo[1,2-b]pyridazine

3-Bromo-6-chloro-imidazo[1,2-b]pyridazine (II) (698 mg; 3 mMol) is dissolved in DMF (10 mL) and treated at RT with 3,4-dimethoxy-boronic acid (708 mg; 3.9 mMol), potassium carbonate (1M solution in H₂O; 7.5 mL) and PdCl₂(PPh₃) (40 mg). The dark yellow reaction mixture is stirred at 120° C. for 60 min. After cooling to RT, EtOAc is added (150 mL), followed by extraction with water (2×). The solvent is removed under reduced pressure and the crude product purified by flash chromatography (40 g silica gel [0.040-0.063 mm] Merck 1.09.385.1000]; eluting with CH₂Cl₂/CH₃OH 99:1) to obtain the title compound as yellow powder; MS(ESI⁺):m/z=392.2 (M+H)⁺; HPLC: tRet=4.425 min (System 2).

From this same reaction mixture two additional compounds are obtained and isolated in the course of an additional chromatography (MPLC Büchi, Büchi Labortechnik AG, Flavil, Switzerland; Lichroprep 15-25 μM (silica packing material, Merck)) eluting with a linear gradient of CH₃CN (0.1% TFA) 1 H₂O (0.1% TFA). The combined fractions are neutralized with NaHCO₃, extracted with EtOAc, freed from solvent, taken up into dioxane and freeze-dried:

### 3-Bromo-6-(3,4-dimethoxy-phenyl)-imidazo[1,2-b]pyridazine

Title compound: White powder; MS(ESI⁺):m/z=336.0 (M+H)⁺; HPLC: tRet=4.480 min (System 2).

### 6-Chloro-3-(3,4-dimethoxy-phenyl)-imidazo[1,2-b]pyridazine

Title compound: Yellow powder; MS(ESI⁺):m/z=290.2 (M+H)⁺; HPLC: tRet=4.542 min (System 2).

The starting materials are prepared as follows:

### Stage 1.1: 6-Chloro-imidazo[1,2-b]pyridazine (I)

3-Amino-6-chloropyridazine (5 g; 38.6 mMol) is suspended in EtOH (5 mL) and treated at RT with chloroacetaldehyde (50% in water; 13.7 mL; 106 mMol) and sodium bicarbonate (5.51 g; 65.6 mMol). The yellow suspension is heated to reflux (bath 95° C.) and stirred for 19 h, followed by stirring at RT for 48 h. Additional chloroacetaldehyde (50% in water; 4.98 mL) and sodium bicarbonate (1.21 g) is added and the brown suspension is refluxed for another 4 h. After cooling to RT, the reaction mixture is freed from solvent under reduced pressure and the residue is taken up into CH₂Cl₂ (400 mL). Some insoluble residue is filtered off, washed with additional CH₂Cl₂ and the organic layer is washed with water (2×200 mL). The organic layer is dried (Na₂SO₄), and concentrated under reduced pressure to obtain the title compound as brownish solid; MS(ESI⁺):m/z=153.9 (M+H)⁺: HPLC: tRet=2.90 min (System 1). The title compound is used in the next step without further purification.

### Stage 1.2: 3-Bromo-6-chloro-imidazo[1,2-b]pyridazine

6-Chloro-imidazo[1,2-b]pyridazine (example 1; stage 1.1) (4.94 g; 29.3 mMol) is dissolved in DMF (50 mL) and cooled to 0° C. At this temperature, N-bromo-succinimide (5.76 g; 30.7 mMol) is added and the brown solution is stirred at 0° C. for 2 h, followed by stirring at RT for 1 h. The brown solution is taken up into EtOAc (400 mL) and washed with water (2×200 mL), followed by back extraction of the aqueous layers with EtOAc (1×200 mL). The combined organic layers are dried (Na₂SO₄), and concentrated under reduced pressure to obtain the title compound as yellowish crystals; mp. 132-137° C.; MS(ESI⁺):m/z=233.8 (M+H)⁺; HPLC: tRet=4.61 min (System 1). The title compound is used in the next step without further purification. Additional material can be isolated from the mother liquor. The structure is confirmed by x-ray analysis; it contains N-bromo-succinimide.

### Example 2

### 4-[6-(3,4-Dimethoxy-phenyl)-imidazo[1,2-b]pyridazin-3-yl]-benzamide

The title compound is prepared as described in example 1, using 3-bromo-6-(3,4-dimethoxy-phenyl)-imidazo[1,2-b]pyridazine (see example 1) and 4-boronic acid benzamide as alternative starting material. The reaction time is reduced to 15 min. Title compound: Lightly yellow powder; MS(ESI⁺):m/z=375.2 (M+H)⁺; HPLC: tRet=4.000 min (System 2).

### Example 3

### 4-[3-(3,4-Dimethoxy-phenyl)-imidazo[1,2-b]pyridazin-6-yl]-benzamide

The title compound is prepared as described in example 1, using 6-chloro-3-(3,4-dimethoxy-phenyl)-imidazo[1,2-b]pyridazine (see example 1) and 4-boronic acid benzamide as alternative starting material. The reaction time is reduced to 15 min. Title compound: Yellow powder; MS(ESI⁺):m/z=375.2 (M+H)⁺; HPLC: tRet=3.967 min (System 2).

### Example 4

### 5-[6-(3,4-Dimethoxy-phenyl)-imidazo[1,2-b]pyridazin-3-yl]-3-trifluoromethyl-pyridin-2-ylamine

The title compound is prepared as described in example 2, using 5-(4,4,5,5-tetramethyl-[1,3,2]dioxaborolan-2-yl)-3-trifluoromethyl-pyridin-2-ylamine as boronic acid equivalent as alternative starting material. The reaction time is 30 min. Title compound: Yellow powder; MS(ESI⁺):m/z=416.1 (M+H)⁺; HPLC: tRet=4.367 min (System 2).

The starting materials are prepared as follows:

### Stage 4.1

### 5-Bromo-3-trifluoromethyl-pyridin-2-ylamine

To a solution of 5.37 g (32.8 mmol) of 3-trifluoromethyl-pyridin-2-ylamine (Fluorochem) in 100 ml of dry CH₃CN, 6.45 g of N-bromosuccinimide are added in 4 equal portions over a period of 1 h at 0-5° C. under argon. The cooling bath is removed and stirring is continued for 3 h. The solvent is evaporated under vacuum, the residue is dissolved in EtOAc and washed with water and brine. The organic phase is dried over Na₂SO₄ and evaporated. The title compound is a reddish-yellow oil which is used after drying in the dark for 5 h at RT and under high vacuum in the next step without further purification. MS(ESI−):m/z=241.0 (M−H)⁻ tRet=4.992 min (System 2).

### Stage 4.2: 5-(4,4,5,5-Tetramethyl-[1,3,2]dioxaborolan-2-yl)-3-trifluoromethyl-pyridin-2-yl-amine

8.04 g (31.7 mmol) of 5-bromo-3-trifluoromethyl-pyridin-2-ylamine (preparation see Stage 24.4.), 10.5 g (41.2 mmol) of 4,4,5,5,4′,4′,5′,5′-octamethyl-[2,2]bi[[1,32]dioxaborolanyl] (Aldrich), and 9.62 g (95.1 mmol) of KOAc in 100 ml dioxane are degassed with argon for 15 min. Then 776 mg (0.951 mmol) of bis(diphenylphosphino)ferrocene dichloro-palladium(II)dichloromethane (ABCR) are added and the mixture is degassed for 15 more min. The reaction mixture is heated at 115° C. for 8 h. After that time, the reaction mixture is filtered and the solvent evaporated. The residue is purified by simple filtration on silicagel (solvent system: t-butyl-methyl ether-EtOAc-NEt₃=50:50:0.1) to yield the title compound as almost colorless solid. MS(ESI⁺):m/z=289.1 (M+H)⁺; HPLC: tRet=3.292 min (System 2).

### Example 5

### 6-(3,4-Dimethoxy-phenyl)-3-(4-methanesulfonyl-phenyl)-imidazo[1,2-b]pyridazine

The title compound is prepared as described in example 2, but using 4-methanesulfonyl-boronic acid. The reaction time is 15 min. Title compound: White powder; MS(ESI⁺):m/z=410.1 (M+H)⁺; HPLC: tRet=4.367 min (System 2).

### Example 6

### 5-[3-(6-amino-5-trifluoromethyl-pyridin-3-y0-imidazo[1,2-b]pyridazin-6-yl]-3-trifluoromethyl-pyridin-2-ylamine

The title compound is prepared as described in example 1, using 5-(4,4,5,5-tetramethyl-[1,3,2]dioxaborolan-2-yl)-3-trifluoromethyl-pyridin-2-ylamine as boronic acid starting material. The reaction time is 30 min. Title compound: Yellow powder; MS(ESI⁺):m/z=440.1 (M+H)⁺; HPLC: tRet=4.283 min (System 2).

From this same reaction mixture two additional compounds are obtained and isolated as described in example 1:

### 5-(3-Bromo-imidazo[1,2-b]pyridazin-6-yl)-3-trifluoromethyl-pyridin-2-ylamine

Title compound: White powder; MS(ESI⁺):m/z=360.0 (M+H)⁺; HPLC: tRet=4.783 min (System 2).

### 5-(6-Chloro-imidazo[1,2-b]pyridazin-3-yl)-3-trifluoromethyl-pyridin-2-ylamine

Title compound: Yellow powder; MS(ESI⁺):m/z=314.1 (M+H)⁺; HPLC: tRet=4.458 min (System 2).

### Example 7

### 4-[3-(4-Carbamoylphenyl)-imidazo[1,2-b]pyridazin-6-yl]-benzamide

The title compound is prepared as described in example 1, but using benzamide-4-boronic acid. The reaction time is reduced to 15 min. Title compound: Yellow powder; MS(ESI⁺):m/z=358.2 (M+H)⁺; HPLC: tRet=3.625 min (System 2).

From this same reaction mixture two additional compounds are obtained and isolated as described in example 1:

### 4-(3-Bromo-imidazo[1,2-b]pyridazin-6-yl)-benzamide

Title compound: Yellow powder; MS(ESI⁺):m/z=319.0 (M+H)⁺; HPLC: tRet=4.108 min (System 2).

4-(6-Chloro-imidazo[1,2-b]pyridazin-3-yl)-benzamide Title compound: Lightly yellow powder; MS(ESI⁺):m/z=273.1 (M+H)⁺; HPLC: tRet=3.958 min (System 2).

### Example 8

### 5-[3-(3,4-Dimethoxy-phenyl)-imidazo[1,2-b]pyridazin-6-yl]-2-methoxy-benzoic acid ethyl ester

The title compound is prepared as described in example 1, but using 6-chloro-3-(3,4-dimethoxy-phenyl)-imidazo[1,2-b]pyridazine (see example 1) and 2-methoxy-5-(4,4,5,5-tetramethyl-[1,3,2]dioxaborolan-2-yl)-benzoic acid ethyl ester as starting materials. The reaction time is 45 min. Title compound: Yellow powder; MS(ESI⁺):m/z=434.1 (M+H)⁺; HPLC: tRet=4.592 min (System 2).

### Example 9

### 4-[6-(2-Methoxy-phenyl)-imidazo[1,2-b]pyridazin-3-yl]-benzamide

The title compound is prepared as described in example 1, using 4-(6-chloro-imidazo[1,2-b]pyridazin-3-yl)-benzamide (see example 7) and 2-methoxyphenyl-boronic acid instead. The reaction time is 15 min. Title compound: Lightly yellow powder; MS(ESI⁺):m/z=345.2 (M+H)⁺; HPLC: tRet=4.092 min (System 2).

### Example 10

### (3-{4-[3-(6-Amino-5-trifluoromethyl-pyridin-3-yl)-imidazo[1,2-b]pyridazin-6-yl]-2-methoxy-phenoxy}-propyl)-carbamic acid tert-butyl ester

The title compound is prepared as described in example 1, but using 5-(6-chloro-imidazo[1,2-b]pyridazin-3-yl)-3-trifluoromethyl-pyridin-2-ylamine (see example 6) and {3-[2-Methoxy-4-(4,4,5,5-tetramethyl-[1,3,2]dioxaborolan-2-yl)-phenoxy]-propyl}-carbamic acid tert-butyl ester as starting materials. The reaction time is 120 min. Title compound: Yellow powder; MS(ESI⁺):m/z=559.1 (M+H)⁺; HPLC: tRet=4.825 min (System 2).

The starting material is prepared as follows:

### Stage 10.1: {3-[2-Methoxy-4-(4,4,5,5-tetramethyl[1,3,2]dioxaborolan-2-yl)-phenoxy]-propyl}-carbamic acid tert-butyl ester

2-Methoxy-4-(4,4,5,5-tetramethyl-[1,3,2]dioxaborolan-2-yI)-phenol (250 mg; 1 mmol) is dissolved in DMF (1 mL) and cooled to 0° C. After addition of (3-bromo-propyl)-carbamic acid tert-butyl ester (286 mg; 1.2 mmol) the mixture is stirred additional 30 min. at 0° C., followed by stirring without cooling for 16 h. EtOAc (150 mL) is added, the reaction mixture is extracted with water (2×50 mL), followed by removal of the solvent under reduced pressure. Title compound: Red oil; MS(ESI⁺):m/z=408.1 (M+H)⁺; HPLC: tRet=5.817 min (System 2).

### Example 11

### 4-[3-(6-Amino-5-trifluoromethyl-pyridin-3-yl)-imidazo[1,2-b]pyridazin-6-yl]-benzamide

The title compound is prepared as described in example 1, but using 4-(3-bromoimidazo[1,2-b]pyridazin-6-yl)-benzamide (see example 7) and 5-(4,4,5,5-Tetramethyl-[1,3,2]dioxaborolan-2-yl)-3-trifluoromethyl-pyridin-2-ylamine (example 4; stage 4.2) as starting materials. The reaction time is 120 min. Title compound: Yellow powder; MS(ESI⁺):m/z=399.1 (M+H)⁺; HPLC: tRet=3.892 min (System 2).

### Example 12

### {2-Carbamoyl-4-[3-(3,4-dimethoxy-phenyl)-imidazo[1,2-b]pyridazin-6-yl]-phenoxy}-acetic acid methyl ester

The title compound is prepared as described in example 1, but using 6-Chloro-3-(3,4-dimethoxy-phenyl)-imidazo[1,2-b]pyridazine (see example 1) and [2-carbamoyl-4-(4,4,5,5-tetramethyl-[1,3,2]dioxaborolan-2-yl)-phenoxy]-acetic acid methyl ester as starting materials. As a catalyst, 1,1′-bis(diphenylphosphino)ferrocene palladium(II) chloride, complex with dichloromethane (1:1) [CAS Nr 72287-26-4] (6 mg) is used. The reaction time is 15 min. Title compound: Yellow powder; MS(ESI⁺):m/z=463.2 (M+H)⁺; HPLC: tRet=4.192 min (System 2).

### Example 13

### 5-{4-[6-(3,4-Dimethoxy-phenyl)-imidazo[1,2-b]pyridazin-3-yl]-phenyl}-pyridine-2-carbonitrile

3-(4-Chloro-phenyl)-6-(3,4-dimethoxy-phenyl)-imidazo[1,2-b]pyridazine (44 mg; 0.12 mMol) is dissolved in THF (5 mL) at RT followed by addition of 5-pyridine-2-carbonitrile boronic acid (56 mg; 0.24 mMol), potassium phosphate (128 mg; 0.6 mmol), Pd(dba)2 (3.5 mg) and SPhos (5 mg). This mixture is stirred at 150° C. for 45 min at 300 W in an EmryOptimizer microwave oven. After cooling to RT, EtOAc is added (50 mL), followed by extraction with water (2×). The solvent is removed under reduced pressure and the crude product purified by flash chromatography (30 g silica gel [0.040-0.063 mm] Merck 1.09.385.1000]; eluting with CH₂Cl₂/CH₃OH 98.5%:1.5%). The combined fractions are freed from solvent, taken up into dioxane and freeze-dried to obtain the title compound as yellow powder; MS(ESI⁺):m/z=434.1 (M+H)⁺; HPLC: tRet=4.983 min (System 2).

The starting material is prepared as follows:

### Stage 13.1: 3-(4-Chloro-phenyl)-6-(3,4-dimethoxy-phenyl)-imidazo[1,2-b]pyridazine

The title compound is prepared as described in example 1, but using 3-bromo-6-(3,4-dimethoxy-phenyl)-imidazo[1,2-b]pyridazine (see example 1) and 4-chloro-boronic acid as starting materials. The reaction time is reduced to 15 min. Title compound: Lightly yellow powder; MS(ESI⁺):m/z=366.1 (M+H)⁺; HPLC: tRet=5.050 min (System 2).

### Example 14

### 5-{6-[4-(3-Amino-propoxy)-3-methoxy-phenyl]-imidazo[1,2-b]pyridazin-3-yl}-3-trifluoromethyl-pyridin-2-ylamine

(3-{4-[3-(6-Amino-5-trifluoromethyl-pyridin-3-yl)-imidazo[1,2-b]pyridazin-6-yl]-2-methoxy-phenoxy}-propyl)-carbamic acid tert-butyl ester (example 10) (28 mg; 0.05 mMol) is dissolved in TFA (0.5 mL) and stirred at RT for 5 min. The reaction mixture is adjusted to pH 8 with NaHCO₃ (5% solution) followed by extraction with butanol. The organic layer is extracted with water (2×) and freed from the solvent under reduced pressure, followed by lyophilisation from dioxan, to obtain the title compound. Title compound: Yellow powder; MS(ESI⁺):m/z=459.1 (M+H)⁺; HPLC: _{tRet}=3.783 min (System 2).

### Example 15

### (3-{4-[3-(4-Carbamoyl-phenyl)-imidazo[1,2-b]pyridazin-6-yl]-2-methoxy-phenoxy}-propyl)-carbamic acid tert-butyl ester

The title compound is prepared as described in example 1, but using 4-(6-chloro-imidazo[1,2-b]pyridazin-3-yl)-benzamide (see example 7) and {3-[2-methoxy-4-(4,4,5,5-tetra-methyl-[1,3,2]dioxaborolan-2-yl)-phenoxy]-propyl}-carbamic acid tert-butyl ester (example 10; stage 10.1) as starting materials. The reaction time is 30 min. Title compound: Lightly yellow powder; MS(ESI⁺):m/z=518.1 (M+H)⁺; HPLC: tRet=4.475 min (System 2).

### Example 16

### 1-[5-[3-(3,4-Dimethoxy-phenyl)-imidazo[1,2-b]pyridazin-6-yl]-2-(2-hydroxy-ethoxy)-phenyl]-ethanone

The title compound is prepared as described in example 1, but using 6-chloro-3-(3,4-dimethoxy-phenyl)-imidazo[1,2-b]pyridazine (see example 1) and 1-[2-(2-hydroxy-ethoxy)-5-(4,4,5,5-tetramethyl-[1,3,2]dioxaborolan-2-yl)-phenyl]-ethanone as starting materials. The reaction time is 60 min. Title compound: Yellow powder; MS(ESI⁺):m/z=434.1 (M+H)⁺; HPLC: tRet=4.158 min (System 2).

### Example 17

### 4-{6-[4-(3-Amino-propoxy)-3-methoxy-phenyl]-imidazo[1,2-b]pyridazin-3-yl}-benzamide

The title compound is, prepared as described in example 14, but starting from (3-{4-[3-(4-carbamoyl-phenyl)-imidazo[1,2-b]pyridazin-6-yl]-2-methoxy-phenoxy}-propyl)-carbamic acid tert-butyl ester (example 15). Title compound: Lightly yellow powder; MS(ESI⁺):m/z=418.2 (M+H)⁺; HPLC: tRet=3.617 min (System 2).

### Example 18

### 5-[3-(4-Methanesulfonyl-phenyl)-imidazo[1,2-b]pyridazin-6-yl]-3-trifluoro-methyl-pyridin-2-ylamine

The title compound is prepared as described in example 1, but using 5-(3-bromo-imidazo[1,2-b]pyridazin-6-yl)-3-trifluoromethyl-pyridin-2-ylamine (see example 6) and 4-methanesulfonyl-boronic acid as starting materials. The reaction time is 120 min. Title compound: Yellow powder; MS(ESI⁺):m/z=434.1 (M+H)⁺; HPLC: tRet=4.283 min (System 2).

### Example 19

### 6-(3,4-Dimethoxy-phenyl)-3-(4-furan-3-yl-phenyl)-imidazo[1,2-b]pyridazine

The title compound is prepared as described in example 13, but using furane-3-boronic acid as starting material. The reaction time 45 min at 150° C. and 300 W in an EmryOptimizer microwave oven. Title compound: Lightly yellow powder; MS(ESI⁺):m/z=398.2 (M+H)⁺; HPLC: tRet=5.017 min (System 2).

### Example 20

### 6-(3,4-Dimethoxy-phenyl)-3-[4-(1H-pyrrol-2-yl)-phenyl]-imidazo[1,2-b]pyridazine

The title compound is prepared as described in example 13, but using 1-(tert-butoxycarbonyl)-1H-pyrrole-2-boronic acid as starting material. The reaction time is 5 h at 150° C. and 300 W in an EmryOptimizer microwave oven. The Boc group is removed with TFA as described in example 14. Title compound: beige powder; MS(ESI⁺):m/z=397.2 (M+H)⁺; HPLC: tRet=4.808 min (System 2).

### Example 21

### (3-{4-[6-(4-Carbamoyl-phenyl)-imidazo[1,2-b]pyridazin-3-yl]-2-methoxy-phenoxy}-propyl)-carbamic acid tert-butyl ester

The title compound is prepared as described in example 1, but using 4-(3-bromo-imidazo[1,2-b]pyridazin-6-yl)-benzamide (see example 7) and {3-[2-methoxy-4-(4,4,5,5-tetramethyl-[1,3,2]dioxaborolan-2-yl)-phenoxy]-propyl}-carbamic acid tert-butyl ester (example 10; stage 10.1) as starting material. The reaction time is 60 min. Title compound: Yellow powder; MS(ESI⁺):m/z=518.2 (M+H)⁺; HPLC: tRet=4.400 min (System 2).

### Example 22

### 6-(3,4-Dimethoxy-phenyl)-3-(4-thiophen-3-yl-phenyl)-imidazo[1,2-b]pyridazine

The title compound is prepared as described in example 13, but using thiophene-3-boronic acid as starting material. The reaction time 90 min at 150° C. and 300 W in an EmryOptimizer microwave oven. Title compound: Lightly yellow powder; MS(ESI⁺):m/z=414.1 (M+H)⁺; HPLC: tRet=5.233 min (System 2).

### Example 23

### 4-{3-[4-(3-Amino-propoxy)-3-methoxy-pheny]-imidazo[1,2-b]pyridazin-6-yl}-benzamide

The title compound is prepared as described in example 14, but starting from (3-{4-[6-(4-carbamoyl-phenyl)-imidazo[1,2-b]pyridazin-3-yl]-2-methoxy-phenoxy}-propyl)-carbamic acid tert-butyl ester (example 21) as starting material. Title compound: Yellow powder; MS(ESI⁺):m/z=418.2 (M+H)⁺; HPLC: tRet=3.567 min (System 2).

### Example 24

### 6-(3,4-Dimethoxy-phenyl)-3-(1H-pyrrolo[2,3-b]pyridin-5-yl)-imidazo[1,2-b]pyridazine

The title compound is prepared as described in example 1, but using 3-bromo-6-(3,4-dimethoxy-phenyl)-imidazo[1,2-b]pyridazine (see example 1) and 5-(4,4,5,5-tetramethyl-[1,3,2]dioxaborolan-2-yI)-1H-pyrrolo[2,3-b]pyridine (Alfa Aesar; named 7-azaindole-5-boronic acid pinacol ester) instead. The reaction time is 90 min. Title compound: Yellow powder; MS(ESI⁺):m/z=372.2 (M+H)⁺; HPLC: tRet=4.183 min (System 2).

### Example 25

### (3-{4-[3-(3,4-Dimethoxy-phenyl)-pyrazolo[1,5-a]pyrimidin-5-yl]-2-methoxy-phenoxy}-propyl)-carbamic acid tert-butyl ester

5-Chloro-3-(3,4-dimethoxy-phenyl)-pyrazolo[1,5-a]pyrimidine (100 mg; 0.324 mMol), {3-[2-methoxy-4-(4,4,5,5-tetramethyl-[1,3,2]dioxaborolan-2-yl)-phenoxy]-propyl}-carbamic acid tert-butyl ester (220 mg; 0.486 mMol) (example 10; stage 10.1) and PdCl₂(PPh₃) (12 mg) are dissolved in DMF (10 mL), followed by addition of potassium carbonate (1M solution in H₂O; (0.81 mL), and the clear yellow reaction solution is stirred at 120° C. for 90 min. After cooling to RT, the mixture is taken up into water (20 mL), and extracted with EtOAc (3×100 mL). The combined organics are washed with NaHCO₃ saturated solution, water, brine and dried over Na₂SO₄, followed by removal of the solvent under reduced pressure. Purification is done by chromatography (40 g RediSep Catalog number 68-2203-027, Teledyne Isco, Inc., Lincoln, Nebr., USA; eluting with EtOAc), to obtain the title compound as yellowish crystals (150 mg); mp. 131-133° C.; MS(ESI⁺):m/z=535.1 (M+H)⁺; HPLC: tRet=7.40 min (System1).

The synthesis of the starting material 5-chloro-3-(3,4-dimethoxy-phenyl)-pyrazolo[1,5-a]pyrimidine is as described up to 4-(3,4-dimethoxy-phenyl)-2H-pyrazol-3-ylamine in published PCT application WO 2005/070431 (incorporated by reference herewith, especially regarding the synthesis; see example 93, stage 93.1); and then up to 5-chloro-3-(3,4-dimethoxyphenyl)-pyrazolo[1,5-a]pyrimidine takes place as follows:

### Stage 25.1: 3-(3,4-Dimethoxy-phenyl)-4H-pyrazolo[1,5-a]pyrimidin-5-one

4-(3,4-Dimethoxy-phenyl)-2H-pyrazol-3-ylamine (see WO 2005/070431) (10 g; 45.6 mMol) is suspended in 1,4-dioxane and treated at RT with propiolic acid methylester (4.10 mL; 45.6 mMol). The reaction mixture is stirred at 110° C. (bath) for 46 h. After cooling to RT, the precipitated product is filtered off, washed with 1,4-dioxane and dried to obtain the title compound as a white solid. Title compound: MS(ESI⁺):m/z=272.0 (M+H)⁺; HPLC: tRet=4.43 min (System 1).

### Stage 25.2: 5-Chloro-3-(3,4-dimethoxy-phenyl)-pyrazolo[1,5-a]pyrimidine

3-(3,4-Dimethoxy-phenyl)-4H-pyrazolo[1,5-a]pyrimidin-5-one (example 1; stage 1.1) (1.0 g; 3.69 mMol) is suspended in POCl₃ (17.2 mL; 184 mMol) and stirred for 2 d at 120° C. After cooling to RT the solvent is removed under reduced pressure, the residue is taken up into NaHCO₃ std. soln (70 mL) and extracted with EtOAc (2×200 mL). The combined organic layers are washed with NaHCO₃ saturated solution water, brine, dried (Na₂SO₄), and concentrated under reduced pressure. After stirring in diethyl ether, and filtering off, the title compound is obtained as brown crystals. Title compound: MS(ESI⁺):m/z=290.0 (M+H)⁺; HPLC: tRet=5.53 min (System 1).

### Example 26

### 3-{4-[3-(3,4-Dimethoxy-phenyl)-pyrazolo[1,5-a]pyrimidin-5-yl]-2-methoxy-phenoxy}-propylamine

(3-{4-[3-(3,4-Dimethoxy-phenyl)-pyrazolo[1,5-a]pyrimidin-5-yl]-2-methoxy-phenoxy}-propyl)-carbamic acid tert-butyl ester (86 mg; 0.156 mMol) (example 25) is suspended in HCl (4M solution in dioxane; 2.2 mL) and stirred at RT for 3 h. The yellow suspension is taken up into NaHCO₃ saturated solution (20 mL) and extracted with EtOAc (3×100 mL) The combined organic phases are washed with water, brine and dried over Na₂SO₄, followed by removal of the solvent under reduced pressure, to obtain the title compound as yellowish crystals (17.2 mg). Title compound: MS(ESI⁺):m/z=435.2 (M+H)⁺; HPLC: tRet=5.36 min (System1).

### Purification and Characterization Conditions for Examples 27 to 32

The compounds and/or intermediates are characterized by high performance liquid chromatography (HPLC) using a Waters Millenium chromatography system with a 2695 Separation Module (Milford, Mass., USA). The analytical columns are reversed phase Phenomenex Luna C18-5p, 4.6×50 mm, from Alltech (Deerfield, Ill., USA). A gradient elution is used (flow 2.5 mL/min), typically starting with 5% acetonitrile/95% water and progressing to 100% acetonitrile over a period of 10 min. All solvents contain 0.1% trifluoroacetic acid (TFA). Compounds are detected by ultraviolet light (UV) absorption at either 220 or 254 nm. HPLC solvents are from Burdick and Jackson (Muskegan, Mich., USA), or Fisher Scientific (Pittsburgh, Pa., USA).

In some instances, purity is assessed by thin layer chromatography (TLC) using glass or plastic backed silica gel plates, such as, for example, Baker-Flex Silica Gel 1 B2-F flexible sheets (Mallinckrodt Baker, Inc., Phillipsburg, N.J., USA). TLC results are readily detected visually under ultraviolet light, or by employing well-known iodine vapor and or other staining techniques.

Mass spectrometric analysis is performed on one of two LC/MS instruments: a Waters System (Alliance HT HPLC and a Micromass ZQ mass spectrometer; Column: Eclipse XDB-C18, 2.1×50 mm; gradient: 5-95% (or 35-95%, or 65-95% or 95-95%) acetonitrile water with 0.05% TFA over a 4 min period; flow rate 0.8 mL/min; molecular weight range 200-1500; cone Voltage 20 V; column temperature 40° C.; Waters Corporation, Milford, Mass., USA) or a Hewlett Packard System (Series 1100 HPLC; Column: Eclipse XDB-C18, 2.1×50 mm; gradient: 5-95% acetonitrile in water with 0.05% TFA over a 4 min period; flow rate 0.8 mL/min; molecular weight range 150-850; cone Voltage 50 V; column temperature 30° C.; now Agilent Technologies, Inc., Santa Clara, Calif., USA). All masses are reported as those of the protonated parent ions.

Preparative separations are carried out using a Flash 40 chromatography system and KP-Sil, 60A (Biotage, Charlottesville, Va., USA), or by flash column chromatography using silica gel (230-400 mesh) packing material, or by HPLC using a Waters 2767 Sample Manager, C-18 reversed phase column, 30×50 mm, flow 75 mL/min. Typical solvents employed for the Flash 40 Biotage system and flash column chromatography are dichloromethane, methanol, ethyl acetate, hexane, acetone, aqueous ammonia (or ammonium hydroxide), and triethyl amine. Typical solvents employed for the reverse phase HPLC are varying concentrations of acetonitrile and water with 0.1% trifluoroacetic acid.

### Example 27

### 5-[3-(6-Fluoro-pyridin-3-yl)-imidazo[1,2-b]pyridazin-6-yl]-3-trifluoromethyl-pyridin-2-ylamine

6-Chloro-3-(6-fluoropyridin-3-yl)imidazo[1,2-b]pyridazine (125 mg, 0.50 mmol) and 5-(4,4,5,5-tetramethyl-1,3,2-dioxaborolan-2-yl)-3-(trifluoromethyl)pyridin-2-amine (230 mg, 0.80 mmol) are mixed with 10 mL of 1,4-dioxane and 2 mL of 2 M Na₂CO₃ aqueous solution in a glass pressure tube. The reaction mixture is degassed by anhydrous N₂ stream for 5 min and of Pd(dppf)Cl₂-DCM (complex with dichloromethane) (41 mg, 0.05 mmol) is added. The reaction mixture is stirred at 80° C. for 2 h, cooled to room temperature and diluted with 100 mL of ethyl acetate. The two phases are separated and the organic phase is washed with water, brine, then dried over MgSO₄. The EtOAc is filtered and evaporated under reduced pressure to give the crude product, which is purified by column chromatography on silica gel (5% MeOH in1:1 EtOAc/Hexane) to give the title compound. Title compound: LC/MS (m/z): 375 (MH+), tRet: 2.14 min.

The starting material is prepared as follows:

### Stage 27.1

### 6-chloro-3-(6-fluoropyridin-3-yl)imidazo[1,2-b]pyridazine

3-Bromo-6-chloro-imidazo[1,2-b]pyridazine (696 mg, 3.0 mmol) (example 1; stage 1.2) and 6-fluoropyridin-3-ylboronic acid (423 mg, 3.0 mmol) are mixed with 15 mL of 1,4-dioxane and 6 mL of 2 M Na₂CO₃ aqueous solution in a glass pressure tube. The reaction mixture is degassed by anhydrous N₂ stream for 5 min and Pd(dppf)Cl₂-DCM (245 mg, 0.30 mmol) is added. The reaction mixture is stirred at 80° C. for 3 h, cooled to room temperature and diluted with 150 mL of ethyl acetate. The two phases are separated and the organic phase is washed with water, then brine, then dried over MgSO₄. The EtOAc is filtered and evaporated under reduced pressure to give the crude product, which is purified by column chromatography on silica gel (1:2 EtOAc/Hexane) to give the title compound. Title compound: LC/MS (m/z): 249:251=3:1 (MH+), tRet: 2.16 min.

### Example 28

### 5-{3-[6-(4-Phenyl-thiazol-2-ylamino)-pyridin-3-yl]-imidazo[1,2-b]pyridazin-6-yl}-3-trifluoromethyl-pyridin-2-ylamine

A mixture of 5-(3-(6-fluoropyridin-3-yl)imidazo[1,2-b]pyridazin-6-yl)-3-(trifiuoromethyl)pyridin-2-amine (12 mg, 0.032 mmol), 4-phenylthiazol-2-amine (11 mg, 0.064 mmol) and cesium carbonate (20.8 mg, 0.064 mmol) in 0.5 mL of 1-methylpyrrolidin-2-one is stirred in a microwave reactor at 160° C. for 600 sec. The crude product is then purified by preparative HPLC to give the title compound. Title compound: LC/MS (m/z): 531.0 (MH+), tRet: 2.66 min.

### Example 29

### 5-{3-[6-(1-Isopropyl-piperidin-4-yloxy)-pyridin-3-yl]-imidazo[1,2-b]pyridazin-6-yl}-3-trifluoromethyl-pyridin-2-ylamine

A solution of the 1-isopropylpiperidin-4-ol (7.6 mg, 0.053 mmol), sodium hydride (2.4 mg, 0.1 mmol) and 5-(3-(6-fluoropyridin-3-yl)imidazo[1,2-b]pyridazin-6-yl)-3-(trifluoromethyl)pyridin-2-amine (10 mg, 0.027 mmol) in 0.7 mL of 1-methylpyrrolidin-2-one is stirred at room temperature overnight. The crude product is then purified by preparative HPLC to give the title compound. Title compound: LC/MS (m/z): 498.2 (MH+), tRet: 1.94 min.

### Example 30

### 5-[3-(6-Benzylamino-pyridin-3-yl)-imidazo[1,2-b]pyridazin-6-yl]-3-trifluoro-methyl-pyridin-2-ylamine

The title compound is prepared as described for example 28, but using benzylamine as starting material. The crude product is purified by preparative HPLC to give the title compound. Title compound: LC/MS (m/z): 462.1 (MH+), tRet: 2.09 min.

### Example 31

### 5-[3-(6-Morpholin-4-yl-pyridin-3-yl)-imidazo[1,2-b]pyridazin-6-yl]-3-trifluoromethyl-pyridin-2-ylamine

A mixture of 5-(3-(6-fluoropyridin-3-yl)imidazo[1,2-b]pyridazin-6-yl)-3-(trifluoromethyl)pyridin-2-amine (10 mg, 0.027 mmol) in 0.2 mL of morpholine is stirred in a microwave reactor at 140° C. for 600 sec. The crude product is then purified by preparative HPLC to give the title compound: LC/MS (m/z): 442.2 (MH+), tRet: 1.87 min.

### Example 32

### 5-[6-(6-Amino-5-trifluoromethyl-pyridin-3-yl)-imidazo[1,2-b]pyridazin-3-yl]-pyridin-2-ol

A mixture of 5-(3-(6-fluoropyridin-3-yl)imidazo[1,2-b]pyridazin-6-yl)-3-(trifluoromethyl)pyridin-2-amine (10 mg, 0.027 mmol), 0.1 mL of acetic acid and 0.5 mL of water is stirred in a microwave reactor at 160° C. for 600 sec. The crude product is then purified by preparative HPLC to give the title compound. Title compound: LC/MS (m/z): 373.1 (MH+), tRet: 1.70 min.

Embodiments of the present invention are also represented by the compounds shown in the following table:

| | | | | | HPLC |
|---|---|---|---|---|---|
| ex- | | | | | (all |
| ample | | | | | system 2 |
| num- | | compound | method of | ESI- | except *) |
| ber | Structure | name | preparation | MS+ | **System 1 |
| 33 | 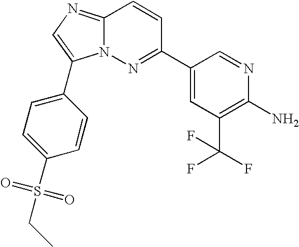 | 5-[3-(4- Ethane- sulfonyl- phenyl)- imidazo [1,2-b] pyridazin-6- yl]-3- trifluoro- methyl- pyridin- 2-ylamine | Method A | 448 | 4.467 |
| 34 | 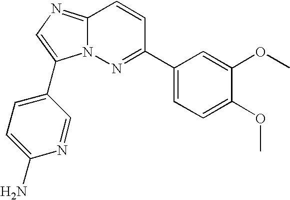 | 5-[6-(3,4- Dimethoxy- phenyl)- imidazo [1,2-b] pyridazin- 3-yl]- pyridin-2- ylamine | Method A using 3,4- dimethoxy- boronic acid and 2- amino- pyridine-5- boronic acid | 348.1 | 3.775 |
| 35 | 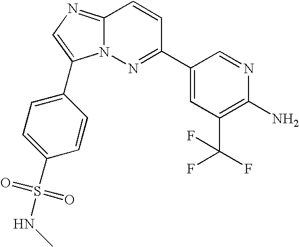 | 4-[6-(6- Amino-5- trifluoro- methyl- pyridin-3- yl)- imidazo [1,2-b] pyridazin- 3-yl]-N- methyl- benzene- sulfon- amide | Method A using 4- methyl- sulfonyl- phenyl- boronic acid | 449.1 | 4.258 |
| 36 | 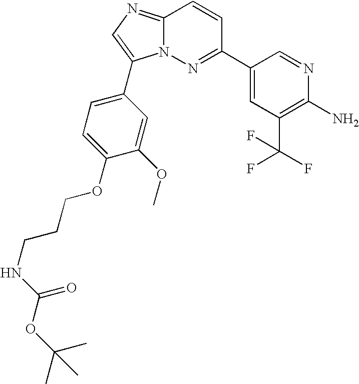 | (3-{4-[6-(6- Amino-5- trifluoro- methyl- pyridin-3- yl)- imidazo [1,2-b] pyridazin- 3-yl]-2- methoxy- phenoxy}- propyl)- carbamic acid tert-butyl ester | Method A using 4-(3-tert- butoxy- carbonyl- amino- propoxy)- 3- methoxy- boronic acid* (*see Method F) | 559.1 | 4.783 |
| 37 | 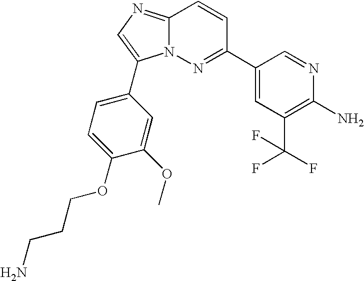 | 5-{3-[4-(3- Amino- propoxy)- 3-methoxy- phenyl]- imidazo[1,2- b]pyridazin- 6-yl}-3- trifluoro- methyl- pyridin-2- ylamine | Method B | 459.1 | 3.775 |
| 38 | 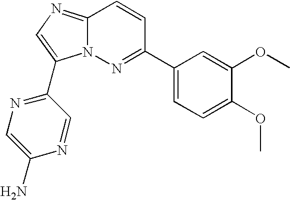 | 5-[6-(3,4- Dimethoxy- phenyl)- imidazo [1,2-b] pyridazin- 3-yl]- pyrazin- 2-ylamine | Method A using 3,4- dimethoxy- boronic acid and 5- amino- pyrazine-2- boronic acid | 349.1 | 3.958 |
| 39 | 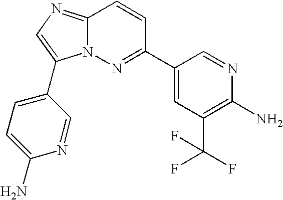 | 5-{3-(6- Amino- pyridin-3- yl)- imidazo [1,2-b] pyridazin- 6-yl]-3- trifluoro- methyl- pyridin-2- ylamine | Method A using 5- (4,4.5,5- Tetra- methyl- [1,3,2] dioxa- borolan-2- yl)- pyridin-2- ylamine | 372.1 | 3.692 |
| 40 | 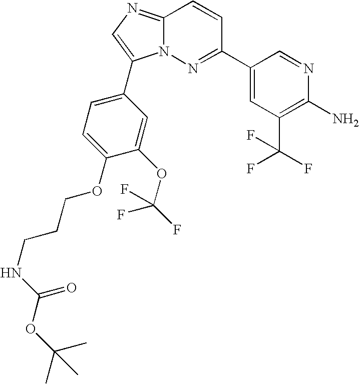 | (3-{4-[6-(6- Amino-5- trifluoro- methyl- pyridin-3- yl)- imidazo [1,2-b] pyridazin-3- yl]- 2- trifluoro- methoxy- phenoxy}- propyl)- carbamic acid tert- butylester | Method A using {3-[4- (4,4,5,5- tetra- methyl- [1,3,2] dioxa- borolan-2- yl)-2- trifluoro- methoxy- phenoxy]- propyl}- carbamic acid tert- butyl ester | 612.9 | 5.175 |
| 41 | 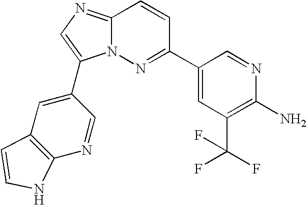 | 5-[3-(1H- Pyrrolo [2,3-b] pyridin-5- yl)- imidazo [1,2-b] pyridazin- 6-yl]-3- trifluoro- methyl- pyridin-2- ylamine | Method A using 5- (4,4,5,5- Tetra- methyl- [1,3,2] dioxa- borolan- 2-yl)-1H- pyrrolo [2,3-b] pyridine (method C) | 396.1 | 4.05 |
| 42 | 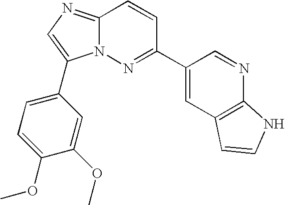 | 3-(3,4- Dimethoxy- phenyl)-6- (1H- pyrrolo[2,3- b]pyridin-5- yl)-imidazo [1,2-b] pyridazine | Method A using 5- (4,4,5,5- tetra- methyl- [1,3,2] dioxa- borolan-2- yl)-1H- pyrrolo [2,3- b] pyridine and 3,4- dimethoxy- boronic acid | 372.2 | 4.158 |
| 43 | 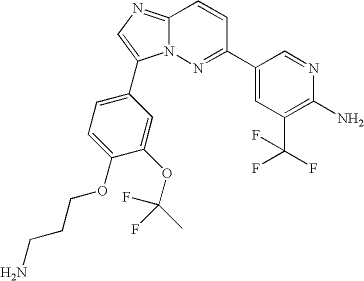 | 5-{3-[4-(3- Amino- propoxy)-3- trifluoro- methoxy- phenyl]- imidazo[1,2- b]pyri- dazin-6-yl}- 3-trifluoro- methyl- pyridin- 2-ylamine | Method B starting from (3-{4- [6-(6- amino-5- trifluoro- methyl- pyridin-3- yl)-imidazo [1,2-b] pyridazin-3- yl]-2- trifluoro- methoxy- phenoxy}- propyl)- | 513.1 | 4.05 |
| 44 | 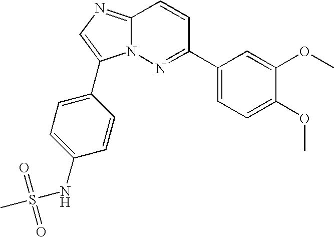 | N-{4-[6-(3,4- Dimethoxy- phenyl)- imidazo [1,2-b] pyridazin- 3-yl]- phenyl}- methane- sulfonamide | Method A using 3,4- dimethoxy- boronic acid and 4- methane- sulfonyl- amino- boronic acid | 425.1 | 4.192 |
| 45 | 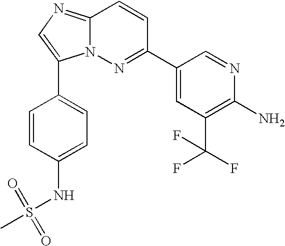 | N-{4-[6-(6- Amino-5- trifluoro- methyl- pyridin-3-yl)- imidazo [1,2-b] pyridazin- 3-yl]- phenyl}- methane- sulfonamide | Method A using 4- methane- sulfonyl- amino- boronic acid | 449 | 4.092 |
| 46 | 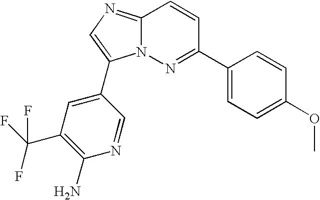 | 5-[6-(4- Methoxy- phenyl)- imidazo[1,2 b] pyridazin-3- yl]-3 trifluoro- methyl- pyridin-2- ylamine | Method A using 4- mcthoxy boronic acid and 5- (4,4,5,5- tetramethyl- [1,3,2] dioxa- borolan- 2-yl)-3- trifluoro- methyl- pyridin-2- ylamine | 386.1 | 4.608 |
| 47 | 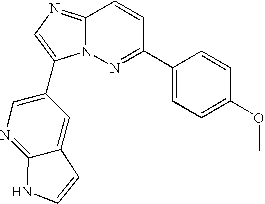 | 6-(4- Methoxy- phenyl)-3- (1H- pyrrolo [2,3-b] pyridin- 5-yl)- imidazo [1,2-b] pyridazine | Method A using 4- methoxy- boronic acid and 5- (4,4,5,5- tetra- methyl- [1,3,2] dioxa- borolan- 2-yl)-1H- pyrrolo [2,3-b] pyridine | 342.2 | 4.325 |
| 48 | 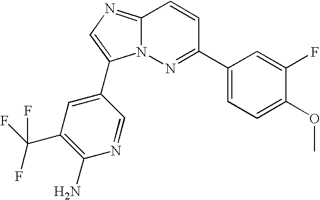 | 5-[6-(3- Fluoro-4- methoxy- phenyl)- imidazo [1,2-b] pyridazin- 3-yl]-3- trifluoro- methyl- pyridin-2- ylamine | Method A using 3- fluoro-4- methoxy- boronic acid and 5- (4,4,5,5- tetramethyl- [1,3,2] dioxa- borolan-2- yl)-3- trifluoro- methyl- pyridin-2- ylamine | 404.1 | 4.675 |
| 49 | 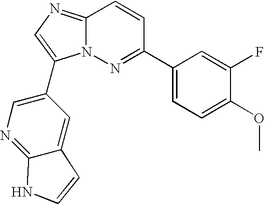 | 6-(3-Fluoro- 4-methoxy- phenyl)-3- (1H- pyrrolo [2,3-b] pyridin-5- yl)- imidazo [1,2-b] pyridazine | Method A using 3- fluoro-4- methoxy- boronic acid and 5- (4,4,5,5- tetra- methyl- [1,3,2] dioxa- borolan- 2-yl)- 1H- pyrrolo [2,3-b] pyridine | 360.1 | 4.392 |
| 50 | 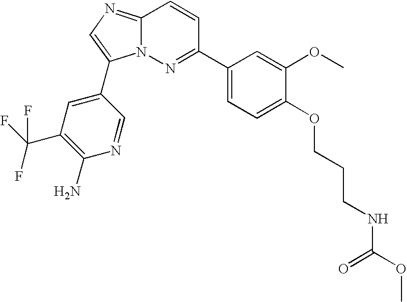 | (3-{4-[3-(6- Amino-5- trifluoro- methyl- pyridin-3- yl)-imidazo [1,2-b] pyridazin-6- yl]-2- methoxy- phenoxy}- propyl)- carbamic acid methyl ester | Method D | 517.1 | 4.375 |
| 51 | 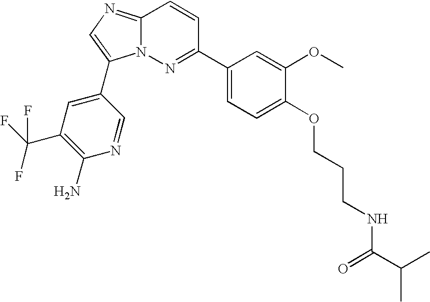 | N-(3-{3-(6- Amino-5- trifluoro- methyl- pyridin-3- yl)-imidazo [1,2-b] pyridazin-6- yl]-2- methoxy- phenoxy}- propyl)- isobutyr- amide | Method D using isobutyryl chloride | 529.1 | 4.375 |
| 52 | 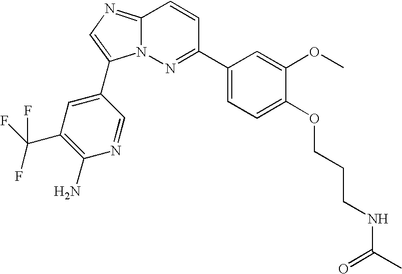 | N-(3-{4-[3- (6- Amino-5- trifluoro- methyl- pyridin-3- yl)- imidazo [1,2-b] pyridazin- 6-yl]-2- methoxy- phenoxy}- propyl)- acetamide | Method D using acetic anhydride, reaction time 30 min. | 500.7 | 4.117 |
| 53 | 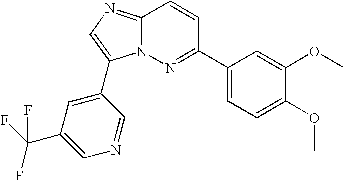 | 6-(3,4- Di- methoxy- phenyl)-3- (5- trifluoro- methyl- pyridin-3- yl)- imidazo [1,2-b] pyridazine | Method A using 3,4- dimethoxy- boronic acid and 3- (4,4,5,5- tetra- methyl- [1,3,2] dioxa- borolan- 2-yl)-5- trifluoro- methyl- pyridine | 401.1 | 5.317 |
| 54 | 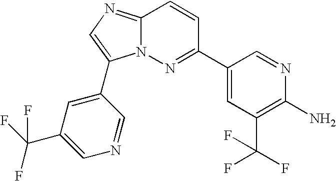 | 3- Trifluoro- methyl- 5-[3-(5- trifluoro- methyl- pyridin-3- yl)-imidazo [1,2-b] pyridazin- 6-yl]- pyridin- 2-ylamine | Method A using 3- (4,4,5,5- tetra- methyl- [1,3,2] dioxa- borolan- 2-yl)-5- trifluoro- methyl- pyridine | 425.1 | 5.1 |
| 55 | 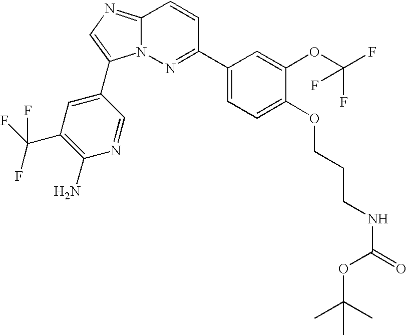 | | Method A using {3-[4- (4,4,5,5- tetra- methyl- [1,3,2] dioxa- borolan- 2-yl)-2- trifluoro- methoxy- phenoxy]- propyl}- carbamic acid tert- butyl ester and 5- (4,4,5,5- tetra- methyl- [1,3,2] dioxa- borolan- 2-yl)-3- trifluoro- methyl- pyridin- 2-ylamine | 613 | 5.242 |
| 56 | 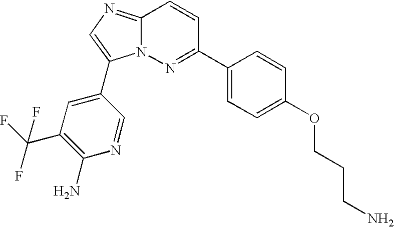 | 5-{6-[4-(3- Amino- propoxy)- phenyl]- imidazo [1,2-b] pyridazin- 3-yl}-3- trifluoro- methyl- pyridin-2- ylamine | Method B starting from (3-{4- [3-(6- amino-5- trifluoro- methyl- pyridin-3- yl)- imidazo [1,2-b] pyridazin- 6-yl]- phenoxy}- propyl)- carbamic acid tert- butyl ester | 429.1 | 3.8 |
| 57 | 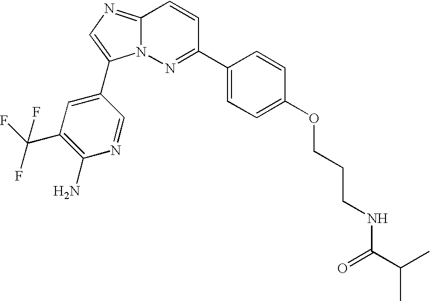 | N-(3-{4-[3-(6- Amino-5- trifluoro- methyl- pyridin-3- yl)-imidazo [1,2-b] pyridazin- 6-yl]- phenoxy}- propyl)- isobutyr- amide | Method D starting from 5-{6- [4-(3- amino- propoxy)- phenyl]- imidazo[1,2- b]pyridazin- 3-yl}-3- trifluoro- methyl- pyridin-2- ylamine (example 56) using isobutyryl chloride | 499.1 | 4.425 |
| 58 | 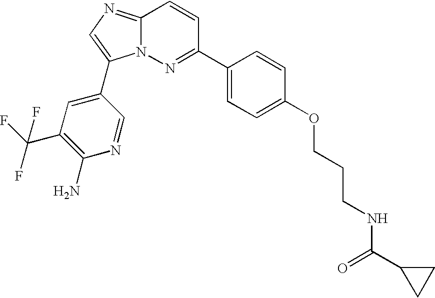 | Cyclo- propane- carboxylic acid (3- {4-[3-(6- amino-5- trifluoro- methyl- pyridin-3- yl)-imidazo [1,2-b] pyridazin- 6-yl]- phenoxy}- propyl)- amide | Method E | 497.1 | 4.392 |
| 59 | 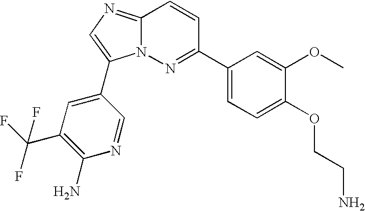 | 5-{6-[4-(2- Amino- ethoxy)-3- methoxy- phenyl]- imidazo [1,2-b] pyridazin-3- yl}-3- trifluoro- methyl- pyridin- 2-ylamine | Method B starting from (2-{4-[3-(6- amino-5- trifluoro- methyl- pyridin-3- yl}- imidazo [1,2-b] pyridazin- 6-yl]-2- methoxy- phenoxy}- ethyl)- carbamic acid tert-butyl ester | 445.1 | 3.75 |
| 60 | 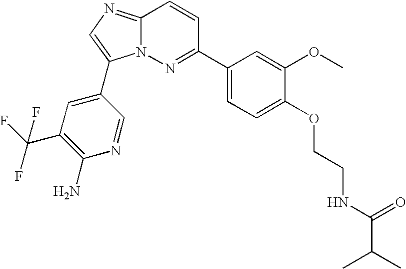 | N-(2-{4-[3-(6- Amino-5- trifluoro- methyl- pyridin-3-yl)- imidazo [1,2-b] pyridazin-6- yl]- 2-methoxy- phenoxy}- ethyl)- isobutyr- amide | Method D starting from 5-{6-[4-(2- amino- ethoxy)-3- methoxy- phenyl]- imidazo[1,2- b]pyridazin- 3-yl}-3- trifluoro- methyl- pyridin- 2-ylamine (example 59) using isobutyryl chloride | 515 | 4.283 |
| 61 | 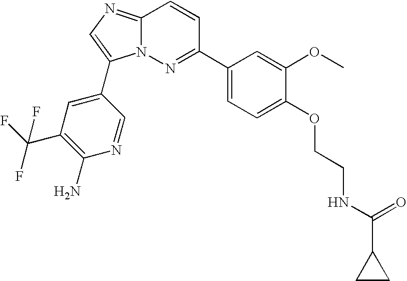 | Cyclo- propane- carboxylic acid (2- {4-[3-(6- amino-5- trifIuoro- methyl- pyridin-3- yl)- imidazo [1,2-b) pyridazin- 6-yl]-2- methoxy- phenoxy}- ethyl)- amide | Method E starting from 5-{6-[4-(2- amino- ethoxy)-3- methoxy- phenyl]- imidazo[1,2- b]pyridazin- 3-yl}-3- trifluoro- methyl- pyridin- 2-ylamine (example 59) | 513.1 | 4.25 |
| 62 | 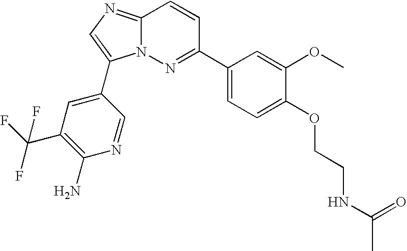 | N-(2-{4-[3-(6- Amino-5- trifluoro- methyl- pyridin-3-yl)- imidazo[1,2-b] pyridazin-6-yl]- 2-methoxy- phenoxy}- ethyl)- acetamide | Method D using acetic anhydride starting from 5-{6-[4- (2-Amino- ethoxy)-3- methoxy- phenyl]- imidazo[1,2- b]pyridazin- 3-yl}-3- trifluoro- methyl- pyridin-2- ylamine (example 59) | 487.1 | 4.033 |
| 63 | 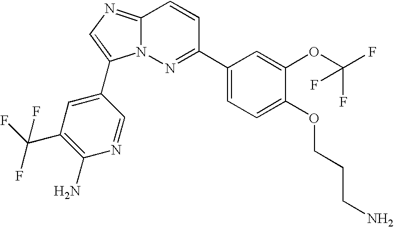 | 5-{6-[4-(3- Amino- propoxy)-3- trifluoro- methoxy- phenyl]- imidazo [1,2-b] pyridazin-3- yl}-3- trifluoro- methyl- pyridin-2- ylamine | Method B starting from (3-{4-[3-(6- Amino-5- trifluoro- methyl- pyridin-3- yl)-imidazo [1,2-b] pyridazin- 6-yl]-2- trifluoro- methoxy- phenoxy}- propyl)- carbamic acid tert- butyl ester Example 55) | 513 | 4.033 |
| 64 | 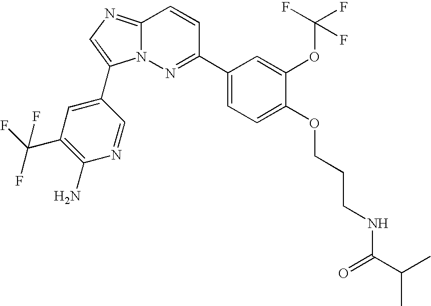 | N-(3-{4-[3-(6- Amino-5- trifluoro- methyl- pyridin-3-yl)- imidazo [1,2-b] pyridazin-6- yl]-2- trifluoro- methoxy- phenoxy}- propyl)- iso- butyramide | Method Method D using isobutyryl chloride, starting from (3-{4-[3-(6- amino-5- trifluoro- methyl- pyridin-3- yl)-imidazo [1,2-b] pyridazin-6- yl]-2- trifluoro- methoxy- phenoxy}- propyl)- carbamic acid tert- | 582.7 | 4.767 |
| 65 | 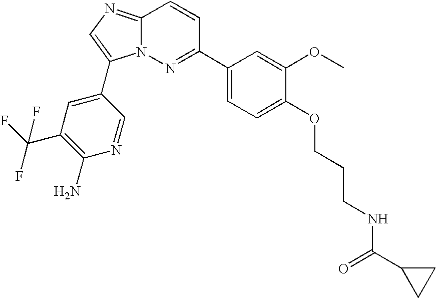 | Cyclo- propane- carboxylic acid (3- {4-[3-(5- amino-5- trifluoro- methyl- pyridin-3- yl)-imidazo [1,2- b]pyridazin- 6-yl]-2- methoxy- phenoxy}- propyl)- amide | Method E starting from 5- {6-[4-(3- amino- propoxy)- 3-methoxy- phenyl]- imidazo[1,2- b]pyridazin- 3-yl}-3- trifluoro- methyl- pyridin-2- ylamine (example 14) | 527.1 | 4.358 |
| 66 | 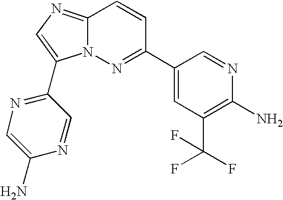 | 5-[6-(6- Amino-5- trifluoro- methyl- pyridin-3- yl)-imidazo [1,2- b]pyridazin- 3-yl]- pyrazin- 2-ylamine | Method A using 5- (4,4,5,5- tetramethyl- [1,3,2] dioxaborolan- 2-yl)- pyrazin-2- ylamine | 373.1 | 3.833 |
| 67 | 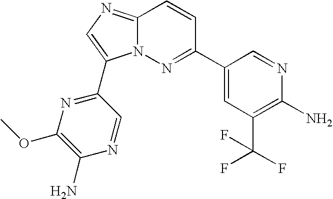 | 5-[6-(6- Amino-5- trifIuoro- methyl- pyridin-3-yl)- imidazo[1,2- b]pyridazin- 3-yl]-3- methoxy- pyrazin- 2-ylamine | Method A using 3- methoxy-5- (4,4,5,5- tetramethyl- [1,3,2] dioxaborolan- 2-yl)- pyrazin-2- ylamine | 403.1 | 3.967 |
| 68 | 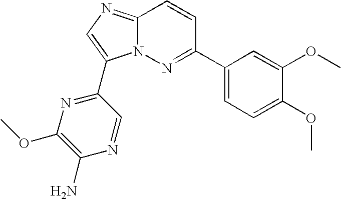 | 5-[6-(3,4- Dimethoxy- phenyl)- imidazo [1,2-b] pyridazin-3- yl]-3- methoxy- pyrazin-2- ylamine | Method A starting from 3,4- dimethoxy- boronic acid and 3- Methoxy-5- (4,4,5,5- tetramethyl- [1,3,2] dioxaborolan- 2- yl)-pyrazin- 2-ylamine | 379.2 | 4.033 |
| 69 | 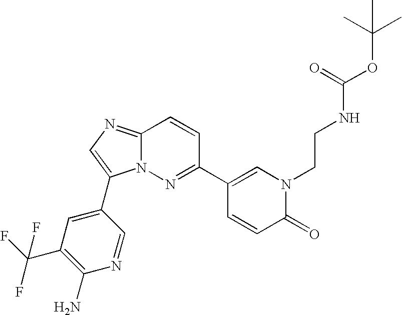 | (2-{5-[3-(6- Amino-5- trifluoro- methyl- pyridin-3- yl)- imidazo [1,2-b] pyridazin-6- yl]- 2-oxo-2H- pyridin-1-yl}- ethyl)- carbamic acid tert-butyl ester | Method A using 5- (4,4,5,5- Tetra- methyl- [1,3,2] dioxa- borolan- 2-yl)- pyridin- 2-ol and 5-(4,4,5,5- tetramethyl- [1,3,2] dioxa- borolan-2- yl)-3- trifluoro- methyl- pyridin-2- ylamine | 515.8 | 4.175 |
| 70 | 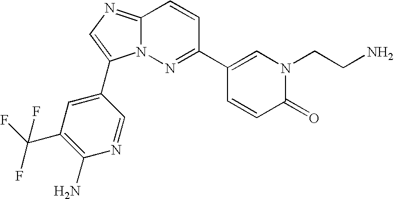 | 1-(2-Amino- ethyl)-5-[3- (6-amino- 5-trifluoro- methyl- pyridin-3- yl)-imidazo [1,2-b] pyridazin-6- yl]-1H- pyridin-2- one | Method B starting from (2-{5-[3-(6- Amino-5- trifluoro- methyl- pyridin-3- yl)- imidazo [1,2-b] pyridazin- 6-yl]-2- oxo-2H- pyridin-1-yl}- ethyl)- carbamic acid tert-butyl ester (example 69) | 416.2 | 3.583 |
| 71 | 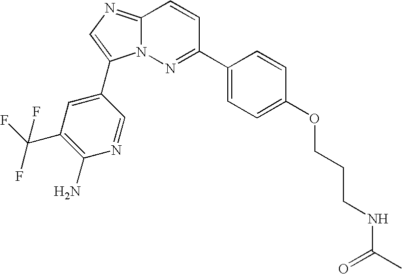 | N-(3-{4-[3-(6- Amino-5- trifluoro- methyl- pyridin-3-yl)- imidazo [1,2-b] pyridazin-6- yl]-phenoxy}- propyl)- acetamide | Method D using acetic anhydride, starting from 5-{6-[4- (3-Amino- propoxy)- phenyl]- imidazo[1,2- b]pyridazin- 3-yl}-3- trifluoro- methyl- pyridin-2- ylamine (example 56) | 471.1 | 4.183 |
| 72 | 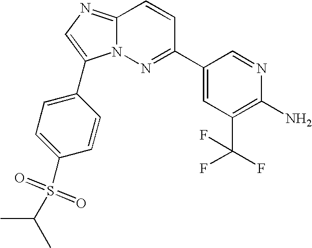 | 5-{3-[4- (Propane-2- sulfonyl)- phenyl]- imidazo[1,2- b]pyridazin- 6-yl}-3- trifluoro- methyl- pyridin-2- lamine | Method A using 4- (propane- 2-sulfonyl)- boronic acid | 462.1 | 4.575 |
| 73 | 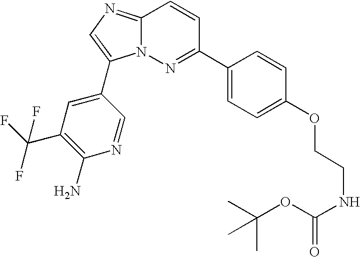 | (2-{4-[3-(6- Amino-5- trifluoro- methyl- pyridin-3- yl)-imidazo [1,2-b] pyridazin- 6-yl]-phenoxy}- ethyl)- carbamic acid tert-butyl ester | Method A using 4- (4,4,5,5- tetra- methyl- [1,3,2] dioxa- borolan- 2-yl)- phenol and 5-(4,4,5,5- tetra- methyl- [1,3,2] dioxa- borolan- 2-yl)-3- trifluoro- methyl- pyridin-2- ylamine | 515.1 | 4.767 |
| 74 | 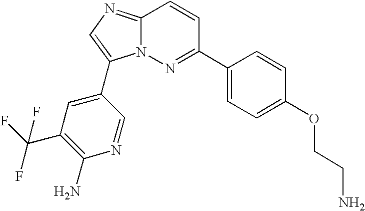 | 5-{6-[4-(2- Amino- ethoxy)- phenyl]- imidazo [1,2-b] pyridazin- 3-yl}-3- trifluoro- methyl- pyridin-2- ylamine | Method B starling from (2-{4-[3-(6- amino-5- trifluoro- methyl- pyridin- 3-yl)- imidazo [1,2-b] pyridazin- 6-yl]- phenoxy}- ethyl)- carbamic acid tert- butyl ester (example 73) | 415.2 | 3.717 |
| 75 | 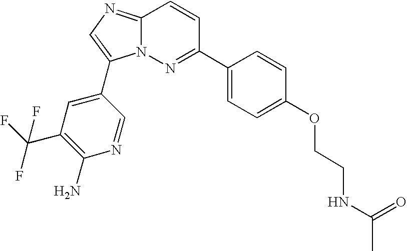 | N-(2-{4-[3-(6- Amino-5- trifluoro- methyl- pyridin-3-yl)- imidazo [1,2-b] pyridazin- 6-yl]- phenoxy}- ethyl)- acetamide | Method D using acetic anhydride, reaction time 90 min., starting from 5-{6-[4- (2-amino- ethoxy)- phenyl]- imidazo [1,2-b] pyridazin- 3-yl]-3- trifluoro- methyl- pyridin-2- ylamine (example 74) | 457.1 | 4.083 |
| 76 | 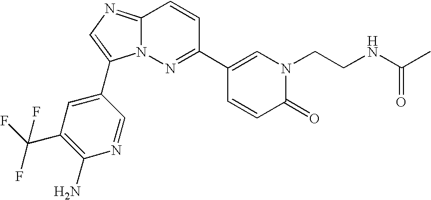 | N-(2-{5-[3-(6- Amino-5- trifluoro- methyl- pyridin- 3-yl)- imidazo [1,2-b] pyridazin- 6-yl]-2- oxo-2H- pyridin- 1-yl}- ethyl)- acetamide | Method D using acetic a nhydride, reaction time 60 min., starting from 1-(2- amino- ethyl)- 5-[3-(6- amino-5- trifluoro- methyl- pyridin-3- yl)- imidazo [1,2-b] pyridazin- 6-yl]-1H- pyridin-2- one (example 70) | 458.1 | 3.75 |
| 77 | 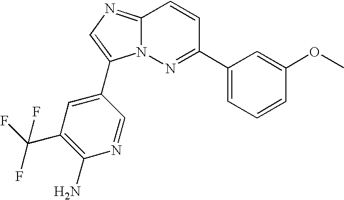 | 5-[6-(3- Methoxy- phenyl)- imidazo [1,2-b] pyridazin-3- yl]-3- trifluoro- methyl- pyridin-2- ylamine | Method A using 3- methoxy- boronic acid and 5-(4,4,5,5- tetra- methyl- [1,3,2] dioxa- borolan-2- yl)-3- trifluoro- methyl- pyridin-2- ylamine | 386.1 | 4.725 |
| 78 | 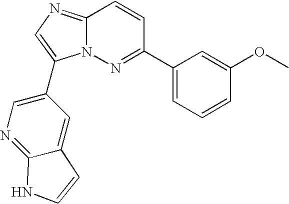 | 6-(3- Methoxy- phenyl)-3- {1H-pyrrolo [2.3-b] pyridin-5- yl)-imidazo [1,2-b] pyridazine | Method A using 3- methoxy- boronic acid and 5-(4,4,5,5- tetra- methyl- [1,3,2] dioxa- borolan- 2-yl)-1H- pyrrolo[2,3- b]pyridine | 342.3 | 4.4 |
| 79 | 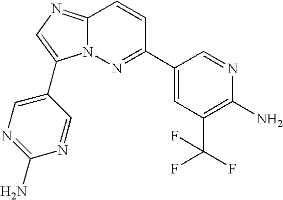 | 5-[6-(6- Amino-5- trifluoro- methyl- pyridin-3- yl)-imidazo [1,2-b] pyridazin- 3-yl]- pyrimidin-2- ylamine | Method A using 5- (4,4,5,5- tetra- methyl- [1,3,2] dioxa- borolan- 2-yl)- pyrimidin-2- ylamine | 373.1 | 3.758 |
| 80 | 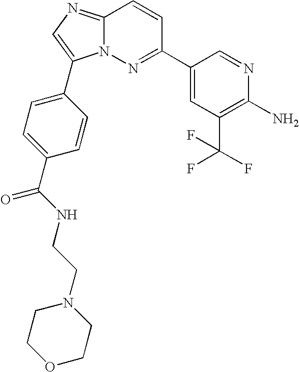 | 4-[6-(6- Amino-5- trifluoro- methyl- pyridin-3- yl)-imidazo [1,2-b] pyridazin- 3-yl]-N- (2-morpholin- 4-yl-ethyl)- benzamide | Method E starting from 4-[6- (6-Amino-5- trifluoro- methyl- pyridin- 3-yl)- imidazo [1,2- b]pyridazin- 3-yl]- benzoic acid and 2- morpholin- 4-yl- ethylamine (The acid is prepared according to method A using 4- | 512.1 | 3.792 |
| 81 | 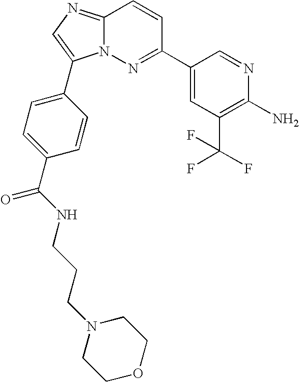 | 4-[6-(6- Amino-5- trifluoromethyl- pyridin-3- yl)-imidazo [1,2-b] pyridazin- 3-yl]-N- (3-morpholin- 4-yl-propyl)- benzamide | same as example 80 but using 3- morpholin- 4-yl- propylamine | 526.2 | 3.783 |
| 82 | 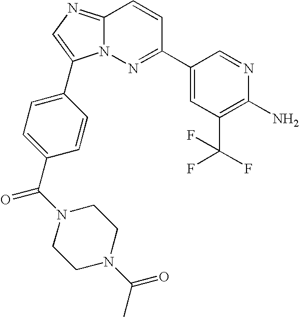 | 1-(4-{4-[6-(6- Amino-5- trifIuoro- methyl- pyridin-3- yl)- imidazo [1,2-b] pyridazin-3- yl]- benzoyl}- piperazin-1-yl)- ethanone | same as example 80 but using 1- piperazin-1- yl-ethanone | 510.1 | 3.942 |
| 83 | 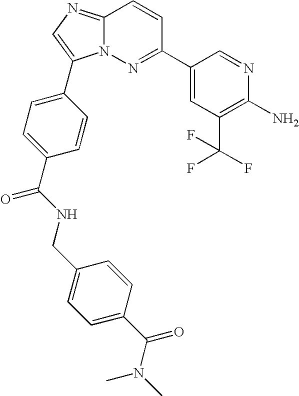 | N-(4-N- dimethylacetyl- benzyl)-4- [6-(6-amino- 5-trifluoro- methyl- pyridin-3- yl)-imidazo [1,2-b] pyridazin-3- yl]- benzamide | same as example 80 but using 4- amino- methyl- N,N- dimethyl- benzamide | 560.2 | 4.242 |
| 84 | 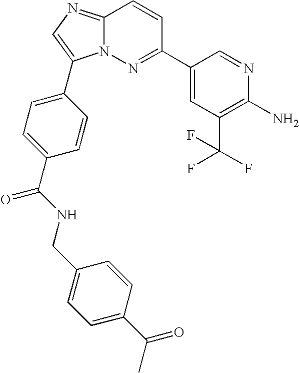 | N-(4-Acetyl- benzyl)- 4-[6-(6- amino-5- trifluoromethyl- pyridin-3- yl)-imidazo [1,2-b] pyridazin-3- yl]-benzamide | same as example 80 but using 1-(4- amino- methyl- phenyl)- ethanone | 531 | 4.467 |
| 85 | 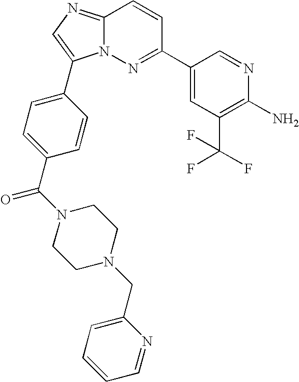 | {4-[6-(6- Amino-5- trifluoromethyl- pyridin-3-yl)- imidazo[1,2- b]pyridazin-3- yl]-phenyl}-(4- pyridin-2- ylmethyl- piperazin-1- yl)-methanone | same as example 80 using 1-pyridin-2- ylmethyl- piperazine instead | 559.1 | 3.833 |
| 86 | 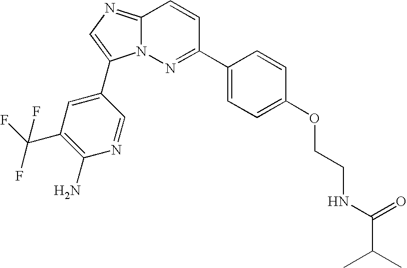 | N-(2-{4-[3-(6- Amino-5- trifluoro- methyl- pyridin- 3-yl)-imidazo [1,2-b] pyridazin- 6-yl]-phenoxy}- ethyl)- isobutyramide | Method D using isobutyryl chloride, reaction time 15 min, starting from 5-{6-[4- (2-amino- ethoxy)- phenyl]- imidazo [1,2-b] pyridazin- 3-yl}-3- trifluoro- methyl- pyridin-2- ylamine (example 74) | 485.1 | 4.325 |
| 87 | 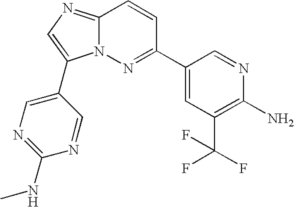 | {5-[6-(6- Amino-5- trifluoro- methyl- pyridin-3- yl)-imidazo [1,2-b] pyridazin-3-yl]- pyrimidin-2-yl)- methylamine | Method A using methyl- [5-(4,4,5,5- tetra- methyl- [1,3,2] dioxa- borolan- 2-yl)- pyrimidin- 2-yl]- amine | 387.2 | 3.933 |
| 88 | 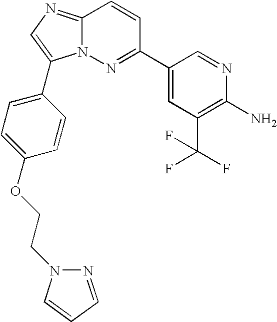 | 5-{3-[4-(2- Pyrazol-1-yl- ethoxy)- phenyl]- imidazo[1,2-b] pyridazin-6- yl}-3- trifluoro- methyl- pyridin-2- ylamine | Method A using 1-{2- [4-(4,4,5,5- Tetra- methyl- [1,3,2] dioxa- borolan- 2-yl)- phenoxy]- ethyl}-1H- pyrazole | 466.1 | 4.425 |
| 89 | 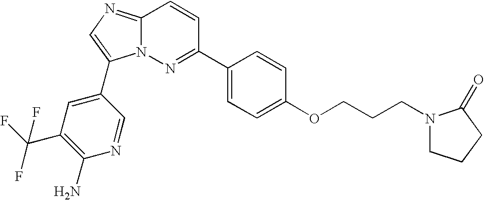 | 1-(3-{4-[3-(6- Amino-5- trifluoro- methyl- pyridin-3- yl)-imidazo [1,2-b] pyridazin-6- yl]-phenoxy}- propyl)- pyrrolidin-2- one | Method A (step A2/A3, using 1-[3-(4- Imidazo [1,2-b] pyridazin- 6-yl- phenoxy)- propyl]- pyrrolidin- 2-one (see Method G) and 5- (4,4,5,5- tetra- methyl- [1,3,2] dioxa- borolan-2- yl)-3- trifluoro- methyl- pyridin-2- ylamine | 497.1 | 4.408 |
| 90 | 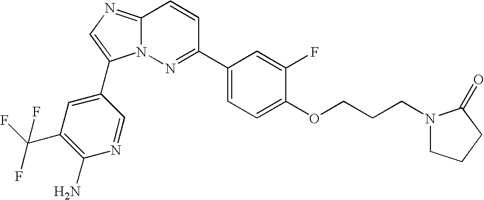 | 1-(3-{4-[3-(6- Amino-5- trifluoro- methyl- pyridin-3- yl)- imidazo [1,2-b] pyridazin-6- yl]-2- fluoro- phenoxy}- propyl)- pyrrolidin- 2-one | same as example 89 using 4-hydroxy- 3-fluoro- phenyl- boronic acid instead | 515.1 | 4.517 |
| 91 | 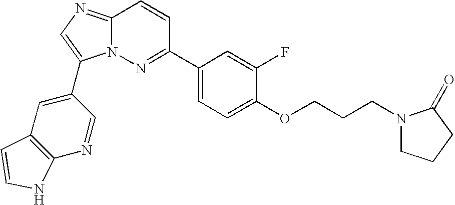 | 1-(3-{2- Fluoro-4- [3-(1H- pyrrolo [2,3-b] pyridin-5- yl)- imidazo [1,2-b] pyridazin-6- yl]- phenoxy}- propyl)- pyrrolidin-2- one | same as example 90 using 5-(4,4,5,5- tetra- methyl- [1,3,2] dioxa- borolan-2- yl)-1H- pyrrolo [2,3-b] pyridine instead | 471.1 | 4.275 |
| 92 | 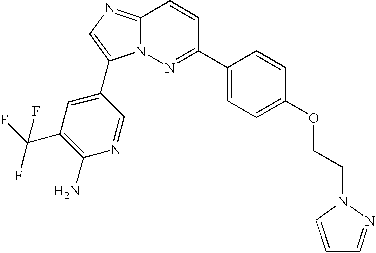 | 5-{6-[4-(2- Pyrazol-1- yl-ethoxy)- phenyl]- imidazo [1,2-b] pyridazin-3- yl}-3- trifluoro- methyl- pyridin-2- ylamine | Method A using 1-{2- [4-(4,4,5,5- Tetra- methyl- [1,3,2] dioxa- borolan-2- yl)- phenoxy]- ethyl}-1H- pyrazole and 5- (4,4,5,5- tetramethyl- [1,3,2] dioxa- borolan-2- yl)-3- trifluoro- methyl- pyridin-2- ylamine | 466.1 | 4.467 |
| 93 | 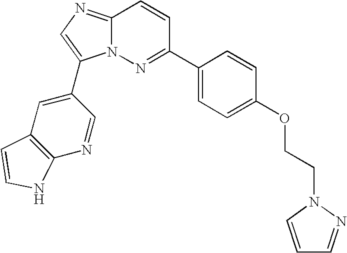 | 6-[4-(2- Pyrazol-1- yl-ethoxy)- phenyl]-3- (1H-pyrrolo [2,3-b] pyridin-5- yl)-imidazo [1,2-b] pyridazine | same as example 92 using 5-(4,4,5,5- tetra- methyl- [1,3,2] dioxa- borolan-2- yl)-1H- pyrrolo [2,3-b] pyridine instead | 422.2 | 4.242 |
| 94 | 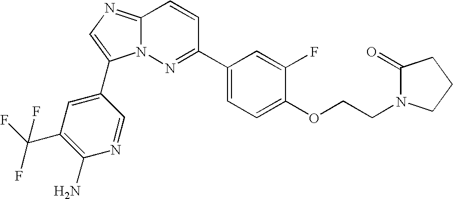 | 1-(2-{4-[3-(6- Amino-5- trifluoro- methyl- pyridin-3- yl)-imidazo [1,2-b] pyridazin-6- yl]-2-fluoro- phenoxy}- ethyl)- pyrrolidin-2- one | same as example 90 using 1-(3- hydroxy- ethyl)-2- pyrrolidon instead | 501.1 | 4.408 |
| 95 | 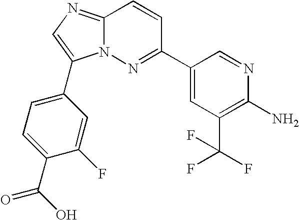 | 4-[6-(6- Amino-5- trifluoro- methyl- pyridin-3- yl)-imidazo [1,2-b] pyridazin-3- yl]-2-fluoro- benzoic acid | Method A using 2- fluoro 4-(4,4,5,6- tetra- methyl- [1,3,2] dioxa- borolan-2- yl)- benzoic acid | 418.1 | 4.342 |
| 96 | 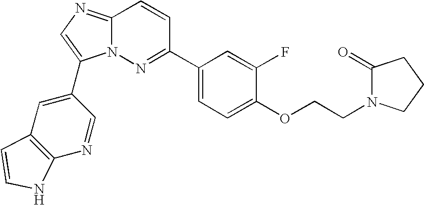 | 1-(2-{2- Fluoro-4-[3- (1H- pyrrolo [2,3-b] pyridin-5- yl)- imidazo [1,2-b] pyridazin-6- yl]- phenoxy)- ethyl)- pyrrolidin-2- one | same as example 94 using 5-(4,4,5,5- tetra- methyl- [1,3,2] dioxa- borolan-2- yl)-1H- pyrrolo[ 2,3-b] pyridine instead | 457.1 | 4.183 |
| 97 | 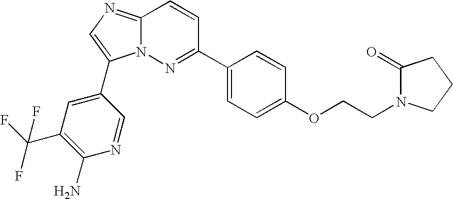 | 1-(2-{4-[3-(6- Amino-5- trifluoro- methyl- pyridin-3- yl)-imidazo [1,2-b] pyridazin-6- yl]- phenoxy}- ethyl)- pyrrolidin-2- one | same as example 89 using 1-(3- hydroxy- ethyl)-2- pyrrolidon instead | 483.1 | 4.3 |
| 98 | 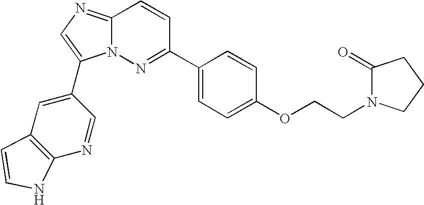 | 1-(2-{4-[3-(1H- Pyrrolo[2,3- b]pyridin-5- yl)-imidazo [1,2-b] pyridazin-6- yl]-phenoxy}- ethyl)- pyrrolidin-2- one | same as example 97 using 5-(4,4,5,5- tetra- methyl- [1,3,2] dioxa- borolan-2- yl)-1H- pyrrolo [2,3-b] pyridine instead | 439.1 | 4.092 |
| 99 | 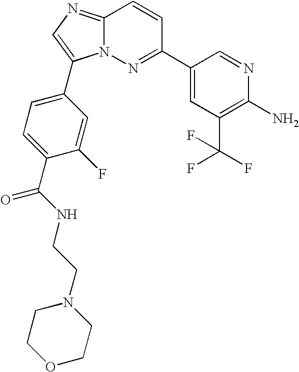 | 4-[6-(6- Amino-5- trifluoro- methyl- pyridin-3- yl)-imidazo [1,2-b] pyridazin-3- yl]-2- fluoro-N-(2- morpholin-4- yl-ethyl)- benzamide | Method E starting from 4-[6-(6- amino-5- trifluoro- methyl- pyridin-3- yl)- imidazo [1,2-b] pyridazin-3- yl]-2- fluoro- benzoic acid (example 95), using 2-morpholin- 4-yl- ethylamine | 530.1 | 3.908 |
| 100 | 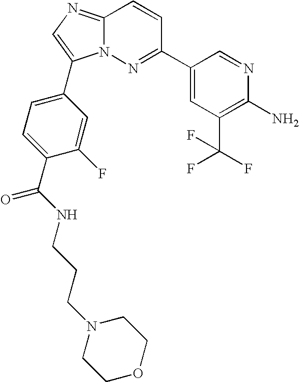 | 4-[6-(6- Amino-5- trifluoro- methyl- pyridin-3- yl)-imidazo [1,2-b] pyridazin-3- yl]-2-fluoro- N-(3- morpholin-4- yl-propyl)- benzamide | Method E starting from 4-[6-(6- amino-5- trifluoro- methyl- pyridin-3- yl)- imidazo [1,2-b] pyridazin-3- yl]-2- fluoro- benzoic acid (example 95), using 2- morpholin- 4-yl- propyl- amine | 544.1 | 3.908 |
| 101 | 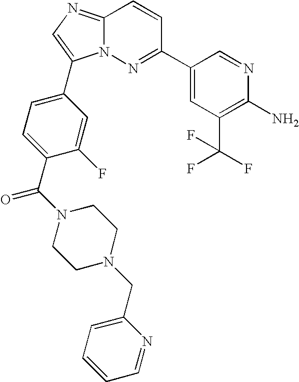 | {4-[6-(6- Amino-5- trifluoro- methyl- pyridin-3- yl)-imidazo [1,2-b] pyridazin- 3-yl]-2- fluoro- phenyl}- (4-pyridin- 2-ylmethyl- piperazin-1- yl)- methanone | same as example 100 using 1-pyridin- 2-ylmethyl- piperazine | 577.1 | 3.95 |
| 102 | 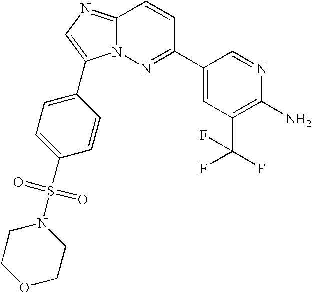 | 5-{3-[4- (Morpholine-4- sulfonyl)- phenyl]- imidazo[1,2-b] pyridazin-6- yl}-3- trifluoromethyl- pyridin-2- ylamine | Method A using 4- (morpholine- 4- sulfony1)- benzoic acid | 505 | 4.575 |
| 103 | 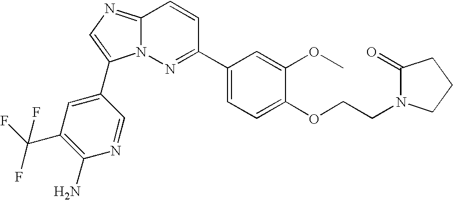 | 1-(2-{4-[3-(6- Amino-5- trifluoro- methyl- pyridin-3- yl)-imidazo [1,2-b] pyridazin-6- yl]-2- methoxy- phenoxy}- ethyl)- pyrrolidin-2- one | same as example 89 using 2- methoxy- 4-(4,4,5,5- tetra- methyl- [1,3,2] dioxa- borolan-2- yl)- phenol instead | 513.1 | 4.25 |
| 104 | 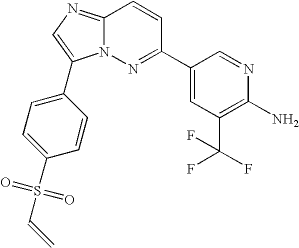 | 5-[3-(4- Ethene- sulfonyl- phenyl)- imidazo [1,2-b] pyridazin-6- yl]-3- trifluoro- methyl- pyridin-2- ylamine | Method A using 2-(4- ethene- sulfonyl- phenyl)- 4,4,5,5- tetra- methyl- [1,3,2] dioxa- borolane instead | 446.1 | 4.525 |
| 105 | 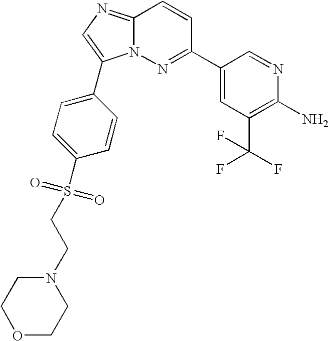 | 5-{3-[4-(2- Morpholin-4- yl-ethane- sulfonyl)- phenyl]- imidazo [1,2-b] pyridazin-6- yl}-3- trifluoro- methyl- pyridin-2- ylamine | Method A using 4-{2- [4-(4,4,5,5- tetra- methyl- [1,3,2] dioxa- borolan-2- yl)- benzene- sulfonyl]- ethyl}- morpholine (NVP- BUY365) for later see method C and H | 533 | 4 |
| 106 | 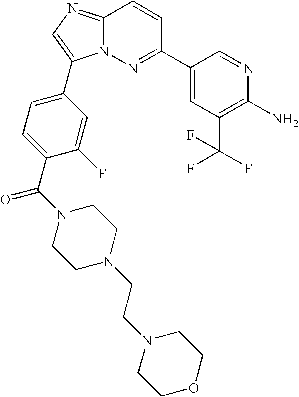 | {4-[6-(6- Amino-5- trifluoro- methyl- pyridin-3- yl)-imidazo [1,2- b]pyridazin- 3-yl]-2- fluoro- phenyl}- [4-(2- morpholin- 4-yl-ethyl)- piperazin-1- yl]- methanone | same as example 100 using 4- (2-piperazin- 1-yl-ethyl)- morpholine | 599.1 | 3.775 |
| 107 | 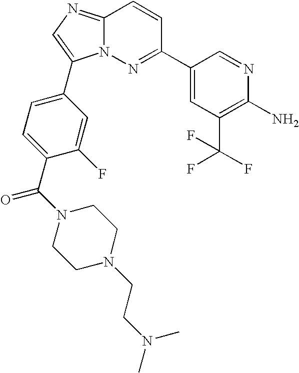 | {4-[6-(6- Amino-5- trifluoro- methyl- pyridin-3- yl)-imidazo [1,2-b] pyridazin-3- yl]-2- fluoro- phenyl}- [4-(2- dimethyl- amino- ethyl)- piperazin-1- yl]- methanone | same as example 100 using dimethyl-(2- piperazin-1- yl-ethyl)- amine | 557.1 | 3.775 |
| 108 | 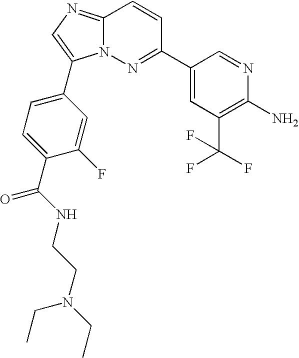 | 4-[6-(6- Amino-5- trifluoro- methyl- pyridin-3- yl)-imidazo [1,2-b] pyridazin-3- yl]-N-(2- diethyl- amino- ethyl)-2- fluoro- benzamide | same as example 100 using N*1*, N*1*- diethyl- ethane- 1,2- diamine | 516.1 | 4.033 |
| 109 | 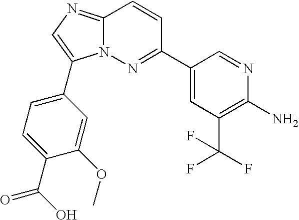 | 4-[6-(6- Amino-5- trifluoromethyl- pyridin-3- yl)-imidazo [1,2-b] pyridazin-3- yl]-2- methoxy- benzoic acid | Method A using 2- methoxy-4- (4,4,5,5- tetramethyl- [1,3,2] dioxa- borolan-2- yl)-benzoic acid | 430.1 | 4.217 |
| 110 | 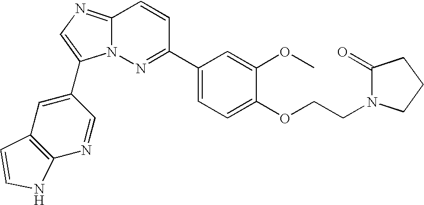 | 1-(2-{2- Methoxy- 4- [3-(1H- pyrrolo [2,3-b] pyridin-5- yl)-imidazo [1,2-b] pyridazin-6- yl]- phenoxy}- ethyl)- pyrrolidin-2- one | same as example 89 using 5-(4,4,5,5- tetra- methyl- [1,3,2] dioxa- borolan-2- yl)-1H- pyrrolo [2,3-b] pyridine instead | 469.2 | 4.092 |
| 111 | 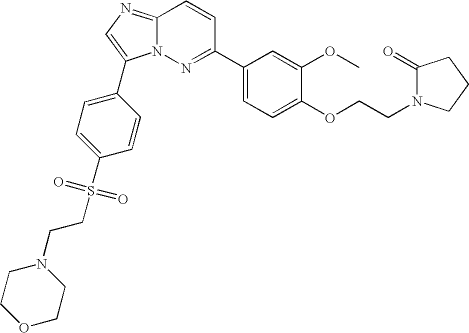 | 1-[2-(2- Methoxy- 4-{3-[4-(2- morpholin- 4-yl-ethane- sulfonyl)- phenyl]- imidazo [1,2-b] pyridazin- 6-yl}- phenoxy)- ethyl]- pyrrolidin-2- one | same as example 89 using 4-{2-[4- (4,4,5,5- tetra- methyl- [1,3,2] dioxa- borolan-2- yl)- benzene- sulfonyl]- ethyl}- morpholine, for later see method C and H | 606.1 | 3.983 |
| 112 | 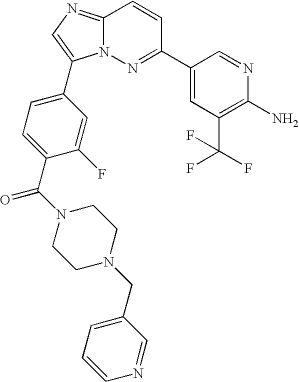 | {4-[6-(6- Amino-5- trifluoro- methyl- pyridin-3- yl)-imidazo [1,2-b] pyridazin-3- yl]-2-fluoro- phenyl}- (4-pyridin-3- ylmethyl- piperazin-1- yl)- methanone | same as example 100 using 1- pyridin-3- ylmethyl- piperazine instead | 577.1 | 3.792 |
| 113 | 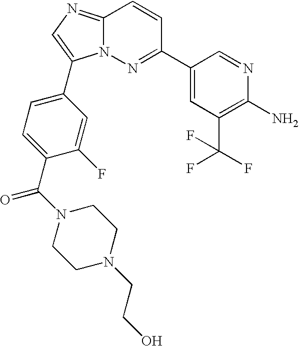 | {4-[6-(6- Amino-5- trifluoro- methyl- pyridin-3- yl)-imidazo [1,2-b] pyridazin-3- yl]-2-fluoro- phenyl}- [4-(2- hydroxy- ethyl)- piperazin-1- yl]- methanone | same as example 100 using 2- piperazin- 1-yl- ethanol instead | 530.1 | 3.792 |
| 114 | 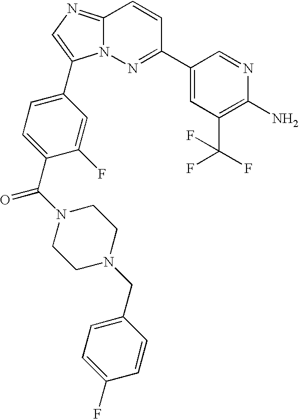 | {4-[6-(6- Amino-5- trifluoromethyl- pyridin-3- yl)-imidazo [1,2-b] pyridazin-3- yl]-2- fluoro- phenyl}- [4-(4- fluoro- benzyl)- piperazin-1- yl]- methanone | same as example 100 using 1-(4- fluoro- benzyl)- piperazine instead | 594 | 4.192 |
| 115 | 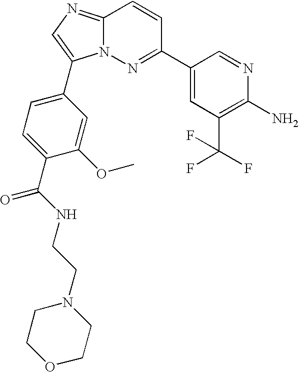 | 4-[6-(6- Amino-5- trifluoro- methyl- pyridin-3- yl)-imidazo [1,2-b] pyridazin-3- yl]-2- methoxy- N-(2- morpholin-4- yl-ethyl)- benzamide | Method E starting from 4-[6-(6- amino-5- trifluoro- methyl- pyridin-3- yl)-imidazo [1,2-b] pyridazin-3- yl]-2- methoxy- benzoic acid (example 109), using 2-morpholin- 4-yl- ethylamine | 542.1 | 3.942 |
| 116 | 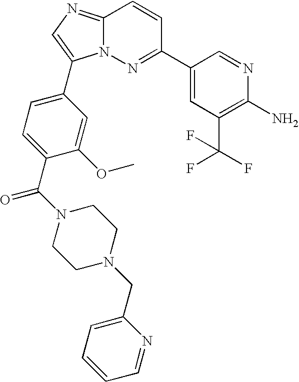 | {4-[6-(6- Amino-5- trifluoro- methyl- pyridin-3- yl)- imidazo [1,2-b] pyridazin- 3-yl]-2- methoxy- phenyl}- (4-pyridin- 2- ylmethyl- piperazin- 1-yl)- methanone | same as example 115 using 1-pyridin- 2-ylmethyl- piperazine instead | 589.1 | 3.9 |
| 117 | 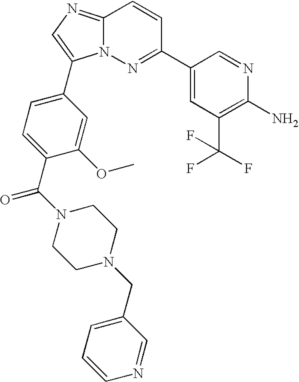 | {4-[6-(6- Amino-5- trifluoro- methyl- pyridin-3- yl)-imidazo [1,2-b] pyridazin-3- yl]-2- methoxy- phenyl}- (4-pyridin-3- ylmethyl- piperazin-1- yl)- methanone | same as example 115 using 1- pyridin-3- ylmethyl- piperazine instead | 589.1 | 3.708 |
| 118 | 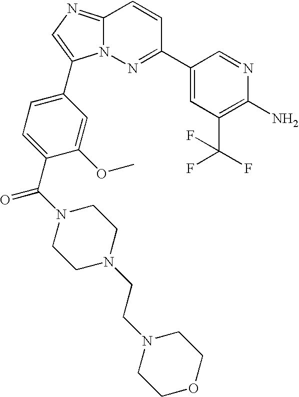 | {4-[6-(6- Amino-5- trifluoro- methyl- pyridin-3- yl)-imidazo [1,2-b] pyridazin-3- yl]-2- methoxy- phenyl}-[4- (2-morpholin- 4-yl-ethyl)- piperazin-1- yl]- methanone | same as example 115 using 4-(2- piperazin- 1-yl- ethyl)- morpholine instead | 611.1 | 3.725 |
| 119 | 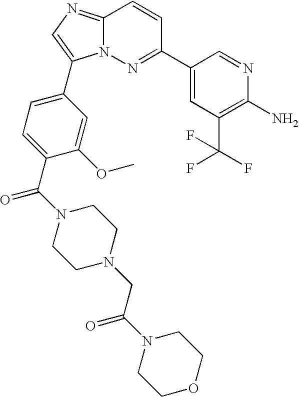 | 2-(4-{4-[6-(6- Amino-5- trifluoro- methyl- pyridin-3- yl)- imidazo [1,2-b] pyridazin- 3-yl]-2- methoxy- benzoyl}- piperazin- 1-yl)-1- morpholin- 4-yl- ethanone | same as example 115 using 1-morpholin- 4-yl-2- piperazin- 1-yl- ethanone instead | 625.1 | 3.808 |
| 120 | 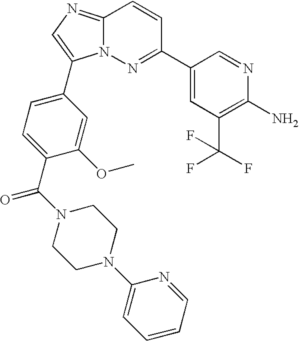 | {4-[6-(6- Amino-5- trifluoro- methyl- pyridin-3- yl)-imidazo [1,2-b] pyridazin- 3-yl]-2- methoxy- phenyl}- (4-pyridin- 2-yl- piperazin- 1-yl)- methanone | same as example 115 using 1-pyridin- 2-yl- piperazine instead | 575.1 | 3.892 |
| 121 | 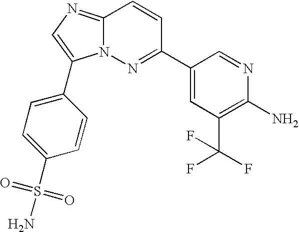 | 4-[6-(6- Amino-5- trifluoro- methyl- pyridin-3- yl)- imidazo [1,2-b] pyridazin- 3-yl]- benzene- sulfon- amide | Method A using 4- sulfamoyl- boronic acid instead | 435.1 | 4.083 |
| 122 | 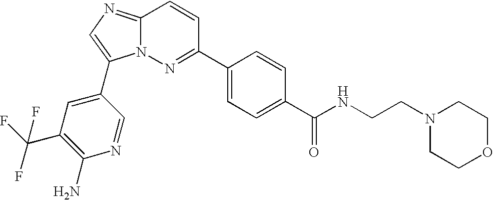 | 4-[3-(6- Amino-5- trifluoro- methyl- pyridin-3- yl)-imidazo [1,2-b] pyridazin-6- yl]-N-(2- morpholin- 4-yl-ethyl)- benzamide | Method A using 4-(3- bromo- imidazo [1,2-b] pyridazin- 6-yl)-N-(2- morpholin- 4-yl-ethyl)- benzamide (see method I) and and 5-(4,4,5,5- tetra- methyl- [1,3,2] dioxa- borolan-2- yl)-3- trifluoro- methyl- pyridin-2- ylamine | 512.1 | 3.8 |
| 123 | 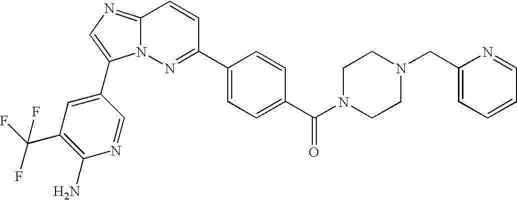 | {4-[3-(6- Amino-5- trifluoro- methyl- pyridin-3- yl)-imidazo [1,2-b] pyridazin-6- yl]-phenyl}- (4-pyridin- 2-ylmethyl- piperazin-1- yl)- methanone | same as example 122 using 1-pyridin-2- ylmethyl- piperazine instead | 599 | 3.842 |
| 124 | 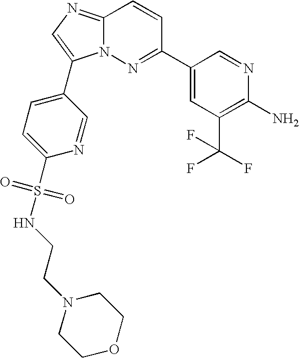 | 4-[6-(6- Amino-5- trifluoro- methyl- pyridin-3- yl)-imidazo [1,2-b] pyridazin-3- yl]-N-(2- morpholin- 4-yl-ethyl)- benzene- sulfonamide | Method A using 4-(2- morpholin- 4-yl- ethyl- sulfamoyl)- boronic acid (see Method J) instead | 548 | 3.942 |
| 125 | 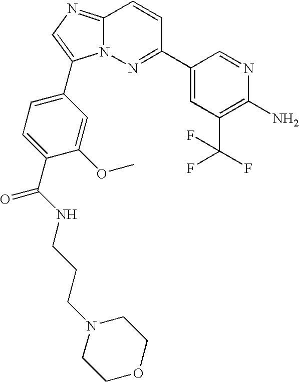 | 4-[6-(6- Amino-5- trifluoro- methyl- pyridin-3- yl)-imidazo [1,2-b] pyridazin-3- yl]-2- methoxy-N- (3-morpholin- 4-yl- propyl)- benzamide | same as example 115 using 3- morpholin- 4-yl- propyl- amine instead | 556.1 | 3.942 |
| 126 | 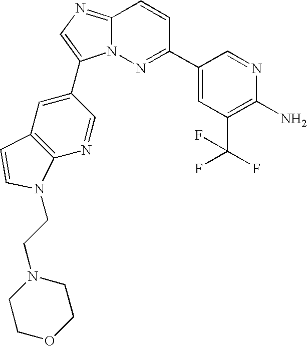 | 5-{3-[1-(2- Morpholin- 4-yl-ethyl)- 1H-pyrrolo [2,3-b] pyridin- 5-yl]- imidazo [1,2-b] pyridazin-6- yl}-3- trifluoro- methyl- pyridin-2- ylamine | Method A using 1-(2- morpholin- 4-yl-ethyl)- 1H-indole- 5-carboxylic acid instead (for later see Method J starting from 5- indolyl- boronic acid) | 509.1 | 3.875 |
| 127 | 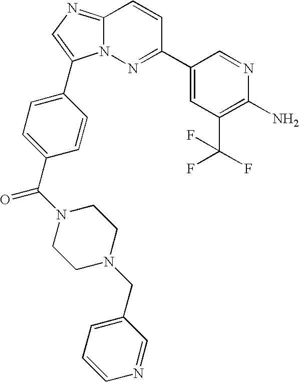 | {4-[6-(6- Amino-5- trifluoro- methyl- pyridin- 3-yl)- imidazo [1,2-b] pyridazin- 3-yl]- phenyl}- (4- pyridin-3- ylmethyl- piperazin- 1-yl)- methanone | same as example 80 using 1- pyridin-3- ylmethyl- piperazine instead | 559.1 | 3.675 |
| 128 | 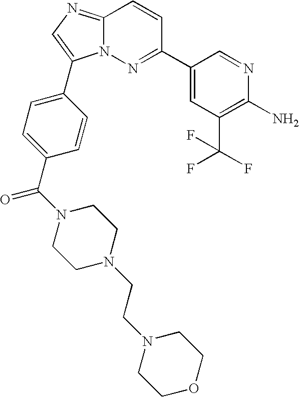 | {4-[6-(6- Amino-5- trifluoro- methyl- pyridin-3- yl)-imidazo [1,2-b] pyridazin-3- yl]-phenyl}- [4-(2- morpholin-4- yl-ethyl)- piperazin-1- yl]- methanone | same as example 80 using 4-(2- piperazin- 1-yl- ethyl)- morpholine instead | 581.1 | 3.686 |
| 129 | 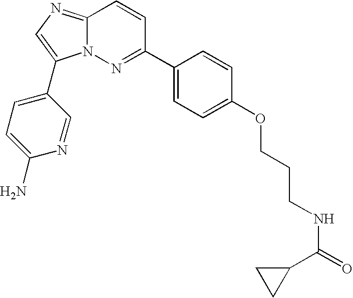 | Cyclo- propane- carboxylic acid (3- {4-[3-(6- amino- pyridin-3- yl)- imidazo [1,2-b] pyridazin-6- yl]- phenoxy}- propyl)- amide | Method E using 5- (4,4,5,5- tetra- methyl- [1,3,2] dioxa- borolan-2- yl)- pyridin-2- ylamine instead | 429 | 3.825 |
| 130 | 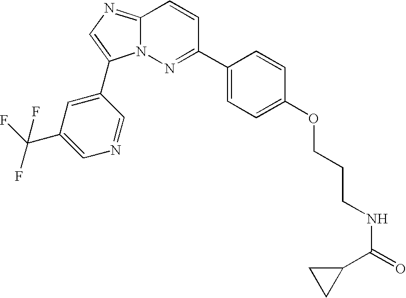 | Cyclo- propane- carboxylic acid (3- {4-[3-(5- trifluoro- methyl- pyridin-3- yl)-imidazo [1,2-b] pyridazin-6- yl]- phenoxy}- propyl)- amide | Method E using 3- (4,4,5,5- tetra- methyl- [1,3,2] dioxa- borolan-2- yl)-5- trifIuoro- methyl- pyridine instead | 482.1 | 5.017 |
| 131 | 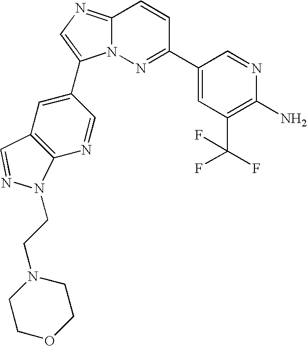 | 5-{3-[1-(2- Morpholin-4- yl-ethyl)- 1H-indazol- 5-yl]-imidazo [1,2-b] pyridazin-6- yl}-3- trifluoro- methyl- pyridin-2- ylamine | Method A using 1-(2- Morpholin-4- yl-ethyl)- 5-(4,4,5,5- tetramethyl- [1,3,2] dioxa- borolan-2- yl)-1H- indazole instead (for later see Method K) | 509.1 | 3.833 |
| 132 | 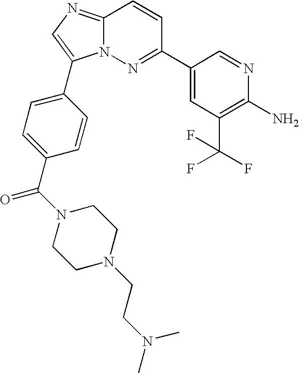 | {4-[6-(6- Amino-5- trifluoro- methyl- pyridin-3- yl)-imidazo [1,2-b] pyridazin-3- yl]-phenyl}- [4-(2- dimethyl- amino- ethyl)- piperazin-1- yl]- methanone | same as example 80 using dimethyl- (2- piperazin- 1-yl- ethyl)- amine instead | 539.1 | 3.658 |
| 133 | 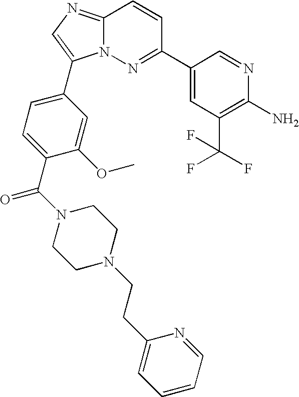 | {4-[6-(6- Amino-5- trifluoro- methyl- pyridin-3- yl)-imidazo [1,2-b] pyridazin-3- yl]-2- methoxy- phenyl)- [4-(2- pyridin-2- yl-ethyl)- piperazin-1- yl]- methanone | same as example 115 using 1-(2- pyridin-2- yl-ethyl)- piperazine instead | 603.1 | 3.717 |
| 134 | 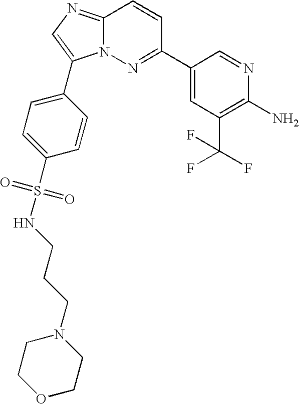 | 4-[6-(6- Amino-5- trifluoro- methyl- pyridin-3- yl)-imidazo [1,2-b] pyridazin-3- yl]-N-(3- morpholin- 4-yl- propyl)- benzene- sulfonamide | Method A using 4-(2- morpholin-4- yl- propyl- sulfamoyl)- boronic acid (see Method J) instead | 562 | 3.933 |
| 135 | 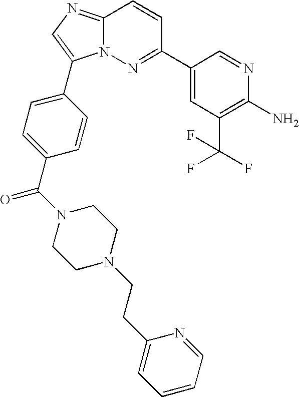 | {4-[6-(6- Amino-5- trifluoromethyl- pyridin-3- yl)-imidazo [1,2-b] pyridazin-3- yl]-phenyl}- [4-(2- pyridin-2- yl-ethyl)- piperazin- 1-yl- methanone | same as example 80 using 1-(2- pyridin-2- yl-ethyl)- piperazine instead | 573.1 | 3.692 |
| 136 | 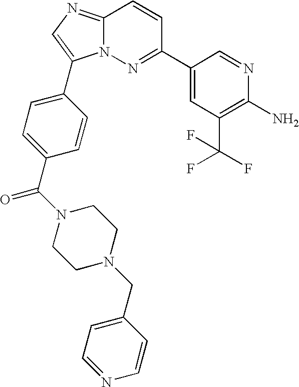 | {4-[6-(6- Amino-5- trifluoro- methyl- pyridin-3- yl)-imidazo [1,2-b] pyridazin-3- yl]-phenyl}- (4- pyridin-4- ylmethyl- piperazin-1- yl)- methanone | same as example 80 using 1-pyridin-4- ylmethyl- piperazine instead | 559.1 | 3.692 |
| 137 | 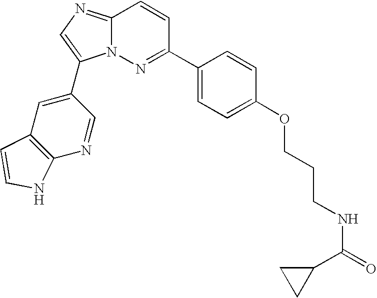 | Cyclo- propane- carboxylic acid (3- {4-[3-(1H- pyrrolo[2,3-b] pyridin-5- yl)-imidazo [1,2-b] pyridazin-6- yl]- phenoxy}- propyl)- amide | Method E using 5- (4,4,5,5- tetra- methyl- [1,3,2] dioxa- borolan-2- yl)-1H- pyrrolo [2,3-b] pyridine instead | 453.2 | 4.192 |
| 138 | 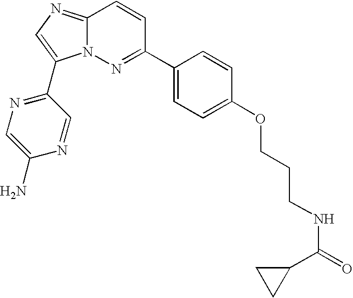 | Cyclo- propane- carboxylic acid (3- {4-[3-(5- amino- pyrazin- 2-yl)- imidazo [1,2-b] pyridazin-6- yl]- phenoxy}- propyl- amide | Method E using 5- (4,4,5,5- tetra- methyl- [1,3,2] dioxa- borolan-2- yl)-pyrazin- 2-ylamine instead | 430.2 | 4.017 |
| 139 | 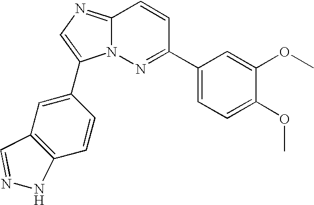 | 6-(3,4- Dimethoxy- phenyl)-3- (1H-indazol- 5-yl)- imidazo[1,2- b]pyridazine | Method A using 3,4- dimethoxy- boronic acid and 1H- indazole-5- carboxylic acid instead | 372.2 | 4.2 |
| 140 | 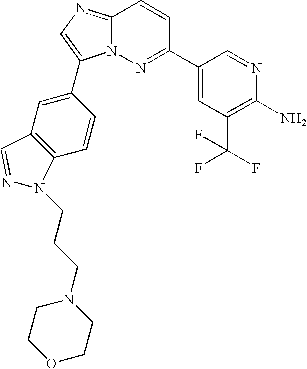 | 5-{3-[1-(3- Morpholin-4- yl-propyl)- 1H-indazol-5- yl]-imidazo [1,2-b] pyridazin-6- yl}-3- trifluoro- methyl- pyridin-2- ylamine | Method A using 1-(2- morpholin- 4-yl- propyl)-5- (4,4,5,5- tetramethyl- [1,3,2] dioxa- borolan-2- yl)-1H- indazole instead (for later see Method K) | 523.1 | 3.858 |
| 141 | 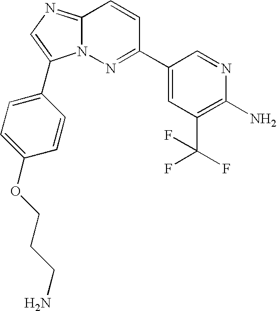 | 5-{3-[4-(3- Amino- propoxy)- phenyl]- imidazo [1,2-b] pyridazin-6- yl}-3- trifluoro- methyl- pyridin-2- ylamine | Method A using (3- {4-[6-(6- amino-5- trifluoro- methyl- pyridin-3- yl)- imidazo [1,2-b] pyridazin-3- yl]- phenoxy}- propyl)- carbamic acid tert- butyl ester instead (for later see Method L) | 429.1 | 3.775 |
| 142 | 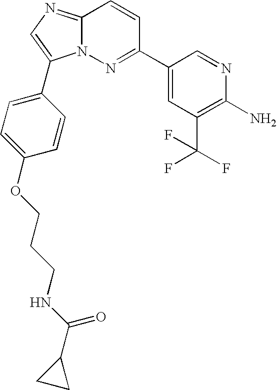 | Cyclo- propane- carboxylic acid (3- {4-[6-(6- amino-5- trifluoro- methyl- pyridin-3- yl)-imidazo [1,2-b] pyridazin-3- yl]-phenoxy}- propyl)-amide | Method E starting from 5-{3-[4-(3- amino- propoxy)- phenyl]- imidazo [1,2-b] pyridazin-6- yl}-3- trifluoro- methyl- pyridin-2- ylamine (example 141) using cyclo- propane- carboxylic acid | 497.1 | 4.333 |
| 143 | 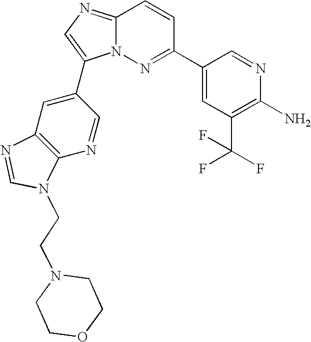 | 5-{3-[3-(2- Morpholin-4- yl-ethyl)- 3H-imidazo [4,5-b] pyridin-6- yl]- imidazo [1,2-b] pyridazin-6- yl}-3- trifluoro- methyl- pyridin-2- ylamine | Method A using 3-(2- morpholin- 4-yl-ethyl)- 6-(4,4,5,5- tetramethyl- [1,3,2] dioxa- borolan-2- yl)-3H- imidazo [4,5-b] pyridine instead (for later see Method K) | 510.1 | 3.692 |
| 144 | 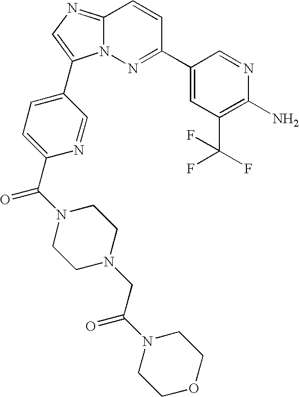 | 2-(4-{4-[6-(6- Amino-5- trifluoro- methyl- pyridin-3- yl)-imidazo [1,2-b] pyridazin-3- yl]- benzoyl}- piperazin-1- yl)-1- morpholin-4- yl-ethanone | same as example 80 using 1- morpholin- 4-yl-2- piperazin- 1-yl- ethanone instead | 595.1 | 3.775 |
| 145 | 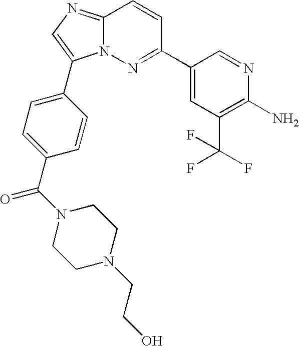 | 2-(4-{4-[6-(6- Amino-5- trifluoro- methyl- pyridin-3- yl)- imidazo [l,2-b] pyridazin-3- yl]- benzoyl}- piperazin-1- yl)-1- morpholin-4- yl-ethanone | same as example 80 using 2- piperazin- 1-yl- ethanol instead | 512.1 | 3.692 |
| 146 | 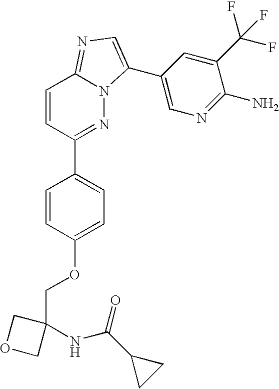 | Cyclo- propane- carboxylic acid (3- {4-[3-{6- amino-5- trifluoro- methyl- pyridin-3- yl)-imidazol [1,2-b] pyridazin-6- yl]-phenoxy- methyl}- oxetan-3- yl)-amide | see text | | |
| 147 | 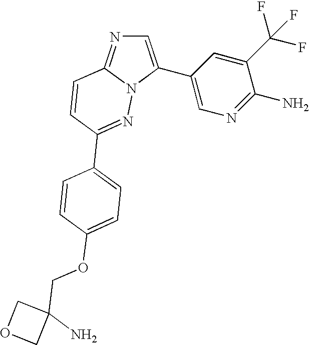 | 5-{6-[4-(3- Amino- oxetan-3- ylmethoxy)- phenyl]- imidazo [1,2-b] pyridazin-3- yl}-3- trifluoromethyl- pyridin-2- yl amine | see text | | see text |
| 148 | 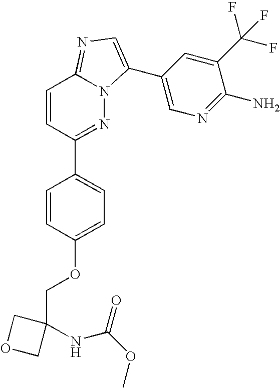 | (3-{4-[3-(6- Amino-5- trifluoro- methyl- pyridin-3-yl)- imidazo [1,2-b] pyridazin-6- yl]- phenoxy- methyl}- oxetan-3- yl)- carbamic acid methyl ester | see text | | |
| 149 | 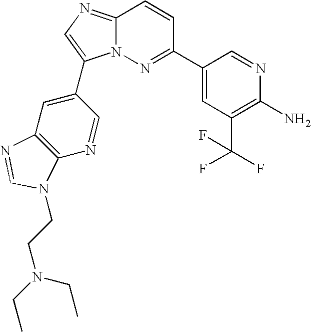 | 5-{3-[3-(2- Diethyl- amino- ethyl)-3H- imidazo [4,5-b]pyridin-6- yl]- imidazo [1,2-b] pyridazin-6- yl}-3- trifluoro- methyl- pyridin-2- ylamine | Method A using diethyl- {2-[6- (4,4,5,5- tetra- methyl- [1,3,2] dioxa- borolan- 2-yl)- imidazo [4,5-b] pyridin-3- yl]-ethyl}- amine instead (for later see Method K ) | 496.1 | 3.742 |
| 150 | 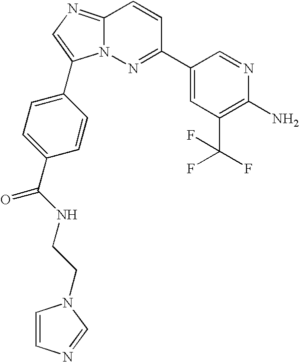 | 4-[6-(6- Amino-5- trifluoro- methyl- pyridin-3- yl)- imidazo [1,2-b] pyridazin- 3-yl]-N- {2- imidazol-1- yl-ethyl)- benzamide | same as example 80 using 2- imidazol- 1-yl- ethyl- amine instead | 492.9 | 3.675 |
| 151 | 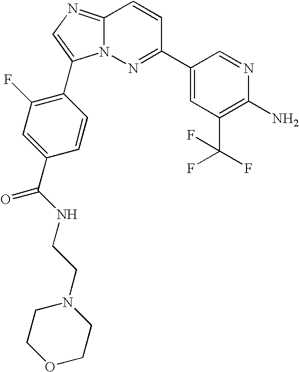 | 4-[6-(6- Amino-5- trifluoro- methyl- pyridin-3- yl)- imidazo [1,2-b] pyridazin-3- yl]-3- fluoro-N-(2- morpholin- 4-yl-ethyl)- benzamide | Method D starting from 4-[6-{6- Amino-5- trifluoro- methyl- pyridin-3- yl)- imidazo [1,2-b] pyridazin- 3-yl]-3- fluoro- benzoic acid (see example 80), using 2- morpholin- 4-yl- ethylamine | 530.1 | 3.775 |
| 152 | 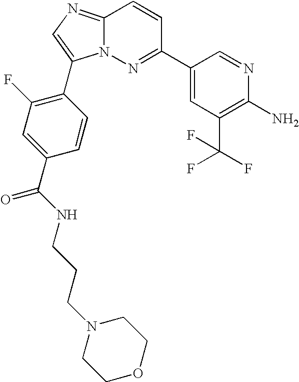 | 4-[6-(6- Amino-5- trifluoro- methyl- pyridin-3-yl)- imidazo [1,2-b] pyridazin-3- yl]-3-fluoro- N-(3- morpholin-4- yl-propyl)- benzamide | same as example 151 using 3- morpholin- 4-yl- propyl- amine instead | 544.1 | 3.775 |
| 153 | 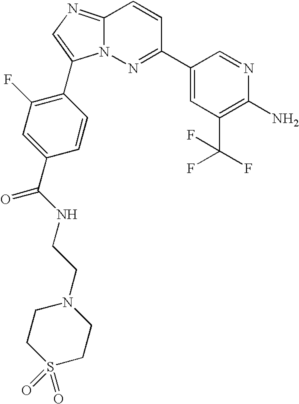 | 4-[6-(6- Amino-5- trifluoro- methyl- pyridin-3-yl)- imidazo [1,2-b] pyridazin-3- yl]-N-[2- (1,1-dioxo- 1lambda*6*- thio- morpholin-4-yl)- ethyl]-3- fluoro- benzamide | same as example 151 using 2-(1,1- dioxo- 1lambda*6*- thio- morpholin- 4-yl)- ethylamine instead | 578 | 3.817 |
| 154 | 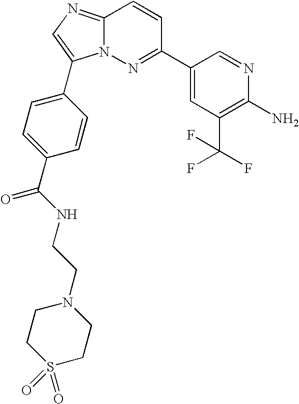 | 4-[6-(6- Amino-5- trifluoro- methyl- pyridin-3-yl)- imidazo [1,2-b] pyridazin-3- yl]-N-[2-(1,1- dioxo- 1lambda*6*- thio- morpholin- 4-yl)- ethyl]- benzamide | same as example 80 using 2-(1,1- dioxo- 1lambda*6*- thio- morpholin- 4-yl)- ethylamine instead | 560 | 3.758 |
| 155 | 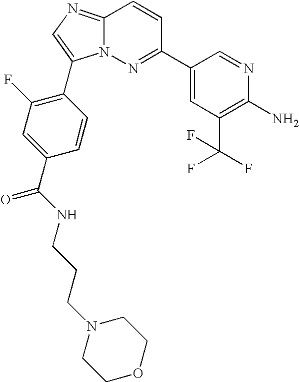 | 5-(6- Benzo[1,3] dioxol-5-yl- imidazo [1,2-b] pyridazin-3- yl)-3- trifluoro- methyl- pyridin-2- ylamine | Method A using benzo [1,3] dioxole-5- boronic acid and 5- (4,4,5,5- tetra- methyl- [1,3,2] dioxa- borolan- 2-yl)-3-t rifluoro- methyl- pyridin-2- ylamine | 400.1 | 4.5 |
| 156 | 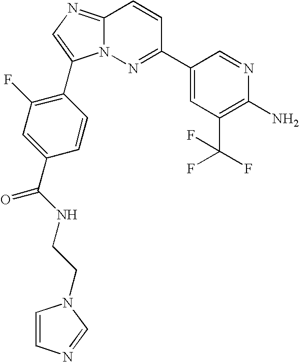 | 4-[6-(6- Amino-5- trifluoro- methyl- pyridin-3- yl)-imidazo [1,2-b] pyridazin- 3-yl]-3- fluoro-N-(2- imidazol-1-yl- ethyl)- benzamide | same as example 151 using 2-imidazol- 1-yl- ethylamine instead | 511 | 3.792 |
| 157 | 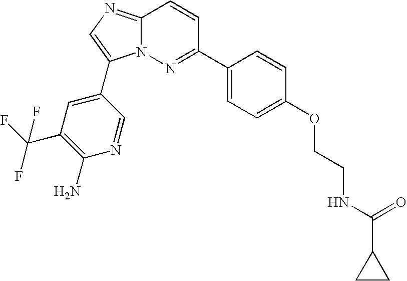 | Cyclo- propane- carboxylic acid (2- {4-[3-(6- amino-5- trifluoro- methyl- pyridin-3- yl)- imidazo [1,2-b] pyridazin- 6-yl]- phenoxy}- ethyl)- amide | Method E using, starting from 5-{6-[4- (2-amino- ethoxy)- phenyl]- imidazo [1,2-b] pyridazin- 3-yl}-3- trifluoro- methyl- pyridin-2- ylamine (example 74) | 483.1 | 4.208 |
| 158 | 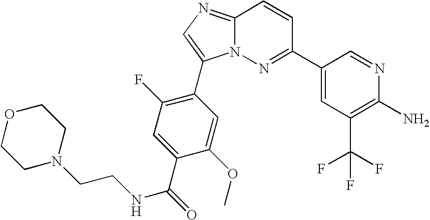 | 4-[6-(6- Amino-5- trifluoro- methyl- pyridin-3- yl)- imidazo [1,2-b] pyridazin- 3-yl]-5- fluoro-2- methoxy- N-(2- morpholin- 4-yl-ethyl)- benzamide | Method E starting from 4-[6-(6- amino-5- trifluoro- methyl- pyridin-3- yl)- imidazo [1,2-b] pyridazin- 3-yl]-5- fluoro-2- methoxy- benzoic acid and 2- morpholin- 4-yl- ethylamine (The acid is prepared according | 560 | 4.78** |
| 159 | 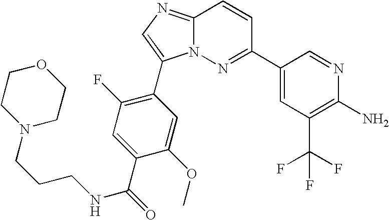 | 4-[6-(6- Amino-5- trifluoro- methyl- pyridin-3- yl)- imidazo [1,2-b] pyridazin- 3-yl]-5- fluoro-2- methoxy- N-(3- morpholin- 4-yl-propyl)- benzamide | same as example 158 using 3- morpholin- 4-yl- propylamine | 574 | 4.81** |
| 160 | 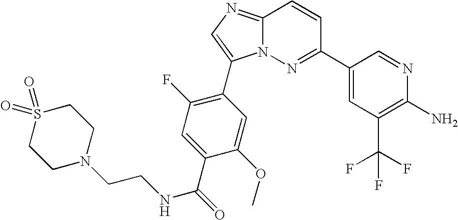 | 4-[6-(6- Amino-5- trifluoro- methyl- pyridin-3- yl)- imidazo [1,2-b] pyridazin- 3-yl]-N-[2- (1,1-dioxo- 1lambda*6*- thio- morpholin-4- yl)-ethyl]-5- fluoro- 2-methoxy- benzamide | same as example 158 using 2-(1,1- dioxo-1 lambda*6*- thio- morpholin- 4-yl)- ethylamine | 608.1 | 4.82** |
| 161 | 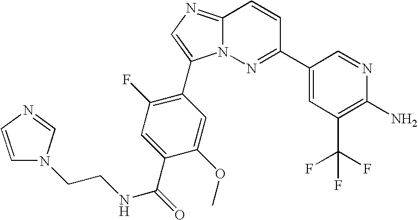 | 4-[6-(6- Amino-5- trifluoro- methyl- pyridin-3- yl)-imidazo (1,2- b]pyridazin- 3-yl]-5- fluoro-N- (2-imidazol- 1-yl- ethyl)-2- methoxy- benzamide | same as example 158 using 2-imidazol- 1-yl- ethylamine | 541 | 4.77 ** |
| 162 | 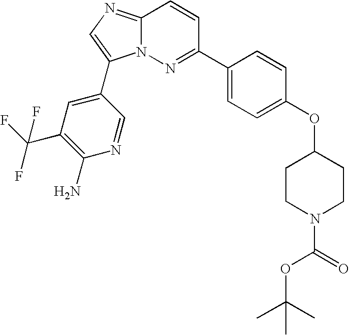 | 4-{4-[3-(6- Amino-5- trifluoro- methyl- pyridin-3-yl)- imidazo [1,2-b] pyridazin-6- yl]- phenoxy}- piperidine-1- carboxylic acid tert- butyl ester | same as example 89, using 4-(4-imidazo [1,2-b] pyridazin-6- yl- phenoxy)- piperidine- 1-carboxylic acid tert- butyl ester (see method G) instead | 555 | 5.258 |
| 163 | 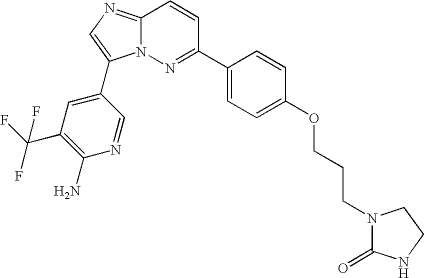 | 1-(3-{4-[3-(6- Amino-5- trifluoro- methyl- pyridin-3- yl)- imidazo [1,2-b] pyridazin- 6-yl]- phenoxy}- propyl)- imidazolidin- 2-one | same as example 89, using 1-[3-(4- imidazo [1,2-b] pyridazin- 6-yl- phenoxy)- propyl]- imidazolidin- 2-one (see method N) instead | 498.1 | 4.167 |
| 164 | 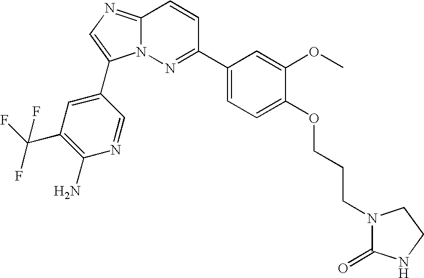 | 1-(3-{4-[3-(6- Amino-5- trifluoro- methyl- pyridin-3-yl)- imidazo [1,2-b] pyridazin-6- yl]-2- methoxy- phenoxy}- propyl)- imidazolidin- 2-one | same as example 89, using 1-[3-(4- imidazo [1,2-b] pyridazin- 6-yl-2- methoxy- phenoxy)- propyl]- imidazolidin- 2-one (see method N ) instead | 528 | 4.083 |
| 165 | 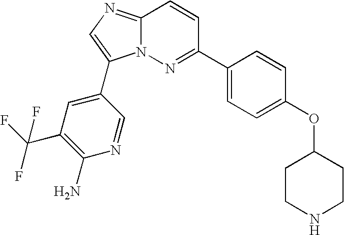 | 5-{6-[4-( Piperidin-4- yloxy)- phenyl]- imidazo [1,2-b] pyridazin-3- yl}-3- trifluoro- methyl- pyridin-2- ylamine | method B starting from 4-{4-[3-(6- amino-5- trifluoro- methyl- pyridin-3- yl)- imidazo [1,2-b] pyridazin- 6-yl]- phenoxy}- piperidine- 1-carboxylic acid tert- butyl ester (example 162) | 455.1 | 3.767 |
| 166 | 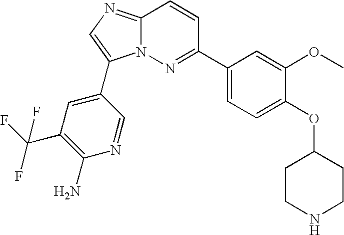 | 5-{6-(3- Methoxy- 4- (piperidin- 4-yloxy)- phenyl]- imidazo [1,2-b] pyridazin- 3-yl}-3- trifluoro- methyl- pyridin-2- ylamine | method B starting from 4-{4-[3-(6- amino-5- trifluoro- methyl- pyridin-3- yl)- imidazo [1,2-b] pyridazin- 6-yl]-2- methoxy- phenoxy}- piperidine- 1-carboxylic acid tert- butyl ester (example | 485 | 3.75 |
| 167 | 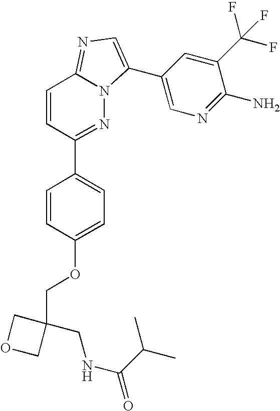 | N-(3-{4-[3-(6- Amino-5- trifluoro- methyl- pyridin-3- yl)- imidazo [1,2-b] pyridazin-6- yl]- phenoxy- methyl}- oxetan-3- ylmethyl)- isobulyramide | see text | | |
| 168 | 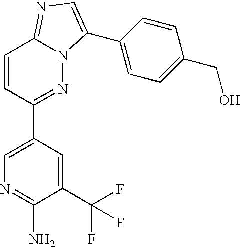 | {4-[6-(6- Amino-5- influoro- methyl- pyridin- 3-yl)- imidazo [1,2-b] pyridazin- 3-yl]- phenyl}- methanol | see text | | |
| 169 | 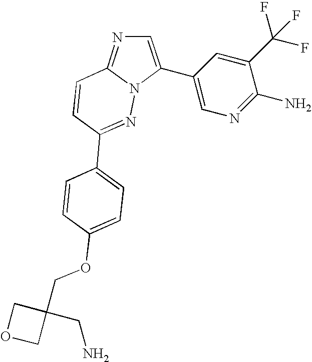 | 5-{6-[4-(3- Amino- methyl- oxetan- 3- ylmethoxy)- phenyl]- imidazo [1,2-b] pyridazin- 3-yl}-3- trifluoro- methyl- pyridin-2- ylamine | see text | | |
| 171 | 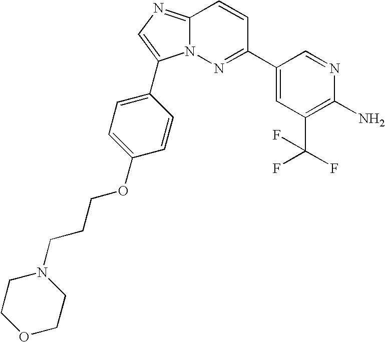 | 5-{3-[4-(3- Morpholin- 4-yl- propoxy)- phenyl]- imidazo [1,2-b] pyridazin-6- yl}-3-trifluoro- methyl- pyridin-2- ylamine | Method L using methane- sulfonic acid 3- morpholin- 4-yl- propyl ester instead | 499 | 3.792 |
| 172 | 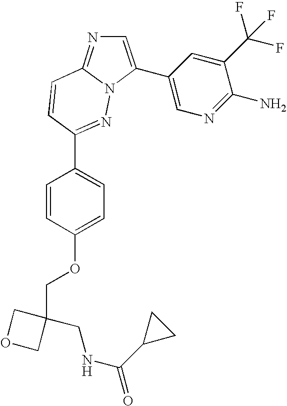 | Cyclo- propane- carboxylic acid (3- {4-[3-(6- amino-5- trifluoro- methyl- pyridin-3- yl)- imidazo [1,2-b] pyridazin- 6-yl]- phenoxy- methyl)- oxetan-3- ylmethyl)- amide | see text | | |
| 173 | 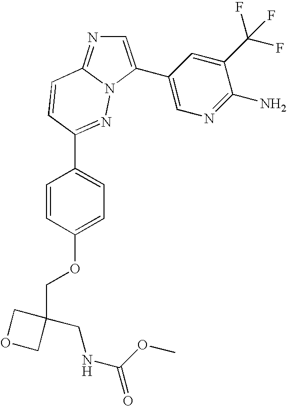 | (3-{4-[3-(6- Amino-5- trifluoro- methyl- pyridin-3- yl)- imidazo [1,2-b] pyridazin- 6-yl]- phenoxy- methyl}- oxetan-3- ylmethyl)- carbamic acid methyl ester | see text | | |
| 174 | 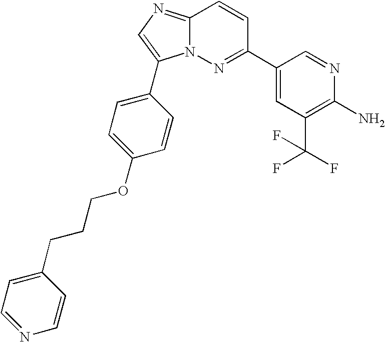 | 5-{3-[4-(3- Pyridin- 4-yl- propoxy)- phenyl]- imidazo [1,2-b] pyridazin- 6-yl}-3- trifluoro- methyl- pyridin- 2- ylamine | Method L using methane- sulfonic acid 3-pyridin- 4-yl- propyl ester instead | 491 | 3.925 |
| 175 | 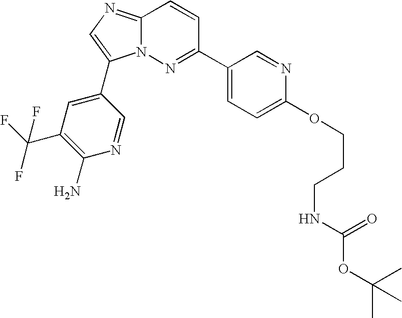 | (3-{5-[3- (G- Amino-5- trifluoro- methyl- pyridin-3- yl)- imidazo [1,2-b] pyridazin- 6-yl]- pyridin-2- yloxy}- propyl)- carbamic acid tert- butyl ester | same as example 89, using 5- imidazo [1,2-b] pyridazin- 6-yl- pyridin- 2-ol and methane- sulfonic cid 3-tert- butoxy- carbonyl- amino- propyl ester (see method G) instead | 530 | 4.767 |
| 176 | 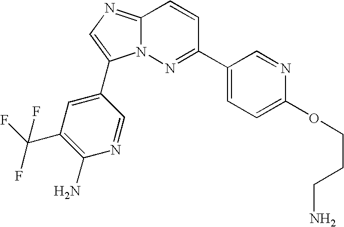 | 5-{6-[6-(3- Amino- propoxy)- pyridin-3- yl]- imidazo- [1,2b] pyridazin- 3-yl}-3- trifluoro- methyl- pyridin-2- ylamine | method B starting from (3-{5-[3-(6- amino-5- trifluoro- methyl- pyridin-3- yl)- imidazo [1,2-b] pyridazin- 6-yl]- pyridin-2- yloxy}- propyl)- carbamic acid tert-butyl ester | 430.1 | 3.65 |
| 177 | 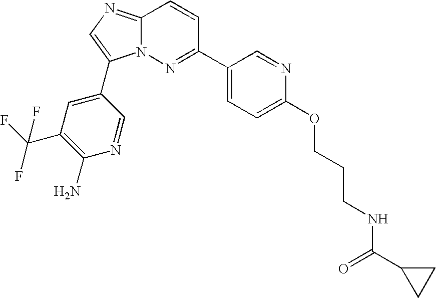 | Cyclo- propane- carboxylic acid (3- {5-[3-(6- amino-5- trifluoro- methyl- pyridin-3- yl)- imidazo [1,2-b] pyridazin- 6-yl]- pyridin- 2-yloxy}- propyl)- amide | Method E sing, starting from 5-{6-[6- (3-amino- propoxy)- pyridin-3- yl]- imidazo [1,2-b] pyridazin- 3-yl}-3- trifluoro- methyl- pyridin-2- ylamine (example 176) | 498 | 4.2 |
| 178 | 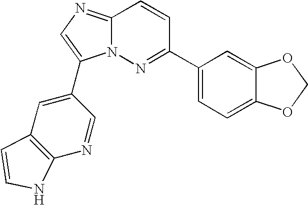 | 6-Benzo [1,3] dioxol- 5-yl-3-(1H- pyrrolo [2,3-b] pyridin-5- yl)- imidazo [1,2-b] pyridazine | method A using benzo [1,3] dioxole-5- boronic acid and 5- (4,4,5,5- tetra- methyl- [1,3,2] dioxa- borolan- 2-yl)-1H- pyrrolo [2,3-b] pyridine | 356.1 | 4.208 |
| 179 | 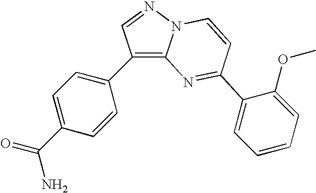 | 4-[5-(2- Methoxy- phenyl)- pyrazolo (1,5-a] pyrimidin-3- yl]- benzamide | see text | | |
| 181 | 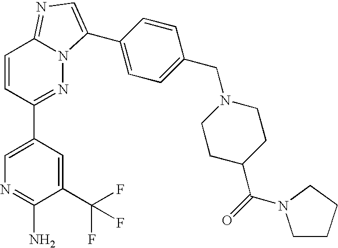 | (1-{4-[6-(6- Amino-5- trifluoro- methyl- pyridin- 3-yl)- imidazo [1,2-b] pyridazin- 3-yl]- benzyl}- piperidin- 4-yl)- pyrrolidin- 1-yl- methanone | see text | | |
| 182 | 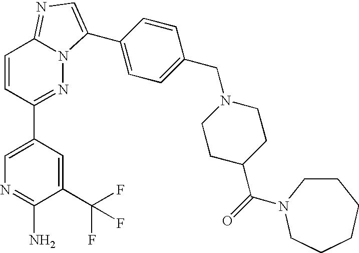 | (1-{4-[6-(6- Amino-5- trifluoro- methyl- pyridin-3- yl)- imidazo [1,2-b] pyridazin- 3-yl]- benzyl}- piperidin- 4-yl)- azepan- 1-yl- methanone | see text | | |
| 183 | 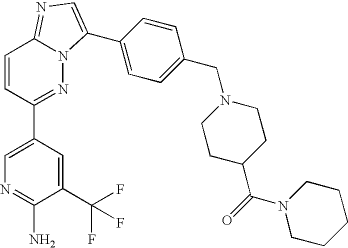 | (1-{4-(6-(6- Amino-5- trifluoro- methyl- pyridin-3- yl)- imidazo [1,2-b] pyridazin-3- yl]- benzyl}- piperidin- 4- yl)- piperidin- 1-yl- methanone | see text | | |
| 184 | 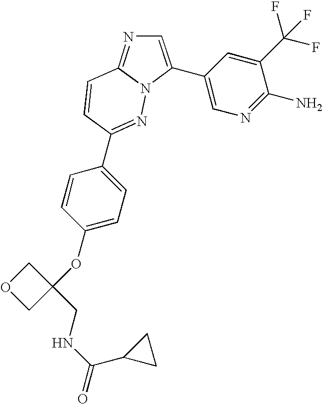 | Cyclo- propane- carboxylic acid (3- {4-[3-(6- amino-5- trifluoro- methyl- pyridin-3- yl)- imidazo [1,2-b] pyridazin- 6- yl]- phenoxy}- oxetan- 3-yl- methyl)- amide | see text | | |

Synthesis Method A

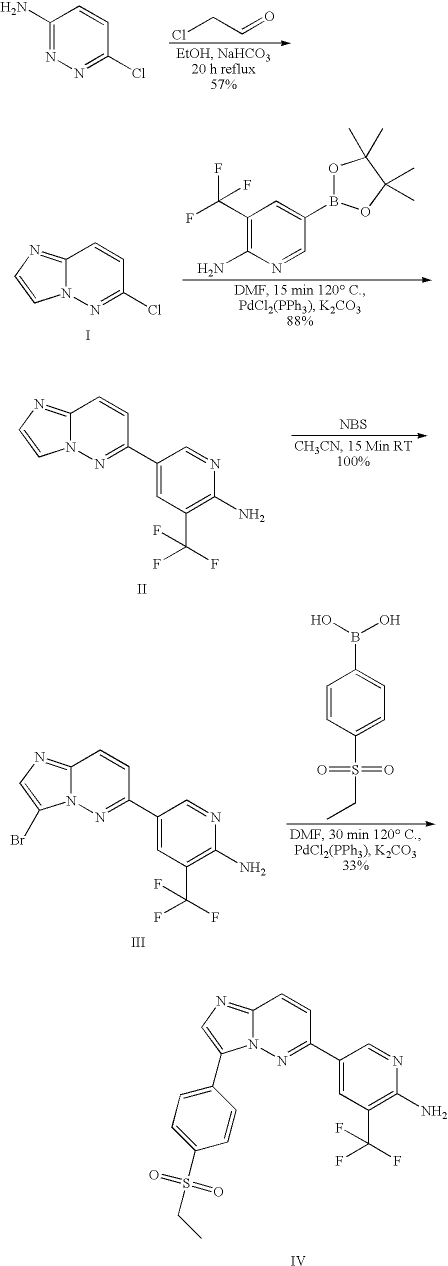

### Stage A.1: 6-Chloro-imidazo[1,2-b]pyridazine (I)

3-Amino-6-chloropyridazine (5 g; 38.6 mMol) is suspended in EtOH (5 mL) and treated at RT with chloroacetaldehyde (50% in water; 13.7 mL; 106 mMol) and sodium bicarbonate (5.51 g; 65.6 mMol). The yellow suspension is heated to reflux (bath 95° C.) and stirred for 19 h, followed by stirring at RT for 48 h. Additional chloroacetaldehyde (50% in water; 4.98 mL) and sodium bicarbonate (1.21 g) is added and the brown suspension is refluxed for another 4 h. After cooling to RT, the reaction mixture is freed from solvent under reduced pressure and the residue is taken up into CH₂Cl₂ (400 mL). Some insoluble residue is filtered off, washed with additional CH₂Cl₂ and the organic layer is washed with water (2×200 mL). The organic layer is dried (Na₂SO₄), and concentrated under reduced pressure to obtain the title compound as brownish solid (4.94 g); MS(ESI⁺):m/z=153.9 (M+H)⁺; HPLC: tRet=2.90 minutes (System 1). The title compound is used in the next step without further purification.

### Stage A.2: 5-Imidazo[1,2-b]pyridazin-6-yl-3-trifluoromethyl-pyridin-2-ylamine (II)

6-Chloro-imidazo[1,2-b]pyridazine (I) (384 mg; 2.5 mMol) is dissolved in DMF (15 mL), followed by addition of 5-(4,4,5,5-Tetramethyl-[1,3,2]dioxaborolan-2-yl)-3-trifluoromethyl-pyridin-2-ylamine (example 4; stage 4.2) (864 mg; 3 mmol), PdCl₂(PPh₃) (30 mg) and potassium carbonate (1M soln. in H₂O; 6.25 mL). The mixture is heated under stirring to 120° C. for 15 min. After cooling to RT EtOAc (150 mL) is added and the organic layer is washed with water (2×). After removal of the solvent under reduced pressure, the crude product is purified by flash chromatography (30 g Silica gel [0.040-0.063 mm] Merck 1.09.385.1000]; eluting with CH₂Cl₂/MeOH 98:2), to obtain the title compound (618 mg) as as yellowish powder (618 mg); MS(ESI⁺):m/z=280.1 (M+H)⁺; HPLC: tRet=3.717 minutes (System 2).

In cases of less reactive boronates, the reaction time is extended up to 120 min, and/or additional 5-(4,4,5,5-Tetramethyl-[1,3,2]dioxaborolan-2-yl)-1H-pyrrolo[2,3-b]pyridine (0.5 eq.), PdCl₂(PPh₃) (50% of original amount) and potassium carbonate (0.5 eq.) is added and the mixture is stirred for 1 h at 120° C.

### Stage A.3: 5-(3-Bromo-imidazo[1,2-b]pyridazin-6-yl)-3-trifluoromethyl-pyridin-2-ylamine (III)

5-Imidazo[1,2-b]pyridazin-6-yl-3-trifluoromethyl-pyridin-2-ylamine (II) (615 mg; 2.2 Mol) is dissolved in CH₃CN (20 mL), followed by addition of N-bromosuccinimide (95%; 433 mg; 2.31 mMol). After stirring the mixture at RT for 15 min. the solvent is removed under reduced pressure and the residue taken up into EtOAc (150 mL). The organic layer is washed with water (2×). followed by removal of the solvent under reduced pressure. The product is freeze-dried from dioxane to obtain the title compound (750 mg) as a brightly yellowish powder (750 mg); MS(ESI⁺):m/z=359.9 (M+H)⁺; HPLC: tRet=4.833 minutes (System 2).

### Example 33

### 5-[3-(4-Ethanesulfonyl-phenyl)-imidazo[1,2-b]pyridazin-6-yl]-3-trifluoro-methyl-pyridin-2-ylamine (IV)

5-(3-Bromo-imidazo[1,2-b]pyridazin-6-yl)-3-trifluoromethyl-pyridin-2-ylamine (III) (54 mg; 0.15 mMol) is dissolved in DMF (5 mL) and treated at RT with 4-ethylsulfonylphenyl-boronic acid (49 mg; 0.225 mMol), PdCl₂(PPh₃) (6 mg) and potassium carbonate (1M soln. in H₂O; 0.375 mL). The reaction mixture is stirred at 120° C. for 60 min. After cooling to RT, EtOAc is added (50 mL), followed by extraction with water (2×). The solvent under is removed under reduced pressure and the crude product purified by flash chromatography (30 g silica gel [0.040-0.063 mm] Merck 1.09.385.1000]; eluting with CH₂Cl₂/CH₃OH 96:4). The title compound (22 mg) is obtained by freeze-drying from dioxane as brightly yellowish powder (22 mg); MS(ESI⁺):m/z=448.0 (M+H)⁺; HPLC: tRet=4.467 minutes (System 2).

Synthesis Method B

### Example 37

### 5-{3-[4-(3-Amino-propoxy)-3-methoxy-phenyl]-imidazo[1,2-b]pyridazin⁻6-yl}-3-trifluoromethyl-pyridin-2-ylamine

(3-{4-[6-(6-Amino-5-trifluoromethyl-pyridin-3-yl)-imidazo[1,2-b]pyridazin-3-yl]-2methoxy-phenoxy}-propyl)-carbamic acid tert-butyl ester (example 36) (25 mg; 0.0448 mMol) is dissolved in TFA (0.2 mL) and kept for 5 min at RT. The reaction mixture is treated with NaHCO₃ (5% soln.) to reach pH 8-9 and extracted with n-butanol. The combined organics are washed with NaHCO₃ (5% soln.; 1×) and water (2×), followed by removal of the solvent under reduced pressure. The title compound (16 mg) is obtained by freeze-drying from dioxane as yellowish powder. Title compound: MS(ESI⁺):m/z=459.1 (M+H)⁺; HPLC: tRet=3.775 minutes (System 2)

Synthesis Method C

### 5-(4,4,5,5-Tetramethyl-[1,3,2]dioxaborolan-2-yl)-1H-pyrrolo[2,3-b]pyridine

5-Bromo-1H pyrrolo[2,3b]pyridine (552 mg; 2.8 mMol) is suspended in dioxane (15 mL), followed by addition of bis(pinacoloato)diboran (854 mg; 3.36 mMol) and potassium acetate (824 mg; 8.40 mMol); this mixture is put under argon for 30 min. 1,1 bis(PPh₂)FePdCl₂×CH₂Cl₂ (82 mg; 0.112 mMol) is added and the mixture heated to reflux for 1 h. After cooling to RT, EtOAc (50 mL) is added. The precipitate is filtered off, washed with EtOAc and the solvent removed under reduced pressure to obtain the title compound. Title compound: MS(ESI⁺):m/z=245.1 (M+H)⁺; HPLC: tRet=3.325 minutes (System 2)

Synthesis Method D

### Example 50

### (3-{4-[3-(6-Amino-5-trifluoromethyl-pyridin-3-yl)-imidazo[1,2-b]pyridazin-6yl]-2-methoxy-phenoxy}-propyl)-carbamic acid methyl ester)

5-{6-[4-(3-Amino-propoxy)-3-methoxy-phenyl]-imidazo[1,2-b]pyridazin-3-yl}-3-trifluoromethyl-pyridin-2-ylamine (example 14) (27.5 mg; 0.060 mMol) is dissolved in DMF (2 mL) followed by addition of methyl-chloroformate (δ1.224; 5.1 mikroL; 0.066 mMol) and N,N-diisopropylamine (δ0.775; 11.2 mikroL; 0.066 mMol) and kept stirring at RT for 10 min. After completion, EtOAc (50 mL) is added, followed by extraction with NaHCO₃ (5% soln.) (2×) and water (2×) and removal of the solvent under reduced pressure. The title compound (20 mg) is obtained by freeze-drying from dioxane as yellow powder. Title compound: MS(ESI⁺):m/z=517.1 (M+H)⁺; HPLC: tRet=4.375 minutes (System 2).

Synthesis Method E

### Example 58

### Cyclopropanecarboxylic acid (3-{4-[3-(6-amino-5-trifluoromethyl-pyridin-3-yl)-imidazo[1,2-b]pyridazin-6-yl]-phenoxy}-propyl)-amide

5-{6-[4-(3-Amino-propoxy)-phenyl]-imidazo[1,2-b]pyridazin-3-yl}-3-trifluoromethyl-pyridin-2-yl-amine (example 56) (30 mg; 0.07 mMol) is dissolved in DMF (2 mL). In a separate vessel, cyclopropane carboxylic acid (δ1.088; 6.2 mikroL; 0.077 mMol), TPTU (23 mg; 0.077 mMol) and N,N-diisopropylamine (δ0.775; 39 mikroL; 224 mMol) is reacted for 5 min at RT and then added to the amine containing solution. After completion of the reaction (15 min), EtOAc (50 mL) is added, followed by extraction with NaHCO₃ (5% soln.) (2×) and water (2×) and removal of the solvent under reduced pressure. The title compound (29 mg) is obtained by freeze-drying from dioxane as yellow powder. Title compound: MS(ESI⁴):m/z=497.1 (M+H)⁺; HPLC: tRet=4.392 minutes (System 2).

Synthesis Method F

### {3-[2-Methoxy-4-(4,4,5,5-tetramethyl[1,3,2]dioxaborolan-2-yl)-phenoxy]-propyl}-carbamic acid tert-butyl ester

2-Methoxy-4-(4,4,5,5-tetramethyl-[1,3,2]dioxaborolan-2-yl)-phenol (250 mg; 1 mMol) is dissolved in DMF (5 mL) and cooled to 0° C. At this temperature, NaH (65 mg; 1.5 mMol) is added and the mixture kept stirring for 30 min at 0° C. 3-(Boc-amino)propylbromide (286 mg; 1.2 mMol) is added, the ice bath is removed and the reaction continued at RT for 18 h. EtOAc is added (150 mL) and the mixture is extracted with water (2×). The solvent is removed under reduced pressure and the title compound (420 mg; red oil) is used without further purification. Title compound: MS(ESI⁺):m/z=408.1 (M+H)⁺; HPLC: tRet=5.817 minutes (System 2).

Synthesis Method G

### 1-[3-(4-Imidazo[1,2-b]pyridazin-6-yl-phenoxy)-propyl]-pyrrolidin-2-one

4-Imidazo[1,2-b]pyridazin-6-yl-phenol (98 mg; 0.46 mMol) is dissolved in DMA (10 mL) followed by addition of methanesulfonic acid 3-(2-oxo-pyrrolidin-1-yl)-propyl ester (154 m; 0.69 mMol) and Cs₂CO₃ (300 mg; 0.92 mMol). The mixture is heated at 50° C. for 16 h, followed by addition of another equivalent of methanesulfonic acid 3-(2-oxo-pyrrolidin-1-yl)-propyl ester (154 m; 0.69 mMol) and Cs₂CO₃ (300 mg; 0.92 mMol). The mixture is stirred another 4 h at 50° C. After cooling to RT, EtOAc (100 mL) is added, followed by extraction with water (2×). The solvent under is removed under reduced pressure and the crude product purified by flash chromatography (30 g silica gel [0.040-0.063 mm] Merck 1.09.385.1000]; eluting with CH₂Cl₂/CH₃OH 98:2). The title compound is obtained by freeze-drying from dioxane as white powder (53 mg); MS(ESI⁺):m/z=337.2 (M+H)⁺; HPLC: tRet=3.983 minutes (System 2).

### 4-Imidazo[1,2-b]pyridazin-6-yl-phenol

6-Chloro-imidazo[1,2-b]pyridazine (I) (230 mg; 1.5 mMol) is dissolved in DMF (10 mL), followed by addition of 4-hydroxyphenylboronic acid (248 mg; 1.8 mMol), PdCl₂(PPh₃) (20 mg) and potassium carbonate (1M soln. in H₂O; 3.75 mL). The reaction mixture is stirred at 120° C. for 15 min. After cooling to RT, EtOAc is added (100 mL), followed by extraction with water (2×). The solvent under is removed under reduced pressure and the crude product purified by flash chromatography (30 g silica gel [0.040-0.063 mm] Merck 1.09.385.1000]; eluting with CH₂Cl₂/CH₃OH 96:4). The title compound (22 mg) is obtained by freeze-drying from dioxane as beige powder (198 mg); MS(ESI⁺):m/z=212.1 (M+H)⁺; HPLC: tRet=3.633 minutes (System 2).

### Methanesulfonic acid 3-(2-oxo-pyrrolidin-1-yl)-propyl ester

1-(3-Hydroxypropyl)-2pyrrolidon (95%; δ1.1; 1.1 mL; 8 mMol) is dissolved in CH₂Cl₂ (20 mL) and cooled to 0° C. At this temperature, methanesulfochloride (δ1.476; 0.69 mL; 8.8 mMol) and triethylamine (δ0.726; 1.68 mL; 12 mMol) is added and kept stirring at 0° C. for 2 h. CH₂Cl₂ (30 mL) is added and the mixture extracted with water (2×). The solvent is removed under reduced pressure and the title compound (1.68 g; colorless oil) is used without further purification. Title compound: MS(ESI⁺):m/z=222.1 (M+H)⁺.

Synthesis Method H

### 4-[2-(4-Bromo-benzenesulfonyl)-ethyl]-morpholine

1-Bromo-4-(2-chloro-ethanesulfonyl)-benzene (226 mg; 0.8 mMol) is dissolved in DMA (5 mL) followed by addition of morpholin (δ1.00; 0.35 mL; 4 mMol) and the mixture is kept stirring at 50° C. for 3 h. After cooling to RT, EtOAc is added 80 mL), followed by extraction with water (2×). The solvent under is removed under reduced pressure and the and the title compound (254 mg; white powder) is used without further purification. Title compound: MS(ESI⁺):m/z=336.0 (M+H)⁺; HPLC: tRet=4.000 minutes (System 2).

Synthesis Method I

### 4-(3-Bromo-imidazo[1,2-b]pyridazin-6-yl)-N-(2-morpholin-4-yl-ethyl)-benzamide

### Example 122.1

### 4-Imidazo[1,2-b]pyridazin-6-yl-benzoic acid I.1

6-Chloro-imidazo[1,2-b]pyridazine (I) (230 mg; 1.5 mMol) is dissolved in DMF (10 mL), followed by addition of 4-(4,4,5,5-tetramethyl-[1,3,2]dioxaborolan-2-yl)-benzoic acid (460 mg; 1.8 mMol); PdCl₂(PPh₃) (20 mg) and potassium carbonate (1M soln. in H₂O; 3.75 mL). The mixture is heated under stirring to 120° C. for 30 min. After cooling to RT EtOAc (100 mL) is added and the organic layer is washed with NaHCO₃ (5% soln.) and water (2 ×). The aqueous layer is adjusted to pH 1-2 using citric acid (5% soln.) followed by extraction with EtOAc. The solvent is removed under reduced pressure, until the product precipitates and is filtered off to obtain the title compound as a brown powder (252 mg). Title compound: MS(ESI⁺):m/z=240.2 (M+H)⁺; HPLC: tRet=3.608 minutes (System 2).

### Example 122.2

### 4-(3-Bromo-imidazo[1,2-b]pyridazin-6-yl)-benzoic acid I.2

The title compound is prepared in analogy to Method A; stage A3; starting from 4-Imidazo[1,2-b]pyridazin-6-yl-benzoic acid (example 122.1). Title compound (grey powder); MS(ESI⁺):m/z=320.0 (M+H)⁺; HPLC: tRet=4.533 minutes (System 2).

### Example 122.3

### 4-(3-Bromo-imidazo[1,2-b]pyridazin-6-yl)-N-(2-morpholin-4-yl-ethyl)-benzamide I.3

The title compound is prepared as described in Method E; starting 4-(3-Bromo-imidazo[1,2-b]pyridazin-6-yl)-benzoic acid 1.2, using 2-morpholin-4-yl-ethylamine instead. Title compound (brown powder); MS(ESI⁺):m/z=431.9 (M+H)⁺; HPLC: tRet=3.867 minutes (System 2).

Synthesis Method J

### 4-(2-Morpholin-4-yl-ethylsulfamoyl)-boronic acid

4-boronbenzenesulfonamide (97%; 207 mg; 1 mMol) is dissolved in DMA (10 mL) followed by addition of N-(2-chloroethyl)morphlin×HCl (372 mg; 2 mMol) and K₂CO₃ (692 mg; 5 mMol). The reaction mixture is kept stirring at 120° C. for 4 h. After cooling to RT, EtOAc (50 mL) is added and extracted with water (2×). The combined aqueous layers are back extracted with butanol followed by removal of the solvent under reduced pressure to obtain the title compound which is used without further purification. Title compound: MS(ESI⁺):m/z=315.1 (M+H)⁺; HPLC: tRet=3.383 minutes (System 2).

Synthesis Method K

### 1-(2-Morpholin-4-yl-ethyl)-5-(4,4,5,5-tetramethyl-[1,3,2]dioxaborolan-2-yl)-1H-indazole

The title compound is prepared in analogy to method J, starting from 5-bromo-1H-indazole, followed by transformation of the bromide to the boronate ester according to method C. Title compound: MS(ESI⁺):m/z=358.1 (M+H)⁺; HPLC: tRet=4.417 minutes (System 2).

Synthesis Method L

### Example 141

### (3-{4-[6-(6-amino-5-trifluoromethyl-pyridin-3-yl)-imidazo[1,2-b]pyridazin-3-yl]-phenoxy}-propyl)-carbamic acid tert-butyl ester

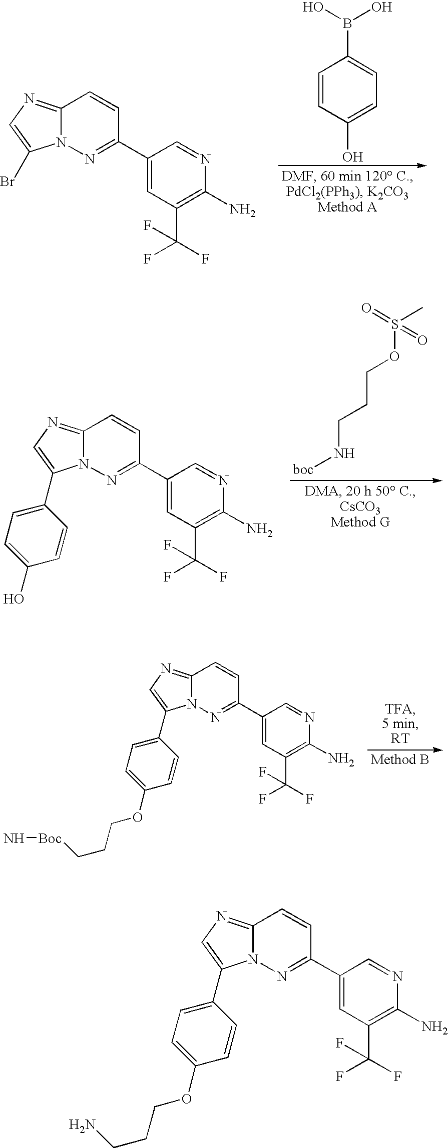

The title compound is prepared in analogy to method A, starting from e (see Method A), followed by alkylation of the phenol in analogy to method G, and finally removal of the Boc protecting group in according to method B. Title compound: MS(ESI⁺):m/z=529.1 (M+H)⁺; HPLC: tRet=4.850 minutes (System 2).

Synthesis Method M

### 5-Fluoro-2-methoxy-4-(4,4,5,5-tetramethyl-[1,3,2]dioxaborolan-2-yl)-benzoic acid methyl ester

The title compound is prepared in analogy to method C, using 4-bromo-5-fluoro-2-methoxy-benzoic acid methyl ester instead. Title compound: brown oil; MS(ESI⁺):m/z=310.0 (M+H)⁺; HPLC: tRet=4.37 minutes (System 1).

The title compound is reacted with 5-(3-Bromo-imidazo[1,2-b]pyridazin-6-yl)-3-trifluoromethyl-pyridin-2-ylamine (III) (Stage A.3) followed by cleavage of the methyl ester to allow for the amide formation described in method E.

### M.1 Trifluoro-methanesulfonic acid 4-bromo-5-fluoro-2-methoxy-phenyl ester

4-Bromo-5-fluoro-2-methoxyphenol (1 g; 4.52 mMol) is dissolved in pyridine (6 mL), cooled to −15 to −20° C., followed by dropwise addition of trifluoro methane sulfonic acid anhydride within 30 min at maximum −10° C. After another 10 min at ca 0° C. stirring is continued at RT for 16 h. the reaction mixture is poured onto cold water (50 mL) and stirred with tert. butyl methyl ether (50 mL). The organic layer is washed twice with HCl solution (1M; 25 mL), brine and dried over Na₂SO₄. After removal of the solvent under reduced pressure, the title compound is isolated as bright beige powder (1.36 g). Title compound: MS(ESI⁺):m/z=351.9 (M+H)⁺; HPLC: tRet=7.27 minutes (System 1).

### M.2 4-Bromo-5-fluoro-2-methoxy-benzoic acid methyl ester

Trifluoro-methanesulfonic acid 4-bromo-5-fluoro-2-methoxy-phenyl ester (M.1) (1.23 g; 3.48 mMol), Pd(OAc)₂ (95.8 mg; 0.418 mMol) and 1,3-bis-(diphenylphosphino)-propane (176 mg; 0.418 mMol) is dissolved under an atmosphere of argon in DMSO (11.3 mL), followed by addition of tributylamine (8.39 mL; 34.8 mMol). After stirring this biphasic mixture for 5 min at RT, DMSO (22.6 mL) and CH3OH (22.6 mL) is added to obtain a clear yellow solution. Under stirring at 40° C. bath temperature, CO gas is bubbled through for 5 h. The reaction mixture is treated with HCl solution (1M; 50 mL) and extracted with tert. butyl methyl ether (2×700 mL). The combined organic layers are washed with water (80 mL), brine (80 mL), dried over Na₂SO₄ and freed from the solvent under reduced pressure. Purification is done by chromatography (120 g Redisep, ISCO Companion; eluting with EtOAc/hexane 2:1), to obtain the title compound as as bright brown powder (721 mg); MS(ESI⁺):m/z=262.8 (M+H)⁺; HPLC: tRet=6.24 minutes (System 1).

Synthesis Method N

4-Imidazo[1,2-b]pyridazin-6-yl-phenol (125 mg; 0.591 mMol) (see method G) is dissolved in DMA (10 mL) followed by addition of 1-(3-chloropropyl)-2-imidazolidinone (116 mg; 0.709 mMol), tetrabutylammonium iodide (2.2 mg) and potassium carbonate (204 mg; 1.477 mMol). The reaction mixture is heated to 120° C. for 3 h. After cooling to RT, EtOAc (100 mL) is added, followed by extraction with water (2×) and removal of the solvent under reduced pressure. The title compound (167 mg) is obtained by freeze-drying from dioxane as bright beige powder. Title compound: MS(ESI⁺):m/z=338.2 (M+H)⁺; HPLC: tRet=3.800 minutes (System 2).

### Example 146

### Cyclopropanecarboxylic acid (3-{4-[3-(6-amino-5-trifluoromethyl-pyridin-3-yl)-imidazo[1,2-b]pyridazin-6-yl]-phenoxymethyl}-oxetan-3-yl)-amide

In a 5 ml vial with magnetic stir bar 50 mg (0.107 mmol) of 5-{6-[4-(3-amino-oxetan-3-ylmethoxy)-phenyl]-imidazo[1,2-b]pyridazin-3-yl}-3-trifluoromethyl-pyridin-2-yl amine (for preparation see example 147) and 40 μL (0.285 mmol) of triethylamine are dissolved in 1 mL of CH2Cl2 under nitrogen. Thereafter a solution of 10 μL (0.11 mmol) of cyclopropanecarbonyl chloride in 0.2 mL of CH2Cl2 is added slowly at room temperature. Since there is still starting material present another 100 μL of triethylamine and 50 μL of cyclopropanecarbonyl chloride is added at RT over 3 hours. After complete addition, no more starting material can be detected in the HPLC or MS. The reaction mixture is filtered and the solvent is evaporated. The crude product is purified by chromatography on 4 g of silica gel on a Combiflash Companion (Isco Inc.) using a gradient of 100% CH2Cl2 to 5% EtOH in CH2Cl2. Fractions containing pure product are combined and evaporated. The residue is triturated with hexanes and filtered to yield the title compound as a yellow solid. MS-ES: (M+1)=525.1, HPLC: t_{R}=4.966 min. Rf (CH2C12/EtOH 95:5)=0.

### Example 147

### 5-{6-[4-(3-Amino-oxetan-3-ylmethoxy)-phenyl]-imidazo[1,2-b]pyridazin-3-yl}-3-trifluoromethyl-pyridin-2-yl amine

Crude (3-{4-[3-(6-amino-5-trifluoromethyl-pyridin-3-yl)-imidazo[1,2-b]pyridazin-6-yl]-phenoxymethyl}-oxetan-3-yl)-carbamic acid benzyl ester (for preparation see stage 147.1), 0.9 g (˜1.30 mmol) is deprotected by hydrogenation at 5 bar and room at temperature with 10% Palladium on carbon (0.2 g) in 10 mL of THF. After 8 hours the hydrogenation is stopped and the catalyst filtered off through a pad of hyflo. The solvent is evaporated, the redidue is taken up in CH2Cl2 and extracted with 10% citric acid. The organic phase is re-extracted with citric acid and the aqueous phase is washed with CH2Cl2. Thereafter the pH of the combined aqueous extracts is adjusted to ˜10 by the addition of sodium hydroxide solution. Extraction with CH2Cl2 (5×) followed by drying over Na₂SO₄, and evaporation of the solvent gives the title compound as a yellow solid. MS-ES: (M+1) 457.1, HPLC: t_{R}=4.187 min. M.p. 198-200° C.

### Stage 147.1 (3-{4-[3-(6-Amino-5-trifluoromethyl-pyridin-3-yl)-imidazo[1,2-b]pyridazin-6-yl]-phenoxymethyl}-oxetan-3-yl)-carbamic acid benzyl ester

A 100 mL flask containing a mixture of 1.5 g (90%, ˜3.07 mmol) {3-[4-(4,4,5,5-tetramethyl-[1,3,2]dioxaborolan-2-yl)-phenoxymethyl]-oxetan-3-yl}-carbamic acid benzyl ester (for preparation see stage 147.2), 1.12 g (95%, 3.39 mmol) 5-(6-chloro-imidazo[1,2-b]pyridazin-3-yl)-3-trifluoromethyl-pyridin-2-ylamine, 138 mg (98%, 0.166 mmol) PdCl2(dppf), 1.52 g (11.0 mmol) potassium carbonate, 20 mL ethanol and 40 mL of toluene is purged with nitrogen. The mixture is then heated under reflux for 16 hours. Only traces of starting material can be detected in the HPLC after this time. The reaction mixture is filtered through a pad of hyflo and the solvent is evaporated. Trituration of the residue with ethyl acetate and filtration followed by trituration with CH2Cl2 and filtration gives the title compound as a yellow solid. MS: (M+1)=591.0; HPLC: t_{R}=5.738 min. M.p. 202-203° C. Rf (CH2Cl2/EtOH 95:5)=0.3.

### Stage 147.2 {3-[4-(4,4,5,5-tetramethyl[1,3,2]dioxaborolan-2-yl)-phenoxymethyl]-oxetan-3-yl}-carbamic acid benzyl ester

A 250 mL flask containing a mixture of 3.1 g (95%, 7.51 mmol) [3-(4-bromo-phenoxymethyl)-oxetan-3-yl]-carbamic acid benzyl ester (for preparation see stage X147.3), 2.16 g (8.25 mmol) bis-(pinacolato)diboron, 271 mg (98%, 0.368) Pd(PPh3)2Cl2, 1.55 g (15.8 mmol) potassium acetate and 80 mL of toluene is purged with nitrogen. The mixture is then heated under reflux for 16 hours. No starting material can be detected in the HPLC and MS after this time. The reaction mixture is filtered through a pad of hyflo and the solvent is evaporated. The brown residue is purified by chromatography on 40 g of silica gel on a Combiflash Companion (Isco Inc.) using a gradient of hexanes/ethyl acetate from 9:1 to 8:2. Pure fractions are combined and the solvent is evaporated to leave the title compound as a colorless resin. MS: (M+1)=440.0; HPLC: t_{R}=4.703 min. Rf (hexanes/EtOAc 2:1)=0.5.

### Stage 147.3 [3-(4-Bromo-phenoxymethyl)-oxetan-3-yl]-carbamic acid benzyl ester

A mixture of 2 g (6.83 mmol) 3-(4-bromo-phenoxymethyl)-oxetan-3-carboxylic acid (for preparation see stage 147.4), 0.79 mL (7.5 mmol) benzyl alcohol, 1.8 mL (-90%, 7.49 mmol) DPPA, 1.06 mL (7.5 mmol) triethylamine and 75 mL of toluene in a 250 mL flask is heated to 100° C. under nitrogen for 5 hours. Only traces of starting material can be detected in the HPLC after this time. After cooling the reaction mixture is washed with NaHCO3 solution. The aqueous phase is extracted with toluene and the combined organic layers are washed with brine and dried with Na2SO4. Evaporation of the solvent gave an oil which is purified by chromatography on 80 g of silica gel on a Combiflash Companion (Isco Inc.) using a gradient of hexanes/ethyl acetate from 9:1 to 8:2. Pure fractions are combined and the solvent is evaporated to leave the title compound as a colorless solid. MS: (M+1)=392.0/393.9; HPLC: t_{R}=7.081 min. Rf (hexanes/EtOAc 2:1)=0.4; M.p. 101-103° C.

### Stage 147.4 3-(4-Bromo-phenoxymethyl)-oxetane-3-carboxylic acid

In a 500 mL three-necked flask equiped with a condenser, stir bar and nitrogen inlet are placed 6 g (95%, 20.9 mmol) of [3-(4-bromo-phenoxymethyl)-oxetan-3-yl]-methanol (for preparation see stage 147.5), 0.333 g (2.09 mmol) TEMPO, 240 mL acetonitrile and 120 mL of phosphate buffer (pH 7). A solution of 5.6 g (49.5 mmol) NaClO2 (sodium chlorite), 0.72 mL (1.04 mmol) of a 11% sodium hypochlorite solution and 30 mL of water is then added at RT and the mixture is heated at 77° C. for 20 hours. After cooling the acetonitrile is evaporated and the aqueous residue washed with ethyl acetate, acidified with 2N HCl and extracted with ethyl acetate. The organic extracts are washed with brine, dried with Na2SO4 and evaporated to give a colorless redisue. The first ethyl acetate washings are extracted with NaHCO3 solution and the aqueous phase is acidified with 2N HCl. This equeous phase is then extracted with CH2Cl2 and the organic phase washed with brine, dried with Na2SO4 and evaporated to give a colorless solid. According to HPLC analysis both residues are identical. They are re-dissolved, combined and the solvent is evaporated to give the title compound as a colorless solid. MS: (M+1)=285/287.2; HPLC: t_{R}=5.845 min.; M.p. 122-124° C.

### Stage 147.5 [3-(4-Bromo-phenoxymethyl)-oxetan-3-yl]-methanol

In a 250 mL three-necked flask equiped with a condenser, stir bar and nitrogen inlet are placed 7.5 g (62.2 mmol) (3-hydroxymethyl-oxetan-3-yl)-methanol (for preparation see stage 147.6), 11 g (62.3 mmol) 4-bromophenol, 16.7 g (62.2 mmol) triphenylphosphin and 120 mL of THE. Thereafter, 12.3 mL (62.2 mmol) diisopropyl azodicarboxylate is added dropwise within 1.5 hours (slightly exothermic). After stirring the solution at RT for 4 hours 1 mL of diisopropyl azodicarboxylate is added (5 minutes) and the solution stirred for one more hour. The THF is then evaporated and the resulting yellow oil is taken up in ethyl acetate and treated with hexanes. Aftre stirring for 10 minutes the precipitate is filtered off and discarded and the filtrate is concentrated to a yellow oil. This is purified by chromatography on 80 g of silica gel on a Combiflash Companion (Isco Inc.) using a gradient of CH2Cl2/EtOAc 9:1 to 1:1. Enriched fractions are combined, evaporated and re-chromatographed on 80 g of silica gel on a Combiflash Companion (Isco Inc.) using a gradient of hexanes/EtOAc from 85:15 to 75:25. Pure fractions are combined and the solvent is evaporated to leave the title compound as a colorless solid. MS: (M−1)=271/273; HPLC: t_{R}=5.77 min.

### Stage 147.6 (3-Hydroxymethyl-oxetan-3-yl)-methanol

A 1 L flask is charged with 100 g (0.727 mol) 2-bis-hydroxymethyl-propane-1,3-diol (pentaerythritol, ABCR), 115 mL (0.92 mol) diethyl carbonate and 13 mL EtOH. Powdered potassium hydroxide, 237 mg (3.63 mmol), is added and the mixture heated under reflux for 4 hours. After addition of another portion of 230 mg of potassium hydroxide the reflux condenser is replaced and EtOH is distilled out of the reaction mixture (bath temperature ˜135° C.). Within 4 hours 90 mL of ethanol are collected. The condenser is replaced again by a solid trap the apparatus is connected to a vacuum pump and the mixture is gradually heated to 240° C. at 0.5 to 1 mbar. The title compound is collected as a colorless solid. MS: (M+1)=119.0; Rf (EtOAc/EtOH 9:1)=0.3.

The example compound in the following table is prepared in analogy to the compound prepared in Example 146:

| Example | Product | data |
|---|---|---|
| 148 | 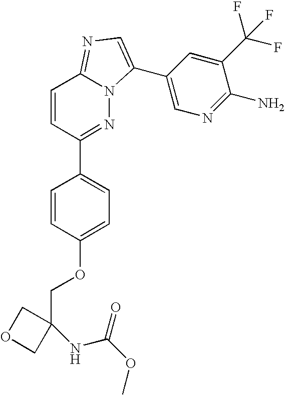 (3-{4-[3-(6-Amino-5- trifluoromethyl-pyridin-3-yl)- imidazo[1,2-b]pyridazin-6-yl]- phenoxymethyl}-oxetan-3- yl)-carbamic acid methyl ester | MS: (M + 1) = 515.1, HPLC: t_{R} = 4.973 min., Rf (CH2Cl2/EtOH 95:5) = 0.4, M.p. 220-222° C. |

### Example 167

### N-(3-{4-[3-(6-Amino-5-trifluoromethyl-pyridin-3-yl)-imidazo[1,2-b]pyridazin-6-yl]-phenoxymethyl}-oxetan-3-ylmethylyisobutyramide

The example compound in the following table is prepared in analogy to the compound prepared in Example 146 (for the preparation of the starting material, 5-{6-[4-(3-aminomethyl-oxetan-3-ylmethoxy)-phenyl]-imidazo[1,2-b]pyridazin-3-yl}-3-trifluoromethyl-pyridin-2-ylamine, see Example 169):

| Example | Product | data |
|---|---|---|
| 167 | 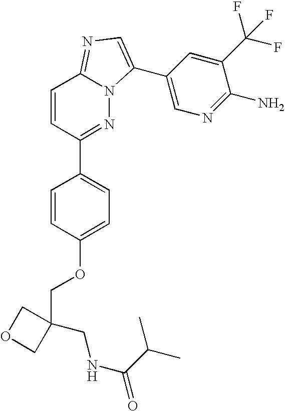 | MS: (M + 1) = 541.0, HPLC: t_{R} = 5.075 min., Rf (CH2Cl2/EtOH 95:5) = 0.3, M.p. 205-207° C. |

### Example 168

### {4-[6-(6-Amino-5-trifluoromethyl-pyridin-3-yl)-imidazo[1,2-b]pyridazin-3-yl]-phenyl}-methanol

In a 50 mL flask are placed 1.55 g (4.11 mmol) 5-(3-bromo-imidazo[1,2-b]pyridazin-6-yl)-3-trifluoromethyl-pyridin-2-ylamine,0.65 g (4.15 mmol) 4-(hydroxymethyl)phenylboronic acid, 5 mL of 2 M K2CO3 solution and 40 mL of DME and the flask is purged with nitrogen. The mixture is heated to 95° C. for 8 hours under stirring. After cooling to RT Na2SO4 is added and the mixture filtered through a pad of Hyflo. The DME is evaporated and gives a brown residue. The Hyflo is washed thoroughly with methanol and the methanol evaporated. Both residues are purified by chromatography on 40 g of silica gel on a Combiflash Companion (Isco Inc.) using a gradient of CH2Cl2/methanol from 98:2 to 9:1. Pure fractions are combined and the solvent is evaporated to leave the title compound as a yellow powder. MS: (M+1)=386; HPLC: t_{R}=4.61 min. Rf (CH2Cl2/EtOH 95:5)=0.3. M.p. 290-292° C.

### Example 169

### 5-{6-[4-(3-Aminomethyl-oxetan-3-ylmethoxy)-phenyl]-imidazo[1,2-b]pyridazin-3-yl}-3-trifluoromethyl-pyridin-2-ylamine

Crude (3-{4-[3-(6-amino-5-trifluoromethyl-pyridin-3-yl)-imidazo[1,2-b]pyridazin-6-yl]-phenoxymethyl}-oxetan-3-ylmethylycarbamic acid benzyl ester (for preparation see stage 169.1), 1.0 g (˜1.3 mmol) is deprotected by hydrogenation at 5 bar and room at temperature with 10% Palladium on carbon (0.4 g) in a mixture of THF (5 mL), methanol (30 mL), and DMF (30 mL). After 2 days the hydrogenation is stopped and the catalyst filtered off through a pad of hyflo. The solvent is evaporated, the redidue is taken up in EtOAc and extracted with 10% citric acid. The organic phase is re-extracted with citric acid and the aqueous phase is washed with EtOAc. Thereafter the pH of the combined aqueous extracts is adjusted to ˜10 by the addition of sodium hydroxide solution. Extraction with CH2Cl2 (3×) followed by drying over Na₂SO₄, and evaporation of the solvent gives the crude product which is further triturated with EtOAc and filtered to give the title compound as a yellow solid. MS-ES.: (M+1) 471.1, HPLC: t_{R}=4.27 min.

### Stage 169.1: (3-{4-[3-(6-Amino-5-trifluoromethyl-pyridin-3-yl)-imidazo[1,2-b]pyridazin-6yl]-phenoxymethyl}-oxetan-3-ylmethyl)-carbamic acid benzyl ester

A 100 mL flask containing a mixture of 1.5 g (95%, 3.14 mmol) {3-[4-(4,4,5,5-tetramethyl-[1,3,2]dioxaborolan-2-yl)-phenoxymethyl]-oxetan-3-ylmethyl}-carbamic acid benzyl ester (for preparation see stage 169.2), 1.14 g (95%, 3.45 mmol) 5-(6-chloro-imidazo[1,2-b]pyridazin-3-yl)-3-trifluoromethyl-pyridin-2-ylamine, 141 mg (98%, 0.169 mmol) PdCl2(dppf), 1.56 g (11.3 mmol) potassium carbonate, 20 mL ethanol and 40 mL of toluene is purged with nitrogen. The mixture is then heated under reflux for 6 hours. No starting material can be detected in the HPLC after this time. The reaction mixture is filtered through a pad of hyflo and the solvent is evaporated. Trituration of the residue with CH2Cl2and filtration followed by trituration with a mixture of methanol/THF/EtOAc and filtration gives crude title compound as a yellow solid. MS: (M+1)=605.0; HPLC: t_{R}=5.76 min.

### Stage 169.2: {3-[4-(4,4,5,5-Tetramethyl[1,3,2]dioxaborolan-2-yl)-phenoxymethyl]oxetan-3-ylmethyl}-carbamic acid benzyl ester

A 100 mL flask containing a mixture of 2.2 g (90%, 4.87 mmol) [3-(4-bromo-henoxymethyl)-oxetan-3-ylmethyl]-carbamic acid benzyl ester (for preparation see stage 169.3), 1.4 g (5.35 mmol) bis-(pinacolato)diboron, 176 mg (98%, 0.248) Pd(PPh3)2Cl2, 1.01 g (10.3 mmol) potassium acetate and 40 mL of DMF is purged with nitrogen. The mixture is then heated to 95° C. for 10 hours. Since there is still some starting material present a small amount of Pd(PPh3)2Cl2 is added and the heating continued for 6 hours. Thereafter the reaction mixture is cooled and filtered through a pad of hyflo and the solvent is evaporated. The brown residue is purified by chromatography on 40 g of silica gel on a Combiflash Companion (Isco Inc.) using a gradient of hexanes/ethyl acetate from 9:1 to 7:3. Pure fractions are combined and the solvent is evaporated to leave the title compound as a colorless solid. MS: (M+1)=454.1; HPLC: t_{R}=4.396 min. Rf (CH2C12/EtOAc 85:15)=0.4.

### Stage 169.3: [3-(4-Bromo-henoxymethyl)-oxetan-3-ylmethyl]-carbamic acid benzyl ester

A 250 mL flask containing a mixture of 1.4 g (4.99 mmol) C-[3-(4-bromo-phenoxymethyl)-oxetan-3-yl]-methylamine (stage 169.4), 40 mL of a saturated solution of Na2CO3 and 80 mL of CH2Cl2 is treated under stirring and at RT with 739 μL (4.99 mmol) Z-chloride. The mixture is stirred 1 hour at RT. At that point no starting material can be detected by HPLC. The reacton mixture is diluted with CH2Cl2 and brine and after separation of the two layers the aqueous phase is extracted with CH2Cl2 (2×). The combined organic extracts are dried with Na2SO4 and the solvent is evaporated to give the title compound as a colorless oil. MS: (M+1)=406.0/407.9; HPLC: t_{R}=6.819 min. Rf (hexanes/EtOAc 2:1)=0.3.

### Stage 169.4: C-[3-(4-Bromo-phenoxymethyl)-oxetan-3-yl]-methylamine

In a 250 mL flask equipped with a reflux condenser are placed 6.3 g (17.4 mmol) of methanesulfonic acid 3-(4-bromo-phenoxymethyl)-oxetan-3-ylmethyl ester (preparation see stage 169.5) and 100 mL of a 7 M methanolic ammonia solution. The solution is heated under reflux for 6 days during which time additional ammonia solution is added periodically (100 mL in total). After cooling the solvent is evaporated and the residue partitioned between EtOAc and ammonium chloride solution. Solids are filtered off, the layers separated and the organic phase is extracted with water and the aqueous phase is washed with EtOAc. To the combined aqueous phases a saturated Na2CO3 solution is added and the resulting basic solution is extracted 2× with CH2Cl2. The CH2Cl2 phase is washed with brine, dried with Na2SO4 and evaporated. The title compound is obtained as a colorless solid. MS: (M+1)=272.0/274.0; HPLC: t_{R}=4.665 min. Rf (CH2Cl2/EtOH 95:5)=0.1.

### Stage 169.5: Methanesulfonic acid 3-(4-bromo-phenoxymethyl)-oxetan-3-ylmethyl ester

In a 250 mL 3-necked flask equipped with a septum, thermometer and nitrogen in/outlet are placed 5.0 g (17.9 mmol) [3-(4-bromo-phenoxymethyl)-oxetan-3-yl]-methanol (preparation see stage 147.5) and 5.1 mL (36.3 mmol) triethylamine in 100 mL of CH2Cl2. To the ice-cooled solution are added 1.69 mL (21.7 mmol) methanesulfonyl chloride with a siringe over 30 minutes. The temperature is maintained below 10° C. during the addition. Thereafter the reaction mixture is allowed to reach RT within 30 minutes under stirring. The solvent is evaporated and the residue is partitioned between EtOAc and ammonium chloride solution. The aqueous phase is extracted with EtOAc and the combined organic layers are washed with NaHCO3 solution and brine. After drying with Na2SO4 the solvent is evaporated to give the title compound as yellow crystals. MS: (M+1)=349/351.0; HPLC: t_{R}=6.401 min. Rf (hexanes/EtOAc 2:1)=0.2. M.p. 89-91° C.

### Example 172

### Cyclopropanecarboxylic acid (3-{4-[3-(6-amino-5-trifluoromethyl-pyridin-3-yl)-imidazo[1,2-b]pyridazin-6-yl]-phenoxymethyl}-oxetan-3-ylmethyl)-amide

In a 5 ml vial with magnetic stir bar 75 mg (0.151 mmol) of 5-{6-[4-(3-aminomethyl-oxetan-3-ylmethoxy)-phenyl]-imidazo[1,2-b]pyridazin-3-yl}-3-trifluoromethyl-pyridin-2-ylamine (for preparation see example 169) and 83.7 mg (0.606 mmol) of potassium carbonate are mixed with 2 mL of acetonitrile under nitrogen. Thereafter a solution of 14 μL (0.151 mmol) of cyclopropanecarbonyl chloride in 0.2 mL of acetonitrile is added slowly at RT. After complete addition and 4 hours of additional stirring, no more starting material can be detected in the HPLC or MS. The reaction mixture is filtered and the solvent is evaporated. The residue is partitioned between CH2Cl2 and ammonium chloride solution and the aqueous phase extracted with CH2Cl2. The combined organic extracts are combined and dried with Na2SO4. Evaporation of the solvent gives the crude product which is triturated with CH2Cl2/hexanes and filtered to yield the title compound as a yellow solid. MS-ES: (M+1)=539.1, HPLC: t_{R}=4.96 min. Rf (CH2Cl2/EtOH 95:5)=0.25. M.p. 223-225° C.

### Example 173

### (3-{4-[3-(6-Amino-5-trifluoromethyl-pyridin-3-yl)-imidazo[1,2-b]Pyridazin-6-yl]-phenoxymethyl}-oxetan-3-ylmethyl)-carbamic acid methyl ester

The title compound is prepared in analogy to the compound prepared in Example 146:

| Example | Product | data |
|---|---|---|
| 173 | 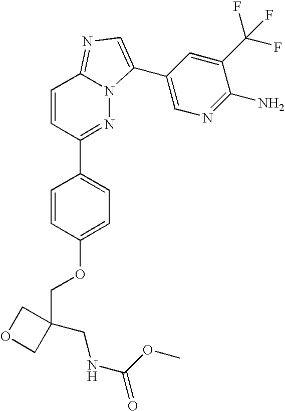 | MS: (M + 1) = 529.0, HPLC: t_{R} = 5.044 min., Rf (CH2Cl2/EtOH 95:5) = 0.3, M.p. 179-181° C. |

### Example 179

### 4-[5-(2-Methoxy-phenyl)-pyrazolo[1,5-a]pyrimidin-3-yl]-benzamide

Flash chromatography is performed by using a CombiFlash® Companion system®, with RediSep® silica gel column. HPLC analysis are performed on a Thermo Finnigan SpectraSYSTEM instrument, UV6000 detector, detection at 216 nm, 100×4.6 mm Chromolith Performance column, RP-18e, linear solvent gradient from 2% B to 100% B in 8 min, then 2 min 100% B, 2.0 mL/min flow rate, solvents: A=0.1% aqueous formic acid and B=0.1% formic acid in acetonitrile; retention time t_{R} given in minutes. Electrospray mass spectra are obtained with a Fisons Instruments VG Platform II. Commercially available solvents and chemicals are used for syntheses.

3-Bromo-5-(2-methoxyphenyl)pyrazolo[1,5-a]pyrimidine (Stage 179.1, 24 mg, 0.08 mmol), 4-carbamoylbenzeneboronic acid (13 mg, 0.08 mmol), and potassium carbonate (0.1 mL, 2 M, 0.21 mmol) are mixed in DME (0.5 mL), flushed with argon and pre-heated to 80° C. Dichloro-bis(triphenylphosphine)palladium (II) (1.7 mg, 0.002 mmol) is then added and the mixture is stirred for 18 h at 80° C. After cooling at RT the reaction mixture is taken in ethyl acetate, washed with brine. The combined organic phases are dried over sodium sulfate, concentrated under reduced pressure and the residue is purified by flash chromatography (Hexane/EtOAc) to afford 7.0 mg of 4-[5-(2-methoxy-phenyl)-pyrazolo[1,5-a]pyrimidin-3-yl]-benzamide as a solid. MH⁺=345.2, HPLC t_{R}: 4.89 min.

### Stage 179.1: 3-Bromo-5-(2-methoxyphenyl)pyrazolo[1,5-a]pyrimidine

3-Bromo-5-chloro-pyrazolo[1,5-a]pyrimidine (Stage 179.2, 56 mg, 0.241 mmol), 2-methoxybenzeneboronic acid (37 mg, 0.241 mmol), and potassium carbonate (0.325 mL, 2 M, 0.65 mmol) are mixed in DME (1.0 mL), and flushed with argon. Dichloro-bis(triphenylphosphine)palladium (II) (5.1 mg, 0.007 mmol) is added and the mixture is stirred for 30 minutes at 80° C. After cooling at RT the reaction mixture is taken in ethyl acetate, washed with brine. The combined organic phases are dried over sodium sulfate, concentrated under reduced pressure and the residue is purified by flash chromatography (Hexane/EtOAc) to afford 57.0 mg of 3-bromo-5-(2-methoxyphenyl)pyrazolo[1,5-a]pyrimidine as a solid. MH⁺=306, HPLC t_{R}: 6.30 min.

### Stage 179.2: 3-Bromo-5-(2-methoxyphenyl)pyrazolo[1,5-a]pyrimidine

5-Chloro-pyrazolo[1,5-a]pyrimidine (432 mg, 2.81 mmol) and TFA (64 4, 0.84 mmol) are dissolved in acetonitrile. N-bromsuccinimide (551 mg, 3.1 mmol) is added and the mixture is stirred for 2 h at RT. The reaction mixture is taken in ethyl acetate, washed with a solution of 10% sodium hydrogen carbonate and brine. The combined organic phases are dried over sodium sulfate, concentrated under reduced pressure and the residue is purified by flash chromatography (Hexane/EtOAc) to afford 560 mg of 3-Bromo-5-(2-methoxyphenyl)pyrazolo[1,5-a]pyrimidine as a yellowish solid. MP 125-128° C., HPLC t_{R}: 4.92 min.

In analogy to example 179 the following compound is prepared:

### Example 179a

Cyclopropanecarboxylic acid (3-{5-[3-(6-amino-5-trifluoromethyl-pyridin-3-yl)-pyrazolo[1,5-a]pyrimidin-5-yl]-pyridin-2-yloxy}-propyl)-amide

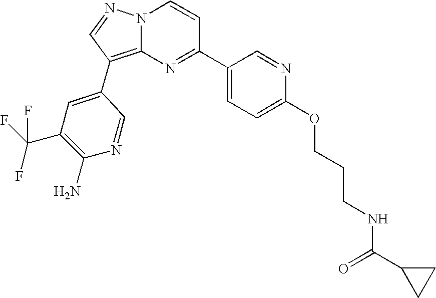

In analogy to methods A-N the following compounds are prepared:

### Examples 180a-d

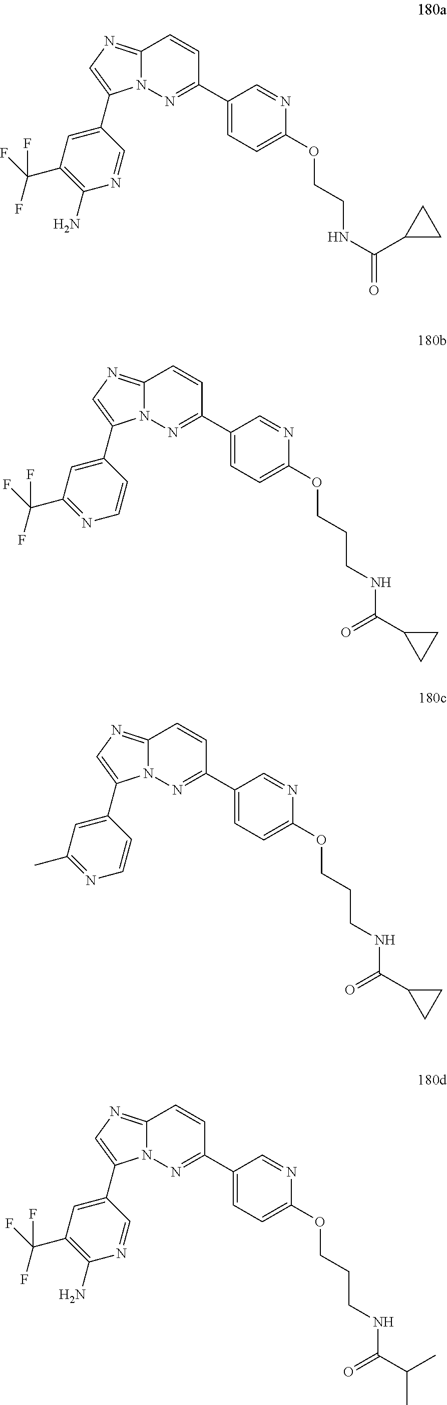

### Example 181

### (1-(4-[6-(6-Amino-5-trifluoromethyl-pyridin-3-yl)-imidazo[1,2-b]pyridazin-3-yl]-benzylypiperidin-4-yl)-pyrrolidin-1-yl-methanone

In a 6 mL vial a mixture of 80 mg (˜60%, 0.107 mmol) 1-{4-[6-(6-amino-5-trifluoromethyl-pyridin-3-yl)-imidazo[1,2-b]pyridazin-3-yl]-benzyl}-pyridinium mesylate (preparation see stage 181.1), 25 mg (0.133 mmol) piperidin-4-yl-pyrrolidin-1-yl-methanone, 60 mg (0.425 mmol) K2CO3, a catalytic amount of potassium iodide in 4 mL of DMF is irradiated under stirring in a microwave oven at 150° C. for 30 minutes. The reaction mixture is evaporated and the residue stirred in 50 mL of EtOAc and filtered. The filtrate is evaporated and the residue is purified by chromatography on 4 g of silica gel on a Combiflash Companion (Isco Inc.) using a gradient of CH2Cl2/methanol/conc NH3 from 98:1.8:0.2 to 95:4.5:0.5. Pure fractions are combined and the solvent is evaporated. The residue is taken up in a small amount of CH2Cl2 and the title compound crystallized by the addition of hexanes and cooling in an ice bath. Yellow powder. MS: (M+1)=550; HPLC: t_{R}=4.52 min. Rf (CH2Cl2/EtOH/conc. NH3 95:4.5:0.5)=0.2. M.p. 209-211° C.

### Stage 181.1: 1-{4-[6-(6-Amino-5-trifluoromethyl-pyridin-3-yl)-imidazo[1,2-b]pyridazin-3-yl]-benzyl}-pyridinium mesylate

A solution of 250 mg (0.629 mmol) {4-[6-(6-amino-5-trifluoromethyl-pyridin-3-yl)-imidazo[1,2-b]pyridazin-3-yl]-phenyl}-methanol (preparation see example 8) and 531 μL (1.54 mmol) triethylamine in 30 mL of acetonitrile is treated at RT with 60 μL (0.75 mmol) methanesulfonyl chloride. After one hour the same amount of methanesulfonyl chloride is added and stirring is continued. Thereafter 10 mL of pyridine and 60 μL (0.75 mmol) methanesulfonyl chloride is added and the mixture heated to reflux for 4 hours. After cooling the solvent is evaporated and the residue stirred in CH2Cl2. The suspension is filtered and the filtrate treated with hexanes. The resulting precipitate is filtered off again. Both brown solids are combined to the crude title compound. MS: (M+1)=447.0; HPLC: tR=4.330 min.

The example compound in the following table is prepared in analogy to the compound prepared in Example 181:

| Example | Product | data |
|---|---|---|
| 182 | 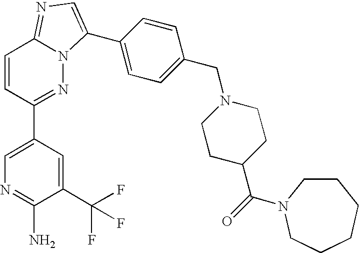 (1-{4-[6-(6-Amino-5- trifluoromethyl-pyridin-3-yl)- imidazo[1,2-b]pyridazin-3-yl]- benzyl}-piperidin-4-yl)- azepan-1-yl-methanone | MS: (M + 1) = 578.1, HPLC: t^{R} = 4.861 min., Rf (CH2Cl2/EtOH/conc. NH3 95:4.5:0.5) = 0.2, M.p. 249-252° C. |

### Example 183

### (1-{4-[6-(6-Amino-5-trifluoromethyl-pyridin-3-yl)-imidazo[1,2-b]pyridazin-3-yl]-benzyl}-piperidin-4-yl)-piperidin-1-yl-methanone

In a 6 mL vial a mixture of 100 mg (0.273 mmol) 4-[4-(piperidine-1-carbonyl)-piperidin-1-ylmethyl]-phenylboronic acid (preparation see stage X11.1), 142 mg (0.337 mmol) 5-(3-bromo-imidazo[1,2-b]pyridazin-6-yl)-3-trifluoromethyl-pyridin-2-ylamine, 410 μL (0.82 mmol) 2 M K2CO3, 11.7 mg (0.0163 mmol) Pd(PPh3)2Cl2 in 4 mL of DME is irradiated under stirring in a microwave oven at 130° C. for 30 minutes. The reaction mixture is evaporated and the residue stirred in 50 mL of EtOAc and filtered. The filtrate is evaporated and the residue is purified by chromatography on 4 g of silica gel on a Combiflash Companion (Isco Inc.) using a gradient of CH2Cl2/methanol/conc NH3 from 98:1.8:0.2 to 95:4.5:0.5. Pure fractions are combined and the solvent is evaporated. The residue is triturated and filtered first with a small amount of water and then with a small amount of CH2Cl2. The title compound is obtained as a yellow powder. MS: (M+1)=564.1; HPLC: t_{R}=4.720 min. Rf (CH2Cl2/EtOH/conc. NH3 95:4.5:0.5)=0.2. M.p. 263-266° C.

### Stage 183.1: 4-[4-(Piperidine-1-carbonyl)-piperidin-1-ylmethyl]-phenylboronic acid

In a 6 mL vial 150 mg (0.677 mmol) 4-bromomethyl)phenylboronic acid, 144 mg (0.712 mmol) piperidin-1-yl-piperidin-4-yl-methanone and 382 mg (2.71 mmol) K2CO3 are mixed with 4 mL of DMF. The mixture is stirred at RT for 1.5 hours. The reaction mixture is evaporated and the residue stirred in 50 mL of CH2Cl2 and filtered. The filtrate is evaporated to a brown foam. This is taken up in a small amount of CH2Cl2 and treated with hexanes until the solution turns turbid and the product precipitates. The solvent is decanted anf the residue dried under vacuum. The title compound is obtained as a brown amorphous material. MS: (M+1)=331.1; HPLC: t_{R}=4.070 min.

### Example 184

### Cyclopropanecarboxylic acid (3-{4-[3-(6-amino-5-trifluoromethyl-pyridin-3-yl)-imidazo[1,2-b]pyridazin-6-yl]-phenoxy}-oxetan-3-ylmethyl)-amide

The title compound is prepared following an analogous procedure as described in stage 169.1. MS: (M+1)=524.9; HPLC: t_{R}=5.05 min.

### Stage 184.1: Cyclopropanecarboxylic acid {3-[4-(4,4,5,5-tetramethyl-[1,3,2]dioxaborolan-2-yl)-phenoxy]-oxetan-3-ylmethyl}-amide

The title compound is prepared following an analogous procedure as described in stage 169.2. MS: (M+1)=374.0; HPLC: t_{R}=6.5 min. Rf (hexanes/EtOAc 2:1)=0.2.

### Stage 184.2: Cyclopropanecarboxylic acid [3-(4-bromo-phenoxy)-oxetan-3-ylmethyl]-amide

The title compound is prepared following an analogous procedure as described in stage 169.3. MS: (M+1)=326/328.0; HPLC: t_{R}=5.94 min.

### Stage 184.3: C-[3-(4-Bromo-phenoxy)-oxetan-3-yl]-methylamine

The title compound is prepared following an analogous procedure as described in stage 169.4. MS: (M+1)=258/260; HPLC: t_{R}=4.5 min.

### Stage 184.4: Methanesulfonic acid 3-(4-bromo-phenoxy)-oxetan-3-ylmethyl ester

In a 100 mL flask a solution of 3.7 g (˜60%, 8 mmol) 2-(4-bromo-phenoxy)-2-hydroxymethyl-propane-1,3-diol in 50 mL of dry THF is treated under nitrogen with 400 mg (10 mmol) of sodium hydride (˜60% in mineral oil). The mixture is stirred for 2 h at RT and then treated with a solution of 0.6 mL (7.7 mmol) methanesulphonyl chloride in 4 portions within 1 h. After stirring for 1 h, 460 mg (11.5 mmol) of sodium hydride (˜60% in mineral oil) are added and the mixture stirred again for 1 h. Thereafter 1.2 mL (15.4 mmol) of methanesulphonyl chloride are added in 5 portions and the mixture is stirred for 2.5 days at RT. The mixture is filtered and the solids washed with CH2Cl2. The filtrate is transferred to a separatory funnel and washed with NaHCO3, dried with Na2SO4 and evaporated. The crude material is purified by chromatography on 40 g of silica gel on a Combiflash Companion (Isco Inc.) using a gradient of 100% CH2Cl2 to CH2Cl2/ethanol 9:1. Pure fractions are combined and the solvent is evaporated to leave the title compound as a colorless powder. MS: (M−1)=335/337; HPLC: t_{R}=6.24 min.

### Stage 184.5: 2-(4-Bromo-phenoxy)-2-hydroxymethyl-propane-1,3-diol

In a 250 mL flask are placed 8.82 g (16.1 mmol) 2-acetoxymethyl-2-(4-bromo-phenoxy)-malonic acid diethyl ester in 80 mL of dry THF and cooled in an ice bath. Lithium borohydride, 1.6 g (69.8 mmol) is then addad in 7 portions within 2 h. The mixture is then stirred 6 h under ice cooling and then 10 h at RT. More Lithium borohydride, 0.6 g is added at RT and the reaction mixture stirred for 3 additional hours. The resulting mixture is filtered and washed with CH2Cl2 and the filtrate is evaporated. The residue (turbid oil) was treated under cooling with EtOAc (exothermic) and stirred. The precipitate is filtered and washed with hexanes. The solid is re-suspended in CH2Cl2, then hexanes is added and the suspension stirred for a few minutes and filtered. The solid is washed with hexanes an dried to give the crude title compound which is used without further purification. MS: (M−1)=275/277; HPLC: t_{R}=4.6 min.

### Stage 184.6: 2-Acetoxymethyl-2-(4-bromo-phenoxy)-malonic acid diethyl ester

A solution of 6 g (16.3 mmol) 2-(4-bromo-phenoxy)-2-hydroxymethyl-malonic acid diethyl ester in 70 mL of pyridine is treated under argon and at RT with 1.537 mL (16.27 mmol) acetic anhydride and stirred at RT for 2.5 days. The pyridine is evaporated and the residue is partitioned between EtOAc and NaHCO3 solution. The aqueous phase is extracted with EtOAc and the combined organic phases are washed with brine, dried with Na2SO4 and evaporated. The title compound is obtained as a brown oil. MS: (M+1)=403/405.0; HPLC: t_{R}=7.2 min. Rf (hexanes/EtOAc 2:1)=0.6.

### Stage 184.7: 2-(4-Bromo-phenoxy)-2-hydroxymethyl-malonic acid diethyl ester

In a 100 mL flask are placed 18 g (48.9 mmol) 2-(4-bromo-phenoxy)-malonic acid diethyl ester, 360 mg (4.29 mmol) NaHCO3 in 15 mL of ethanol and 10 mL of water. Aqueous formaldehyde (37%, 3.8 mL, 51 mmol) is added dropwise under stirring at such a rate that the temperature stays below 30° C. After complete addition the mixture is stirred 4 h. The solvents are evaporated and the residue is triturated with EtOAc and filtered. The filtrate is evaporated and the residue is purified by chromatography on 80 g of silica gel on a Combiflash Companion (Isco Inc.) using a gradient of hexanes/EtOAc 9:1 to 7:3. Pure fractions are combined and the solvent is evaporated to leave the title compound as a colorless oil. MS: (M+1)=361/363; HPLC: t_{R}=6.53 min.

### Stage 184.8: 2-(4-Bromo-phenoxy)-malonic acid diethyl ester

To a solution of 20 mL (115 mmol) diethyl chloromalonate in 200 mL of acetonitrile is added K2CO3 (39.8 g, 288 mmol) and 4-bromophenol (21.4 g, 121 mmol). The mixture is stirred at RT for 16 h. The mixture is filtered and the solvent evaporated. The residue is purified by chromatography on 80 g of silica gel on a Combiflash Companion (Isco Inc.) using a gradient of hexanes/CH2Cl2 7:3 to 1:1. Pure fractions are combined and the solvent is evaporated to leave the title compound as a colorless oil. HPLC: t_{R}=7.1 min. Rf (hexanes/CH2Cl2 7:3)=0.14.

### Example 185

### Soft Capsules

5000 soft gelatin capsules, each comprising as active ingredient 0.05 g of one of the compounds of formula I mentioned in the preceding Examples, are prepared as follows:

Composition

| | | |
|---|---|---|
| Active ingredient | 250 | g |
| Lauroglycol | 2 | litres |

Preparation process: The pulverized active ingredient is suspended in Lauroglykol® (propylene glycol laurate, Gattefossä S. A., Saint Priest, France) and ground in a wet pulverizer to produce a particle size of about 1 to 3 μm. 0.419 g portions of the mixture are then introduced into soft gelatin capsules using a capsule-filling machine.

### Example 186

### Tablets Comprising Compounds of the Formula I

Tablets, comprising, as active ingredient, 100 mg of any one of the compounds of formula I of Examples 1 to 32 are prepared with the following composition, following standard procedures:

Composition

| | | |
|---|---|---|
| Active Ingredient | 100 | mg |
| crystalline lactose | 240 | mg |
| Avicel | 80 | mg |
| PVPPXL | 20 | mg |
| Aerosil | 2 | mg |
| magnesium stearate | 5 | mg |
| | 447 | mg |

Manufacture: The active ingredient is mixed with the carrier materials and compressed by means of a tabletting machine (Korsch EKO, Stempeldurchmesser 10 mm).

Avicel® is microcrystalline cellulose (FMC, Philadelphia, USA). PVPPXL is polyvinylpoly-pyrrolidone, cross-linked (BASF, Germany). Aerosil® is silicium dioxide (Degussa, Germany).

### Example 187

### Biological Assay

Testing with the following kinases in the test system mentioned above, with the kinases given in the following tables, the following IC 50 data can be obtained:

| Compound from | IC₅₀ (μm) |
|---|---|
| Example | PI3K-alfa |
| 1 | 0.003 |
| 5 | 0.001638 |
| 6 | 0.003 |
| 7 | 0.014 |
| 8 | 0.006 |
| 9 | 0.854 |
| 10 | 0.118 |
| 11 | 0.372 |
| 14 | 0.039 |
| 16 | 0.135 |
| 17 | 0.053 |
| 18 | 0.0095 |
| 19 | 0.12 |
| 20 | 0.122 |
| 21 | 0.292 |
| 27 | 0.0014 |
| 28 | 0.012 |
| 29 | 0.007 |
| 30 | 0.0023 |
| 31 | 0.0045 |
| 32 | 0.0049 |

| | Enzymatic Data | | | |
|---|---|---|---|---|
| example | PI3K alfa | PI3K beta | PI3K delta | PI3K gamma |
| number | IC50 [umol] | IC50 [umol] | IC50 [umol] | IC50 [umol] |
| 33 | 0.063 | 0.240 | 0.103 | 1.223 |
| 34 | 0.103 | 0.718 | 0.143 | 1.306 |
| 35 | 0.122 | 0.186 | 0.171 | >9.100 |
| 36 | 0.255 | 2.162 | 0.328 | 4.828 |
| 37 | 0.110 | 0.054 | 0.072 | 1.124 |
| 38 | 0.092 | 0.226 | 0.121 | 1.693 |
| 39 | 0.054 | 0.019 | 0.040 | 1.293 |
| 40 | 6.904 | >9.100 | >9.100 | >9.100 |
| 41 | 0.264 | 0.212 | 0.207 | 1.605 |
| 42 | 0.635 | >9.100 | 0.624 | >9.100 |
| 43 | 0.472 | 0.528 | 0.403 | 8.441 |
| 44 | 0.297 | 2.221 | 0.336 | 3.504 |
| 45 | 0.154 | 0.378 | 0.282 | 7.072 |
| 46 | 1.620 | 6.282 | 2.982 | >9.100 |
| 47 | 2.716 | >9.100 | 3.845 | >9.100 |
| 48 | 1.122 | 6.793 | 2.439 | >9.100 |
| 49 | 1.615 | >9.100 | 4.809 | 6.039 |
| 50 | 0.165 | 2.186 | 1.011 | 2.745 |
| 51 | 0.092 | 1.394 | 0.886 | 6.047 |
| 52 | 0.022 | 0.364 | 0.295 | 2.217 |
| 53 | 0.275 | 1.243 | 0.401 | 1.081 |
| 54 | 0.147 | 0.274 | 0.231 | 0.814 |
| 55 | 6.122 | >9.100 | >9.100 | |
| 56 | 0.203 | 0.183 | 0.167 | 0.056 |
| 57 | 0.023 | 3.371 | 0.384 | 1.035 |
| 58 | 0.087 | >9.100 | 2.145 | >9.100 |
| 59 | 0.033 | 0.024 | 0.060 | 0.401 |
| 60 | 0.036 | 0.267 | 0.223 | 0.159 |
| 61 | 0.205 | 1.001 | 0.428 | 0.884 |
| 62 | 0.028 | 0.156 | 0.138 | 1.355 |
| 63 | 0.153 | 0.176 | 0.180 | 0.039 |
| 64 | 0.562 | >9.100 | 6.806 | 0.316 |
| 65 | 0.026 | 0.505 | 0.345 | 0.483 |
| 66 | 0.008 | 0.003 | 0.017 | 0.479 |
| 67 | 0.034 | 0.021 | 0.085 | 2.627 |
| 68 | 0.009 | 0.051 | 0.026 | 1.295 |
| 69 | 0.050 | 1.433 | 0.156 | 2.434 |
| 70 | 0.058 | 0.049 | 0.063 | 0.491 |
| 71 | 0.170 | 2.650 | 0.739 | 8.513 |
| 72 | 0.028 | 0.233 | 0.056 | 2.017 |
| 73 | 0.725 | >9.100 | 3.950 | >9.100 |
| 74 | 0.159 | 0.147 | 0.162 | 1.434 |
| 75 | 0.075 | 0.651 | 0.135 | 3.093 |
| 76 | 0.158 | 0.670 | 0.151 | 3.219 |
| 77 | 0.190 | 1.119 | 0.623 | 2.564 |
| 78 | 0.130 | 2.058 | 1.058 | 4.035 |
| 79 | 0.015 | 0.004 | 0.009 | 0.737 |
| 80 | 0.015 | 0.210 | 0.057 | 1.160 |
| 81 | 0.021 | 0.082 | 0.013 | 0.638 |
| 82 | 0.018 | 0.169 | 0.007 | 0.526 |
| 83 | 0.027 | 0.166 | 0.023 | 0.558 |
| 84 | 0.087 | 5.555 | 0.308 | 3.238 |
| 85 | 0.019 | 0.340 | 0.015 | 0.129 |
| 86 | 0.094 | 0.701 | 0.097 | 0.111 |
| 87 | 0.029 | 0.010 | 0.018 | 1.229 |
| 88 | 0.128 | 0.993 | 0.148 | 2.433 |
| 89 | 0.074 | 2.763 | 0.088 | 0.543 |
| 90 | 1.136 | >9.100 | 1.111 | 4.693 |
| 91 | 0.155 | 7.620 | 0.530 | 6.537 |
| 92 | 0.573 | 4.109 | 1.235 | >9.100 |
| 93 | 0.157 | 6.934 | 1.274 | 5.835 |
| 94 | 0.062 | 0.441 | 0.146 | 2.148 |
| 95 | 0.006 | 0.059 | 0.020 | 0.928 |
| 96 | 0.501 | 4.710 | 1.088 | 7.664 |
| 97 | 0.075 | 1.551 | 0.056 | 1.024 |
| 98 | 0.028 | 2.943 | 0.120 | 2.206 |
| 99 | 0.009 | 0.082 | 0.015 | 0.584 |
| 100 | 0.024 | 0.094 | 0.026 | 0.419 |
| 101 | 0.008 | 0.168 | 0.030 | 1.170 |
| 102 | 0.043 | 1.273 | 0.017 | 1.061 |
| 103 | 0.004 | 0.134 | 0.011 | 0.573 |
| 104 | 0.027 | 0.143 | 0.021 | 1.420 |
| 105 | 0.024 | 0.463 | 0.018 | 0.362 |
| 106 | 0.171 | >9.100 | 0.291 | 0.788 |
| 107 | 0.026 | 0.497 | 0.030 | 0.827 |
| 108 | 0.091 | 0.575 | 0.142 | 0.565 |
| 109 | 0.006 | 0.062 | 0.010 | 0.405 |
| 110 | 0.040 | 1.184 | 0.077 | 1.370 |
| 111 | 0.954 | 7.547 | 0.427 | >9.100 |
| 112 | 0.012 | 0.623 | 0.014 | 0.956 |
| 113 | 0.011 | 0.068 | 0.013 | 0.555 |
| 114 | 0.140 | 4.069 | 0.829 | 1.442 |
| 115 | 0.009 | 0.078 | 0.022 | 0.397 |
| 116 | 0.003 | 0.042 | 0.004 | 0.410 |
| 117 | 0.009 | 0.217 | 0.010 | 0.446 |
| 118 | 0.008 | 0.178 | 0.006 | 0.405 |
| 119 | 0.009 | 0.137 | 0.013 | 0.406 |
| 120 | 0.016 | 0.389 | 0.025 | 0.757 |
| 121 | 0.024 | 0.019 | 0.026 | 3.306 |
| 122 | 0.043 | 0.986 | 0.199 | 1.402 |
| 123 | 0.016 | 0.979 | 0.093 | 1.269 |
| 124 | 0.018 | 0.157 | 0.029 | 0.529 |
| 125 | 0.026 | 0.182 | 0.055 | 0.490 |
| 126 | 0.066 | 0.139 | 0.022 | 1.028 |
| 127 | 0.019 | 0.416 | 0.059 | 1.052 |
| 128 | 0.012 | 0.518 | 0.038 | 0.938 |
| 129 | 0.223 | 4.145 | 0.676 | 2.025 |
| 130 | 0.347 | >9.100 | 0.576 | 1.298 |
| 131 | 0.026 | 0.112 | 0.015 | 0.703 |
| 132 | 0.026 | 1.262 | 0.052 | 2.023 |
| 133 | 0.038 | 0.302 | 0.033 | 1.281 |
| 134 | 0.071 | 0.443 | 0.079 | >9.100 |
| 135 | 0.046 | 0.587 | 0.059 | 2.006 |
| 136 | 0.036 | 1.197 | 0.052 | 1.528 |
| 137 | 0.138 | >9.100 | 0.280 | >9.100 |
| 140 | 0.037 | 0.135 | 0.027 | 0.712 |
| 141 | 0.151 | 0.134 | 0.201 | 7.251 |
| 142 | 0.045 | 3.165 | 0.319 | >9.100 |
| 143 | 0.044 | 0.072 | 0.028 | 1.179 |
| 144 | 0.175 | 0.533 | 0.266 | 5.903 |
| 145 | 0.097 | 0.508 | 0.151 | 6.921 |
| 146 | 0.054 | 2.540 | 0.280 | 1.727 |
| 147 | 0.061 | 0.496 | 0.227 | 1.312 |
| 148 | 0.096 | 3.289 | 0.369 | 0.810 |
| 149 | 0.019 | 0.040 | 0.014 | 0.404 |
| 150 | 0.013 | 0.132 | 0.031 | >9.100 |
| 151 | 0.008 | 0.141 | 0.027 | 0.946 |
| 152 | 0.009 | 0.040 | 0.013 | 0.485 |
| 153 | 0.008 | 0.271 | 0.013 | 1.255 |
| 154 | 0.005 | 0.071 | 0.006 | 0.267 |
| 155 | 0.070 | 0.639 | 0.174 | >9.100 |
| 156 | 0.008 | 0.073 | 0.014 | 0.400 |
| 157 | 0.021 | 0.842 | 0.202 | 2.035 |
| 158 | 0.038 | 0.388 | 0.113 | >9.100 |
| 159 | 0.018 | 0.036 | 0.013 | 0.617 |
| 160 | 0.022 | 0.144 | 0.039 | 1.264 |
| 161 | 0.003 | 0.028 | 0.009 | 2.396 |
| 162 | 0.379 | >9.100 | >9.100 | >9.100 |
| 163 | 0.069 | 0.908 | 0.086 | 1.893 |
| 164 | 0.006 | 0.154 | 0.026 | 0.453 |
| 165 | 0.131 | 0.355 | 0.207 | 0.371 |
| 166 | 0.050 | 0.204 | 0.041 | 0.556 |
| 167 | 0.035 | 1.479 | 0.231 | 2.041 |
| 168 | 0.011 | 0.025 | 0.014 | 0.585 |
| 169 | 0.045 | 0.082 | 0.053 | 0.324 |
| 171 | 0.049 | 0.185 | 0.061 | 0.480 |
| 172 | 0.048 | 1.054 | 0.139 | 0.461 |
| 173 | 0.039 | 1.025 | 0.113 | 0.516 |
| 174 | 0.106 | 2.063 | 0.224 | 2.073 |
| 175 | 0.119 | 7.101 | 0.421 | >9.100 |
| 176 | 0.082 | 0.081 | 0.041 | 2.438 |
| 177 | 0.011 | 0.331 | 0.092 | >9.100 |
| 178 | 0.793 | 6.187 | 0.495 | >9.100 |
| 179 | 3.895 | >9.100 | 0.448 | 2.925 |
| 181 | 0.012 | 0.180 | 0.016 | 1.081 |

1. A method of treating one or more diseases or disorders where the disease(s) or disorder(s) respond or responds to an inhibition of one or more kinases of the PI3-kinase-related protein kinase family to a warm-blooded animal requiring such treatment,.comprising administering one or more compounds of the formula I, 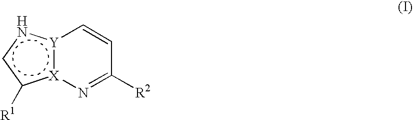
wherein
either X is N and Y is C, or X is C and Y is N,
the broken circle represents two conjugated double bonds within the five-membered ring with the proviso that the first of said bonds starts from either X═C or Y═C;
and each of R¹ and R² is, independently of the other, unsubstituted or substituted aryl or unsubstituted or substituted heterocyclyl;
and/or an N-oxide thereof, a solvate and/or a pharmaceutically acceptable salt thereof, to said warm-blooded animal in an effective amount for the treatment of said disease(s) or disorder(s).

2. The method according to claim 1 where the warm-blooded animal to be treated is a human.

3. The method according to claim 1 wherein the compound of the formula I is a compound of the formula IB 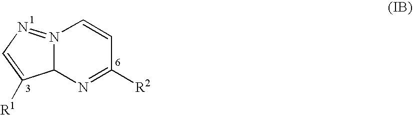
wherein R¹ and R² are as defined in claim 1, and/or an N-oxide thereof, a solvate and/or a pharmaceuticall acceptable salt thereof.

4. The method according to claim 1, where the disease to be treated is a disease selected from the group consisting of proliferative diseases selected from the group consisting of a benign or malignant tumor, carcinoma of the brain, kidney, liver, adrenal gland, bladder, breast, stomach, gastric tumors, ovaries, colon, rectum, prostate, pancreas, lung, vagina or thyroid, sarcoma, glioblastomas, multiple myeloma or gastrointestinal cancer, a neoplasia, lymphomas, a mammary carcinoma or a leukemia, or Cowden syndrome, Lhermitte-Dudos disease or Bannayan-Zonana syndrome.

5. (canceled)

6. A compound of the formula IA, 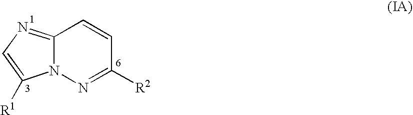
wherein
each of R¹ and R², independently of the other, is unsubstituted or substituted aryl or unsubstituted or substituted heterocyclyl, with the proviso that R¹ and R² are not both unsubstituted 4-pyridyl and the proviso that R¹ is 4-pyridyl and R² is morpholino;
or an N-oxide thereof, a solvate and/or a salt thereof.

7. A compound of the formula IA according to claim 6, wherein at least one of R¹ and R² is substituted aryl or substituted heterocyclyl or 2- or 3-pyridyl, while the other is selected from the group consisting or unsubstituted or substituted aryl and unsubstituted or substituted heterocyclyl, or an N-oxide thereof, a solvate and/or a salt thereof.

8. (canceled)

9. A compound of the formula IA according to claim 6, wherein each of R¹ and R², independently of the other, is phenyl, pyridinyl, or pyrrolo[2,3-b]pyridinyl, each of which is unsubstituted or substituted by one or more substituents independently selected from the group consisting of C₁-C₇-alkyl, halo-C₁-C₇-alkyl, furanyl, pyrrolyl, thiophenyl, unsubstituted or cyano-substituted pyridinyl, morpholinyl, thiomorpholinyl, S-oxo-thiomorpholinyl, S,S-dioxo-thiomorpholinyl, hydroxyl, C₁-C₇-alkoxy, which is unsubstituted or substituted by one or more substituents selected from pyrrolidinyl, piperazinyl, amino, N-mono- and/or N,N-di-C₁-C₇-alkylamino, halo, hydroxyl, C₁-C₇-alkoxy, halo-C₁-C₇-alkyl, and/or by a cyclic ether radical wherein each cyclic ether radical being unsubstituted or substituted at the same carbon which is attached to said C₁-C₇-alkoxy group with a substituent independently selected from, pyrrolidinyl, piperazinyl amino, N-mono- and/or N,N-di-C₁-C₇-alkylamino, N-mono- and/or N,N-di-C₁-C₇-alkanecarbonylamino, N-mono- and/or N,N-di-C₃-C₇-cycloalkanecarbonylamino, N-mono- and/or N,N-di-C₁-C₇-halo-alkanecarbonylamino, N-mono- and/or N,N-di-C₁-C₇-alkanoxycarbonylamino, wherein the alkyl group of the N-mono- and/or N,N-di-C₁-C₇-alkanoxycarbonylamino radical is unsubstituted or substituted by phenyl or naphthyl, pyrrolidinyl, piperazinyl, amino, N-mono- and/or N,N-di-C₁-C₇-alkylamino, by halo, by hydroxyl, by C₁-C₇alkoxy, and/or halo-C₁-C₇-alkyl, hydroxyl, C₁-C₇alkoxy, halo-C₁-C₇-alkyl, hydroxyl-C₂-C₇-alkoxy, amino-C₂-C₇-alkoxy, C₁-C₇-alkoxycarbonylamino-C₁-C₇alkoxy, C₁-C₇-alkoxycarbonyl-C₁-C₇alkoxy, unsubstituted or C₁-C₇-alkyl-substituted piperidinyloxy, halo, amino, phenyl-C₁-C₇-alkylamino, unsubstituted or phenyl-substituted thiazolylamino, C₁-C₇-alkanoyl, carboxy, C₁-C₇-alkoxycarbonyl, carbamoyl, C₁-C₇-alkanesulfonyl and sulfamoyl, with the proviso that R¹ and R² are not both unsubstituted is 4-pyridyl and/or an N-oxide thereof, a solvate and/or a salt thereof.

10. A compound of the formula IA according to claim 6, wherein R¹ is 1 H-pyrrol-2-yl)-phenyl, 4-furan3-yl-phenyl, 4-thiophen-3-yl-phenyl, 4-methoxyphenyl, 3,4-dimethoxyphenyl, 4-(3-amino-propoxy)-3-methoxyphenyl, 4-(3-tert-butoxycarbonylamino-propoxy)-3-methoxyphenyl, 6-(4-phenyl-thiazol-2-ylamino)-pyridin-3-yl, 4-carbamoylphenyl, 4-methanesulfonyl-phenyl, 4-(2-cyanopyridin-5-yl)-phenyl, 6-fluoro-pyridin-3-yl, 6-amino-5-trifluormethyl-pyridin-3-yl, 6-hydroxy-pyridin-3-yl, 6-(1-isopropyl-piperidin-4-yloxy)-pyridin-3-yl, 6-benzylamino-pyridin-3-yl, 6-morpholin-4-yl-pyridin-3-yl or 1H-pyrrolo[2,3-b]pyridin-5-yl, 4-[N-(2-morpholin-4-yl-ethyl)]benzamide, 4-[3-fluoro-N-(2-morpholin-4-yl-ethyl)]benzamide;
and
R² is 2-methoxyphenyl, 3,4-dimethoxyphenyl, 4-(3-amino-propoxy)-3-methoxyphenyl, 4-(3-tert-butoxycarbonylamino-propoxy)-3-methoxyphenyl, 3-carbamoyl-4-methoxycarbonylmethoxy-phenyl, 5-ethoxycarbonyl-4-methoxy-phenyl, 3-acetyl-4-(2-hydroxyethoxy)-phenyl, 4-carbamoylphenyl, 3-carbamoyl-4-methoxycarbonylmethyl-phenyl, 4-sulfamoyl-phenyl or 6-amino-5-trifluormethyl-pyridin-3-yl; 4[3-(cyclopropylcarbonylamino)-propoxy]phenyl, 2-[3-(cyclopropylcarbonylamino)-propoxy]pyridin-5-yl, 3-[phenoxymethyl-4-yl]-oxetan-3-ylamine, cyclopropanecarboxylic acid [3-(phenoxymethyl-4-yl)-oxetan-3-yl]-amide, N-[3-(phenoxymethyl-4-yl)-oxetan-3-yl]-isobutyramide, cyclopropanecarboxylic acid [3-(phenoxymethyl-4-yl)-oxetan-3-ylmethyl]-amide, C-[3-(phenoxymethyl-4-yl)-oxetan-3-yl]-methylamine, cyclopropanecarboxylic acid ((3-phenoxy-4-yl)-oxetan-3-ylmethyl)-amide;
and/or an N-oxide thereof, a solvate and/or salt thereof.

11. A compound of the formula IA according to claim 6, selected from the group of compounds with the following names:
3,6-bis-(3,4-dimethoxy-phenyl)-imidazo[1,2-b]pyridazine;
4-[6-(3,4-dimethoxy-phenyl)-imidazo[1,2-b]pyridazin-3-yl]-benzamide;
4-[3-(3,4-dimethoxy-phenyl)-imidazo[1,2-b]pyridazin-6-yl]-benzamide;
5-[6-(3,4-dimethoxy-phenyl)-imidazo[1,2-b]pyridazin-3-yl]-3-trifluoromethyl-pyridin-2-ylamine;
6-(3,4-dimethoxy-phenyl)-3-(4-methanesulfonyl-phenyl)-imidazo[1,2-b]pyri-dazine;
5-[3-(6-amino-5-trifluoromethyl-pyridin-3-yl)-imidazo[1,2-b]pyridazin-6-yl]-3-trifluoromethyl-pyridin-2-ylamine;
4-[3-(4-carbamoylphenyl)-imidazo[1,2-b]pyridazin-6-yl]-benzamide;
5-[3-(3,4-dimethoxy-phenyl)-imidazo[1,2-b]pyridazin-6-yl]-2-methoxy-benzoic acid ethyl ester;
4-[6-(2-methoxy-phenyl)-imidazo[1,2-b]pyridazin-3-yl]-benzamide;
(3-{4-[3-(6-amino-5-trifluoromethyl-pyridin-3-yl)-imidazo[1,2-b]pyridazin-6-yl]-2-methoxy-phenoxy}-propyl)-carbamic acid tert-butyl ester;
4-[3-(6-amino-5-trifluoromethyl-pyridin-3-yl)-imidazo[1,2-b]pyridazin-6-yl]-benzamide;
{2-carbamoyl-4-[3-(3,4-dimethoxy-phenyl)-imidazo[1,2-b]pyridazin-6-yl]-phenoxy}-acetic acid methyl ester;
5-{4-[6-(3,4-dimethoxy-phenyl)-imidazo[1,2-b]pyridazin-3-yl]-phenyl}-pyridine-2-carbonitrile;
5-{6-[4-(3-amino-propoxy)-3-methoxy-phenyl]-imidazo[1,2-b]pyridazin-3-yl}-3-trifluoromethyl-pyridin-2-ylamine;
(3-{4-[3-(4-carbamoyl-phenyl)-imidazo[1,2-b]pyridazin-6-yl]-2-methoxy-phenoxy}-propyl)-carbamic acid tert-butyl ester;
1-[5-[3-(3,4-dimethoxy-phenyl)-imidazo[1,2-b]pyridazin-6-yl]-2-(2-hydroxy-ethoxy)-phenyl]-ethanone;
4-{6-[4-(3-amino-propoxy)-3-methoxy-phenyl]-imidazo[1,2-b]pyridazin-3-yl}-benzamide;
5-[3-(4-methanesulfonyl-phenyl)-imidazo[1,2-b]pyridazin-6-yl]-3-trifluoro-methyl-pyridin-2-ylamine;
6-(3,4-dimethoxy-phenyl)-3-(4-furan-3-yl-phenyl)-imidazo[1,2-b]pyridazine;
6-(3,4-dimethoxy-phenyl)-3-[4-(1H-pyrrol-2-yl)-phenyl]-imidazo[1,2-b]pyridazine;
(3-{4-[6-(4-carbamoyl-phenyl)-imidazo[1,2-b]pyridazin-3-yl]-2-methoxy-phenoxy}-propyl)-carbamic acid tert-butyl ester;
6-(3,4-dimethoxy-phenyl)-3-(4-thiophen-3-yl-phenyl)-imidazo[1,2-b]pyridazine;
4-{3-[4-(3-amino-propoxy)-3-methoxy-phenyl]-imidazo[1,2-b]pyridazin-6-yl}-benzamide;
6-(3,4-dimethoxy-phenyl)-3-(1H-pyrrolo[2,3-b]pyridin-5-yl)-imidazo[1,2-b]pyridazine;
5-[3-(6-fluoro-pyridin-3-yl)-imidazo[1,2-b]pyridazin-6-yl]-3-trifluoromethyl-pyridin-2-ylamine;
5-{3-[6-(4-phenyl-thiazol-2-ylamino)-pyridin-3-yl]-imidazo[1,2-b]pyridazin-6-yl}-3-trifluoromethyl-pyridin-2-ylamine;
5-{3-[6-(1-isopropyl-piperidin-4-yloxy)-pyridin-3-yl]-imidazo[1,2-b]pyridazin-6yl}-3trifluoromethyl-pyridin-2-ylamine;
5-[3-(6-benzylamino-pyridin-3-yl)-imidazo[1,2-b]pyridazin-6-yl]-3-trifluoro-methyl-pyridin-2-ylamine;
5-[3-(6-morpholin-4-yl-pyridin-3-yl)-imidazo[1,2-b]pyridazin-6-yl]-3-trifluoromethyl-pyridin-2-ylamine;
5-[6-(6-amino-5-trifluoromethyl-pyridin-3-yl)-imidazo[1,2-b]pyridazin-3-yl]-pyridin-2-ol;
5-[3-(4-Ethanesulfonyl-phenyl)-imidazo[1,2-b]pyridazin-6-yl]-3-trifluoro-methyl-pyridin-2-ylamine;
5-[6-(3,4-Dimethoxy-phenyl)-imidazo[1,2-b]pyridazin-3-yl]-pyridin-2-ylamine;
4-[6-(6-Amino-5-trifluoromethyl-pyridin-3-yl)-imidazo[1,2-b]pyridazin-3-yl]-N-methyl-benzenesulfonamide;
(3-{4-[6-(6-Amino-5-trifluoromethyl-pyridin-3-yl)-imidazo[1,2-b]pyridazin-3-yl]-2-methoxy-phenoxy}-propyl)-carbamic acid tert-butyl ester;
5-{3-[4-(3-Amino-propoxy)-3-methoxy-phenyl]-imidazo[1,2-b]pyridazin-6-yl}-3-trifluoromethyl-pyridin-2-ylamine;
5-[6-(3,4-Dimethoxy-phenyl)-imidazo[1,2-b]pyridazin-3-yl]-pyrazin-2-ylamine;
5-[3-(6-Amino-pyridin-3yl)-imidazo[1,2-b]pyridazin-6-yl]-3-trifluoromethyl-pyridin-2-ylamine;
(3-{4-[6-(6-Amino-5-trifluoromethyl-pyridin-3-yl)-imidazo[1,2-b]pyridazin-3-yl]-2-trifluoromethoxy-phenoxy}-propyl)-carbamic acid tert-butylester;
5-[3-(1H-Pyrrolo[2,3-b]pyridin-5-yl)-imidazo[1,2-b]pyridazin-6-yl]-3-trifluoromethyl-pyridin-2-ylamine;
3-(3,4-Dimethoxy-phenyl)-6-(1H-pyrrolo[2,3-b]pyridin-5-yl)-imidazo[1,2-b]pyridazine;
5-{3-[4-(3-Amino-propoxy)-3-trifluoromethoxy-phenyl]-imidazo[1,2-b]pyri-dazin-6-yl}-3-trifluoromethyl-pyridin-2-ylamine;
N-{4-[6-(3,4-Dimethoxy-phenyl)-imidazo[1,2-b]pyridazin-3-yl]-phenyl}-methanesulfonamide;
N-(4-[6-(6-Amino-5-trifluoromethyl-pyridin-3-yl)-imidazo[1,2-b]pyridazin-3-yl]-phenyl)-methanesulfonamide;
5-[6-(4-Methoxy-phenyl)-imidazo[1,2-b]pyridazin-3-yl]-3-trifluoromethyl-pyridin-2-ylamine;
6-(4-Methoxy-phenyl)-3-(1H-pyrrolo[2,3-b]pyridin-5-yl)-imidazo[1,2-b]pyridazine;
5-[6-(3-Fluoro-4-methoxy-phenyl)-imidazo[1,2-b]pyridazin-3-yl]-3-trifluoromethyl-pyridin-2-ylamine;
6-(3-Fluoro-4-methoxy-phenyl)-3-(1H-pyrrolo[2,3-b]pyridin-5-yl)-imidazo[1,2-b]pyridazine;
(3-{4-[3-(6-Amino-5-trifluoromethyl-pyridin-3-yl)-imidazo[1,2-b]pyridazin-6-yl]-2-methoxy-phenoxy}-propyl)-carbamic acid methyl ester; N-(3-{4-[3-(6-Amino-5-trifluoromethyl-pyridin-3-yl)-imidazo[1,2-b]pyridazin-6-yl]-2-methoxy-phenoxy}-propyl)-isobutyramide;
N-(3-{4-[3-(6-Amino-5-trifluoromethyl-pyridin-3-yl)-imidazo[1,2-b]pyridazin-6-yl]-2-methoxy-phenoxy}-propyl)-acetamide;
6-(3,4-Dimethoxy-phenyl)-3-(5-trifluoromethyl-pyridin-3-yl)-imidazo[1,2-b]pyridazine;
3-Trifluoromethyl-5-[3-(5-trifluoromethyl-pyridin-3-yl)-imidazo[1,2-b]pyridazin-6-yl]-pyridin-2-ylamine;
(3-{4-[3-(6-Amino-5-trifluoromethyl-pyridin-3-yl)-imidazo[1,2-b]pyridazin-6-yl]-2-trifluoromethyoxy-phenoxy}-propyl)-carbamic acid tert-butyl ester;
5-{6-[4-(3-Amino-propoxy)-phenyl]imidazo[1,2-b]pyridazin-3-yl}-3-trifluoromethyl-pyridin-2-ylamine;
N-(3-{4-[3-(6-Amino-5-trifluoromethyl-pyridin-3-yl)-imidazo[1,2-b]pyridazin-6-yl]-phenoxy}-propyl)-isobutyramide;
Cyclopropanecarboxylic acid (3-{4-[3-(6-amino-5-trifluoromethyl-pyridin-3-yl)-imidazo[1,2-b]pyridazin-6-yl]phenoxy}-propyl)-amide;
5-{6-[4-(2-Amino-ethoxy)-3-methoxy-phenyl]imidazo[1,2-b]pyridazin-3-yl}-3-trifluoromethyl-pyridin-2-ylamine;
N-(2-{4-[3-(6-Amino-5-trifluoromethyl-pyridin-3-yl)-imidazo[1,2-b]pyridazin-6-yl]-2-methoxy-phenoxy}-ethyl)-isobutyramide;
Cyclopropanecarboxylic acid (2-{4-[3-(6amino-5-trifluoromethyl-pyridin-3-yl)-imidazo[1,2-b]pyridazin-6-yl]-2-methoxy-phenoxyl}ethyl)-amide;
N-(2-{4-[3-(6-Amino-5-trifluoromethyl-pyridin-3-yl)-imidazo[1,2-b]pyridazin-6-yl]-2-methoxy-phenoxy}-ethyl)acetamide;
5-{6-[4-(3-Amino-propoxy)-3-trifluoromethoxy-phenyl]imidazo[1,2-b]pyridazin-3-yl}-3-trifluoromethyl-pyridin-2-ylamine;
N-(3-{4-[3-(6-Amino-5-trifluoromethyl-pyridin-3-yl)-imidazo[1,2-b]pyridazin-6yl]-2-trifluoromethoxy-phenoxy}-propyl)isobutyramide;
Cyclopropanecarboxylic acid (3-{4-[3-(6-amino-5-trifluoromethyl-pyridin-3-yl)-imidazo[1,2-b]pyridazin-6-yl]-2-methoxy-phenoxy}-propyl)-amide;
5-[6-(6-Amino-5-trifluoromethyl-pyridin-3-yl)-imidazo[1,2-b]pyridazin-3-yl]-pyrazin-2-ylamine;
5-[6-(6-Amino-5-trifluoromethyl-pyridin-3-yl)-imidazo[1,2-b]pyridazin-3-yl]-3-methoxy-pyrazin-2-ylamine;
5-[6-(3,4-Dimethoxy-phenyl)-imidazo[1,2-b]pyridazin-3-yl]-3-methoxy-pyrazin-2-ylamine;
(2-{5-[3(6-Amino-5trifluoromethyl-pyridin-3-yl)-imidazo[1,2-b]pyridazin-6yl]-2-oxo-2H-pyridin-1-yl}-ethyl)-carbamic acid tert-butyl ester;
1-(2-Amino-ethyl)-5-[3-(6-amino-5-trifluoromethyl-pyridin-3-yl)-imidazo[1,2-b]pyridazin-6-yl]-1H-pyridin-2-one;
N-(3-{4-[3-(6-Amino-5-trifluoromethyl-pyridin-3-yl)-imidazo[1,2-b]pyridazin-6-yl]-phenoxy}-propyl)-acetamide;
5-{3-[4-(Propane-2-sulfonyl)-phenyl]-imidazo[1,2-b]pyridazin-6-yl}-3-trifluoromethyl-pyridin-2-ylamine;
(2-{4-[3-(6Amino-5-trifluoromethyl-pyridin-3yl)-imidazo[1,2-b]pyridazin-6-yl]-phenoxy}-ethyl)-carbamic acid tert-butyl ester;
5-{6-[4-(2-Amino-ethoxy)-phenyl]imidazo[1,2-b]pyridazin-3-yl}-3-trifluoromethyl-pyridin-2-ylamine;
N-(2-{4-[3-(6-Amino-5-trifluoromethyl-pyridin-3-yl)-imidazo[1,2-b]pyridazin-6-yl]-phenoxy}-ethyl)-acetamide;
N-(2-{5-[3-(6-Amino-5-trifluoromethyl-pyridin-3-yl)-imidazo[1,2-b]pyridazin-6-yl]-2-oxo-2H-pyridin-1-yl}-ethyl)-acetamide;
5-[6-(3-Methoxy-phenyl)-imidazo[1,2-b]pyridazin-3-yl]-3-trifluoromethyl-pyridin-2-ylamine;
6-(3-Methoxy-phenyl)-3-(1H-pyrrolo[2,3-b]pyridin-5yl)-imidazo[1,2-b]pyridazine;
5-[6-(6-Amino-5-trifluoromethyl-pyridin-3-yl)-imidazo[1,2-b]pyridazin-3-yl]-pyrimidin-2-ylamine;
4-[6-(6-Amino-5-trifluoromethyl-pyridin-3-yl)-imidazo[1,2-b]pyridazin-3-yl]-N-(2-morpholin-4-yl-ethyl)-benzamide;
4-[6-(6-Amino-5-trifluoromethyl-pyridin-3-yl)-imidazo[1,2-b]pyridazin-3-yl]-N-(3-morpholin-4-yl-propyl)-benzamide;
1-(4-{4-[6-(6-Amino-5-trifluoromethyl-pyridin-3-yl)-imidazo[1,2-b]pyridazin-3-yl]-benzoyl}-piperazin-1-yl)-ethanone;
N-(4-N-dimethylacetyl-benzyl)-4-[6-(6-amino-5-trifluoromethyl-pyridin-3-yl)-imidazo[1,2-b]pyridazin-3-yl]-benzamide;
N-(4-Acetyl-benzyl)-4-[6-(6-amino-5-trifluoromethyl-pyridin-3-yl)-imidazo[1,2b]pyridazin-3-yl]-benzamide;
{4-[6-(6-Amino-5-trifluoromethyl-pyridin-3-yl)-imidazo[1,2b]pyridazin-3-yl]-phenyl}-(4-pyridin-2-ylmethyl-piperazin-1-yl)-methanone;
N-(2-{4-[3-(6-Amino-5-trifluoromethyl-pyridin-3-yl)-imidazo[1,2-b]pyridazin-6-yl]-phenoxy}-ethyl)-isobutyramide;
{5-[6-(6-Amino-5-trifluoromethyl-pyridin-3-yl)-imidazo[1,2-b]pyridazin-3-yl]-pyrimidin-2-yl}-methyl-amine;
5-{3-[4-(2-Pyrazol-1-yl-ethoxy)-phenyl]-imidazo[1,2-b]pyridazin-6-yl}-3-trifluoromethyl-pyridin-2-ylamine;
1-(3-{4-[3-(6-Amino-5-trifluoromethyl-pyridin-3-yl)-imidazo[1,2-b]pyridazin-6-yl]-phenoxy}-propyl)-pyrrolidin-2-one;
1-(3-{4-[3-(6-Amino-5-trifluoromethyl-pyridin-3-yl)-imidazo[1,2-b]pyridazin-6-yl]-2-fluoro-phenoxy}-propyl)-pyrrolidin-2-one;
1-(3-{2-Fluoro-4-[3-(1H-pyrrolo[2,3-b]pyridin-5-yl)-imidazo[1,2-b]pyridazin-6-yl]-phenoxy}-propyl)-pyrrolidin-2-one;
5-{6-[4-(2-Pyrazol-1-yl-ethoxy)-phenyl]-imidazo[1,2-b]pyridazin-3-yl}-3-trifluoromethyl-pyridin-2-ylamine;
6-[4-(2-Pyrazol-1-yl-ethoxy)-phenyl]-3-(1H-pyrrolo[2,3-b]pyridin-5-yl)-imidazo[1,2-b]pyridazine;
1-(2-{4-[3-(6-Amino-5-trifluoromethyl-pyridin-3-yl)-imidazo[1,2-b]pyridazin-6-yl]-2-fluoro-phenoxy}-ethyl)-pyrrolidin-2-one;
4-[6-(6-Amino-5-trifluoromethyl-pyridin-3-yl)-imidazo[1,2-b]pyridazin-3-yl]-2-fluoro-benzoic acid;
1-(2-{2-Fluoro-4-[3-(1H-pyrrolo[2,3-b]pyridin-5-yl)-imidazo[1,2-b]pyridazin-6-yl]-phenoxy}-ethyl)-pyrrolidin-2-one;
1-(2-{4-[3-(6-Amino-5-trifluoromethyl-pyridin-3-yl)-imidazo[1,2-b]pyridazin-6-yl]-phenoxy}-ethyl-pyrrolidin-2-one;
1-(2-{4-[3-(1H-Pyrrolo[2,3-b]pyridin-5-yl)-imidazo[1,2-b]pyridazin-6-yl]-phenoxy}-ethyl)-pyrrolidin-2-one;
4-[6-(6-Amino-5-trifluoromethyl-pyridin-3-yl)-imidazo[1,2-b]pyridazin-3-yl]-2-fluoro-N-(2-morpholin-4-yl-ethyl)-benzamide;
4-[6-(6-Amino-5-trifluoromethyl-pyridin-3-yl)-imidazo[1,2-b]pyridazin-3-yl]-2-fluoro-N-(3-morpholin-4-yl-propyl)-benzamide;
{4-[6-(6-Amino-5-trifluoromethyl-pyridin-3-yl)-imidazo[1,2-b]pyridazin-3-yl]-2-fluoro-phenyl}-(4-pyridin-2-ylmethyl-piperazin-1-yl)-methanone;
5-{3-[4-(Morpholine-4-sulfonyl)-phenyl]imidazo[1,2-b]pyridazin-6-yl}-3-trifluoromethyl-pyridin-2-ylamine;
1-(2-{4-[3-(6-Amino-5-trifluoromethyl-pyridin-3-yl)-imidazo[1,2-b]pyridazin-6-yl]-2-methoxy-phenoxy}-ethyl)-pyrrolidin-2-one;
5-[3-(4-Ethenesulfonyl-phenyl)-imidazo[1,2-b]pyridazin-6-yl]-3-trifluoromethyl-pyridin-2-ylamine;
5-{3-[4-(2-Morpholin-4-yl-ethanesulfonyl)-phenyl]-imidazo[1,2-b]pyridazin-6-yl}-3-trifluoromethyl-pyridin-2-ylamine;
{4-[6-(6-Amino-5-trifluoromethyl-pyridin-3-yl)-imidazo[1,2-b]pyridazin-3-yl]-2-fluoro-phenyl}-[4-(2-morpholin-4-yl-ethyl)-piperazin-1-yl]-methanone;
{4-[6-(6-Amino-5-trifluoromethyl-pyridin-3-yl)-imidazo[1,2-b]pyridazin-3-yl]-2-fluoro-phenyl}-[4-(2-dimethylamino-ethyl)-piperazin-1-yl]-methanone;
4-[6-(6-Amino-5-trifluoromethyl-pyridin-3-yl)-imidazo[1,2-b]pyridazin-3-yl]-N-(2-diethylamino-ethyl)-2-fluoro-benzamide;
4-[6-(6-Amino-5-trifluoromethyl-pyridin-3-yl)-imidazo[1,2-b]pyridazin-3-yl]-2-methoxy-benzoic acid;
1-(2-{2-Methoxy-4-[3-(1H-pyrrolo[2,3-b]pyridin-5-yl)-imidazo[1,2-b]pyridazin-6-yl]-phenoxy}-ethyl)-pyrrolidin-2-one;
1-[2-(2-Methoxy-4-{3-[4-(2-morpholin-4-yl-ethanesulfonyl)-phenyl]-imidazo[1,2-b]pyridazin-6-yl}-phenoxy)-ethyl]-pyrrolidin-2-one;
{4-[6-(6-Amino-5-trifluoromethyl-pyridin-3-yl)-imidazo[1,2-b]pyridazin-3-yl]-2-fluoro-phenyl}-(4-pyridin-3-ylmethyl-piperazin-1-yl)-methanone;
{4-[6-(6-Amino-5-trifluoromethyl-pyridin-3-yl)-imidazo[1,2-b]pyridazin-3-yl]-2-fluoro-phenyl}-[4-(2-hydroxy-ethyl)-piperazin-1-yl]-methanone;
{4-[6-(6-Amino-5-trifluoromethyl-pyridin-3-yl)-imidazo[1,2-b]pyridazin-3-yl]-2-fluoro-phenyl}-[4-(4-fluoro-benzyl)-piperazin-1-yl]-methanone;
4-[6-(6-Amino-5-trifluoromethyl-pyridin-3-yl)-imidazo[1,2-b]pyridazin-3-yl]-2-methoxy-N-(2-morpholin-4-yl-ethyl)-benzamide;
{4-[6-(6-Amino-5-trifluoromethyl-pyridin-3-yl)-imidazo[1,2-b]pyridazin-3-yl]-2-methoxy-phenyl}-(4-pyridin-2-ylmethyl-piperazin-1-yl)-methanone;
{4-[6-(6-Amino-5-trifluoromethyl-pyridin-3-yl)-imidazo[1,2-b]pyridazin-3-yl]-2-methoxy-phenyl}-(4-pyridin-3-ylmethyl-piperazin-1-yl)-methanone;
{4-[6-(6-Amino-5-trifluoromethyl-pyridin-3-yl)-imidazo[1,2-b]pyridazin-3-yl]-2-methoxy-phenyl}-[4-(2-morpholin-4-yl-ethyl)-.piperazin-1-yl]-methanone;
2-(4-{4-[6-(6-Amino-5-trifluoromethyl-pyridin-3-yl)-imidazo[1,2-b]pyridazin-3-yl]-2-methoxy-benzoyl}-piperazin-1-yl)-1-morpholin-4-yl-ethanone;
{4-[6-(6-Amino-5-trifluoromethyl-pyridin-3-yl)-imidazo[1,2-b]pyridazin-3-yl]-2-methoxy-phenyl}-(4-pyridin-2-yl-piperazin-1-yl)-methanone;
4-[6-6-Amino-5-trifluoromethyl-pyridin-3-yl)-imidazo[1,2-b]pyridazin-3-yl]-benzenesulfonamide;
4-[3-(6-Amino-5-trifluoromethyl-pyridin-3-yl)-imidazo[1,2-b]pyridazin-6-yl]-N-(2-morpholin-4-yl-ethyl)-benzamide;
{4-[3-(6-Amino-5-trifluoromethyl-pyridin-3-yl)-imidazo[1,2-b]pyridazin-6-yl]-phenyl}-(4-pyridin-2-ylmethyl-piperazin-1-yl)-methanone;
4-[6-(6-Amino-5-trifluoromethyl-pyridin-3-yl)-imidazo[1,2-b]pyridazin-3-yl]-N-(2-morpholin-4-yl-ethyl)-benzenesulfonamide;
4-[6-(6-Amino-5-trifluoromethyl-pyridin-3-yl)-imidazo[1,2-b]pyridazin-3-yl]-2-methoxy-N-(3-morpholin-4-yl-propyl)-benzamide;
5-{3-[1-(2-Morpholin-4-yl-ethyl)-1H-pyrrolo[2,3-b]pyridin-5-yl]imidazo[1,2-b]pyridazin-6-yl}-3-trifluoromethyl-pyridin-2-ylamine;
{4-[6-(6-Amino-5-trifluoromethyl-pyridin-3-yl)-imidazo[1,2-b]pyridazin-3-yl]-phenyl}-(4-pyridin-3-ylmethyl-piperazin-1-yl)-methanone;
{4-[6-(6-Amino-5-trifluoromethyl-pyridin-3-yl)-imidazo[1,2-b]pyridazin-3-yl]-phenyl}-[4-(2-morpholin-4-yl-ethyl)-piperazin-1-yl]-methanone;
Cyclopropanecarboxylic acid (3-{4-[3-(6-amino-pyridin-3-yl)-imidazo[1,2-b]pyridazin-6-yl]-phenoxy}-propyl)amide;
Cyclopropanecarboxylic acid (3-{4-[3-(5-trifluoromethyl-pyridin-3-yl)-imidazo[1,2-b]pyridazin-6-yl]-phenoxy}-propyl)-amide;
5-{3-[1-(2-Morpholin-4-yl-ethyl)-1H-indazol-5-yl]-imidazo[1,2-b]pyridazin-6-yl}-3-trifluoromethyl-pyridin-2-ylamine;
{4-[6-(6-Amino-5-trifluoromethyl-pyridin-3-yl)-imidazo[1,2-b]pyridazin-3-yl]-phenyl}-[4-(2-dimethylamino-ethyl)-piperazin-1-yl]-methanone;
{4-[6-(6-Amino-5-trifluoromethyl-pyridin-3-yl)-imidazo[1,2-b]pyridazin-3-yl]-2-methoxy-phenyl}-[4-(2-pyridin-2-yl-ethyl)-piperazin-1-yl]methanone;
4-[6-(6-Amino-5-trifluoromethyl-pyridin-3-yl)-imidazo[1,2-b]pyridazin-3-yl]-N-(3-morpholin-4-yl-propyl)-benzenesulfonamide;
{4-[6-(6-Amino-5-trifluoromethyl-pyridin-3-yl)-imidazo[1,2-b]pyridazin-3-yl]-phenyl}-[4-(2-pyridin-2-yl-ethyl)-piperazin-1-yl]-methanone;
{4-[6-(6-Amino-5-trifluoromethyl-pyridin-3-yl)-imidazo[1,2-b]pyridazin-3-yl]phenyl}-(4-pyridin-4-ylmethyl-piperazin-1-yl)-methanone;
Cyclopropanecarboxylic acid (3-{4-[3-(1H-pyrrolo[2,3-b]pyridin-5-yl)-imidazo[1,2-b]pyridazin-6-yl]-phenoxy}-propyl)-amide;
Cyclopropanecarboxylic acid (3-{4-[3-(5-amino-pyrazin-2-yl)-imidazo[1,2-b]pyridazin-6-yl]-phenoxy}-propyl)-amide;
6-(3,4-Dimethoxy-phenyl)-3-(1H-indazol-5-yl)-imidazo[1,2-b]pyridazine;
5-{3-[1-(3-Morpholin-4-yl-propyl)-1H-indazol-5-yl]-imidazo[1,2-b]pyridazin-6-yl}-3-trifluoromethyl-pyridin-2-ylamine;
5-{3-[4-(3-Amino-propoxy)-phenyl]-imidazo[1,2-b]pyridazin-6-yl}-3-trifluoromethyl-pyridin-2-ylamine;
Cyclopropanecarboxylic acid (3-{4-[6-(6-amino-5-trifluoromethyl-pyridin-3-yl)-imidazo[1,2-b]pyridazin-3-yl]-phenoxy}-propyl)-amide;
5-{3-[3-(2-Morpholin-4-yl-ethyl)-3H-imidazo[4,5-b]pyridin-6-yl]-imidazo[1,2-b]pyridazin-6-yl}-3-trifluoromethyl-pyridin-2-ylamine;
2-(4-{4-[6-(6-Amino-5-trifluoromethyl-pyridin-3-yl)-imidazo[1,2-b]pyridazin-3-yl]-benzoyl}-piperazin-1-yl)-1-morpholin-4-yl-ethanone;
2-(4-{4-[6-(6-Amino-5-trifluoromethyl-pyridin-3-yl)-imidazo[1,2-b]pyridazin-3-yl]-benzoyl}-piperazin-1-yl)-1-morpholin-4-yl-ethanone;
Cyclopropanecarboxylic acid (3-{4-[3-(6-amino-5-trifluoromethyl-pyridin-3-yl)-imidazo[1,2-b]pyridazin-6-yl]-phenoxymethyl}-oxetan-3-yl)-amide;
5-{6-[4-(3-Amino-oxetan-3-ylmethoxy)-phenyl]-imidazo[1,2-b]pyridazin-3-yl}-3-trifluoromethyl-pyridin-2-yl amine;
(3-{4-[3-(6-Amino-5-trifluoromethyl-pyridin-3-yl)-imidazo[1,2-b]pyridazin-6-yl]phenoxymethyl}-oxetan-3-yl)-carbamic acid methylester; 5-{3-[3-(2-Diethylamino-ethyl)-3H-imidazo[4,5-b]pyridin-6-yl]imidazo[1,2-b]pyridazin-6yl}-3-trifluoromethyl-pyridin-2-ylamine;
4-[6-(6-Amino-5-trifluoromethyl-pyridin-3-yl)-imidazo[1,2-b]pyridazin-3-yl]-N-(2-imidazol-1-yl-ethyl)-benzamide;
4-[6-(6-Amino-5-trifluoromethyl-pyridin-3-yl)-imidazo[1,2-b]pyridazin-3-yl]-3-fluoro-N-(2-morpholin-4-yl-ethyl)-benzamide;
4-[6-(6-Amino-5-trifluoromethyl-pyridin-3-yl)-imidazo[1,2-b]pyridazin-3-yl]-3-fluoro-N-(3-morpholin-4-yl-propyl)-benzamide;
4-[6-(6-Amino-5-trifluoromethyl-pyridin-3-yl)-imidazo[1,2-b]pyridazin-3-yl]-N-[2-(1,1-dioxo-1lambda*6*-thiomorpholin-4-yl)-ethyl]-3-fluoro-benzamide;
4-[6-(6-Amino-5-trifluoromethyl-pyridin-3-yl)-imidazo[1,2-b]pyridazin-3-yl]-N-[2-(1,1-dioxo-1lambda*6*-thiomorpholin-4-yl)-ethyl]-benzamide;
5-(6-Benzo[1,3]dioxol-5-yl-imidazo[1,2-b]pyridazin-3-yl)-3-trifluoromethyl-pyridin-2-ylamine;
4-[6-(6-Amino-5-trifluoromethyl-pyridin-3-yl)-imidazo[1,2-b]pyridazin-3-yl]-3-fluoro-N-(2-imidazol-1-yl-ethyl)-benzamide;
Cyclopropanecarboxylic acid (2-{4-[3-(6-amino-5-trifluoromethyl-pyridin-3-yl)-imidazo[1,2-b]pyridazin-6-yl]-phenoxy}-ethyl)amide;
4-[6-(6-Amino-5-trifluoromethyl-pyridin-3-yl)-imidazo[1,2-b]pyridazin-3-yl]-5-fluoro-2-methoxy-N-(2-morpholin-4-yl-ethyl)-benzamide;
4-[6-(6-Amino-5-trifluoromethyl-pyridin-3-yl)-imidazo[1,2-b]pyridazin-3-yl]-5-fluoro-2-methoxy-N-(3-morpholin-4-yl-propyl)-benzamide;
4-[6-(6-Amino-5-trifluoromethyl-pyridin-3-yl)-imidazo[1,2-b]pyridazin-3-yl]-N-[2-(1,1-dioxo-1lambda*6*-thiomorpholin-4-yl)-ethyl]-5-fluoro-2-methoxy-benzamide;
4-[6-(6-Amino-5-trifluoromethyl-pyridin-3-yl)-imidazo[1,2-b]pyridazin-3-yl]-5-fluoro-N-(2-imidazol-1-yl-ethyl)-2-methoxy-benzamide;
4-{4-[3-(6-Amino-5-trifluoromethyl-pyridin-3yl)-imidazo[1,2-b]pyridazin-6-yl]-phenoxy}-piperidine-1-carboxylic acid tert-butyl ester;
1-(3-{4-[3-(6-Amino-5-trifluoromethyl-pyridin-3-yl)-imidazo[1,2-b]pyridazin-6-yl]-phenoxy}-propyl)-imidazolidin-2-one;
1-(3-{4-[3-(6-Amino-5-trifluoromethyl-pyridin-3-yl)-imidazo[1,2-b]pyridazin-6-yl]-2-methoxy-phenoxy}-propyl)imidazolidin-2-one;
5-{6-[4-(Piperidin-4-yloxy)-phenyl]-imidazo[1,2-b]pyridazin-3-yl}-3-trifluoromethyl-pyridin-2-ylamine;
5-{6-[3-Methoxy-4-(piperidin-4-yloxy)-phenyl]-imidazo[1,2-b]pyridazin-3-yl}-3-trifluoromethyl-pyridin-2-ylamine;
N-(3-{4-[3-(6-Amino-5-trifluoromethyl-pyridin-3-yl)-imidazo[1,2-b]pyridazin-6-yl]-phenoxymethyl}-oxetan-3-ylmethylyisobutyramide;
{4-[6-(6-Amino-5-trifluoromethyl-pyridin-3-yl)-imidazo[1,2-b]pyridazin-3-yl]-phenyl}-methanol;
5-{6-[4-(3-Aminomethyl-oxetan-3-ylmethoxy)-phenyl]-imidazo[1,2-b]pyridazin-3-yl}-3-trifluoromethyl-pyridin-2-ylamine;
5-{3-[4(3-Morpholin-4-yl-propoxy)-phenyl]-imidazo[1,2-b]pyridazin-6-yl}-3-trifluoromethyl-pyridin-2-ylamine;
Cyclopropanecarboxylic acid (3-{4-[3-(6-amino-5-trifluoromethyl-pyridin-3-yl)-imidazo[1,2-b]pyridazin-6-yl]-phenoxymethyl}-oxetan-3-ylmethyl)-amide;
(3-{4-[3-(6-Amino-5-trifluoromethyl-pyridin-3-yl)-imidazo[1,2-b]pyridazin-6-yl]-phenoxymethyl}-oxetan-3-ylmethyl)-carbamic acid methyl ester;
5-{3-[4-(3-Pyridin-4-yl-propoxy)-phenyl]-imidazo[1,2-b]pyridazin-6-yl}-3-trifluoromethyl-pyridin-2-ylamine;
(3-{5-[3-(6-Amino-5-trifluoromethyl-pyridin-3-yl)-imidazo[1,2-b]pyridazin-6-yl]-pyridin-2yloxy}-propyl)-carbamic acid tert-butyl ester;
5-{6-[6-(3-Amino-propoxy)-pyridin-3-yl]-imidazo[1,2-b]pyridazin-3-yl}-3-trifluoromethyl-pyridin-2-ylamine;
Cyclopropanecarboxylic acid (3-{5-[3-(6-amino-5-trifluoromethyl-pyridin-3-yl)-imidazo[1,2-b]pyridazin-6-yl]-pyridin-2-yloxy}-propyl)-amide;
6-Benzo[1,3]dioxol-5-yl-3-(1 H-pyrrolo[2,3-b]pyridin-5-yl)-imidazo[1,2-b]pyridazine;
(1-{4-[6-(6-Amino-5-trifluoromethyl-pyridin-3-yl)imidazo[1,2-b]pyridazin-3-yl]-benzyl}-piperidin-4-yl)-pyrrolidin-1-yl-methanone;
(1-{4-[6-(6-Amino-6-trifluoromethyl-pyridin-3yl)-imidazo[1,2-b]pyridazin-3-yl]-benzyl}-piperidin-4-yl)-azepan-1-yl-methanone; and
(1-{4-[6-(6-Amino-5-trifluoromethyl-pyridin-3-yl)-imidazo[1,2-b]pyridazin-3-yl]-benzyl}-piperidin-4-yl)-piperidin-1-yl-methanone;
and/or an N-oxide thereof, a solvate and/or salt thereof.

12. A method of treating one or more diseases or disorders where the disease(s) or disorder(s) respond or responds to an inhibition of one or more kinases of the PI3-kinase-related protein kinase family to a warm-blooded animal requiring such treatment, comprising administering one or more compounds of the formula IA as described in claim 6, and/or an N-oxide thereof, a solvate and/or a pharmaceutically acceptable salt thereof; to said warm-blooded animal in an effective amount for the treatment of said disease(s) or disorder(s).

13. The method of claim 12, wherein the compound of claim 62 is selected from the compounds of claim 11.

14. A pharmaceutical composition comprising a compound of the formula IA, and/or an N-oxide thereof, a solvate and/or a pharmaceutically acceptable salt thereof, according to claim 6, and at least one pharmaceutically acceptable carrier material.

15. A compound of the formula IB, 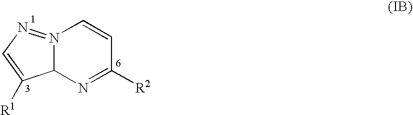
wherein
R¹ is phenyl, pyridinyl or pyrrolo[2,3-b]pyridinyl, each of which is substituted by one or more substituents independently selected from the group consisting of C₁-C₇-alkyl, halo-C₁-C₇-alkyl, furanyl, pyrrolyl, thiophenyl, unsubstituted or cyano-substituted pyridinyl, morpholinyl, thiomorpholinyl, S-oxo-thiomorpholinyl, S,S-dioxo-thiomorpholinyl, hydroxyl, C₁-C₇-alkoxy, hydroxyl-C₂-C₇-alkoxy, amino-C₂-C₇-alkoxy, C₁-C₇-alkoxycarbonylamino-C₁-C₇-alkoxy, C₁-C₇-alkoxycarbonyl-C₁-C₇-alkoxy, unsubstituted or C₁-C₇-alkyl-substituted piperidinyloxy, amino, phenyl-C₁-C₇-alkylamino, unsubstituted or phenyl-substituted thiazolylamino, C₁-C₇-alkanoyl, carboxy, C₁-C₇-alkoxycarbonyl, carbamoyl, C₁-C₇-alkanesulfonyl, sulfamoyl and, in the case of substituted pyridinyl or pyrrolo[2,3-b]pyridinyl, halo, and
R² is phenyl or pyridinyl, each of which is substituted by one or more substituents independently selected from the group consisting of C₁-C₇-alkyl, halo-C₁-C₇-alkyl, furanyl, pyrrolyl, thiophenyl, unsubstituted or cyano-substituted pyridinyl, morpholinyl, thiomorpholinyl, S-oxo-thiomorpholinyl, S,S-dioxo-thiomorpholinyl, C₁-C₇-alkoxy, hydroxyl-C₂-C₇-alkoxy, amino-C₂-C₇-alkoxy, C₁-C₇-alkoxycarbonylamino-C₁-C₇-alkoxy, C₁-C₇-alkoxycarbonyl-C₁-C₇-alkoxy, unsubstituted or C₁-C₇-alkyl-substituted piperidinyloxy, amino, phenyl-C₁-C₇-alkylamino, unsubstituted or phenyl-substituted thiazolylamino, C₁-C₇-alkanoyl, carboxy, C₁-C₇-alkoxycarbonyl, carbamoyl, C₁-C₇-alkanesulfonyl, sulfamoyl and, in the case of substituted pyridyl, from hydroxyl and halo, and/or an N-oxide thereof, a solvate and/or a salt thereof.

16. A compound of the formula IB according to claim 15, wherein
R¹ is 1H-pyrrol-2-yl)-phenyl, 4-furan-3-yl-phenyl, 4-thiophen-3-yl-phenyl, 4-methoxyphenyl, 3,4-dimethoxyphenyl, 4-(3-amino-propoxy)-3-methoxyphenyl, 4-(3-tert-butoxycarbonylamino-propoxy)-3-methoxyphenyl, 6-(4-phenyl-thiazol-2-ylamino)-pyridin-3-yl, 4-carbamoylphenyl, 4-methanesulfonyl-phenyl, 4-(2-cyanopyridin-5-yl)-phenyl, 6-fluoro-pyridin-3-yl, 6-amino-5-trifluormethyl-pyridin-3-yl, 6-hydroxy-pyridin-3-yl, 6-(1-isopropyl-piperidin-4-yloxy)-pyridin-3-yl, 6-benzylamino-pyridin-3-yl, 6-morpholin-4-yl-pyridin-3-yl or 1H-pyrrolo[2,3-b]pyridin-5-yl,
and
R² is 2-methoxyphenyl, 3,4-dimethoxyphenyl, 4-(3-amino-propoxy)-3-methoxyphenyl, 4-(3-tert-butoxycarbonylamino-propoxy)-3-methoxyphenyl, 3-carbamoyl-4-methoxycarbonylmethoxy-phenyl, 5-ethoxycarbonyl-4-methoxy-phenyl, 3-acetyl-4-(2-hydroxyethoxy)-phenyl, 4-carbamoylphenyl, 3-carbamoyl-4-methoxycarbonylmethoxy-phenyl, 4-sulfamoyl-phenyl or 6-amino-5-trifluormethyl-pyridin-3-yl
and/or an N-oxide thereof, a solvate and/or a salt thereof.

17. A, compound of the formula IB, selected from the group of compounds with the following names:
(3-{4-[3-(3,4-dimethoxy-phenyl)-pyrazolo[1,5-a]pyrimidin-5-yl]-2-methoxy-phenoxy}-propyl)-carbamic acid tert-butyl ester;
3-{4-[3-(3,4-dimethoxy-phenyl)-pyrazolo[1,5-a]pyridin-5-yl]-2-methoxy-phenoxy}-propylamine; and
4-[5-(2-Methoxy-phenyl)-pyrazolo[1,5-a]pyrimidin-3-yl]-benzamide;
and/or an N-oxide thereof, a solvate and/or a salt thereof.

18. A method of therapeutic and/or diagnostic treatment of one or more diseases or disorders where the disease(s) or disorder(s) respond or responds to an inhibition of one or more kinases of the PI3-kinase-related protein kinase family to a warm-blooded animal requiring such treatment, comprising administering one or more compounds of the formula IB as described in claim 15, and/or an N-oxide thereof, a solvate and/or a pharmaceutically acceptable salt thereof, to said warm-blooded animal in an effective amount for the treatment of said disease(s) or disorder(s).

19. A pharmaceutical composition comprising a compound of the formula IB, and/or an N-oxide thereof, a solvate and/or a pharmaceutically acceptable salt thereof, according to claim 15, and at least one pharmaceutically acceptable carrier material.

20. A method for the manufacture of a compound of the formula IA according to claim 6 or a compound of the formula IB according to claim 15, comprising
a) reacting a compound of the formula II, 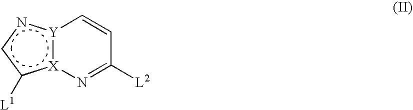
wherein
X is N and Y is C, or X is C and Y is N,
the broken circle represents two conjugated double bonds within the five-membered ring with the proviso that the first of said bonds starts from either X═C or Y═C;
and
each of L¹ and L², independently of the other, is halo or trifluoromethansulfonyloxy, under cross coupling conditions with a boronic acid or boronic acid ester or organotin compound of the formula III,
R^{1,2}-D (III)
wherein R^{1,2} is unsubstituted or substituted aryl or unsubstituted or substituted heterocyclyl; and D is —B(OH₂) in free form or in esterified form or is —Sn(alk)₃ wherein alk is alkyl, or
b) reacting a compound of the formula IV, 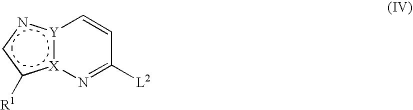
wherein
X is N and Y is C, or X is C and Y is N,
the broken circle represents two conjugated double bonds within the five-membered ring with the proviso that the first of said bonds starts from either X═C or Y═C;
and R¹ is unsubstituted or substituted aryl or unsubstituted or substituted heterocyclyl;
and
L² is halo or trifluoromethansulfonyloxy,
under cross coupling conditions with a boronic acid or boronic acid ester or organotin compound of the formula V,
R²-D (V)
wherein R² is unsubstituted or substituted aryl or unsubstituted or substituted heterocyclyl and D is —B(OH₂) in free form or in esterified form or is —Sn(alk)₃ wherein alk is alkyl, or
c) reacting a compound of the formula VI, 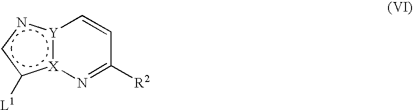
wherein
X is N and Y is C, or X is C and Y is N,
the broken circle represents two conjugated double bonds within the five-membered ring with the proviso that the first of said bonds starts from either X═C or Y═C;
and R² is unsubstituted or substituted aryl or unsubstituted or substituted heterocydyl;
and
L¹ is halo or trifluoromethansulfonyloxy,
under cross coupling conditions with a boronic acid or boronic acid ester or organotin compound of the formula VII,
R¹-D (VII)
wherein R¹ is unsubstituted or substituted aryl or unsubstituted or substituted heterocyclyl and D is —B(OH₂) in free form or in esterified form or is —Sn(alk)₃ wherein alk is alkyl, or
d) reacting a compound of the formula VIII, 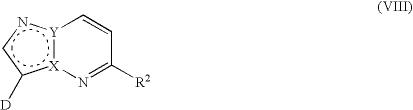
wherein
X is N and Y is C, or X is C and Y is N,
the broken circle represents two conjugated double bonds within the five-membered ring with the proviso that the first of said bonds starts from either X═C or Y═C;
R² is unsubstituted or substituted aryl or unsubstituted or substituted heterocyclyl; and
D is —B(OH₂) in free form or in esterified form or is —Sn(alk)₃ wherein alk is alkyl; under cross coupling conditions with a compound of the formula IX,
R¹-L¹ (IX)
wherein
L¹ is halo or trifluoromethansulfonyloxy, and
R¹ is unsubstituted or substituted aryl or unsubstituted or substituted heterocyclyl;
and, if desired, a compound of the formula I obtainable according to any one of the reactions a) to d) given above is converted into a different compound of the formula I, an obtainable salt of a compound of the formula I is converted into a different salt thereof, an obtainable free compound of the formula I is converted into a salt thereof, and/or an obtainable isomer of a compound of the formula I is separated from one or more different obtainable isomers of the formula I.


SEQUENCE LISTING (21 sequences)

SEQ ID NO: 1 - DNA, 28 bp, human
cgagaatatgatagattatatgaagaat

SEQ ID NO: 2 - DNA, 30 bp, human
tggtttaatgctgttcatacgtttgtcaat

SEQ ID NO: 3 - DNA, 76 bp, human
gggacaagtttgtacaaaaaagcaggctacgaaggagatatacatatgcgagaatatgatagattatatgaagaat

SEQ ID NO: 4 - DNA, 66 bp, human
taccataattccaccaccaccaccggaaattccccctggtttaatgctgttcatacgtttgtcaat

SEQ ID NO: 5 - DNA, 26 bp, human
ctagtggaatgtttactaccaaatgg

SEQ ID NO: 6 - DNA, 26 bp, human
gttcaatgcatgctgtttaattgtgt

SEQ ID NO: 7 - DNA, 63 bp, human
gggggaatttccggtggtggtggtggaattatggtactagtggaatgtttactaccaaatgga

SEQ ID NO: 8 - DNA, 56 bp, human
agctccgtgatggtgatggtgatgtgctccgttcaatgcatgctgtttaattgtgt

SEQ ID NO: 9 - DNA, 61 bp, human
gggaccactttgtacaagaaagctgggtttaagctccgtgatggtgatggtgatgtgctcc

SEQ ID NO: 10 - DNA, 42 bp, human
gctagcatgcgagaatatgatagattatatgaagaatatacc

SEQ ID NO: 11 - DNA, 45 bp, human
gcctccaccacctccgcctggtttaatgctgttcatacgtttgtc

SEQ ID NO: 12 - DNA, 42 bp, human
tactagtccgcctccaccacctccgcctccaccacctccgcc

SEQ ID NO: 13 - DNA, 54 bp, human
actgaagcatcctcctcctcctcctcctggtttaatgctgttcatacgtttgtc

SEQ ID NO: 14 - DNA, 57 bp, human
agctccgtgatggtgatggtgatgtgctccagatctgtagtctttccgaactgtgtg

SEQ ID NO: 15 - DNA, 61 bp, human; identical to SEQ ID NO: 9

SEQ ID NO: 16 - DNA, 45 bp, human
tcctcctcctcctcctcctggtttaatgctgttcatacgtttgtc

SEQ ID NO: 17 - DNA, 26 bp, human
atgccccctggggtggactgccccat

SEQ ID NO: 18 - DNA, 26 bp, human
ctactgcctgttgtctttggacacgt

SEQ ID NO: 19 - DNA, 53 bp, human
attaaaccaggaggaggaggaggaggaccccctggggtggactgccccatgga

SEQ ID NO: 20 - DNA, 56 bp, human
agctccgtgatggtgatggtgatgtgctccctgcctgttgtctttggacacgttgt

SEQ ID NO: 21 - DNA, 61 bp, human; identical to SEQ ID NO: 9